(19) United States
(12) Patent Application Publication
(10) Pub. No.: US 2008/0287371 A1
(43) Pub. Date: Nov. 20, 2008

(54) MACROCYCLIC ANTAGONISTS OF THE MOTILIN RECEPTOR FOR MODULATION OF THE MIGRATING MOTOR COMPLEX

(75) Inventors: Graeme L. Fraser, Rixensart (BE); Eric Marsault, Quebec (CA)

Correspondence Address: MYERS BIGEL SIBLEY & SAJOVEC, PO BOX 37428, RALEIGH, NC, 27627, US

(73) Assignee: Tranzyme Pharma Inc.

(21) Appl. No.: 12/122,094

(22) Filed: May 16, 2008

Related U.S. Application Data
(60) Provisional application No. 60/938,655, filed on May 17, 2007.
(60) Provisional application No. 60/939,280, filed on May 21, 2007.

(51) Int. Cl. A61K 38/06; A61P 1/12

(52) U.S. Cl. 514/18

(57) ABSTRACT

The present invention relates to novel conformationally-defined macrocyclic compounds that bind to and/or are functional modulators of the motilin receptor including subtypes, isoforms and/or variants thereof. These macrocyclic compounds are useful as therapeutics for a range of gastrointestinal disorders, in particular those in which suppression or inhibition of the migrating motor complex (MMC) is effective or malfunction of gastric motility or increased motilin secretion is observed, such as hypermotilinemia, imitable bowel syndrome, dyspepsia, including gallbladder dyspepsia, diarrhea, cancer treatment-related diarrhea, cancer-induced diarrhea, chemotherapy-induced diarrhea, radiation enteritis, radiation-induced diarrhea, stress-induced diarrhea, chronic diarrhea, AIDS-related diarrhea, C. difficile associated diarrhea, traveller's diarrhea, acute infectious diarrhea, diarrhea induced by graph versus host disease, other types of diarrhea, functional gastrointestinal disorders, chemotherapy-induced nausea and vomiting (emesis), post-operative nausea and vomiting, cyclic vomiting syndrome and functional vomiting. Accordingly, methods of treating such disorders with such macrocyclic compounds and pharmaceutical compositions thereof are also provided in addition to methods of modulating the migrating motor complex.

### RELATED APPLICATION DATA

This application claims the benefit of U.S. Provisional Patent Application Ser. No. 60/938,655, filed May 17, 2007, and U.S. Provisional Patent Application Ser. No. 60/939,280, filed May 21, 2007. The disclosures of each of which are incorporated herein by reference in their entireties.

### FIELD OF THE INVENTION

The present invention relates to novel conformationally-defined macrocyclic compounds that bind to and/or are functional modulators of the motilin receptor including subtypes, isoforms and/or variants thereof. These macrocyclic compounds are useful as therapeutics for a range of gastrointestinal disorders, in particular those in which suppression or inhibition of the migrating motor complex (MMC) is effective or malfunction of gastric motility or increased motilin secretion is observed, such as hypermotilinemia, irritable bowel syndrome, dyspepsia, including gallbladder dyspepsia, functional gastrointestinal disorders, diarrhea, cancer treatment-related diarrhea, cancer-induced diarrhea, chemotherapy-induced diarrhea, radiation enteritis, radiation-induced diarrhea, stress-induced diarrhea, chronic diarrhea, AIDS-related diarrhea, C. difficile associated diarrhea, traveller's diarrhea, acute infectious diarrhea, diarrhea induced by graph versus host disease, other types of diarrhea, chemotherapy-induced nausea and vomiting (emesis), post-operative nausea and vomiting, cyclic vomiting syndrome and functional vomiting. In addition, the compounds possess utility for the treatment of diseases and disorders characterized by poor stomach or intestinal absorption, such as malabsorption syndrome, short bowel syndrome, celiac disease and cachexia.

### BACKGROUND OF THE INVENTION

A number of peptide hormones are involved in the control of the different functions in the gastrointestinal (GI) tract, including absorption, secretion, blood flow and motility (Mulvihill, S. J.; et al. in Basic and Clinical Endocrinology, 4^{th} edition, Greenspan, F. S.; Baxter, J. D., Eds., Appleton & Lange. Norwalk, Conn., 1994, pp 551-570). Since interactions between the brain and GI system are critical to the proper regulation of these functions, these peptides can be produced locally in the GI tract or distally in the CNS. The role of these peptides has resulted in investigation of their modulation for therapeutic purposes in treating GI disorders. (Sanger, G. J. Drug. Disc. Today 2008, 13, 234-239.)

One of these peptide hormones, motilin, a linear 22-amino acid peptide, plays a regulatory role in the GI physiological system through governing of fasting gastrointestinal motor activity. As such, the peptide is periodically released from the duodenal mucosa during fasting in mammals, including humans. More precisely, motilin exerts a powerful effect on gastric motility through the contraction of gastrointestinal smooth muscle to stimulate gastric emptying, decrease intestinal transit time and initiate phase III of the migrating motor complex (MMC) in the small bowel. (Itoh, Z., Ed., Motilin, Academic Press: San Diego, Calif., 1990, ASIN: 0123757304; Poitras, P.; Peeters, T. L. Curr. Opin. Endocrinol. Diab. 2008, 15, 54-57; Itoh, Z. Peptides 1997, 18, 593-608; Nelson, D. K. Dig. Dis. Sci. 1996, 41, 2006-2015; Peeters, T. L.; Vantrappen, G.; Janssens, J. Gastroenterology 1980, 79, 716-719; Luiking, Y. C.; Itoh, Z.; Sekiguchi, T. Scand. J. Gastroenterol. Suppl. 1983, 82, 121-134; Itoh, Z.; Aizawa, I.; Sekiguchi, T. Clin. Gastroenterol. 1982, 11, 497-521; Peeters, T. L.; Stolk, M. F.; Nieuwenhuijs, V. B.; Portincasa, P.; Depoortere, I.; Van Berge Henegouwen, G. P.; Akkermans, L. M. A. Gut 1998, 42, 830-835.)

Motilin can exert these effects through receptors located predominantly on the human antrum and proximal duodenum, although its receptors are found to some degree along the entire GI tract. (Peeters, T. L.; Bormans, V.; Vantrappen, G. Regul. Pept. 1988, 23, 171-182; Poitras, P.; Miller, P.; Dickner, M.; Mao, Y. K.; Daniel, E. E.; St-Pierre, S.; Trudel, L. Peptides 1996, 17, 701-707; Miller, P.; Trudel, L.; St-Pierre, S.; Takanashi, H.; Poitras, P. Peptides 2000, 21, 283-287; Takeshita E, Matsuura B, Dong M, Miller L J, Matsui H, Onji M. J. Gastroenterol. 2006, 41, 223-230.) Therefore, the hormone is involved in motility of both the upper and lower parts of the GI system. Motilin and its receptors have been found in the CNS and periphery and has been demonstrated to activate neurons in the amygdala (Feng, X.; Peeters, T. L.; Tang, M. Peptides 2007, 28, 625-631) and in the hippocampus (Xu, L.; Sun, X.; Depoortere, I.; Lu, J.; Guo, F.; Peeters, T. L. Peptides 2008, 29, 585-592) in rats to stimulate GI motility, as does administration of motilin directly to the hippocampus (Guan, Y.; Tang, M.; Jiang, Z.; Peeters, T. L. Brain Res. 2003, 984, 33-41). In addition, other physiological roles in the nervous system for motilin that have not yet been definitively elucidated have been suggested and the potential involvement of as yet unidentified motilin receptor subtypes has been postulated. (Chen, H.; Chen, L.; Wang, J. J.; Wei, H. J.; Yung, W. H. NeuroReport 2007, 18, 1345-1349; Thielemans, L.; Depoortere, I.; Van Assche, G.; Bender, E.; Peeters, T. L. Brain Res. 2001, 895, 119-128; Depoortere, I.; Peeters, T. L. Am. J. Physiol. 1997, 272, G994-G999 and O'Donohue, T. L.; et al. Peptides 1981, 2, 467-477.) For example, motilin receptors in the brain have been suggested to play a regulatory role in a number of CNS functions, including feeding and drinking behavior, micturition reflex, central and brain stem neuronal modulation and pituitary hormone secretion (Itoh, Z. Peptides 1997, 18, 593-608; Asakawa, A.; Inui, A.; Momose, K. M.; et al. Peptides 1998, 19, 987-990 and Rosenfeld, D. J.; Garthwaite, T. L. Physiol. Behav. 1987, 39, 753-756).

The recent identification and cloning of the human motilin receptor (Intl. Pat. Appl. Publ. WO 99/64436; Feighner, S. D.; Tan, C. P.; McKee, K. K.; et al. Science 1999, 284, 2184-2188) has simplified and accelerated the search for agents which can modulate its activity for specific therapeutic purposes. Due to the involvement of motilin in control of gastric motility, agents that either diminish (in the case of hypomotility disorders) or enhance (in the case of hypermotility disorders) the activity at the motilin receptor, are a particularly attractive area for further investigation in the search for new effective pharmaceuticals towards a number of GI indications.

Two primary avenues have been pursued to discover and develop motilin agonists as therapeutic agents to enhance motility. (Peeters, T. L. Neurogastroenterol. Motil. 2006, 18, 1-5.) The first of these, peptidic agonists of the motilin receptor, have clinical application for the treatment of hypomotility disorders, in particular gastroparesis, (Haramura, M.; Tsuzuki, K.; Okamachi, A.; et al. Bioorg. Med. Chem. 2002, 10, 1805-1811; U.S. Pat. Nos. 5,422,341; 5,432,261; 5,459,049; 5,695,952; 5,721,353; 5,734,012; 6,018,037; 6,380,158; 6,420,521, 6,838,438; U.S. Pat. Appl. Publ. 2001/041791; 2003/176640; 2004/254345; 2005/065156; 2005/080116, 2005/106146; 2005/208626; Intl. Pat. Appl. Publ. WO 98/42840; WO 01/00830; WO 02/059141). Structural studies (Massad, T.; Jarvet, J.; Tanner, R.; Tomson, K.; Smirnova, J.; Palumaa, P.; Sugai, M.; Kohno, T.; Vanatalu, K.; Damberg, P. J. Biomol. NMR 2007, 38, 107-123), structure-activity investigations (Peeters, T. L.; Macielag, M. J.; Depoortere, I.; et al. Peptides 1992, 13, 1103-1107; Haramura, M.; Tsuzuki, K.; Okamachi, A.; et al. Chem. Pharm. Bull. 1999, 47, 1555-1559) and mutational analyses (Tokunaga, H.; Matsuura, B.; Dong, M.; Miller, L. J.; Ueda, T.; Furukawa, S.; Hiasa, Y.; Onji, M. Am. J. Physiol. 2008, 294, G460-G466) have determined the active conformation, key residues and critical interactions involved in the interaction of the native peptide with its receptor. Atilmotin, a peptide analogue derived from the C-terminal 14 residues of motilin, has shown promising results in early human clinical studies. (Park, M. I.; Ferber, I.; Camilleri, M.; et al. Neurogastroenterol. Motil. 2006, 18, 28-36; Intl. Pat. Appl. Publ. WO 2006/138023; WO 2006/138026; U.S. Pat. Appl. Publ. 2006/287243.)

The macrolide antibiotic erythromycin has long been known to have stimulation of GI motility as a side effect and, hence, has been utilized as a treatment for gastroparesis. This effect was subsequently shown to be mediated through interaction at the motilin receptor. (Hasler, W. L.; Heldsinger, A.; Chungal, O. Y. Am. J. Physiol. 1992, 262, G50-G55; Peeters, T. L. Gastroenterology 1993, 105, 1886-1899; Weber, F. H., Jr.; Richards, R. D.; McCallum, R. W. Am. J. Gastroenterol. 1993, 88, 485-490.) However, use of erythromycin therapy can be associated with nausea, diarrhea, cramping and abdominal pain and, further, must be limited in duration to avoid development of bacterial resistance. As another strategy aimed at motilin agonist therapeutics, the development of derivatives of erythromycin, which have little or no antibiotic activity, but maintain the GI stimulatory effects (commonly referred to as motilides), has been the subject of a considerable number of research efforts. (Faghih, R.; Nellans, H. N.; Plattner, J. J. Drugs of the Future 1998, 23, 861-872; Salat, P.; Parikh, V. Ind. J. Pharmacol. 1999, 31, 333-339; Wu, Y. J. Curr. Pharm. Des. 2000, 6, 181-223; Inatomi, N.; Sato, F.; Itoh, Z.; Omura, S. Mode of action of macrolides with motilin agonistic activity—motilides. Macrolide Antibiotics, 2^{nd} edition, Omura, S., ed., Academic Press: San Diego, Calif., 2002, pp 501-531; U.S. Pat. Nos. 4,677,097; 4,920,102; 5,008,249; 5,175,150; 5,418,224; 5,470,961; 5,523,401; 5,523,418; 5,538,961; 5,554,605; 5,578,579; 5,658,888; 5,712,253; 5,854,407; 5,912,235; 5,922,849; 6,077,943; 6,084,079; 6,100,239; 6,165,985; 6,403,775; 6,562,795; 6,750,205; 6,939,861; 6,946,482; 7,211,568; U.S. Pat. Appl. Publ. 2002/025936; 2002/094962; 2003/220271; 2004/138150; 2004/147461; 2005/119195; 2006/270616; Intl. Pat. Appl. Publ. WO 01/60833; WO 02/051855; WO 2004/19879; WO 2005/18576; WO 2006/070937; WO 2006/127252) Generally disappointing results in clinical trials have been observed for such motilides as EM-574 (Satoh, M.; Sakai, T.; Sano, I.; et al. J. Pharmacol. Exp. Ther. 1994, 271, 574-579; Choi, M. G.; Camilleri, M.; Burton, D. D.; Johnson, S.; Edmonds, A. J. Pharmacol. Exp. Ther. 1998, 285, 37-40), ABT-229 (alemcinal, Talley, N. J.; Verlinden, M.; Snape, W.; et al. Aliment. Pharmacol. Ther. 2000, 14, 1653-1661; Talley, N. J.; Verlinden, M.; Geenan, D. J.; et al. Gut 2001, 49, 395-401; Chen, C. L.; Orr, W. C.; Verlinden, M. H.; et al. Aliment. Pharmacol. Ther. 2002, 16, 749-757; Netzer, P.; Schmitt, B.; Inauen, W. Aliment. Pharmacol. Ther. 2002, 16, 1481-1490) and GM-611 (miterncinal, Peeters, T. L. Curr. Opin. Investig. Drugs. 2001, 2, 555-557; Koga, H.; Takanashi, H.; Itoh, Z.; Omura, S. Drugs of the Future 2002, 27, 255-272; Takanashi, H.; Yogo, K.; Ozaki, K.; Koga, H.; Itoh, Z.; Omura, S. Pharmacology 2007, 79, 137-148; Ozaki, K. I.; Yogo, K.; Sudo, H.; Onoma, M.; Kamei, K.; Akima, M.; Koga, H.; Itoh, Z.; Omura, S.; Takanashi, H. Pharmacology 2007, 79, 223-235; Ozaki, K,; Sudo, H.; Muramatsu, H.; Yogo, K.; Kamei, K.; Koga, H.; Itoh, Z.; Omura, S.; Takanashi, H. Inflammopharmacology 2007, 15, 36-42; McCallum, R. W.; Cynshi, O. Aliment. Pharmacol. Ther. 2007, 26, 107-116; Yogo, K.; Ozaki, K.; Takanishi, H.; Koto, M.; Itoh, Z.; Omura, S. Dig. Dis. Sci. 2007, 52, 3112-3122; Sudo, H.; Ozaki, K.; Muramatsu, H.; Kamei, K.; Yogo, K.; Cynshi, O.; Koga, H.; Itoh, Z.; Omura, S.; Takanashi, H. Neurogastro. Motil 2007, 19, 318-326; Kimura, K.; Tabo, M.; Itoh, M.; Mizoguchi, K.; Kato, A.; Suzuki, M.; Itoh, Z.; Omura, S.; Takanashi, H. J. Toxicol. Sci. 2007, 32, 217-230; Kimura, K.; Tabo, M.; Mizoguchi, K.; Kato, A.; Suzuki, M.; Itoh, Z.; Omura, S.; Takanashi, H. J. Toxicol. Sci. 2007, 32, 231-239; McCallum, R. W.; Cynshi, O. Aliment. Pharmacol. Ther. 2007, 26, 1121-1130; Saitoh, R.; Miyayama; T.; Mitsui, T.; Akiba, Y.; Higashida, A.; Takata, S.; Kawanishi, T.; Aso, Y.; Itoh, Z.; Omura, S. Xenobiotica 2007, 37, 1421-1432; Onoma, M.; Yogo, K.; Ozaki, K.; Kamei, K.; Akima, M.; Koga, H.; Itoh, Z.; Omura, S.; Takanashi, H. Clin Exp. Pharmacol. Physiol. 2008, 35, 35-42; Yogo, K.; Onoma, M.; Ozaki, K.; Koto, M.; Itoh, Z.; Omura, S.; Takanashi, H. Dig. Dis. Sci. 2008, 53, 912-918; Fuji, E.; Kimura, K.; Mizoguchi, K.; Kato, A.; Takanashi, H.; Itoh, Z.; Omura, S.; Suzuki, M. Tox. Appl. Pharm. 2008, 228, 1-7), primarily due to issues such as poor bioavailability, chemical instability and tachyphylaxis. (Thielemans, L.; Depoortere, I.; Perret, J.; et al. J. Pharmacol. Exp. Ther. 2005, 313, 1397-1405; Mitselos, A.; Depoortere, I.; Peeters, T. L. Biochem. Pharmacol. 2007, 73, 115-124; Mitselos, A.; Vanden Berghe, P.; Peeters, T. L.; Depoortere, I. Biochem. Pharmacol. 2008, 75, 1115-1128.) Nonetheless, due to the therapeutic potential of such agents, the search for motilin agonists in this class has continued and, recently, KOS-2187 (Carreras, C. W.; Liu, Y.; Chen, Y.; et al. Gastroenterology 2005, 128, A464; Carreras, C. W.; Burlingame, M.; Carney, J.; et al. Can. J. Gastroenterol. 2005, 19, 15.) has been described in an effort to circumvent many of these problems. A method useful for analyzing the therapeutic efficiency of these types of molecules has also been formulated (U.S. Pat. No. 6,875,576; U.S. Pat. Appl. Publ. 2002/192709; Intl. Pat. Appl. Publ. WO 02/64092).

Similarly, non-peptide, non-motilide motilin agonists have been reported (U.S. Pat. Appl. Publ. No. 2004/152732; 2005/065156; 2005/080116; Intl. Pat. Appl. Publ. WO 02/137127; WO 02/92592; WO 2005/027908; WO 2005/027637; Jap. Pat. Abstr. Publ. No. 09249620). Of these, BMS-591348 has been described as possessing a pharmacological profile that avoids the tachyphylaxis issues that plagued many of the previous motilin agonist efforts. (Li, J. J.; Chao, H. G.; Wang, H.; et al. J. Med. Chem. 2004, 47, 1704-1708; Lamian, V.; Rich, A.; Ma, Z.; Li, J. Seethala, R.; Gordon, D.; Dubaquie, Y. Mol. Pharmacol. 2006, 69, 109-118.)

On the other hand, antagonists of the motilin receptor are potentially useful as therapeutic treatments for diseases associated with hypermotilinemia and/or gastrointestinal hypermotility, including diarrhea, cancer treatment-related diarrhea, cancer-induced diarrhea, chemotherapy-induced diarrhea, radiation enteritis, radiation-induced diarrhea, stress-induced diarrhea, chronic diarrhea, AIDS-related diarrhea, C. difficile associated diarrhea, traveller's diarrhea, acute infectious diarrhea, diarrhea induced by graph versus host disease, other types of diarrhea, dyspepsia, including gallbladder dyspepsia, irritable bowel syndrome, functional gastrointestinal disorders, chemotherapy-induced nausea and vomiting (emesis), post-operative nausea and vomiting, cyclic vomiting syndrome and functional vomiting. Current treatments for these conditions are ineffective in many cases.

Diarrhea is a common and serious side-effect experienced by cancer patients resulting from surgery, bone marrow transplantation, chemotherapy and radiation treatment. (Stern, J.; Ippoliti, C. Sem. Oncol. Nurs. 2003, 19, 11-16; Benson, A. B., III; Ajani, J. A.; Catalano, R. B.; et al. J. Clin. Oncol. 2004, 22, 2918-2926; O'Brien, B. E.; Kaklamani, V. G.; Benson, A. B. III Clin. Colorectal Canc. 2005, 4, 375-381; Keefe, D. M. Curr. Opin. Oncol. 2007, 19, 323-327; Richardson, G.; Dobish, R. J. Oncol. Pharm. Pract. 2007, 13, 181-198.) Certain chemotherapeutic regimens, particularly those including fluoropyrimidines and irinotecan, result in chemotherapy-induced diarrhea (CID) rates as high as 50-80%. (Arbuckle, R. B.; Huber, S. L.; Zacker, C. The Oncologist 2000, 5, 250-259; Saltz, L. B. J. Support. Oncol. 2003, 1, 35-46; Goldberg-Arnold, R. J.; Gabrail, N.; Raut, M.; Kim, R.; Sung, J. C. Y.; Zhou, Y. J. Support. Oncol. 2005, 3, 227-232; Sharma, R.; Tobin, P.; Clarke, S. J. Lancet Oncol. 2005, 6, 93-102; Gibson, R. J.; Keefe, D. M. K. Support. Care Cancer 2006, 14, 890-900.) The implications of CID include increased morbidity and mortality. This is a significant problem as, in 2001, over 1.4 million individuals in the U.S. were undergoing cancer chemotherapy. A large heterogeneous study of cancer patients at all stages of treatment placed the prevalence of diarrhea at 14%. (M. D. Anderson Symptom Inventory, Cancer 2000, 89(7), 1634-1646). However, for certain types of cancer, the occurrence is higher In colorectal cancer, for example, more than half of patients experienced diarrhea rated serious (grade 3) or higher. Resulting from tissue damage in the intestine caused by drugs designed to thwart the rapid growth of tumor cells, it also affects the cells lining the intestinal wall. No effective therapy exists for this damage nor for the associated diarrhea.

In general, from 10-20% of patients experience CID, although for some chemotherapeutic agents the incidence can be as high as 90%. In approximately 20% of patients, the adverse effect is so severe, it requires a break in or reduction of the treatment regimen and, often, hospitalization. In addition, parenteral nutrition often must be taken due to the inability of patients to take nourishment normally. Hence, this has a negative effect on the efficacy of the chemotherapy. Indeed, a review of clinical trials in colorectal cancer revealed higher death rates primarily due to gastrointestinal toxicity. (Rothenberg, M. L.; Meropol, N. J.; Poplin, E. A.; VanCutsem, E.; Wadler, S. J. Clin. Oncol. 2001, 19, 3801-3807.) Current pharmacological treatments only work in some patients and are much less effective against the more serious grades of diarrhea. (MacNaughton, W. K. Aliment. Pharmacol. Ther. 2000, 14, 523-528).

Acute radiation enteritis (ARE) or radiation induced intestinal dysfunction occurs in 75% of patients undergoing radiation therapy, typically occurring in the second or third week of therapy. Characterized by abdominal cramping and diarrhea, this is a serious and feared side effect that results in increased overall treatment time as well as reduced quality of life and can even result in death. In 5-15% of patients, the condition becomes chronic. In addition to discomfort and reduced quality of life, this side effect decreases the therapeutic benefit from radiation treatment by increasing the overall treatment time. (MacNaughton, W. K. Aliment. Pharmacol. Ther. 2000, 14, 523-528; Nguyen, N. P.; Antoine, J. E.; Dutta, S.; Karlsson, U.; Sallah, S. Cancer 2002, 95, 1151-1163; Gwede, C. K. Sem. Nursing Oncol. 2003, 19, 6-10.)

Indeed, chronic diarrhea can arise as a result of numerous medical conditions. (Schiller, L. R. Curr. Treat. Options Gastroenterol. 2005, 8, 259-266; Spiller, R. Neurogastroenterol. Motil. 2006, 18, 1045-1055.) For example, chronic diarrhea is a common problem for patients with human immunodeficiency virus infection, especially those with advanced disease. This is a debilitating side effect that occurs in 60-90% of AIDS patients. (Cohen, J.; West, A. B.; Bini, E. J. Gastroenterol. Clin. North Am. 2001, 30, 637-664; Oldfield, E. C., III Rev. Gastroenterol. Disord. 2002, 2, 176-88; Sestak, K.; Curr. HIV Res. 2005, 3, 199-205; Thom, K.; Forrest, G. Curr. Opin. Gastroenterol. 2006, 22, 18-23.) Additionally, psychological factors, such as stress, are known to play a role in adversely affecting the proper functioning of the GI tract (North, C. S.; Alpers, D. H.; Thompson, S. J.; Spitznagel, E. L. Dig. Dis. Sci. 1996, 41, 633-640; Kamm, M. A. Eur. J. Surg. Suppl. 1998, 583, 37-40; Botha, C.; Libby, G. Br. J. Hosp. Med. (Lond.) 2006, 67, 344-349.)

Travellers diarrhea affects over 50% of travellers to some destinations, particularly tropical ones, and is estimated to afflict over 11 million individuals annually. Apart from the disruption to business, travel and vacations schedules, this condition is often accompanied by other clinical manifestations such as nausea, vomiting, abdominal pain, fecal urgency, bloody stools, and fever. (Lima, A. A. M. Curr. Opin. Infect. Dis. 2001, 14, 547-552; Al-Abri, S. S.; Beeching, N. J.; Nye, F. J. Lancet Infect. Dis. 2005, 5, 349-360; DuPont, H. L. Gastroenterol. Clin. North Am. 2006, 35, 337-353.) Other acute infectious diarrheas, from mild to severe, can result from a range of etiological agents and is particularly dangerous for infants. (McMahan, Z. H.; DuPont, H. L. Aliment. Pharmacol. Ther. 2007, 25, 759-769.)

Clostridium difficile is the etiological agent responsible for about one-third of cases of antibiotic associated diarrhea and is estimated to have a $1 billion annual cost in the U.S. Antibiotic associated diarrhea is more common in the hospital setting with up to 29% of patients developing the condition, resulting in increased length of stay, increased cost of care, and increased mortality. (Bartlett, J. G. N. Engl. J. Med. 2002, 346, 334-339; Kelly, C. P.; Pothoulakis, C.; LaMont, J. T. N. Engl. J. Med. 1994, 330, 257-262; Kyne, L.; Farrell, R. J.; Kelly, C. P. Gastroenterol. Clin. N. Am. 2001, 30, 753-777; Malnick, S. D. H.; Zimhony, O. Ann. Pharmacother. 2002, 36, 1767-1775; Hull, M. W.; Beck, P. L. Can. Fam. Phys. 2004, 50, 1536-1540; Schroeder, M. S. Am. Fam. Phys. 2005, 71, 921-928; Voth, D. E.; Ballard, J. D. Clin. Microbiol. Rev. 2005, 18, 247-263; Halsey, J. Am. J. Health Syst. Pharm. 2008, 65, 705-715.) It is a serious condition with a mortality rate as high as 25% in frail elderly patients. Recently, the incidence and severity of C. difficile-associated diarrhea (CDAD) has begun to increase dramatically. (Frost. F.; Craun, G. F.; Calderon, R. L. Emerg. Infect. Dis. 1998, 4, 619-625; Olfield, E. C. Rev. Gastroenterol. Disord. 2006, 6, 79-96; McFarland, L. V. Nat. Clin. Pract. Gastroenterol. Hepatol. 2008, 5, 40-48.)

Diarrhea is also induced in patients with graft versus host disease (GVHD). GVHD is a common, potentially life-threatening complication of allogenic hematopoietic stem cell transplantation. Gastrointestinal GVHD frequently involves the colon and complicates management of these seriously ill patients. (Flowers, M. E.; Kansu, E.; Sullivan, K. M. Hematol. Oncol. Clin. North Am. 1999, 13, 1091-1112; Ross, W. A.; Couriel, D. Curr. Opin. Gastroenterol. 2005, 21, 64-69.) In addition, diarrhea is a common side effect after other types of transplantation with an incidence ranging from 10% to 43%. Diarrhea is also a frequent side effect of immtnosuppressive medications. (Ginsburg, P. M.; Thuluvath, P. J. Liver Transpl. 2005, 11, 881-890.)

Generally effective treatments for diarrhea have remained elusive. (Schiller, L. R. Rev. Gastroenterol. Disord. 2007, 7, S27-S38.) Loperamide, an opioid agonist, is only useful for milder diarrhea and does not work in a high percentage of patients. Octreotide, a somatostatin agonist, is used off-label as a diarrheal treatment, but is expensive, given by injection, and also not effective in many instances.

Irritable bowel syndrome (IBS) is the most common functional GI disorder with an estimated worldwide prevalence of 10-15%. (Saito, Y. A.;, Schoenfeld, P.; Locke, G. R. Am. J. Gastroenterol. 2002, 97, 1910-1915; Gilkin, R. J., Jr. Clin. Ther. 2005, 27, 1696-1709; Lacy, B. E.; De Lee, R. J. Clin. Gastroenterol. 2005, 39, S230-S242; Talley, N. J. Intern. Med. J. 2006, 36, 724-728; Ohman, L.; Simren, M. Dig. Liver Dis. 2007, 39, 201-215; Saad, R. J.; Chey, W. D. Exp. Opin. Invest. Drugs 2008, 17, 117-130.) The total annual cost attributable to IBS is estimated to be $30 billion, including $10 billion in direct costs from physician visits and prescription pharmaceuticals, as well as a significant cost from missed work days. (Talley, N. J.; Gabriel, S. E,; Harmsen, W. S.; et al. Gastroenterology 1995, 109, 1736-1741; Maxion-Bergemann, S.; Thielecke, F.; Abel, F.; Bergemann, R. Pharmacoeconomics 2006, 24, 21-37; Videlock, E. J.; Chang, L. Gastroenterol. Clin. North Am. 2007, 36, 665-685.) IBS patients are sub-classified into diarrhea-predominant (IBS-d), constipation-predominant (IBS-c) or those alternating between these two patterns (IBS-m). Treatments for these various subsets generally must be approached with separate and specific therapies. Antispasmodics, tricyclic antidepressants, selective serotonin reuptake inhibitors, laxatives, antidiarrheals, and bulking agents have not proven to be widely effective and tend to treat symptoms, rather than underlying pathophysiology. (Schoenfeld, P. Gastroenterol. Clin. North Am. 2005, 34, 319-335; Cremonini, F.; Talley, N. J. Nat. Clin. Pract. Gastroenterol. Hepatol. 2005, 2, 82-88; Andresen, V.; Camilleri, M. Drugs 2006, 66, 1073-1088; Spiller, R.; Aziz, Q.; Creed, F.; Emmanuel, A.; Houghton, L.; Hungin, P.; Jones, R.; Kumar, D.; Rubin, G.; Trudgill, N.; Whorwell, P. Gut 2007, 56, 1770-1798.) The plasma levels of motilin have been shown to be elevated in patients with IBS. (Simren, M.; Bjornsson, E. S.; Abrahamsson, H. Neurogastroenterol. Motil. 2005, 174 51-57.) Motilin antagonists, hence, would be a useful treatment for patients with IBS. They would likely be more suited to IBS-d and to a lesser extent, IBS-m. IBS-d is manifested by fecal urgency and frequent loose bowel movements (>3 per day). Individuals suffering from IBS-d account for approximately one-third of the entire IBS patient population.

Another extremely common GI disorder, dyspepsia, is characterized by chronic or recurrent upper GI distress with no obvious physical cause. (Tack, J.; Bisschops, R.; Sarnelli, G. Gastroenterology 2004, 127, 1239-1255; Kleibeuker, J. H.; Thijs, J. C. Curr. Opin. Gastroenterol. 2004, 20, 546-550; Talley, N. J.; Vakil, N.; et al. Am. J. Gastroenterol. 2005, 100, 2324-2337; Talley, N. J.; Vakil, N.; Moayyedi, P. Gastroenterology 2005, 129, 1756-1780; Smith, M. L. Dig. Liver Dis. 2005, 37, 547-558; Saad, R. J.; Chey, W. D. Aliment. Pharmacol. Ther. 2006, 24, 475-492; Suzuki, H.; Nishizawa, T.; Hibi, T. J. Gastroenterol. 2006, 41, 513-523.; Mahadeva, S.; Goh, K. L. World J. Gastroenterol. 2006, 12, 2661-2666; Monkemuller K, Malfertheiner P. World J. Gastroenterol. 2006, 12, 2694-2700; Mizuta, Y.; Shikuwa, S.; Isomoto, H.; Mishima, R.; Akazawa, Y.; Masuda, J.; Omagari, K.; Takeshima, F.; Kohno, S. J. Gastroenterol. 2006, 41, 1025-1040; Chua, A. S. World J. Gastroenterol. 2006, 12, 2656-2659; Camilleri, M. Gastroenterol. Clin. North. Am. 2007, 36, 649-664; Halder, S. L. S.; Talley, N. J. Curr. Treat. Options Gastroenterol. 2007, 10, 259-272.) Typical symptoms include gastric fullness, bloating, pain, nausea and vomiting. This disease has prevalence as high as 20% annually in Western countries. It accounts for up to 5% of all visits to primary care physicians and 30% of visits to GI specialists. As with IBS, the patient population can be categorized into various subsets based upon symptoms. However, the largest patient subset (up to 60%) suffers from dyspepsia with no known organic cause, otherwise known as “functional dyspepsia (FD).” FD has a major impact on quality of life and health care resources. In analogy with IBS, no widely-accepted therapy for the treatment of FD currently exists. (Stanghellini, V.; De Ponti, F.; De Giorgio, R.; et al. Drugs 2003, 63, 869-892; Cremonini, F.; Delgado-Aros, S.; Talley, N. J. Best Pract. Res. Clin. Gastroenterol. 2004, 18, 717-733.) Circulating plasma motilin levels are also seen to be raised in patients suffering from dyspepsia. (Kusano, M.; Sekiguchi, T.; Kawamura, O.; Kikuchi, K.; Miyazaki, M.; Tsunoda, T.; Horikoshi, T.; Mori, M. Am. J. Gastroenterol. 1997, 92, 481-484; Kamerling, I. M.; Van Haarst, A. D.; Burggraaf, J.; Schoemaker, R. C.; Biemond, I.; Heinzerling, H.; Jones, R.; Cohen, A. F.; Masclee, A. A. Am. J. Physiol. Gastrointest. Liver Physiol. 2003, 284, G776-G781.) As with IBS, motilin antagonists would mitigate the effects of motilin in such patients.

Chemotherapy-induced nausea and vomiting (CINV), or emesis, is one of the most severe adverse effects resulting from cancer treatment and is often cited as the side effect most feared by patients. From 70-80% of patients receiving cancer chemotherapy experience CINV. In addition to a significant deterioration in quality of life, this condition often requires modification or delay of chemotherapeutic regimens with concomitant negative impact on the effectiveness of treatment. Despite recent progress in the development and availability of new approaches to mitigating the effects of CINV, there remains a compelling need for alternative strategies for patients for whom current treatments are inadequate. (Lindley, C. M.; Hirsch, J. D.; O'Neill, C. V.; Transau, M. D.; Gilbert, C. S.; Osterhaus, J. T. Qual. Life Res. 1992, 1, 331-340; Martin, M. Oncology 1996, 53, 26-31; Kovac, A. L. Drug Safety 2003, 26, 227-259; Grunberg, S. M. J. Support. Oncol. 2004, 2, 1-12; Jordan, K.; Kasper, C.; Schmoll, H.-J. Eur. J. Canc. 2005, 41, 199-205; Herrstedt, J.; Dombernowsky, P. Basic Clin. Pharmacol. Toxicol. 2007, 101, 143-150; Jordan, K.; Sippel, C.; Schmoll, H.-J. The Oncologist 2007, 12, 1143-1150.)

Post-operative nausea and vomiting (PONV) is a common complication from surgery, occurring in 30-50% of patients. PONV can lead to unintended or extended hospitalization, electrolyte abnormalities and strain on surgical sutures, plus a substantial negative effect on quality of life. As such, it increases health care costs and decreases patient satisfaction. The importance of dealing with PONV has become well-recognized in the medical community and there is a need for effective treatments. (Osoba, D.; Zee, B.; Warr, D.; et al. Support. Care Cancer 1997, 5, 303-313; Kovac, A. L. Drugs 2000, 59, 213-243; Gan, T. J. J. Am. Med. Assoc. 2002, 287, 1233-1236; Tramèr, M. R. Best Pract. Res. Clin. Anaesthesiol. 2004, 18, 693-701; Habib, A. S.; Gan, T. J. Can. J. Anesth. 2004, 51, 326-341; Golembiewski, J.; Chernin, E.; Chopra, T. Am. J. Health-Syst. Pharm. 2005, 62, 1247-1260.)

In addition, nausea and vomiting are symptoms resulting from an emetic reflex, which can occur due to a variety of other reasons, and includes cyclic vomiting syndrome and functional vomiting, although sometimes no clear cause can be determined. (Chepyala, P.; Olden, K. W. Curr. Treat. Options Gastroenterol. 2008, 11, 135-144.)

Elevated motilin levels have been demonstrated in patients suffering from gallbladder motility problems, including gallstones. (Zhang, Z.-H.; Wu, S.-D.; Su, Y.; Jin, J.-Z.; Fan, Y.;Yu, H.; Zhang, L.-K. Hepatobil. Pancr. Dis. Intl. 2008, 7, 58-64.) Hypermotilinemia and disturbance of interdigestive migrating contractions due to nonsteroidal anti-inflammatory drugs has been reported in dogs. (Narita, T.; Okabe, N.; Hane, M.; Yamamoto, Y.; Tani, K.; Naito, Y.; Hera, S. J. Vet. Pharmacol. Ther. 2006, 29, 569-577.)

In addition to treatment of disorders characterized by hypermotility, the use of motilin antagonists would also be useful in the treatment of diseases and disorders characterized by poor stomach or intestinal absorption. A motilin antagonist would slow gastrointestinal motility thereby permitting longer GI exposure time for absorption of necessary nutrients. In general terms, malabsorption syndrome is an alteration in the ability of the intestine to absorb nutrients adequately into the bloodstream. This can refer to malabsorption of one specific nutrient or for specific carbohydrates, fats, or trace elements (micronutrients). Malabsorption syndrome can be characterized by anemia, bloating, diarrhea, cramping, edema, weight loss, muscle atrophy or wasting, skin disorders and heart irregularities. Several disorders can lead to malabsorption syndrome, including, but not limited to, cystic fibrosis, chronic pancreatitis, lactose intolerance, and gluten enteropathy (non-tropical sprue). (Owens, S. R.; Greenson, J. K. Histopathology 2007, 50, 64-82.)

Among related diseases and disorders is celiac disease, a chronic disorder afflicting almost 1% of the population. Celiac disease is a GI disorder characterized by inflammation, leading to injury to the mucosal lining of the small intestine. The inflammation results when gliadin, a protein found in gluten-containing foods, is ingested by genetically susceptible individuals. The mucosal damage and subsequent malabsorption of nutrients can lead to numerous complications. (Alaedini, A.; Green, P. H. R. Am. Intern. Med. 2005. 142, 289-298; Koning, F. Gastroenterology 2005, 129, 1294-1301; Chand, N.; Mihas, A. A. J. Clin. Gastroenterol. 2006, 40, 3-14; Westerberg, D. P.; Gill, J. M.; Dave, B.; et al. J. Am. Osteopath. Assn. 2006, 106, 145-151; Jones, R. B.; Robins, G. G.; Howdle, P. D. Curr. Opin. Gastroenterol. 2006, 22, 117-123; Green, P. H. R.; Jabri, B. Ann. Rev. Med. 2006, 57, 207-221; Hill, I. D. Curr. Treat. Options Gastroenterol. 2006, 9, 399-408.) The only current treatment is modification to a gluten-free diet.

Short bowel syndrome is a medical condition that occurs after resection of a substantial portion of small intestine and is characterized by malnutrition. (Parekh, N. R.; Steiger, S. R. Curr. Treat. Options Gastroenterol. 2007, 10, 10-23; Misiakos, E. P.; Macheras, A.; Kapetanakis, T.; Liakakos, T. J. Clin. Gastroenterol. 2007, 41, 5-18.; Buchman, A. L. Gastroenterology. 2006, 130 (Suppl. 1), S5-S15; Jackson, C.; Buchman, A. L. Curr. Gastroenterol. Rep. 2005, 7, 373-378; Scolapio, J. S. Curr. Opin. Gastroenterol. 2004, 20, 143-145; Buchman, A. L.; Scolapio, J.; Fryer, J. Gastroenterology 2003, 124, 1111-1134; Westergaard, H. Sem. Gastrointest. Dis. 2002, 13, 210-220.) The syndrome is particularly distressing in children, where mortality and morbidity are very high. (Vanderhoof, J. A.; Young, R. J.; Thompson, J. S. Pediatric Drugs 2003, 5, 625-631; Vanderhoof, J. A. J. Ped. Gastroenterol. Nutri. 2004, 39, 5768-5771; Sukhotnik, I.; Coran, A. G.; et al. Pediatr. Surg. Int. 2005, 21, 947-953.) No current pharmacological agents are currently approved for SBS, which is typically treated through intestinal adaptation or rehabilitation in order to improve the nutritional status of SBS patients. (DiBaise, J. K.; Young, R. J.; Vanderhoof, J. A. Am. J. Gastroenterol. 2004, 99, 1823-1832.)

Additionally, the potential for improving nutrient absorption through the use of motilin antagonists could be useful in the treatment of cachexia, a wasting disorder common in serious illnesses such as cancer, AIDS, chronic heart failure and other cardiovascular diseases, and renal disease, as well as in the aged. Cancer cachexia is a therapeutic condition characterized by weight loss and muscle wasting and afflicts approximately 50% of all cancer patients and is the main cause of death in more than 20% of patients. Additionally, this condition has been shown to be a strong independent risk factor for mortality. Kern, K. A.; Norton, J. A. JPEN 1980, 12, 286-298; Tisdale, M. J. J. Natl Cancer Inst. 1997, 89, 1763-1773; Gagnon, B.; Bruera, E. Drugs 1998, 55, 675-688; Inui, A. CA Cancer J. Clin. 2002, 52, 72-91; Bossola, M.; Pacelli, F.; Doglietto, G. B. Exp. Opin. Invest. Drugs 2007, 16, 1241-1253; Argilyés, J. M.; Lopéz-Soriano, F. J.; Busquets, S. Exp. Opin. Emerging Drugs 2007, 12, 555-570.) Likewise, patients suffering from chronic heart failure are at serious risk from a similar wasting syndrome. (von Haehling, S.; Doehner, W.; Anker, S. D. Cardiovasc. Res. 2007, 73, 298-309; Springer, J.; Filippatos, G.; Akashi, Y. J.; Anker, S. D. Curr. Opin. Cardiol. 2006, 21, 229-233; Akashi, Y. J.; Springer, J.; Anker, S. D. Curr. Heart Fail. Rep. 2005, 2, 198-203; Anker, S. D.; Steinborn, W.; Strassburg, S. Ann. Med. 2004, 36, 518-529.) This condition also affects an increasing proportion of the elderly. (Yeh, S. S.; Lovitt, S.; Schuster, M. W. J. Ann. Med. Dir. Assoc. 2007, 8, 363-377; Morley, J. E. J. Gerontology. Ser. A: Biol. Sci. Med. Sci. 2003, 58A, 131-137.)

Despite the potential offered by motilin antagonists as a novel approach to treat hypermotility and malabsorption disorders, efforts have lagged those directed at agonists. A variety of peptidic compounds have been described as antagonists of the motilin receptor [(ANQ-11125; Peeters, T. L.; Depoortere, I.; Macielag, M. J.; Marvin, M. S.; Florance, J. R.; Galdes, A. Biochem. Biophys. Res. Comm. 1994, 198, 411-416); (OHM-11526: Farrugia, G.; Macielag, M. J.; Peeters, T. L.; Sarr, M. G.; Galdes, A.; Szurszewski, J. H. Am. J. Physiol. 1997, 273, G404-G412; Depoortere, I.; Macielag, M. J.; Galdes, A.; Peeters, T. L. Eur. J. Pharmacol. 1995, 286, 241-247); (M-2029: Sudo, H.; Yoshida, S.; Ozaki, K.; Muramatsu, H.; Onoma, M.; Yogo, K.; Kamei, K.; Cynshi, O.; Kuromaru, O.; Peeters, T. L.; Takanashi, H. Eur. J. Pharmacol. 2008, 581, 296-305; Mitselos, A.; Depoortere, I.; Peeters, T. L. Biochem. Pharmacol. 2007, 73, 115-124); Poitras, P.; Miller, P.; Gagnon, D.; St-Pierre, S. Biochem. Biophys. Res. Comm. 1994, 205, 449-454; U.S. Pat. Nos. 5,470,830; 6,255,285; 6,586,630; 6,720,433; U.S. Pat. Appl. Publ. 2003/176643; Intl. Pat. Appl. Publ. WO 99/09053; WO 00/17231; WO 00/44770; WO 02/64623]. These peptidic antagonists suffer from the known limitations of peptides as drug molecules, in particular poor oral bioavailability and degradative metabolism.

Cyclization of peptidic derivatives is a method that can be employed to improve the properties of a linear peptide both with respect to metabolic stability and conformational freedom. Cyclic molecules tend to be more resistant to metabolic enzymes. Some cyclic peptides are known to have motilin agonist activity (U.S. Pat. No. 5,734,012). In addition, cyclic peptide motilin antagonists have been reported, highlighted by GM-109. (Takanashi, H.; Yogo, K.; Ozaki, M.; Akima, M.; Koga, H.; Nabata, H. J. Pharm. Exp. Ther. 1995, 273, 624-628; Haramura, M.; Okamachi, A.; Tsuzuki, K.; Yogo, K.; Ikuta, M.; Kozono, T.; Takanashi, H.; Murayama, E. Chem. Pharm. Bull. 2001, 49, 40-43; Haramura, M.; Okamachi, A.; Tsuzuki, K.; Yogo, K.; Ikuta, M.; Kozono, T.; Takanashi, H.; Murayama, E. J. Med. Chem. 2002, 45, 670-675; U.S. Pat. No. 7,018,981; U.S. Pat. Appl. Publ. 2003/191053; Intl. Pat. Appl. Publ. WO 02/16404; Jap. Pat. Abstr. Publ. No. 07138284)

Macrocyclic peptidomimetics have been previously described as antagonists of the motilin receptor and their uses for the treatment of a variety of GI disorders summarized. (Marsault, E.; Hoveyda, H. R.; Peterson, M. L.; Saint-Louis, C.; Landry, A.; Vézina, M.; Ouellet, L.; Wang, Z.; Ramaseshan, M.; Beaubien, S.; Benakli, K.; Beauchemin, S.; Déziel, R.; Peeters, T.; Fraser, G. L. J. Med. Chem. 2006, 49, 7190-7197; Marsault, E.; Benakli, K.; Beaubien, S.; St-Louis, C.; Deziel, R.; Fraser, G. Bioorg. Med. Chem. Lett. 2007, 17, 4187-4190; Intl. Pat. Appl. Publ. WO 2004/111077; U.S. Pat. Appl. Publ. 2005/054562, Intl. Pat. Appl. Publ. WO 2008/033328). These peptidomimetic macrocyclic motilin antagonists are distinguished from the aforementioned cyclic peptide motilin antagonists in that it was found that such peptidic derivatives containing D-amino acids were devoid of activity. In contrast, for the tripeptidomimetic compounds of the present invention, the D-stereochemistry is beneficial for two of the three building elements. Further, the tether portion of the molecule provides a non-peptidic component and, hence, distinct structures. These peptidomimetic macrocycles were demonstrated to have binding and functional activity at the motilin receptor.

Other motilin antagonists, which are non-peptidic and non-cyclic in nature have also been reported. [(RWJ-68023: Beavers, M. P.; Gunnet, J. W.; Hageman, W.; Miller, W.; Moore, J. B.; Zhou, L.; Chen, R. H. K.; Xiang, A.; Urbanski, M.; Combs, D. W.; Mayo, K. H.; Demarest, K. T. Drug Design Disc. 2001, 17, 243-251); Johnson, S. G.; Gunnet, J. W.; Moore, J. B.; et al. Bioorg. Med. Chem. Lett. 2006, 16, 3362-3366; U.S. Pat. Nos. 5,972,939; 6,384,031; 6,392,040; 6,423,714; 6,511,980; 6,624,165; 6,667,309; 6,967,199; U.S. Pat. Appl. Publ. 2001/041701; 2001/056106, 2002/002192; 2002/013352; 2002/103238; 2002/111484; 2003/203906; 2005/148584; 2007/054888; Intl. Pat. Appl. Publ. WO 99/21846; WO 01/68620; WO 01/68621; WO 01/68622; WO 01/85694) Of these, RWJ-68023 has been examined in humans, but with a poor outcome, likely due to the level of potency of this molecule. (Kamerling, I. M. C.; van Haarst, A. D.; Burggraaf, J.; et al. Br. J. Clin. Pharmacol. 2003, 57, 393-401.)

Modulation of the migrating motor complex (MMC) is a characteristic of pharmaceutical agents that has proven useful in the treatment of gastrointestinal disorders, such as gastroparesis, irritable bowel syndrome (IBS) and dyspepsia. (Itoh, Z.; Aizawa, I.; Sekiguchi, T. Clin. Gastroenterol. 1982, 11, 497-521; Itoh, Z.; Sekiguchi, T. Scand. J. Gastroenterol. Suppl. 1983, 82, 121-134; Fiorenza, V.; Yee, Y. S.; Zfass, A. M. Am. J. Gastroenterol. 1987, 82, 1111-1114; Bueno, L.; Frexinos, J.; Fioramonti, J. Pharmacology 1988, 36, 15-22; Plaza, M. A. Curr. Opin. Invest. Drugs 2001, 2, 539-544; Husebye, E. Neurogastroenterol. Motil. 1999, 11, 141-161.) In humans, cisapride, a potent prokinetic agent, increased the mean contractile amplitude and incidence of distally propagated clustered activity, but does not alter the duration of the MMC cycle. (Benson, M. J.; Castillo, F. D.; Deeks, J. J.; Wingate, D. L. Dig. Dis. Sci. 1992, 37, 1569-1575.) However, cisapride was found to have severe cardiac side effects, which resulted in its removal from the market in 2000. Alosetron, a 5-HT₃ antagonist used to treat GI disorders, has been shown to inhibit the MMC cycle and was an effective therapeutic for IBS-d. (Bush, T. G.; Spencer, N. J.; Watters, N.; Sanders, K. M.; Smith, T. K. Am. J. Physiol. Gastroenterol. Liver Physiol. 2001, 281, 974-983; Kawano, K.; Mori, T.; Fu. I.; et al. Neurogastroenterol. Motil. 2005, 17, 290-301.) Unfortunately, the appearance of a high incidence of ischemic colitis resulted in its removal from the market, also in 2000, and it is currently only approved for highly restricted use. Muscarinic receptor antagonists have been demonstrated to have low efficacy in modulating MMC in rats. (Axelsson, L.-O.; Wallin, B.; Gillberg, P.-G.; Sjöberg, B.; Söderberg, C.; Hellström, P. M. Eur. J. Pharmacol. 2003, 467 211-218.) J

The motilin antagonists of the present invention unexpectedly have been found to suppress and, in some cases, even completely inhibit, the spontaneous migrating motor complex (MMC). With the role of the MMC in normal regulation and proper functioning of the GI tract, these antagonists would be useful for the treatment of a range of GI disorders. Further, this suppression may provide for utility in the treatment of disorders, including short bowel syndrome and celiac disease, characterized by poor intestinal absorption. Suppression or inhibition of MMCs may result in delay of excretion and a longer time for absorption of nutrients.

Moreover, other small molecule motilin antagonists have not been seen to have an effect on the migrating motor complex (MMC). For example, RWJ 68023 produced no effect on spontaneous gastric or small intestinal motor activity in rabbit models. (Otterson, M. F.; Leming, S.; Gunnet, J.; Hageman, W. The effect of RWJ-68023, a novel motilin antagonist, on rabbit small intestinal motility, Digestive Disease Week, Orlando, Fla., 17-22 May 2003, Abstract S1150.) Similarly, GM-109 does not inhibit the MMC in either rabbit or dog models except at very high dose levels (equivalent to 10 mmol/kg/h). (Jin, C.; Naruse, S.; Kitagawa, M.; et al. Gastroenterology 2002, 123, 1578-1587.)

As such, the present invention provides a unique and previously unknown utility for these macrocyclic motilin antagonists in the treatment of disorders involving disturbed MMC or poor intestinal absorption. The suppressive effect of the motilin antagonists of the present invention on the MMC makes them uniquely suitable for use in the treatment of these conditions.

### SUMMARY OF THE INVENTION

The present invention provides novel conformationally-defined macrocyclic compounds that can function as antagonists of the motilin receptor.

According to aspects of the present invention, the present invention is directed to compounds of formula I:

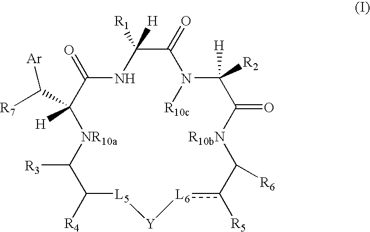

or pharmaceutically acceptable salts, hydrates or solvates thereof wherein:

Y is

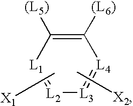

wherein (L₅) and (L₆) indicate the bonds to L₅ and L₆ of formula I, respectively;

Ar is selected from the group consisting of:

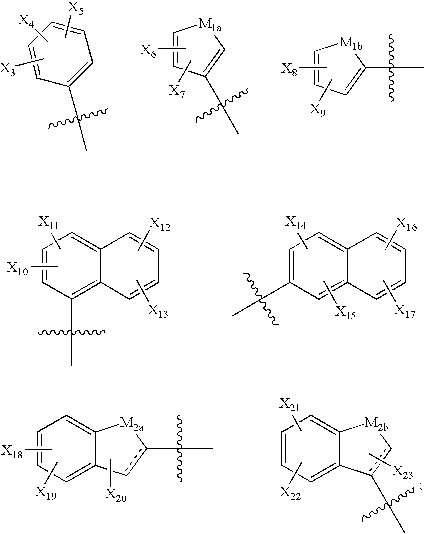

R₁ is selected from the group consisting of: —(CH₂)₅CH₃, —CH(CH₃)(CH₂)ₜCH₃, —(CH₂)ᵤCH(CH₃)₂, —C(CH₃)₃, and

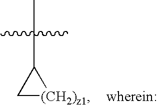

s is 0, 1, 2 or 3;

t is 1 or 2;

u is 0 or 1; and

z1 is 1, 2, 3 or 4;

R₂ is selected from the group consisting of hydrogen, —(CH₂)ₐₐCH₃, —CH₂SCH₃, —CH₂CH₂SCH₃, —(CH₂)_{bb}CH(CH₃)₂, —CH(CH₃)(CH₂)_{cc}CH₃, —(CH₂)_{dd}—NR₁₁R₁₂, and —(CH₂)_{cc}R₁₃; wherein

aa and bb are independently 0, 1, 2 or 3;

cc and dd are independently 1, 2, 3 or 4;

ee is 0, 1, 2, 3 or 4;

R₁₁ is selected from the group consisting of hydrogen, lower alkyl, formyl, acyl, carboxyalkyl, carboxyaryl, amido, amidino, sulfonyl and sulfonamido;

R₁₂ is selected from the group consisting of hydrogen and lower alkyl;

R₁₃ is selected from the group consisting of:

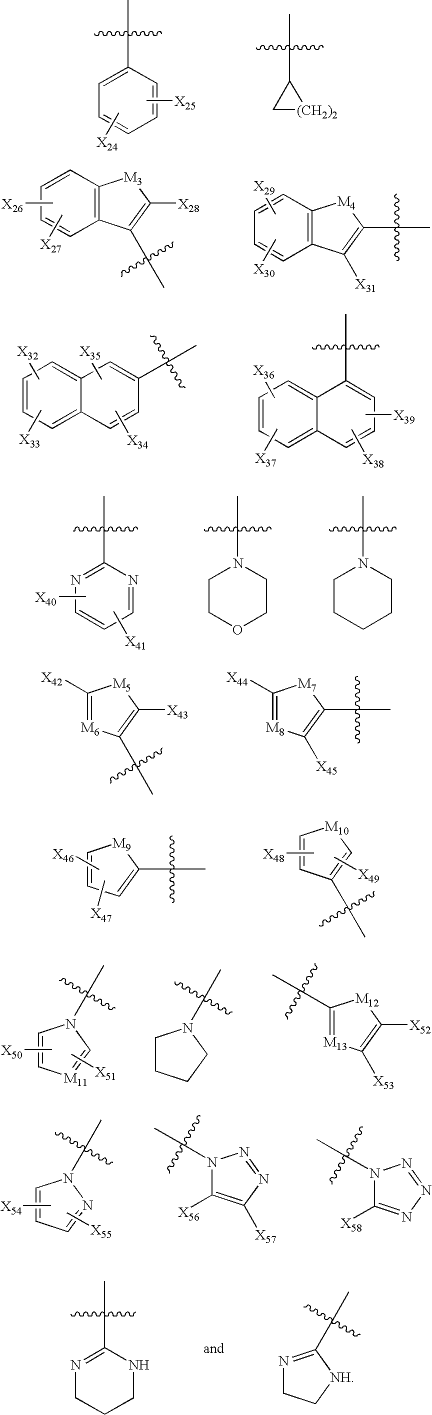

wherein z2 is 1, 2, 3 or 4;

and, when ee is 1, 2, 3 or 4, R₁₃ is farther selected from the group consisting of hydroxy, alkoxy, amidino, and azido;

R₃, R₄, R₅, and R₆ are independently selected from the group consisting of hydrogen, methyl, ethyl, isopropyl and hydroxymethyl;

R₇ is selected from the group consisting of hydrogen, methyl, hydroxy and amino;

R₁₀ₐ and R_{10b} are independently selected from the group consisting of hydrogen and methyl;

X₁, X₂, X₆, X₇, X₈ and X₉ are independently selected from the group consisting of hydrogen, halogen, trifluoromethyl and lower alkyl;

X₃, X₄, X₅, X₁₀, X₁₁, X₁₂, X₁₃, X₁₄, X₁₅, X₁₆, X₁₇, X₁₈, X₁₉, X₂₁, X₂₂, X₂₄, X₂₅, X₂₆, X₂₇, X₂₈, X₂₉, X₃₀, X₃₁, X₃₂, X₃₃, X₃₄, X₃₅, X₃₆, X₃₇, X₃₉, X₄₀, X₄₁, X₄₂, X₄₃, X₄₄, X₄₅, X₄₆, X₄₇, X₄₈, X₄₉, X₅₀, X₅₁, X₅₂, X₅₃, X₅₄ and X₅₅ are independently selected from the group consisting of hydrogen, hydroxy, alkoxy, amino, halogen, trifluoromethyl and lower alkyl;

X₂₀ and X₂₃ are independently selected from the group consisting of hydrogen, trifluoromethyl and lower alkyl;

X₅₆, X₅₇ and X₅₈ are independently selected from the group consisting of hydrogen and lower alkyl;

L₁, L₂, L₃ and L₄ are independently selected from the group consisting of CH and N; with the proviso that the total number of nitrogens in the ring must be 0 or 1;

L₅ and L₆ are independently selected from the group consisting of O, CR₈ₐR_{8b} and NR₉ₐ; wherein R₈ₐ and R_{8b} are independently selected from the group consisting of hydrogen and methyl; and R₉ₐ is selected from the group consisting of hydrogen, lower alkyl, formyl, acyl and sulfonyl; with the proviso that when L₆ is CR₈ₐ when a double bond is present between L₆ and CHR₅;

M₁ₐ, M_{1b}, M₂ₐ, M_{2b}, M₃, M₄, M₅, M₇, M₉, M₁₀ and M₁₂ are independently selected from the group consisting of O, S and NR_{9b} wherein R_{9b} is selected from the group consisting of hydrogen, lower alkyl, formyl, acyl and sulfonyl; and

M₆, M₈, M₁₁ and M₁₃ are independently selected from the group consisting of N and CR_{9c}, wherein R_{9c} is selected from the group consisting of hydrogen and lower alkyl.

In particular aspects of the invention, Ar of formula I is selected from:

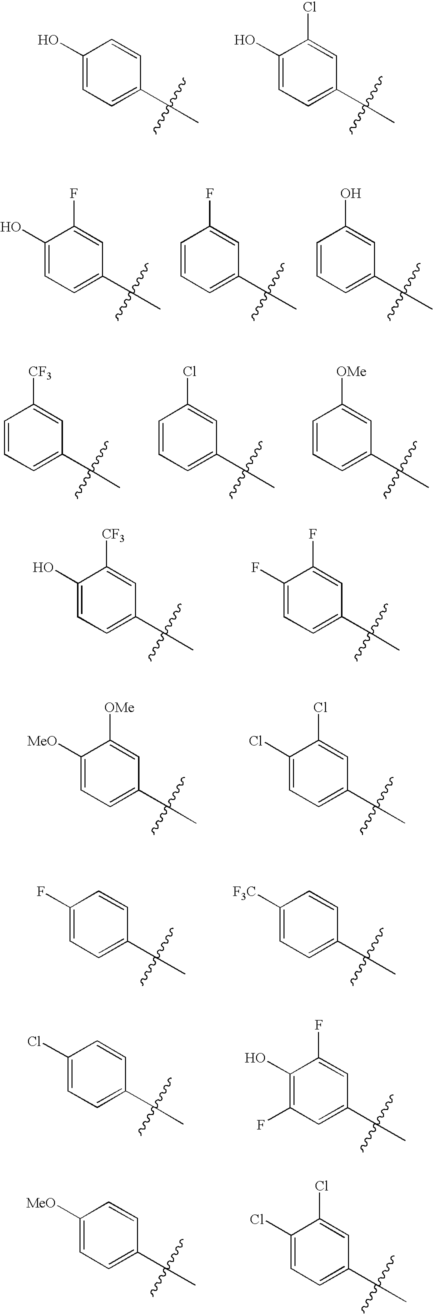

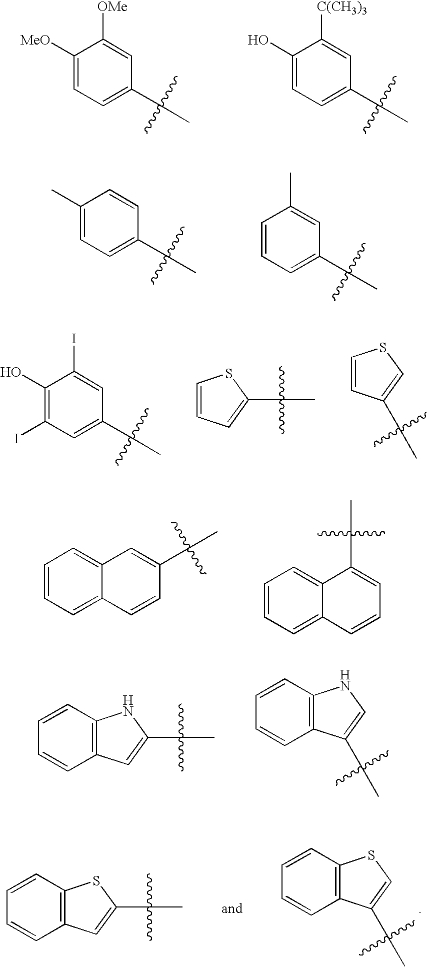

In another aspect of the invention, R₁ of formula I is selected from methyl, ethyl, isopropyl and cyclopropyl.

In other aspects of the invention, R₂ of formula I is selected from (CH₂)ₘ₁CH₃, (CH₂)ₘ₂CH(CH₃)₂, CH(CH₃)(CH₂)ₘ₃CH₃, (CH₂)ₙ₁OR₂₁, (CH₂)ₙ₂N₃, (CH₂)ₙ₃NR₂₂R₂₃, (CH₂)ₙ₄C(═NR₂₄)NR₂₅R₂₆, (CH₂)ₙ₅NR₂₇C(═NR₂₈)NR₂₉R₃₀, and (CH₂)ₙ₆NR₄₀C(═O)NR₄₁R₄₂ wherein m1 and m2 are independently 0, 1, 2, or 3; m3, n1, n2, n3, n4, n5 and n6 are independently 1, 2, 3 or 4; R₂₁ is selected from hydrogen, lower alkyl and acyl; R₂₂, R₂₅, R₂₇, R₂₉ and R₄₀ are independently selected from hydrogen, lower alkyl and sulfonyl; R₂₃, R₂₆, R₃₀, R₄₁ and R₄₂ re independently selected from hydrogen and lower alkyl; R₂₄ and R₂₈ are independently selected from hydrogen, lower alkyl, sulfonyl and cyano.

In additional aspects of the invention, R₂ of formula I is selected from:

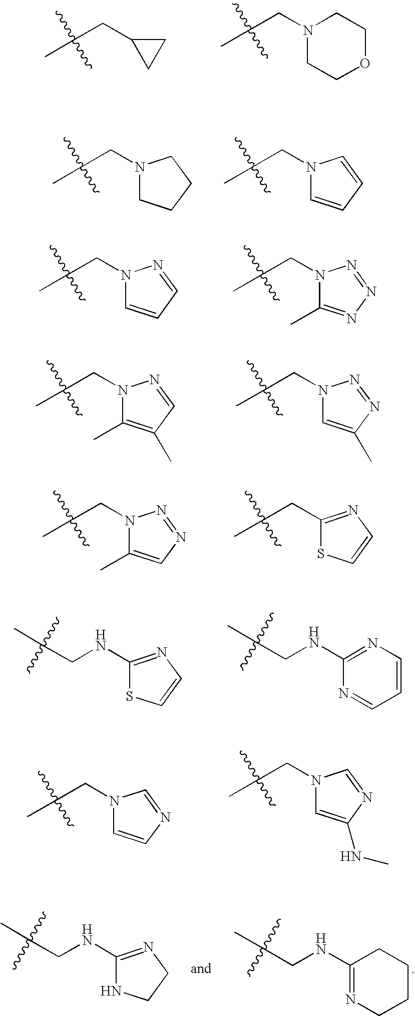

In another aspect of the invention, L₁, L₂, L₃ and L₄ in formula I are each CH, L₅ is O and L₆ is CH₂.

In other aspects of the invention, R₃, R₄, R₅ and R₆ in formula I are each hydrogen; or R₃ is methyl and R₄, R₅ and R₆ are each hydrogen; or R₃ is hydroxymethyl and R₄, R₅ and R₆ are each hydrogen; or R₃, R₄ and R₆ are each hydrogen and R₅ is methyl; or R₃ and R₅ are each methyl and R₄ and R₆ are each hydrogen.

In yet another aspect, Y in formula I is selected from the group consisting of:

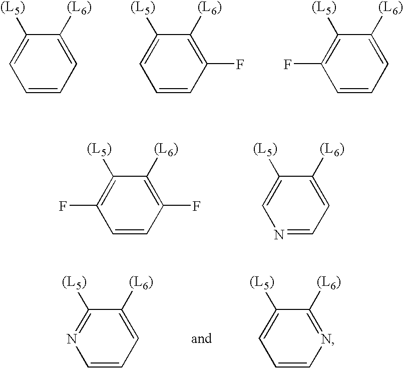

wherein (L₅) and (L₆) indicates the bond to L₅ and L₆, respectively.

Further aspects of the present invention provide methods of modulating the migrating motor complex in humans and other mammals.

Aspects of the present invention also provide methods of treating at least one disorder associated with dysfunction of the migrating motor complex in humans and other mammals, comprising administering an effective amount of a compound of formula I. In particular aspects of the present invention, the at least one disorder is characterized by hypermotility and/or absorptive disorders of the gastrointestinal tract.

The foregoing and other aspects of the present invention are explained in greater detail in the specification set forth below,

### BRIEF DESCRIPTION OF THE DRAWINGS

FIG. 1 shows the relationship between normal motilin plasma concentrations (A) and the migrating motor complex in dog (B).

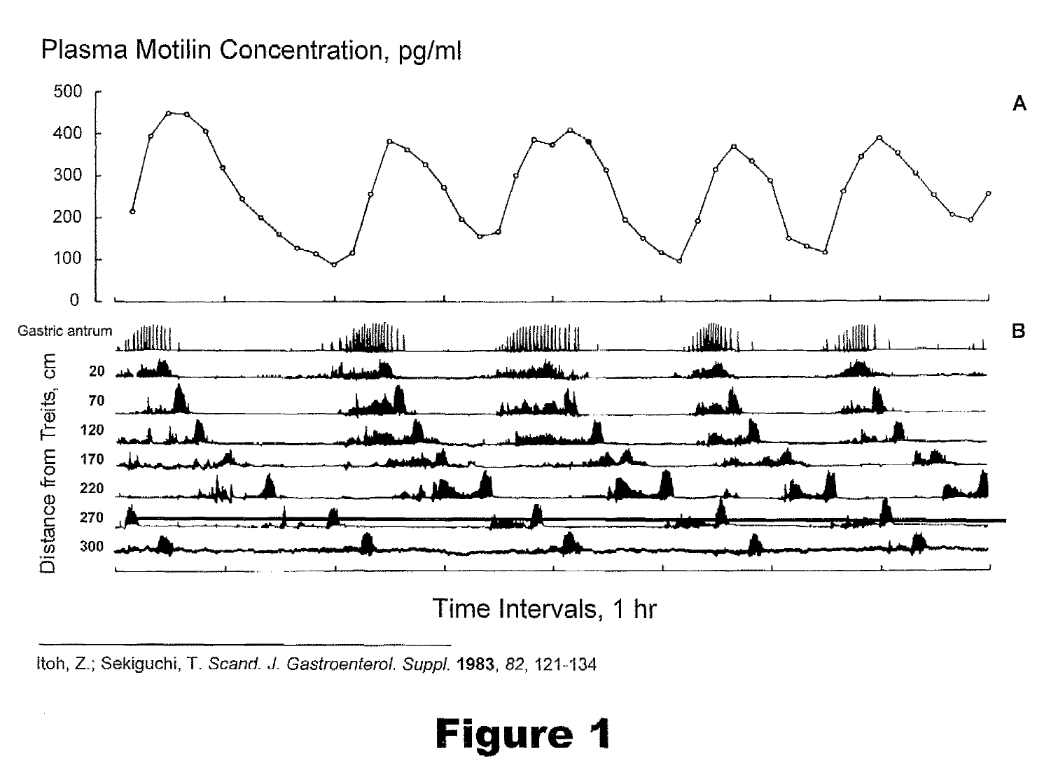

FIG. 2 shows the effects on motilin-induced increase in fundic tone in an animal model for an exemplary compound of the present invention. These graphs show that the exemplary compound (B) antagonizes motilin-induced increase in fundic tone in dog as compared to the the vehicle control (A).

FIG. 3 shows additional results of experiments characterizing the effects on motilin-induced increase in fundic tone in an animal model for an exemplary compound of the present invention (cross-over design, N=6 dogs). The mean data (A) and the individual AUC data of drug vs. vehicle (B) provide farther illustration of the ability of the exemplary compound to antagonize motilin-induced increase in fundic tone in dog.

FIG. 4 shows the effects on the motilin-induced migrating motor complex (MMC) in an animal model for an exemplary compound of the present invention. These graphs show that the exemplary compound (B) antagonizes motilin-induced migrating motor complex in dog as compared to the vehicle control (A).

FIG. 5 shows the effects on the natural MMC in an animal model for an exemplary compound of the present invention. (A) Straight gauge tracing of MMC. (B) Difference in contraction amplitude in the presence of the exemplary compound vs. vehicle control. (C) MMC interval time before and after treatment. These graphs show that the exemplary compound blocks natural migrating motor complex in conscious fasted dogs.

FIG. 6 shows the effects on postprandial gastrointestinal activity in an animal model by an exemplary compound of the present invention. These data show that the exemplary compound (B), compared to the vehicle control (A), decreases postprandial gastrointestinal activity in dogs.

FIG. 7 shows additional results of experiments characterizing the dose-dependent effects on postprandial gastrointestinal activity in an animal model by an exemplary compound of the present invention. (A) Difference in contraction amplitude in the presence of the exemplary compound vs. vehicle control. (B) Interval between contractions pre- and post-treatment with the exemplary compound. These graphs provide further illustration of the ability of the exemplary compound to bring about a dose-dependent decrease in postprandial gastrointestinal activity in dogs.

### DETAILED DESCRIPTION

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. Additionally, as used herein, the term “and/or” includes any and all combinations of one or more of the associated listed items and may be abbreviated as “/”.

Unless otherwise defined, all technical and scientific terms used herein have the same meaning as commonly understood by one of ordinary skill in the art to which this invention belongs.

All publications, U.S. patent applications, U.S. patents and other references cited herein are incorporated by reference in their entireties.

The term “alkyl” refers to straight or branched chain saturated or partially unsaturated hydrocarbon groups having from 1 to 20 carbon atoms, in some instances 1 to 8 carbon atoms. The term “lower alkyl” refers to alkyl groups containing 1 to 6 carbon atoms. Examples of alkyl groups include, but are not limited to, methyl, ethyl, isopropyl, tert-butyl, 3-hexenyl, and 2-butynyl. By “unsaturated” is meant the presence of 1, 2 or 3 double or triple bonds, or a combination of the two. Such alkyl groups may also be optionally substituted as described below.

When a subscript is used with reference to an alkyl or other hydrocarbon group defined herein, the subscript refers to the number of carbon atoms that the group may contain. For example, C₂-C₄ alkyl indicates an alkyl group with 2, 3 or 4 carbon atoms.

The term “cycloalkyl” refers to saturated or partially unsaturated cyclic hydrocarbon groups having from 3 to 15 carbon atoms in the ring, in some instances 3 to 7, and to alkyl groups containing said cyclic hydrocarbon groups. Examples of cycloalkyl groups include, but are not limited to, cyclopropyl, cyclopropylmethyl, cyclopentyl, 2-(cyclohexyl)ethyl, cycloheptyl, and cyclohexenyl. Cycloalkyl as defined herein also includes groups with multiple carbon rings, each of which may be saturated or partially unsaturated, for example decalinyl, [2.2.1]-bicycloheptanyl or adamantanyl. All such cycloalkyl groups may also be optionally substituted as described below.

The term “aromatic” refers to an unsaturated cyclic hydrocarbon group having a conjugated pi electron system that contains 4n+2 electrons where n is an integer greater than or equal to 1. Aromatic molecules are typically stable and are depicted as a planar ring of atoms with resonance structures that consist of alternating double and single bonds, for example benzene or naphthalene.

The term “aryl” refers to an aromatic group in a single or fused carbocyclic ring system having from 6 to 15 ring atoms, in some instances 6 to 10, and to alkyl groups containing said aromatic groups. Examples of aryl groups include, but are not limited to, phenyl, 1-naphthyl, 2-naphthyl and benzyl. Aryl as defined herein also includes groups with multiple aryl rings which may be fused, as in naphthyl and anthracenyl, or unfused, as in biphenyl and terphenyl. Aryl also refers to bicyclic or tricyclic carbon rings, where one of the rings is aromatic and the others of which may be saturated, partially unsaturated or aromatic, for example, indanyl or tetrahydronaphthyl (tetralinyl). All such aryl groups may also be optionally substituted as described below.

The term “heterocycle” or “heterocyclic” refers to saturated or partially unsaturated monocyclic, bicyclic or tricyclic groups having from 3 to 15 atoms, in some instances 3 to 7, with at least one heteroatom in at least one of the rings, said heteroatom being selected from O, S or N. Each ring of the heterocyclic group can contain one or two O atoms, one or two S atoms, one to four N atoms, provided that the total number of heteroatoms in each ring is four or less and each ring contains at least one carbon atom. The fused rings completing the bicyclic or tricyclic heterocyclic groups may contain only carbon atoms and may be saturated or partially unsaturated. The N and S atoms may optionally be oxidized and the N atoms may optionally be quaternized. Heterocyclic also refers to alkyl groups containing said monocyclic, bicyclic or tricyclic heterocyclic groups. Examples of heterocyclic rings include, but are not limited to, 2- or 3-piperidinyl, 2- or 3-piperazinyl, 2- or 3-morpholinyl. All such heterocyclic groups may also be optionally substituted as described below

The term “heteroaryl” refers to an aromatic group in a single or fused ring system having from 5 to 15 ring atoms, in some instances 5 to 10, which have at least one heteroatom in at least one of the rings, said heteroatom being selected from O, S or N. Each ring of the heteroaryl group can contain one or two O atoms, one or two S atoms, one to four N atoms, provided that the total number of heteroatoms in each ring is four or less and each ring contains at least one carbon atom. The fused rings completing the bicyclic or tricyclic groups may contain only carbon atoms and may be saturated, partially unsaturated or aromatic. In structures where the lone pair of electrons of a nitrogen atom is not involved in completing the aromatic pi electron system, the N atoms may optionally be quaternized or oxidized to the N-oxide. Heteroaryl also refers to alkyl groups containing said cyclic groups. Examples of monocyclic heteroaryl groups include, but are not limited to pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, and triazinyl. Examples of bicyclic heteroaryl groups include, but are not limited to indolyl, benzothiazolyl, benzoxazolyl, benzothienyl, quinolinyl, tetrahydroisoquinolinyl, isoquinolinyl, benzimidazolyl, indolizinyl, benzofuranyl, isobenzofuranyl, chromonyl, coumarinyl, benzopyranyl, cinnolinyl, quinoxalinyl, indazolyl, purinyl, pyrrolopyridinyl, furopyridinyl, thienopyridinyl, dihydroisoindolyl, and tetrahydroquinolinyl. Examples of tricyclic heteroaryl groups include, but are not limited to carbazolyl, benzindolyl, phenanthrollinyl, acridinyl, phenanthridinyl, and xanthenyl. All such heteroaryl groups may also be optionally substituted as described below.

The term “hydroxy” refers to the group —OH.

The term “alkoxy” refers to the group —ORₐ, wherein Rₐ is alkyl, cycloalkyl or heterocyclic. Examples include, but are not limited to methoxy, ethoxy, tert-butoxy, cyclohexyloxy and tetrahydropyranyloxy.

The term “aryloxy” refers to the group —OR_{b} wherein R_{b} is aryl or heteroaryl. Examples include, but are not limited to phenoxy, benzyloxy and 2-naphthyloxy.

The term “acyl” refers to the group —C(═O)—R_{c} wherein R_{c} is alkyl, cycloalkyl, heterocyclic, aryl or heteroaryl. Examples include, but are not limited to, acetyl, benzoyl and furoyl.

The term “amino acyl” indicates an acyl group that is derived from an amino acid.

The term “amino” refers to an —NR_{d}Rₑ group wherein R_{d} and Rₑ are independently selected from the group consisting of hydrogen, alkyl, cycloalkyl, heterocyclic, aryl and heteroaryl. Alternatively, R_{d} and Rₑ together form a heterocyclic ring of 3 to 8 members, optionally substituted with unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N.

The term “amido” refers to the group —C(═O)—NR_{f}R_{g} wherein R_{f} and R_{g} are independently selected from the group consisting of hydrogen, alkyl, cycloalkyl, heterocyclic, aryl and heteroaryl. Alternatively, R_{f} and R_{g} together form a heterocyclic ring of 3 to 8 members, optionally substituted with unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N.

The term “amidino” refers to the group —C(═NRₕ)NRᵢRⱼ wherein Rₕ is selected from the group consisting of hydrogen, cyano, alkyl, cycloalkyl, heterocyclic, aryl and heteroaryl; and Rᵢ and Rⱼ are independently selected from the group consisting of hydrogen, alkyl, cycloalkyl, heterocyclic, aryl and heteroaryl. Alternatively, Rᵢ and Rⱼ together form a heterocyclic ring of 3 to 8 members, optionally substituted with unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N.

The term “azido” refers to the group —N₃.

The term “carboxy” refers to the group —CO₂H.

The term “carboxyalkyl” refers to the group —CO₂Rₖ, wherein Rₖ is alkyl, cycloalkyl or heterocyclic.

The term “carboxyaryl” refers to the group —CO₂Rₘ, wherein Rₘ is aryl or heteroaryl.

The term “cyano” refers to the group —CN.

The term “formyl” refers to the group —C(═O)H, also denoted —CHO.

The term “halo,” “halogen” or “halide” refers to fluoro, fluorine or fluoride, chloro, chlorine or chloride, bromo, bromine or bromide, and iodo, iodine or iodide, respectively.

The term “oxo” refers to the bivalent group ═O, which is substituted in place of two hydrogen atoms on the same carbon to form a carbonyl group.

The term “mercapto” refers to the group —SRₙ wherein Rₙ is hydrogen, alkyl, cycloalkyl, heterocyclic, aryl or heteroaryl.

The term “nitro” refers to the group —NO₂.

The term “trifluoromethyl” refers to the group —CF₃.

The term “sulfinyl” refers to the group —S(═O)—Rₚ wherein Rₚ is alkyl, cycloalkyl, heterocyclic, aryl or heteroaryl.

The term “sulfonyl” refers to the group —S(O)₂—R_{q1} wherein R_{q1} is alkyl, cycloalkyl, heterocyclic, aryl or heteroaryl.

The term “aminosulfonyl” refers to the group —NR_{q2}—S(═O)₂—R_{q3} wherein R_{q2} is hydrogen, alkyl, cycloalkyl, heterocyclic, aryl or heteroaryl; and R_{q3} is alkyl, cycloalkyl, heterocyclic, aryl or heteroaryl.

The term “sulfonamido” refers to the group —S(═O)₂—NRᵣRₛ wherein Rᵣ and Rₛ are independently selected from the group consisting of hydrogen, alkyl, cycloalkyl, heterocyclic, aryl or heteroaryl. Alternatively, Rᵣ and Rₛ together form a heterocyclic ring of 3 to 8 members, optionally substituted with unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N.

The term “carbamoyl” refers to a group of the formula —N(Rₜ)—C(═O)—ORᵤ wherein Rₜ is selected from hydrogen, alkyl, cycloalkyl, heterocyclic, aryl or heteroaryl; and Rᵤ is selected from alkyl, cycloalkyl, heterocyclic, aryl or heteroaryl.

The term “guanidino” refers to a group of the formula —N(Rᵥ)—C(═NR_{w})—NRₓR_{y} wherein Rᵥ, R_{w}, Rₓ and R_{y} are independently selected from hydrogen, alkyl, cycloalkyl, heterocyclic, aryl or heteroaryl. Alternatively, Rₓ and R_{y} together form a heterocyclic ring or 3 to 8 members, optionally substituted with unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N.

The term “ureido” refers to a group of the formula —N(R_{z})—C(═O)—NRₐₐR_{bb} wherein R_{z}, Rₐₐ and R_{bb} are independently selected from hydrogen, alkyl, cycloalkyl, heterocyclic, aryl or heteroaryl. Alternatively, Rₐₐ and R_{bb} together form a heterocyclic ring of 3 to 8 members, optionally substituted with unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N.

The term “optionally substituted” is intended to expressly indicate that the specified group is unsubstituted or substituted by one or more suitable substituents, unless the optional substituents are expressly specified, in which case the term indicates that the group is unsubstituted or substituted with the specified substituents. As defined above, various groups may be unsubstituted or substituted (i.e., they are optionally substituted) unless indicated otherwise herein (e.g., by indicating that the specified group is unsubstituted).

The term “substituted” when used with the terms alkyl, cycloalkyl, heterocyclic, aryl and heteroaryl refers to an alkyl, cycloalkyl, heterocyclic, aryl or heteroaryl group having one or more of the hydrogen atoms of the group replaced by substituents independently selected from unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, halo, oxo, mercapto, sulfinyl, sulfonyl, sulfonamido, amidino, carbamoyl, guanidino, ureido and groups of the formulas —NR_{cc}C(═O)R_{dd}, —NRₑₑC(═NR_{ff})R_{gg}, —OC(═O)NRₕₕRᵢᵢ, —OC(═O)Rⱼⱼ, —OC(═O)ORₖₖ, —NRₘₘSO₂Rₙₙ, or —NRₚₚSO₂NR₁₁Rᵣᵣ wherein R_{cc}, R_{dd}, Rₑₑ, R_{ff}, R_{gg}, Rₕₕ, Rᵢᵢ, Rⱼⱼ, Rₘₘ, Rₚₚ, R_{qq} and Rᵣᵣ are independently selected from hydrogen, unsubstituted alkyl, unsubstituted cycloalkyl, unsubstituted heterocyclic, unsubstituted aryl or unsubstituted heteroaryl; and wherein Rₖₖ and Rₙₙ are independently selected from unsubstituted alkyl, unsubstituted cycloalkyl, unsubstituted heterocyclic, unsubstituted aryl or unsubstituted heteroaryl. Alternatively, R_{gg} and Rₕₕ, Rⱼⱼ and Rₖₖ or Rₚₚ and R_{qq} together form a heterocyclic ring of 3 to 8 members, optionally substituted with unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N. In addition, the term “substituted” for aryl and heteroaryl groups includes as an option having one of the hydrogen atoms of the group replaced by cyano, nitro or trifluoromethyl.

A substitution is made provided that any atom's normal valency is not exceeded and that the substitution results in a stable compound. Generally, when a substituted form of a group is present, such substituted group is preferably not further substituted or, if substituted, the substituent comprises only a limited number of substituted groups, in some instances 1, 2, 3 or 4 such substituents.

When any variable occurs more than one time in any constituent or in any formula herein, its definition on each occurrence is independent of its definition at every other occurrence. Also, combinations of substituents and/or variables are permissible only if such combinations result in stable compounds.

A “stable compound” or “stable structure” refers to a compound that is sufficiently robust to survive isolation to a useful degree of purity and formulation into an efficacious therapeutic agent.

The term “amino acid” refers to the common natural (genetically encoded) or synthetic amino acids and common derivatives thereof, known to those skilled in the art. When applied to amino acids, “standard” or “proteinogenic” refers to the genetically encoded 20 amino acids in their natural configuration. Similarly, when applied to amino acids, “unnatural” or “unusual” refers to the wide selection of non-natural, rare or synthetic amino acids such as those described by Hunt, S. in Chemistry and Biochemistry of the Amino Acids, Barrett, G. C., Ed., Chapman and Hall: New York, 1985.

The term “residue” with reference to an amino acid or amino acid derivative refers to a group of the formula:

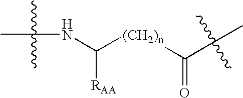

wherein R_{AA} is an amino acid side chain, and n=0, 1 or 2 in this instance.

The term “fragment” with respect to a dipeptide, tripeptide or higher order peptide derivative indicates a group that contains two, three or more, respectively, amino acid residues.

The term “amino acid side chain” refers to any side chain from a standard or unnatural amino acid, and is denoted R_{AA}. For example, the side chain of alanine is methyl, the side chain of valine is isopropyl and the side chain of tryptophan is 3-indolylmethyl.

The term “agonist” refers to a compound that duplicates at least some of the effect of the endogenous ligand of a protein, receptor, enzyme or the like.

The term “antagonist” refers to a compound that inhibits at least some of the effect of the endogenous ligand of a protein, receptor, enzyme or the like.

The term “modulator” refers to a compound that imparts an effect on a biological or chemical process or mechanism. For example, a modulator may increase, facilitate, upregulate, activate, inhibit, decrease, block, prevent, delay, desensitize, deactivate, down regulate, or the like, a biological or chemical process or mechanism. Accordingly, a modulator can be an “agonist” or an “antagonist.” Exemplary biological processes or mechanisms affected by a modulator include, but are not limited to, receptor binding and hormone release or secretion. Exemplary chemical processes or mechanisms affected by a modulator include, but are not limited to, catalysis and hydrolysis.

The term “peptide” refers to a chemical compound comprised of two or more amino acids covalently bonded together.

The term “peptidomimetic” refers to a chemical compound designed to mimic a peptide, but which contains structural differences through the addition or replacement of one of more functional groups of the peptide in order to modulate its activity or other properties, such as solubility, metabolic stability, oral bioavailability, lipophilicity, permeability, etc. This can include replacement of the peptide bond, side chain modifications, truncations, additions of functional groups, etc. When the chemical structure is not derived from the peptide, but mimics its activity, it is often referred to as a “non-peptide peptidomimetic.”

The term “peptide bond” refers to the amide [—C(═O)—NH—] functionality with which individual amino acids are typically covalently bonded to each other in a peptide.

The term “protecting group” refers to any chemical compound that may be used to prevent a potentially reactive functional group, such as an amine, a hydroxyl or a carboxyl, on a molecule from undergoing a chemical reaction while chemical change occurs elsewhere in the molecule. A number of such protecting groups are known to those skilled in the art and examples can be found in “Protective Groups in Organic Synthesis,” Theodora W. Greene and Peter G. Wuts, editors, John Wiley & Sons, New York, 3^{rd} edition, 1999 [ISBN 0471160199]. Examples of amino protecting groups include, but are not limited to, phthalimido, trichloroacetyl, benzyloxycarbonyl, tert-butoxycarbonyl, and adamantyloxycarbonyl. In some embodiments, amino protecting groups are carbamate amino protecting groups, which are defined as an amino protecting group that when bound to an amino group forms a carbamate. In other embodiments, amino carbamate protecting groups are allyloxycarbonyl (Alloc), benzyloxycarbonyl (Cbz), 9-fluorenylmethoxycarbonyl (Fmoc), tert-butoxycarbonyl (Boc) or α,α-dimethyl-3,5-dimethoxybenzyloxycarbonyl (Ddz). For a recent discussion of newer nitrogen protecting groups: Theodoridis, G. Tetrahedron 2000, 56, 2339-2358. Examples of hydroxyl protecting groups include, but are not limited to, acetyl, tert-butyldimethylsilyl (TBDMS), trityl (Trt), tert-butyl, and tetrahydropyranyl (THP). Examples of carboxyl protecting groups include, but are not limited to methyl ester, tert-butyl ester, benzyl ester, trimethylsilylethyl ester, and 2,2,2-trichloroethyl ester.

The term “solid phase chemistry” refers to the conduct of chemical reactions where one component of the reaction is covalently bonded to a polymeric material (solid support as defined below). Reaction methods for performing chemistry on solid phase have become more widely known and established outside the traditional fields of peptide and oligonucleotide chemistry.

The term “solid support,” “solid phase” or “resin” refers to a mechanically and chemically stable polymeric matrix utilized to conduct solid phase chemistry. This is denoted by “Resin,” “P—” or the symbol at the right:

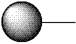

Examples of appropriate polymer materials include, but are not limited to, polystyrene, polyethylene, polyethylene glycol, polyethylene glycol grafted or covalently bonded to polystyrene (also termed PEG-polystyrene, TentaGel™, Rapp, W.; Zhang, L.; Bayer, E. In Innovations and Perspectives in Solid Phase Synthesis. Peptides, Polypeptides and Oligonucleotides; Epton, R., Ed.; SPCC Ltd.: Birmingham, UK; p 205), polyacrylate (CLEAR™), polyacrylamide, polyurethane, PEGA [polyethyleneglycol poly(N,N-dimethylacrylamide) co-polymer, Meldal, M. Tetrahedron Lett. 1992, 33, 3077-3080], cellulose, etc. These materials can optionally contain additional chemical agents to form cross-linked bonds to mechanically stabilize the structure, for example polystyrene cross-linked with divinylbenezene (DVB, usually 0.1-5%, preferably 0.5-2%). This solid support can include as non-limiting examples aminomethyl polystyrene, hydroxymetlhyl polystyrene, benzhydrylamine polystyrene (BHA), methylbenzhydrylamine (MBHA) polystyrene, and other polymeric backbones containing free chemical functional groups, most typically, —NH₂ or —OH, for further derivatization or reaction. The term is also meant to include “Ultraresins” with a high proportion (“loading”) of these functional groups such as those prepared from polyethyleneimines and cross-linking molecules (Barth, M.; Rademann, J. J. Comb. Chem. 2004, 6, 340-349). At the conclusion of the synthesis, resins are typically discarded, although they have been shown to be able to be reused such as in Frechet, J. M. J.; Haque, K. E. Tetrahedron Lett. 1975, 16, 3055.

In general, the materials used as resins are insoluble polymers, but certain polymers have differential solubility depending on solvent and can also be employed for solid phase chemistry. For example, polyethylene glycol can be utilized in this manner since it is soluble in many organic solvents in which chemical reactions can be conducted, but it is insoluble in others, such as diethyl ether. Hence, reactions can be conducted homogeneously in solution, then the product on the polymer precipitated through the addition of diethyl ether and processed as a solid. This has been termed “liquid-phase” chemistry.

The term “linker” when used in reference to solid phase chemistry refers to a chemical group that is bonded covalently to a solid support and is attached between the support and the substrate typically in order to permit the release (cleavage) of the substrate from the solid support. However, it can also be used to impart stability to the bond to the solid support or merely as a spacer element. Many solid supports are available commercially with linkers already attached.

Abbreviations used for amino acids and designation of peptides follow the rules of the IUPAC-IUB Commission of Biochemical Nomenclature in J. Biol. Chem. 1972, 247, 977-983. This document has been updated. Biochem. J., 1984, 219, 345-373; Eur. J. Biochem., 1984, 138, 9-37; 1985, 152, 1; Internat. J. Pept. Prof. Res., 1984, 24, following p 84; J. Biol. Chem., 1985, 260, 14-42; Pure Appl. Chem., 1984, 56, 595-624; Amino Acids and Peptides, 1985, 16, 387-410; and in Biochemical Nomenclature and Related Documents, 2nd edition, Portland Press, 1992, pp 39-67. Extensions to the rules were published in the JCBN/NC-IUB Newsletter 1985, 1986, 1989; see Biochemical Nomenclature and Related Documents, 2nd edition, Portland Press, 1992, pp 68-69.

The term “effective amount” or “effective” is intended to designate a dose that causes a relief of symptoms of a disease or disorder as noted through clinical testing and evaluation, patient observation, and/or the like, and/or a dose that causes a detectable change in biological or chemical activity. The detectable changes may be detected and/or further quantified by one skilled in the art for the relevant mechanism or process. As is generally understood in the art, the dosage will vary depending on the administration routes, symptoms and body weight of the patient but also depending upon the compound being administered.

Administration of two or more compounds “in combination” means that the two compounds are administered closely enough in time that the presence of one alters the biological effects of the other. The two compounds can be administered simultaneously (concurrently) or sequentially. Simultaneous administration can be carried out by mixing the compounds prior to administration, or by administering the compounds at the same point in time but at different anatomic sites or using different routes of administration. The phrases “concurrent administration”, “administration in combination”, “simultaneous administration” or “administered simultaneously” as used herein, means that the compounds are administered at the same point in time or immediately following one another. In the latter case, the two compounds are administered at times sufficiently close that the results observed are indistinguishable from those achieved when the compounds are administered at the same point in time.

The term “pharmaceutically active metabolite” is intended to mean a pharmacologically active product produced through metabolism in the body of a specified compound.

The term “hydrate”, as understood by one skilled in the art, is intended to mean a pharmaceutically acceptable hydrate form of a specified compound that retains the biological effectiveness of such compound. A hydrate form of a solid compound has water in the form of H₂O molecules associated with it in a definite amount. When the solid is crystalline, the H₂O molecules can be an integral part of the crystal structure of the solid. The H₂O molecules can also be present in definite proportions in the solid or crystal without being associated directly with the components.

The term “solvate”, as understood by one skilled in the art, is intended to mean a pharmaceutically acceptable solvate form of a specified compound that retains the biological effectiveness of such compound. A solvate form of a solid compound has solvent molecules associated with it in a definite amount. When the solid is crystalline, the solvent molecules can be an integral part of the crystal stricture of the solid. The solvent molecules can also be present in definite proportions in the solid or crystal without being associated directly with the components. Examples of solvates, without limitation, include compounds of the invention in combination with isopropanol, ethanol, methanol, DMSO, ethyl acetate, acetic acid, dioxane, tetrahydrofuran or ethanolamine. The formation of hydrates and solvates of the present invention is clearly within the knowledge of one skilled in the art without the necessity of undue experimentation.

### 1. Compounds

Novel macrocyclic compounds of the present invention include macrocyclic compounds comprising a building block structure including a tether component that undergoes cyclization to form the macrocyclic compound. The building block structure can comprise amino acids, including standard α-amino acids, and a tether component as described herein. The tether component can be selected from compounds that result in the following structures:

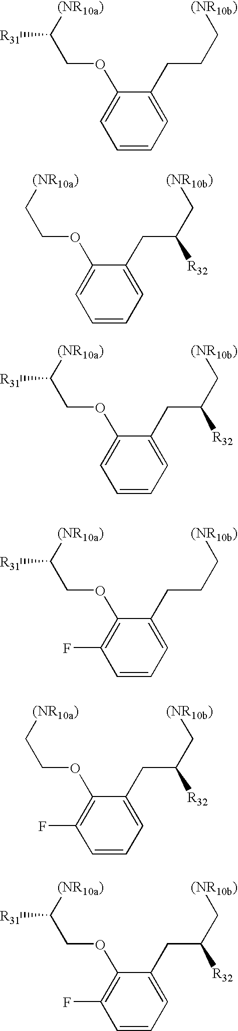

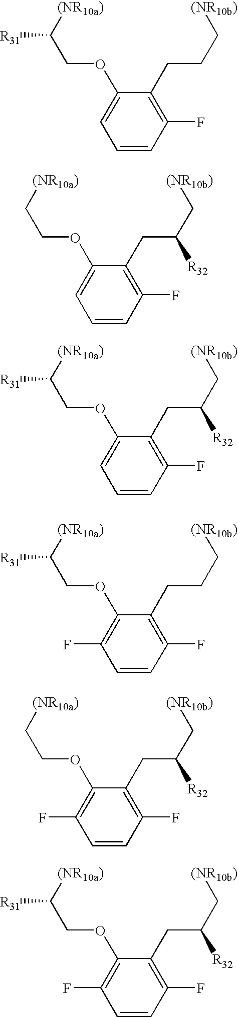

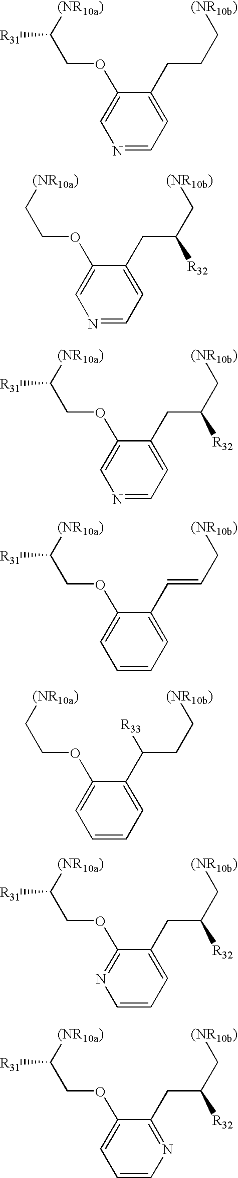

wherein (NR₁₀ₐ) indicates the bond to the nitrogen atom of NR₁₀ₐ in formula I; (NR_{10b}) indicates the bond to the nitrogen atom of NR_{10b} in formula I; R₃₁ is selected from hydrogen, methyl, ethyl, isopropyl and hydroxymethyl; and R₃₂ and R₃₃ are selected from hydrogen, methyl and hydroxy.

Macrocyclic compounds of the present invention further include those of formula I:

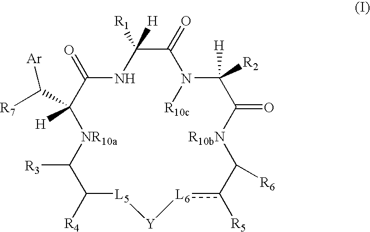

or pharmaceutically acceptable salts, hydrates or solvates thereof, wherein:

Y is

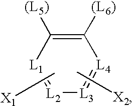

wherein (L₅) and (L₆) indicate the bonds to L₅ and L₆ of formula I, respectively;

Ar is selected from the group consisting of:

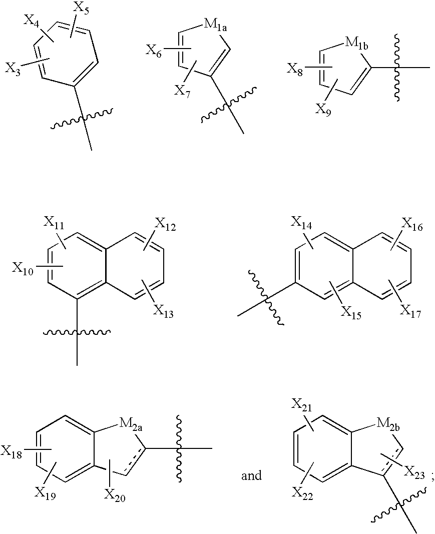

R₁ is selected from the group consisting of: —(CH₂)ₛCH₃, —CH(CH₃)(CH₂)ₜCH₃, —(CH₂)ᵤCH(CH₃)₂, —C(CH₃)₃, and

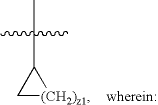

s is 0, 1, 2 or 3;

t is 1 or 2;

u is 0 or 1; and

z1 is 1, 2, 3 or 4;

R₂ is selected from the group consisting of hydrogen, —(CH₂)ₐₐCH₃, —CH₂SCH₃, —CH₂CH₂SCH₃, —(CH₂)_{bb}CH(CH₃)₂, —CH(CH₃)(CH₂)_{cc}CH₃, —(CH₂)_{dd}—NR₁₁R₁₂, and —(CH₂)_{dd}R₁₃; wherein

aa and bb are independently 0, 1, 2 or 3;

cc and dd are independently 1, 2, 3 or 4;

ee is 0, 1, 2, 3 or 4;

R₁₁ is selected from the group consisting of hydrogen, lower alkyl, formyl, acyl, carboxyalkyl, carboxyaryl, amido, amidino, sulfonyl and sulfonamido;

R₁₂ is selected from the group consisting of hydrogen and lower alkyl;

R₁₃ is selected from the group consisting of;

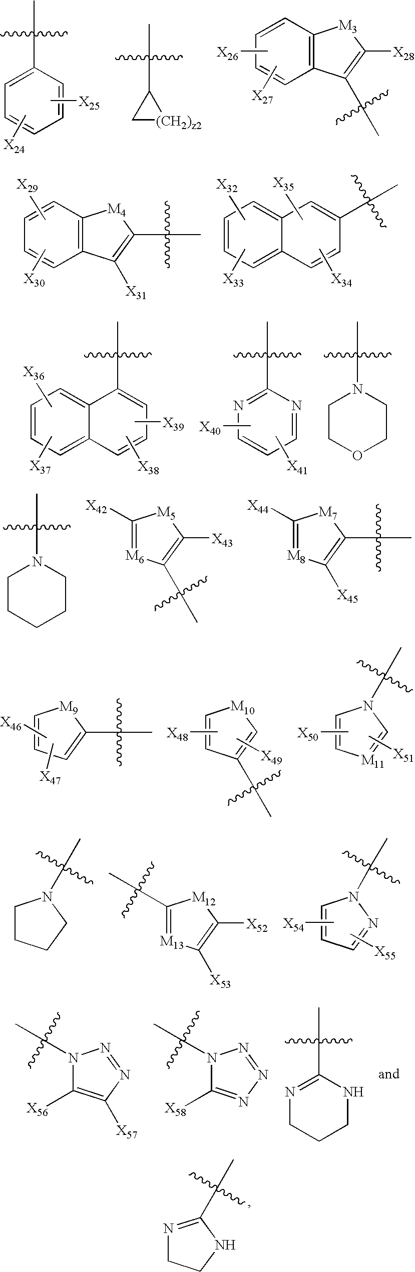

wherein z2 is 1, 2, 3 or 4;

and, when ee is 1, 2, 3 or 4, R₁₃ is further selected from the group consisting of hydroxy, alkoxy, amidino, and azido;

R₃, R₄, R₅, and R₆ are independently selected from the group consisting of hydrogen, methyl, ethyl, isopropyl and hydroxymethyl;

R₇ is selected from the group consisting of hydrogen, methyl, hydroxy and amino;

R₁₀ₐ and R_{10b} are independently selected from the group consisting of hydrogen and methyl;

X₁, X₂, X₆, X₇, X₈ and X₉ are independently selected from the group consisting of hydrogen, halogen, trifluoromethyl and lower alkyl;

X₃, X₄, X₅, X₁₀, X₁₁, X₁₂, X₁₃, X₁₄, X₁₅, X₁₆, X₁₇, X₁₈, X₁₉, X₂₁, X₂₂, X₂₄, X₂₅, X₂₆, X₂₇, X₂₈, X₂₉, X₃₀, X₃₁, X₃₂, X₃₃, X³⁴, X₃₅, X₃₆, X₃₇, X₃₈, X₃₉, X₄₀, X₄₁, X₄₂, X₄₃, X₄₄, X₄₅, X₄₆, X₄₇, X₄₈, X₄₉, X₅₀, X₅₁, X₅₂, X₅₃, X₅₄ and X₅₅ are independently selected from the group consisting of hydrogen, hydroxy, alkoxy, amino, halogen, trifluoromethyl and lower alkyl;

X₂₀ and X₂₃ are independently selected from the group consisting of hydrogen, trifluoromethyl and lower alkyl;

X₅₆, X₅₇ and X₅₈ are independently selected from the group consisting of hydrogen and lower alkyl;

L₁, L₂, L₃ and L₄ are independently selected from the group consisting of CH and N; with the proviso that the total number of nitrogens in the ring must be 0 or 1;

L₅ and L₆ are independently selected from the group consisting of O, CR₈ₐR_{8b} and NR₉ₐ; wherein R₈ₐ and R_{8b} are independently selected from the group consisting of hydrogen and methyl; and R₉ₐ is selected from the group consisting of hydrogen, lower alkyl, formyl, acyl and sulfonyl; with the proviso that when L₆ is CR₈ₐ when a double bond is present between L₆ and CHR₅;

M₁ₐ, M_{1b}, M₂ₐ, M_{2b}, M₃, M₄, M₅, M₇, M₉, M₁₀ and M₁₂ are independently selected from the group consisting of O, S and NR_{9b} wherein R_{9b} is selected from the group consisting of hydrogen, lower alkyl, formyl, acyl and sulfonyl; and

M₆, M₈, M₁₁ and M₁₃ are independently selected from the group consisting of N and CR_{9c}, wherein R_{9c} is selected from the group consisting of hydrogen and lower alkyl.

The present invention includes isolated compounds. An isolated compound refers to a compound that, in some embodiments, comprises at least 10%, at least 25%, at least 50% or at least 70% of the compounds of a mixture. In some embodiments, the compound, plhamiaceutically acceptable salt thereof or pharmaceutical composition containing the compound exhibits a statistically significant binding and/or antagonist activity when tested in biological assays at the human motilin receptor.

In the case of compounds, salts, hydrates or solvates that are solids, it is understood by those skilled in the art that the inventive compounds, salts, hydrates and solvates may exist in different crystal or polymorphic forms, all of which are intended to be within the scope of the present invention and specified formulas.

The compounds of formula I disclosed herein have asymmetric centers. The inventive compounds may exist as single stereoisomers, racemates, and/or mixtures of enantiomers and/or diastereomers. All such single stereoisomers, racemates, and mixtures thereof are intended to be within the scope of the present invention. In particular embodiments, however, the inventive compounds are used in optically pure form. The terms “S” and “R” configuration as used herein are as defined by the IUPAC 1974 Recommendations for Section E, Fundamentals of Stereochemistry (Pure Appl. Chem. 1976, 45, 13-30.)

Unless otherwise depicted to be a specific orientation, the present invention accounts for all stereoisomeric forms. The compounds may be prepared as a single stereoisomer or a mixture of stereoisomers. The non-racemic forms may be obtained by either synthesis or resolution. The compounds may, for example, be resolved into the component enantiomers by standard techniques, for example formation of diastereomeric pairs via salt formation. The compounds also may be resolved by covalently bonding to a chiral moiety. The diastereomers can then be resolved by chromatographic separation and/or crystallographic separation. In the case of a chiral auxiliary moiety, it can then be removed. As an alternative, the compounds can be resolved through the use of chiral chromatography. Enzymatic methods of resolution could also be used in certain cases.

As generally understood by those skilled in the art, an “optically pure” compound is one that contains only a single enantiomer. As used herein, the term “optically active” is intended to mean a compound comprising at least a sufficient excess of one enantiomer over the other such that the mixture rotates plane polarized light. Optically active compounds have the ability to rotate the plane of polarized light. The excess of one enantiomer over another is typically expressed as enantiomeric excess (e.e.). In describing an optically active compound, the prefixes D and L or R and S are used to denote the absolute configuration of the molecule about its chiral center(s). The prefixes “d” and “l” or (+) and (−) are used to denote the optical rotation of the compound (i.e., the direction in which a plane of polarized light is rotated by the optically active compound). The “l” or (−) prefix indicates that the compound is levorotatory (i.e., rotates the plane of polarized light to the left or counterclockwise) while the “d” or (+) prefix means that the compound is dextrarotatory (i.e., rotates the plane of polarized light to the right or clockwise). The sign of optical rotation, (−) and (+), is not related to the absolute configuration of the molecule, R and S.

A compound of the invention having the desired pharmacological properties will be optically active and, can be comprised of at least 90% (80% e.e.), at least 95% (90% e.e.), at least 97.5% (95% e.e.) or at least 99% (98% e.e.) of a single isomer.

Likewise, many geometric isomers of double bonds and the like can also be present in the compounds disclosed herein, and all such stable isomers are included within the present invention unless otherwise specified. Also included in the invention are tautomers and rotamers of formula I.

The use of the following symbols at the right refers to

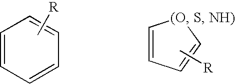

substitution of one or more hydrogen atoms of the indicated ring with the defined substituent R.

The use of the following symbol indicates a single bond or an optional double bond:

[symbol]

In an embodiment, the present invention is directed to a method of treating irritable bowel syndrome, dyspepsia, including gallbladder dyspepsia, Crohn's disease, gastroesophogeal reflux disorders, ulcerative colitis, pancreatitis, infantile hypertrophic pyloric stenosis, carcinoid syndrome, malabsorption syndrome, postgastroenterectomy syndrome, atrophic colitis or gastritis, gastrointestinal dumping syndrome, short bowel syndrome, celiac disease, cachexia, chemotherapy-induced nausea and vomiting (emesis), post-operative nausea and vomiting, cyclic vomiting syndrome and functional vomiting in humans and other mammals comprising administering a therapeutically effective amount of a compound of formula I.

In another embodiment of the invention, the present invention is directed to a method of treating diarrhea, cancer treatment-related diarrhea, cancer-induced diarrhea, chemotherapy-induced diarrhea, radiation enteritis, radiation-induced diarrhea, stress-induced diarrhea, chronic diarrhea, AIDS-related diarrhea, C. difficile associated diarrhea, traveller's diarrhea, acute infectious diarrhea, diarrhea induced by graph versus host disease, or other types of diarrhea.

### 2. Synthetic Methods

The compounds of formula I can be synthesized using traditional solution synthesis techniques or solid phase chemistry methods. Synthetic methods for this type of macrocyclic structure are described in Intl. Pat. Appl. Publ. Nos. WO 01/25257, WO 2004/111077, WO 2005/012331, WO 2005/012332, WO 2006/009645, WO 2006/009674 and WO 2008/033328 including purification procedures described in WO 2004/111077 and WO 2005/012331.

### 3. Biological Methods

The compounds of the present invention were evaluated for their ability to interact at the human motilin receptor utilizing a competitive radioligand binding assay, fluorescence assay or Aequorin functional assay as described below. Such methods can be conducted in a high throughput manner to permit the simultaneous evaluation of many compounds.

Specific assay methods and their use in generally identifying agonists and antagonists of the motilin receptor are known. (Peeters, T. L.; Macielag, M. J.; Depoortere, I.; et al. Peptides 1992, 13, 1103-1107; Peeters, T. L.; Depoortere, I.; Macielag, M. J.; Marvin, M. S.; Florance, J. R.; Galdes, A. Biochem. Biophys. Res. Comm. 1994, 198, 411-416; Poitras, P.; Miller, P.; Gagnon, D.; St-Pierre, S. Biochem. Biophys. Res. Comm. 1994, 205, 449-454; Takanishi, H.; Yogo, K.; Ozaki, M.; Akima, M.; Koga, H.; Nabata, H. J. Pharm. Exp. Ther. 1995, 273, 624-628; Depoortere, I.; Macielag, M. J.; Galdes, A.; Peeters, T. L. Eur. J. Pharmacol. 1995, 286, 241-247; Farrugia, G.; Macielag, M. J.; Peeters, T. L.; Sarr, M. G.; Galdes, A.; Szurszewski, J. H. Am. J. Physiol. 1997, 273, G404-G412; Haramura, M.; Tsuzuki, K.; Okamachi, A.; et al. Chem. Pharm. Bull. 1999, 47, 1555-1559; Haramura, M.; Okamachi, A.; Tsuzuki, K.; Yogo, K.; Ikuta, M.; Kozono, T.; Takanashi, H.; Murayama, E. Chem. Pharm. Bull. 2001, 49, 40-43; Beavers, M. P.; Gunnet, J. W.; Hageman, W.; Miller, W.; Moore, J. B.; Zhou, L.; Chen, R. H. K.; Xiang, A.; Urbanski, M.; Combs, D. W.; Mayo, K. H.; Demarest, K. T. Drug Des. Disc. 2001, 17, 243-251; Haramura, M.; Okamachi, A.; Tsuzuki, K.; Yogo, K.; Ikuta, M.; Kozono, T.; Takanashi, H.; Murayama, E. J. Med. Chem. 2002, 45, 670-675.)

Appropriate methods for determining the functional activity of compounds of the present invention that interact at the human motilin receptor are also described below, as are representative studies to determine the effects of compounds of the present invention in relevant animal models. Other relevant animal models suitable for examining the efficacy of compounds of the present invention for the treatment of functional GI disorders, such as IBS and dyspepsia, are known. (Mayer, E. A.; Bradesi, S.; Chang, L.; Spiegel, B. M. R.; Bueller, J. A.; Naliboff, B. D. Gut 2008, 57, 384-404.) Additionally, zebrafish can be employed to evaluate the effects of compounds on GI motility. (Olsson, C.; Holbrook, J. D.; Bompadre, G.; Joensson, E.; Hoyle, C. H. V.; Sanger, G. J.; Holmgren, S.; Andrews, P. L. R. Gen. Comp. Endocrinol. 2008, 155, 217-226.)

Gastric motility is generally measured in the clinical setting as the time required for gastric emptying and subsequent transit time through the GI tract. Gastric emptying scans are well known to those skilled in the art. (Lin, H. C.; Prather, C.; Fisher, R. S.; et al. Dig. Dis. Sci 2005, 50, 989-1004; Camilleri M. Neurogastroenterol. Motil. 2006, 18, 805-812.) For one example, an appropriate method comprises use of an oral contrast agent, such as barium or a radiolabeled meal. Solid and liquids can be measured independently. A test food or liquid is radiolabeled with an appropriate isotope (^{99m}Tc, for example) and after ingestion or administration, transit time through the GI tract and gastric emptying is measured by visualization using gamma cameras. These studies are performed before and after the administration of the therapeutic agent to quantify the efficacy of the compound.

A. Competitive Radioligand Binding Assay (Motilin Receptor)

The competitive binding assay at the human motilin receptor was carried out analogously to assays described in the literature.

### Materials:

Membranes were prepared from CHO cells stably transfected with the human motilin receptor and utilized at a quantity of 1.5 μg/assay point. [PerkinElmer™ SignalScreen Product #6110544]
[¹²⁵I]-Motilin (PerkinElmer, # NEX-378); final concentration: 0.04-0.06 nM
Motilin (Bachem™, #H-4385); final concentration: 1 μM
Multiscreen Harvest plates-GF/B (Millipore™, #MAHFB1H60)
Deep-well polypropylene titer plate (Beckman Coulter™, #267006)
TopSeal-A (PerkinElmer, #6005185)
Bottom seal (Millipore, #MATAH0P00)
MicroScint-0 (PerkinElmer, #6013611)
Binding Buffer: 50 mM Tris-HCl (pH 7.4), 10 mM MgCl₂, 1 mM EDTA, 0.1% BSA

### Assay Volumes:

150 μL of membranes diluted in binding buffer
10 μL of compound diluted in binding buffer
10 μL of radioligand ([¹²⁵I]-Motilin) diluted in binding buffer

### Final Test Concentrations (N=11) for Compounds:

10, 5.0, 2.0, 1.0, 0.50, 0.20, 0.10, 0.050, 0.020, 0.010, 0.0050 μM.

In some cases, a lower number of concentrations, such as eight (8), were employed.

### Compound Handling:

Compounds were provided frozen on dry ice at a stock concentration of 10 mM diluted in 100% DMSO and stored at −20° C. until the day of testing. On the test day, compounds were allowed to thaw at room temperature and than diluted in assay buffer according to the desired test concentrations. Under these conditions, the maximum final DMSO concentration in the assay was 0.5%.

### Assay Protocol;

In deep-well plates, diluted cell membranes (1.5 μg/mL) are combined with 10 μL of either binding buffer (total binding, N=5), 1 μM motilin (non-specific binding, N=3) or the appropriate concentration of test compound. The reaction is initiated by addition of 10 μL of [¹²⁵I]-motilin (final conc. 0.04-0.06 nM) to each well. Plates are sealed with TopSeal-A, vortexed gently and incubated at room temperature for 2 hours. The reaction is arrested by filtering samples through pre-soaked (0.3% polyethyleneimine, 2 h) Multiscreen Harvest plates using a Tomtec Harvester, washed 9 times with 500 μL of cold 50 mM Tris-HCl (pH 7.4), and than plates are air-dried in a fumehood for 30 minutes. A bottom seal is applied to the plates prior to the addition of 25 μL of MicroScint-0 to each well. Plates are than sealed with TopSeal-A and counted for 30 sec per well on a TopCount Microplate Scintillation and Luminescence Counter (PerkinElmer) where results are expressed as counts per minute (cpm).

Data are analyzed by GraphPad™ Prism® (GraphPad Software, San Diego, Calif., USA) using a variable slope non-linear regression analysis. Kᵢ values were calculated using a K_{d} value of 0.16 nM for [¹²⁵I]-motilin (previously determined during membrane characterization).

$${D}_{max} = 1 - \frac{\begin{matrix}test concentration with maximal displacement - \\ non \text{-} specific binding\end{matrix}}{total binding - non \text{-} specific binding} \times 100$$

where total and non-specific binding represent the cpm obtained in the absence or presence of 1 μM motilin, respectively.

B. Aequorin Functional Assay (Motilin Receptor)

The evaluation of compounds of the invention for functional activity can be conducted according to literature methods or as described below. (Carreras, C. W.; Siani, M. A.; Santi, D. V.; Dillon, S. B. Anal. Biochem. 2002, 300, 146-151; U.S. Pat. No. 6,872,538; Intl. Pat. Appl. No. WO 00/002045.) This assay can be adapted to high throughput for simultaneous evaluation of large numbers of compounds. (Le Poul, E.; Hisada, S.; Mizuguchi, Y.; Dupriez, V. J.; Burgeon, E.; Detheux, M. J. Biomol. Screen. 2002, 7 57-65.)

### Materials:

Membranes were prepared using AequoScreen™ (EUROSCREEN, Belgium) cell lines expressing the human motilin receptor (cell line ES-380-A; receptor accession #AF034632). This cell line is constructed by transfection of the human motilin receptor into CHO-K1 cells co-expressing G_{αI6} and the mitochondrially targeted Aequorin (Ref. #ES-WT-A5).
Motilin (Bachem, #H-4385)
Assay buffer: DMEM-F12 (Dulbeccoe's Modified Eagles Medium) with 15 mM HEPES and 0.1% BSA (pH 7.0)
Coelenterazine (Molecular Probes™, Leiden, The Netherlands)

### Final Test Concentrations (N=5) for Compounds:

10, 3.16, 1.0, 0.316, 0.10 μM.

### Compound Handling:

Compounds were provided as dry films at a quantity of approximately 1.2 μmol in pre-formatted 96-well plates. Compounds were dissolved in 100% DMSO at a concentration of 10 mM and stored at −20° C. until further use. Daughter plates were prepared at a concentration of 500 μM in 30% DMSO with 0.1% BSA and stored at −20° C. until testing. On the test day, compounds were allowed to thaw at room temperature and than diluted in assay buffer according to the desired test concentrations. Under these conditions, the maximum final DMSO concentration in the assay was 0.6%.

### Cell Preparation:

Cells are collected from culture plates with Ca²⁺ and Mg²⁺-free phosphate buffered saline (PBS) supplemented with 5 mM EDTA, pelleted for 2 minutes at 1000×g, resuspended in assay buffer (see above) at a density of 5×10⁶ cells/mL and incubated overnight in the presence of 5 μM coelenterazine. After loading, cells were diluted with assay buffer to a concentration of 5×10⁵ cells/mL.

### Assay Protocol:

For agonist testing, 50 μL of the cell suspension was mixed with 50 μL of the appropriate concentration of test compound or motilin (reference agonist) in 96-well plates (duplicate samples). The emission of light resulting from receptor activation was recorded using the Functional Drug Screening System 6000 ‘FDSS 6000’ (Hamamatsu Photonics K.K., Japan).

For antagonist testing, an approximate EC₈₀ concentration of motilin (i.e. 0.5 nM; 100 μL) was injected onto the cell suspension containing the test compounds (duplicate samples) 15-30 minutes after the end of agonist testing and the consequent emission of light resulting from receptor activation was measured as described in the paragraph above.

Results are expressed as Relative Light Units (RLU). Concentration response curves were analyzed using GraphPad™ Prism® (GraphPad Software, San Diego, Calif., USA) by non-linear regression analysis (sigmoidal dose-response) based on the equation E=Eₘₐₓ/(1+EC₅₀/C)n where E is the measured RLU value at a given agonist concentration (C), Eₘₐₓ is the maximal response, EC₅₀ is the concentration producing 50% stimulation and n is the slope index. For agonist testing, results for each concentration of test compound were expressed as percent activation relative to the signal induced by motilin at a concentration equal to the EC₈₀ (i.e. 0.5 nM). For antagonist testing, results for each concentration of test compound were expressed as percent inhibition relative to the signal induced by motilin at a concentration equal to the EC₈₀ (i.e. 0.5 nM).

C: FlashPlate Motilin [³⁵S]-GTPγS Functional Assay

### Materials:

Membranes were prepared from CHO cells stably transfected with the human motilin receptor and utilized at a quantity of 1.5 μg/assay point. [PerkinElmer SignalScreen Product #6110544]
GTPγS (Sigma, #G-8634)
[³⁵S]-GTPγS (PerkinElmer, #NEX-030H)
Motilin (Bachem, #H-4385)
96-well FlashPlate microplates (PerkinElmer, #SMP200)
Deep-well polypropylene titer plate (Beckman Coulter, #267006)
TopSeal-A (PerkinElmer, #6005185)
Assay Buffer: 50 mM Tris (pH 7.4), 100 mM NaCl, 10 mM MgCl₂, 1 mM EDTA, 1 μM GDP, 0.1% BSA

### Assay Volumes:

25 μL of compound diluted in assay buffer
25 μL of assay buffer (agonist assay) or 0.6 μM motilin (0.1 μM final concentration) diluted in assay buffer (antagonist assay)
100 μL of [³⁵S]-GTPγS diluted in assay buffer

### Final Test Concentrations (N=12) for Compounds;

50, 20, 10, 5.0, 2.0, 1.0, 0.50, 0.20, 0.10, 0.050, 0.020, 0.010 μM.

In some cases, a lower number of concentrations, such as eight (8), were employed.

### Compound Handling:

Compounds were provided frozen on dry ice at a stock concentration of 10 mM diluted in 100% DMSO and stored at −20° C. until the day of testing. On the test day, compounds were allowed to thaw at room temperature and than diluted in assay buffer according to the desired test concentrations. Under these conditions, the maximum final DMSO concentration in the assay was 0.5%.

### Assay Protocol:

CHO membranes were immobilized into 96-well FlashPlate microplates. Test compound, GTPγS, motilin and [³⁵S]-GTPγS were combined in each well according to the Assay Volumes described above.

For the assay to measure agonist activity, an additional 25 μL of buffer was added to each well in addition to 25 μL of either buffer (basal value, N=4), 1.0 μM (final conc.) motilin (Eₘₐₓ value, N=3), 25 μM (final conc.) GTPγS (non-specific value, N=4), or the appropriate concentration of test compound (N=3).

For the assay to measure antagonist activity, an additional 25 μL of either buffer (unstimulated control) or motilin (0.10 μM final conc.) is added to each well, in addition to either 25 μL of buffer (basal value, N=3), 1.0 μM (final conc.) motilin (Eₘₐₓ value, N=3), 25 μM (final conc.) GTPγS (non-specific value, N=4), or the appropriate concentration of test compound (N=3).

The reaction is initiated by addition of 100 mL of [³⁵S]-GTPγS to each well. Each plate is sealed (TopSeal-A) and incubated in the dark at room temperature for 150 min. Then, plates are counted for 30 seconds per well on the TopCount NXT.

Data were analyzed by GraphPad™ Prism® 3.0 (GraphPad Software, San Diego, Calif., USA) using non-linear regression analysis (sigmoidal dose-response) for the calculation of IC₅₀/EC₅₀ values.

$${E}_{max} ( agonist ) or {D}_{max} ( antagonist ) = \frac{Top - Bottom}{Bottom} \times 100$$

Where Top and Bottom correspond to the top and bottom values of the dose-response curve calculated by GraphPad™ Prism® (GraphPad Software, San Diego, Calif., USA).

D. Rabbit Duodenum Contractility Assay

Evaluation of compounds of the invention for ear vivo activity was conducted on strips of rabbit duodenum according to literature methods. (Van Assche, G.; Depoortere, I.; Thijs, T.; Janssens, J. J.; Peeters, T. L. Eur. J. Pharmacol. 1997, 337, 267-274; Matthijs, G.; Peeters, T. L.; Vantrappen, G. Naunyn-Schmiedeberg's Arch. Pharmacol. 1989, 339, 332-339.) Related methods can also be employed for this type of study. (Tomomasa, T.; Yagi, H.; Kimura, S.; Snape, W. J., Jr.; Hyman, P. E. Pediatric Res. 1989, 26, 458-461; Takanishi, H.; Yogo, K.; Ozaki, M.; Akima, M.; Koga, H.; Nabata, H. J. Pharm. Exp. Ther. 1995, 273, 624-628.)

Duodenal segments were vertically suspended in organ chambers of 10 mL filled with Krebs buffer and connected to an isotonic force transducer, with a preload of 1 g. After a stabilization period, the muscle strips were challenged with 10⁻⁴ M acetylcholine and washed. This was repeated until a stable maximal contraction was obtained (2-3 times), with an interval of at least 20 minutes.

After a stable base line was reached, test compounds were added to the bath. After a 15 minute incubation, a dose response to motilin was recorded by adding logarithmically increasing concentrations of motilin to the bath (final concentration 10⁻⁹ to 10⁻⁶ M). A blank experiment (no test compound present) was also performed. At the end of the dose response curve, a supramaximal dose of acetylcholine (10⁻⁴ M) was given and this response was used as a reference (100% contraction).

The results of experiments at different concentrations of test compound were combined and analyzed to derive the pA₂ value from the Schild plot.

E. Animal Model of Fundic Accommodation

Fundic accommodation in response to a 200 mL milk meal was measured using a barostat. This can be considered as a model for functional dyspepsia, at least some occurrences of which is known to be a result of poor gastric accommodation (Tack, J.; Piessevaux, H.; Coulie, B.; Caenepeel, P.; Janssens, J. Gastroenterology 1998, 115, 1346-1352; DiStefano, M.; Micelli, E.; Mazzocchi, S.; Tana, P.; Corazza, G. R. Eur. Rev. Med. Pharmacol. Sci. 2005, 9, 23-28; Kindt, S.; Tack, J. Gut 2006, 55, 1685-1691; van den Elzen, B. D.; Boeckxstaens, G. E. Aliment. Pharmacol. Ther. 2006, 23, 1499-1510.). The described method is based upon a literature method. (Meulemans A. Schuurkes J. Neurogastroenterol. Motil. 1995, 7, 151-155.) The use of gallbladder volume has been linked to the motilin effect in functional dyspepsia and also can be used as a biomarker to test the effect of an antagonist on FD in humans. (Kamerling, I. M. C.; Van Haarst, A. D.; De Kam, M. L.; Cohen, A. F.; Masclee, A. A. M.; Burggraaf, J. Aliment, Pharmacol. Ther. 2004, 19, 797-804.) Other methods can also be utilized for this assessment and have been reviewed. (DeSchepper, H. U.; Cremonini, F.,; Chitkara, D.; Camilleri, M. Neurogastroenterol. Motil. 2004, 16, 275-285.) The dog is often used as an appropriate model for GI diseases due to the similarity in the alimentary tracts between dogs and humans. Additionally, the dog motilin receptor has been identified and characterized with the expression profile determined. It shares 71% homology to the human receptor and 72% to the rabbit receptor. (Ohshiro, H.; Nonaka, M.; Ichikawa, K. Regul. Pept. 2008, 146, 80-87.)

Beagle dogs were trained to stand quietly in Pavlov frames. Each was implanted using the following procedure with a gastric and duodenal cannula under general anesthesia with standard aseptic precautions. After a medium laparotomy, an incision was made through the gastric wall in the longitudinal direction between the greater and the lesser curvature, 2 cm above the nerves of Latarjet. The gastric cannula (diameter 18 mm) was secured to the gastric wall by means of an appropriate suture and then brought out via a stab wound at the left quadrant of the hypochondrium. The duodenal cannula (diameter 4 mm) was implanted at about 12 cm distal to the pylorum. An incision of approximately 1 cm was made through the duodenal wall 3 cm distally from the site of implantation. The cannula was brought into the duodenum though this incision. The top of the cannula was brought out of the duodenal wall at the place of implantation and secured to the wall via suture. Afterwards, the duodenum was closed and the duodenal cannula was brought out via a stab wound at the right quadrant of the hypochondrium. Dogs were allowed a recovery period of 2 weeks during which they were treated with antibiotics.

Variations in gastric tone were measured by recording changes in the volume of air within an intragastric bag maintained at constant pressure (6 mm Hg) by a barostat. The barostat consists of an air injection system which is connected by a double-lumen polyvinyl tube to an ultrathin flaccid polyethylene bag with maximum volume of approximately 700 mL. Gastric tonic contractions cause the barostat to withdraw air to maintain the pressure, while relaxation triggers air injection to maintain pressure. In that manner, intrabag volume is an inverse measure of proximal gastric tone. Differences in gastric tone were recorded using a computer.

Experiments were conducted after a fasting period of 24 h, during which water was available ad libitum. At the beginning of the experiment, the cannulas were opened in order to remove any gastric juice or food remnants. If necessary, the stomach was cleansed with 40-50 mL of lukewarm water. After calibration, the bag was positioned into the fundus of the stomach through the gastric cannula. A rubber stopper was used to close the space between the tubing and the wall of the cannula. To ensure easy unfolding of the bag during the experiment, a volume of 150-200 mL was injected into the bag by raising the pressure very briefly from 4 to 14 mm Hg. This was repeated twice, then a stabilization period of 1 h passed prior to initiation of the experiment.

Experiments were performed at a constant pressure of 6 mm Hg. Test solution or saline was administered via the duodenal cannula in a volume of 5 mL. Each injection was followed by an injection of saline as a correction of the dead space in the system. Experiments were performed on six different dogs.

The effects of Compound 502 at various concentrations (0.1, 0.3 and 1.0 mg/kg, i.v.) were examined in this gastric accommodation model. In healthy subjects, the stomach relaxes naturally in response to a meal. In a preliminary experiment, compound 502 did not disturb this baseline phenomenon.

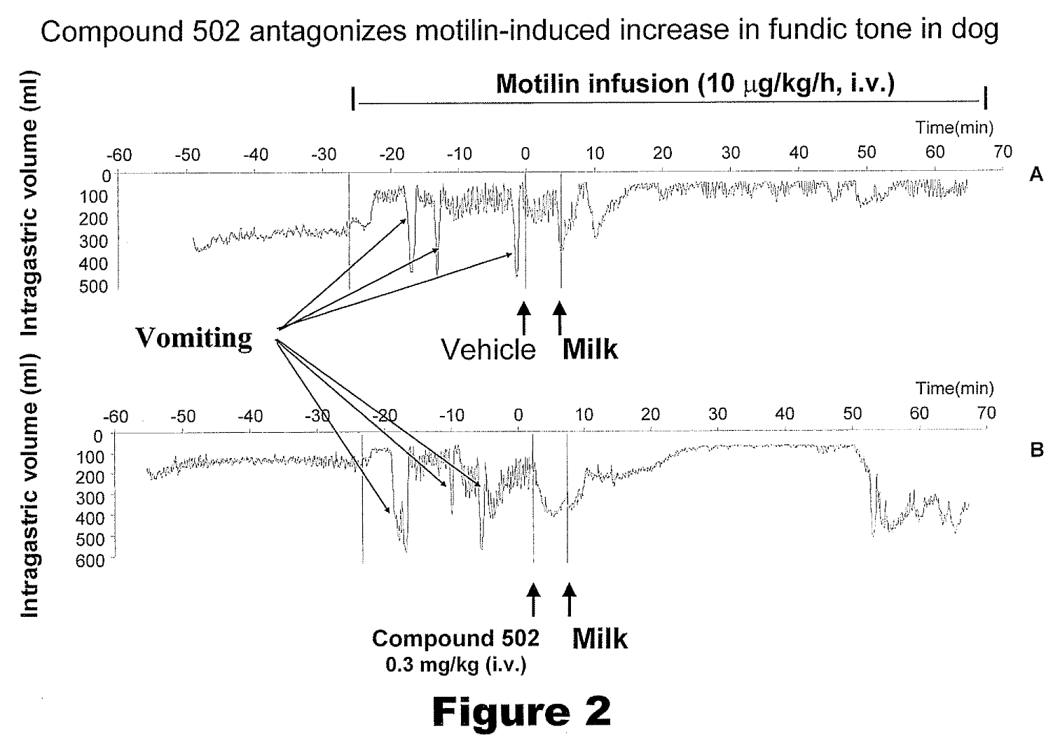

Motilin is known to reduce gastric volume (increase fundic tone). Thus, a high dose of motilin (0.01 mg/kg/h, i.v.) was infused throughout the duration of the experiment. In FIG. 2, it is shown that this dose of motilin caused the dog to vomit three times as indicated and further prevented any relaxation of the fundus in response to the milk meal, as indicated by the fact that there were no major changes in the amplitude of the tracing representing changes in gastric pressure.

### F. Strain Gauge Animal Model

Gastrointestinal motility was measured for 24 hours by telemetry in freely moving beagle dogs instrumented with strain gauges on both the antrum and duodenum. The method utilized for the strain gauges was based upon that in the literature. (Edelbroek, M.; Schuurkes, J.; De Ridder, W.; Horowitz, M.; Dent, J.; Akkermans, L. Dig. Dis. Sci. 1995, 40, 901-911; Briejer, M. R.; Prins, N. H.; Schuurkes, J. A. Neurogastroenterol. Motil. 2001 13, 465-472.) Analogous methods that can be used are known. (Dog: Itohb, Z.; Honda, R.; Takeuchi, S.; Aizawa, I.; Takayanagi, R. Gastroenterol. Jpn. 1977, 12, 275-283; Iwai, T.; Nakamura, H.; Takanashi, H.; et al. Can. J. Physiol. Pharmacol. 1998, 76, 1103-1109; Dog and guinea pig, Ohno, T.; Kamiyama, Y.; Aihara, R.; Nakabayashi, T.; Mochiki, E.; Asao, T.; Kuwano, H. Neurogastroenterol. Motil. 2006, 18, 129-135; Rat: Bunce, K. T.; Elswood, C. J.; Ball, M. T. Br. J. Pharmacol. 1991, 102, 811-816; Guinea pig: Cooke, H. J.; Wang, Y. Z.; Frieling, T.; Wood, J. D. Am. J. Physiol. 1991, 261, G833-G840.)

### Animal Preparation

Calibrated strain gauges (Schuurkes, J. A. J.; Van der Schee, E. J.; Grashuis, J. L.; Charbon, G. R. A. in Gastrointestinal Motility in Health and Disease; Duthie, H. L., Ed., MTP Press, Lancaster (UK), 1978, pp 647-654) and bipolar extracellular electrodes were implanted under aseptic conditions in each of four female beagle dogs. (Bass, P.; Wiley, J. N. J. Appl. Physiol. 1972, 32, 567-570.) The strain gauges were sutured onto the seromuscular layer parallel to the axis of the circular muscle, on the distal antrum (3 cm orad to the distal pyloric loop), proximal duodenum (3 cm and 6 cm aborad to the pylorus) and distal pyloric muscle loop. The bipolar extracellular electrodes were implanted onto the scrosal side of the distal antrum and proximal duodenum 2 cm orad and aborad to the pylorus. The lead wires were brought out via a subcutaneous tunnel in the left costal flank through a stab wound between the scapulae. Postsurgically, the lead wires were soldered to the connectors and protected appropriately.

In the same surgical incision, a gastrostomy and duodenostomy were performed. The gastrostomy was situated anteriorly in the midventral region between the lesser and greater curvature 2 cm orad to the nerve of Latarget and kept patent with a stainless steel cannula (internal diameter 1.4 cm, length 7.0 cm). The duodenostomy was situated 10 cm aborad to the pylorus and was kept patent with a cannula of smaller diameter (internal diameter 0.9 cm, length 7.5 cm). A thin tubing (outer diameter 2.2 mm) was pulled across the pylorus and attached to the caps of the gastric and duodenal cannulae in order to facilitate placement of the manometric assembly postsurgically. The greater omentum was wrapped around the cannulae to secure positioning and then the cannulae were tightly capped between experiments to permit normal feeding. Dogs were allowed to recover for at least two weeks prior to conduct of any experiments.

### Pressure Measurements

Intraluminal pressures were recorded with a manometric assembly incorporating a sleeve sensor with a 4.8 cm length (outer diameter 5.4 mm) to monitor pyloric pressure waves. (Dent, J. Gastroenterology 1976, 71, 263-267.) Four side holes were spaced at 1.6-cm intervals along the sleeve length. Side holes at each end of the sleeve were used to measure antral and duodenal pressures and transmucosal potential difference (TMPD) simultaneously. (Arndorfer, R. C.; Stef, J. J.; Doss, W. J.; Linehan, J. H.; Hogan, W. J. Gastroenterol. 1977, 73, 23-27.) Two additional side holes were placed 3.3 and 1.6 cm orad to the proximal end of the sleeve (antral sites) and another two side holes were placed 1.6 and 3.3 cm aborad to the distal end of the sleeve (duodenal sites). All channels were perfused with degassed distilled water at 0.3 mL/min, except for the side holes used to measure the antroduodenal TMPD, which were perfused with saline. (Geall, M. G.; Code, C. F.; McBrath, D. C.; Summerskill, W. H. J. Gut 1970, 11, 34-37) At the start of each study, the manometric catheter was attached to the gastric end of a thin transpyloric tubing and slowly pulled into position from the duodenostomy end. When the antroduodenal TMPD indicated a potential difference of ≧20 mV, both ends of the catheter were secured to the gastric and duodenal cannulae with rubber stoppers. The dead space in each cannula was filled with gauze soaked in saline to prevent pooling of gastric secretions or intraduodenal infusates.

A schematic representation is presented in the literature to assist with this procedure. (Edelbroek M. Schuurkes J. De Ridder W. Horowitz M. Dent J. Akkermans L. Dig. Dis. Sci. 1995, 40, 901-911.)

Analog signals from pressure and strain-gauge force transducers were recoded on an appropriate chart recording device. Calibration was set at 5 mm Hg/mm chart deflection (100 mV scale) for the manometric signals and at 2 mN/mm chart deflection (100 mV scale) for the strain gauge transducers. These settings enabled measurement of peak amplitudes of the antral and pyloric signals and did not permit accurate measurement of basal pyloric pressure. Antroduodenal transmucosal potential signals were read with two separate calomel electrodes (HgCl/KCl) from two different DC amplifiers and recorded manually every 5 minutes.

### Protocol

The effect of intravenous administration of test compounds on myoelectric and motor activities was compared with placebo of intravenous saline. Dogs were fasted for 12 h prior to each experiment with water permitted ad libitum. The dogs stood quietly in Pavlov frames while recordings were taken. In each experiment, an intraduodenal infusion of a triglyceride emulsion (for example 10% Intralipid) was delivered 5 cm distal to the pylorus at a rate of 0.45 mL/min for 40 min. Normal saline infusions were delivered at the same rate for 40 min before and after the intraduodenal lipid infusion. The first intraduodenal saline infusion was begun during phase I of the interdigestive motor cycle, 10 min after the preceding episode of duodenal phase III activity. Test compound or saline was injected as an intravenous bolus at the start of the first intraduodenal saline infusion, i.e. 40 min before the start of the triglyceride emulsion infusion.

Minimum values for pressure waves (≧8 mm Hg) or contractions (≧2 mN) represented ≧2.5% of the median maximum deflection during phase III of the interdigestive migrating motor complex (MMC) of each experiment. The numbers of antral, pyloric and duodenal motor events are based upon combined signals from manometry and strain gauges. (Edelbroek, M.; Schuurkes, J.; deRidder, W.; Horowitz, M.; Dent, J.; Akkermans, L. Dig. Dis. Sci. 1994, 39, 577-586.)

Comparison between the effects of intraduodenal lipid versus saline infusion and the effects of test compound versus placebo during intraduodenal lipid versus saline infusion on myoelectrical and motor activities of the antropyloroduodenal region were assessed with Koch's nonparamagnetic split-plot analysis, assuming that multiple experiments performed per dog were independent observations. The effects of test compound versus placebo on motor patterns during periods of antral tachygastria were compared with periods of antral eugastria of equal duration in a total of four paired experiments in two dogs with the Wilcoson rank-sum test. Pressure data are shown as median values and interquartile range, electrical activity data as mean values±SEM; a p<0.05 was considered significant in all analyses.

Dogs with strain gauge transducers were fed a standard meal during the quiescent phase of the interdigestive state, 30 min after passage of phase III of a migrating motor complex (MMC). Two hours post meal, solvent or test compound at various concentrations was administered intravenously in a maximum volume of 5 mL via an orogastric tube, preceded and followed by 2.5 mL water. Antroduodenal motility was then followed for 24 h.

### 4. Pharmaceutical Compositions

The macrocyclic compounds of the present invention or pharmacologically acceptable salts thereof according to the invention may be formulated into pharmaceutical compositions of various dosage forms. To prepare the pharmaceutical compositions of the invention, one or more compounds, including optical isomers, enantiomers, diastereomers, racemates or stereochemical mixtures thereof, or pharmaceutically acceptable salts thereof as the active ingredient is intimately mixed with appropriate carriers and additives according to techniques known to those skilled in the art of pharmaceutical formulations.

A pharmaceutically acceptable salt refers to a salt form of the compounds of the present invention in order to permit their use or formulation as pharmaceuticals and which retains the biological effectiveness of the free acids and bases of the specified compound and that is not biologically or otherwise undesirable. Such salts are described in Handbook of Pharmaceutical Salts: Properties, Selection, and Use, Wermuth, C. G. and Stahl, P. H. (eds.), Wiley-Verlag Helvetica Acta, Zürich, 2002 [ISBN 3-906390-26-8]. Examples of such salts include alkali metal salts and addition salts of free acids and bases. Examples of pharmaceutically acceptable salts, without limitation, include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, xylenesulfonates, phenylacetates, phenylpropionates, phenylbutyrates, citrates, lactates, γ-hydroxybutyrates, glycollates, tartrates, methanesulfonates, ethane sulfonates, propanesulfonates, toluenesulfonates, naphthalene-1-sulfonates, naphthalene-2-sulfonates, and mandelates.

If an inventive compound is a base, a desired salt may be prepared by any suitable method known to those skilled in the art, including treatment of the free base with an inorganic acid, such as, without limitation, hydrochloric acid, hydrobromic acid, hydroiodic, carbonic acid, sulfuric acid, nitric acid, phosphoric acid, and the like, or with an organic acid, including, without limitation, formic acid, acetic acid, propionic acid, maleic acid, succinic acid, mandelic acid, fumaric acid, malonic acid, pyruvic acid, oxalic acid, stearic acid, ascorbic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid, 2-hydroxyethanesulfonic acid, benzenesulfonic acid, cyclohexyl-aminosulfonic acid or the like.

If an inventive compound is an acid, a desired salt may be prepared by any suitable method known to the art, including treatment of the free acid with an inorganic or organic base, such as an amine (primary, secondary, or tertiary); an alkali metal or alkaline earth metal hydroxide; or the like. Illustrative examples of suitable salts include organic salts derived from amino acids such as glycine, lysine and arginine; ammonia; primary, secondary, and tertiary amines such as ethylenediamine, N,N′-dibenzylethylenediamine, diethanolamine, choline, and procaine, and cyclic amines, such as piperidine, morpholine, and piperazine; as well as inorganic salts derived from sodium, calcium, potassium, magnesium, manganese, iron, copper, zinc, aluminum, and lithium.

The carriers and additives used for such pharmaceutical compositions can take a variety of forms depending on the anticipated mode of administration. Thus, compositions for oral administration may be, for example, solid preparations such as tablets, sugar-coated tablets, hard capsules, soft capsules, granules, powders and the like, with suitable carriers and additives being-, starches, sugars, binders, diluents, granulating agents, lubricants, disintegrating agents and the like. Because of their ease of use and higher patient compliance, tablets and capsules represent the most advantageous oral dosage forms for many medical conditions.

Similarly, compositions for liquid preparations include solutions, emulsions, dispersions, suspensions, syrups, elixirs, and the like with suitable carriers and additives being water, alcohols, oils, glycols, preservatives, flavoring agents, coloring agents, suspending agents, and the like. Typical preparations for parenteral administration comprise the active ingredient with a carrier such as sterile water or parenterally acceptable oil including polyethylene glycol, polyvinyl pyrrolidone, lecithin, arachis oil or sesame oil, with other additives for aiding solubility or preservation may also be included. In the case of a solution, it can be lyophilized to a powder and then reconstituted immediately prior to use. For dispersions and suspensions, appropriate carriers and additives include aqueous gums, celluloses, silicates or oils.

The pharmaceutical compositions according to embodiments of the present invention include those suitable for oral, rectal, topical, inhalation (e.g., via an aerosol) buccal (e.g., sub-lingual), vaginal, topical (i.e., both skin and mucosal surfaces, including airway surfaces), transdermal administration and parenteral (e.g., subcutaneous, intramuscular, intradermal, intraarticular, intrapleural, intraperitoneal, intrathecal, intracerebral, intracranially, intraarterial, or intravenous), although the most suitable route in any given case will depend on the nature and severity of the condition being treated and on the nature of the particular active agent which is being used.

Compositions for injection will include the active ingredient together with suitable carriers including propylene glycol-alcohol-water, isotonic water, sterile water for injection (USP), emulPhor™-alcohol-water, cremophor-EL™ or other suitable carriers known to those skilled in the art. These carriers may be used alone or in combination with other conventional solubilizing agents such as ethanol, propylene glycol, or other agents known to those skilled in the art.

Where the macrocyclic compounds of the present invention are to be applied in the form of solutions or injections, the compounds may be used by dissolving or suspending in any conventional diluent. The diluents may include, for example, physiological saline, Ringer's solution, an aqueous glucose solution, an aqueous dextrose solution, an alcohol, a fatty acid ester, glycerol, a glycol, an oil derived from plant or animal sources, a paraffin and the like. These preparations may be prepared according to any conventional method known to those skilled in the art.

Compositions for nasal administration may be formulated as aerosols, drops, powders and gels. Aerosol formulations typically comprise a solution or fine suspension of the active ingredient in a physiologically acceptable aqueous or non-aqueous solvent. Such formulations are typically presented in single or multidose quantities in a sterile form in a sealed container. The sealed container can be a cartridge or refill for use with an atomizing device. Alternatively, the sealed container may be a unitary dispensing device such as a single use nasal inhaler, pump atomizer or an aerosol dispenser fitted with a metering valve set to deliver a therapeutically effective amount, which is intended for disposal once the contents have been completely used. When the dosage form comprises an aerosol dispenser, it will contain a propellant such as a compressed gas, air as an example, or an organic propellant including a fluorochlorohydrocarbon or fluorohydrocarbon.

Compositions suitable for buccal or sublingual administration include tablets, lozenges and pastilles, wherein the active ingredient is formulated with a carrier such as sugar and acacia, tragacanth or gelatin and glycerin.

Compositions for rectal administration include suppositories containing a conventional suppository base such as cocoa butter.

Compositions suitable for transdermal administration include ointments, gels and patches.

Other compositions known to those skilled in the art can also be applied for percutaneous or subcutaneous administration, such as plasters.

Further, in preparing such pharmaceutical compositions comprising the active ingredient or ingredients in admixture with components necessary for the formulation of the compositions, other conventional pharmacologically acceptable additives may be incorporated, for example, excipients, stabilizers, antiseptics, wetting agents, emulsifying agents, lubricants, sweetening agents, coloring agents, flavoring agents, isotonicity agents, buffering agents, antioxidants and the like. As the additives, there may be mentioned, for example, starch, sucrose, fructose, dextrose, lactose, glucose, mannitol, sorbitol, precipitated calcium carbonate, crystalline cellulose, carboxymethylcellulose, dextrin, gelatin, acacia, EDTA, magnesium stearate, talc, hydroxypropylmethylcellulose, sodium metabisulfite, and the like.

In some embodiments, the composition is provided in a unit dosage form such as a tablet or capsule.

In further embodiments, the present invention provides kits including one or more containers comprising pharmaceutical dosage units comprising an effective amount of one or more compounds of the present invention.

The present invention further provides prodrugs comprising the compounds described herein. The term “prodrug” is intended to mean a compound that is converted under physiological conditions or by solvolysis or metabolically to a specified compound that is pharmaceutically active. The “prodrug” can be a compound of the present invention that has been chemically derivatized such that, (i) it retains some, all or none of the bioactivity of its parent drug compound, and (ii) it is metabolized in a subject to yield the parent drug compound. The prodrug of the present invention may also be a “partial prodrug” in that the compound has been chemically derivatized such that, (i) it retains some, all or none of the bioactivity of its parent drug compound, and (ii) it is metabolized in a subject to yield a biologically active derivative of the compound. Known techniques for derivatizing compounds to provide prodrugs can be employed. Such methods may utilize formation of a hydrolyzable coupling to the compound.

The present invention further provides that the compounds of the present invention may be administered in combination with a therapeutic agent used to prevent and/or treat metabolic and/or endocrine disorders, gastrointestinal disorders, cardiovascular disorders, obesity and obesity-associated disorders, central nervous system disorders, genetic disorders, hyperproliferative disorders and inflammatory disorders. Exemplary agents include analgesics (including opioid analgesics), anesthetics, antifungals, antibiotics, antiinflammatories (including nonsteroidal anti-inflammatory agents), anthelmintics, antiemetics, antihistamines, antihypertensives, antipsychotics, antiarthritics, antitussives, antivirals, cardioactive drugs, cathartics, chemotherapeutic agents (such as DNA-interactive agents, antimetabolites, tubulin-interactive agents, hormonal agents, and agents such as asparaginase or hydroxyurea), corticoids (steroids), antidepressants, depressants, diuretics, hypnotics, minerals, nutritional supplements, parasympathomimetics, hormones (such as corticotrophin releasing hormone, adrenocorticotropin, growth hormone releasing hormone, growth hormone, thyrotropin-releasing hormone and thyroid stimulating hormone), sedatives, sulfonamides, stimulants, sympathomimetics, tranquilizers, vasoconstrictors, vasodilators, vitamins and xanthine derivatives.

Subjects suitable to be treated according to the present invention include, but are not limited to, avian and mammalian subjects, and are preferably mammalian. Mammals of the d present invention include, but are not limited to, canines, felines, bovines, caprines, equines, ovines, porcines, rodents (e.g. rats and mice), lagomorphs, primates, humans, and the like, and mammals in utero. Any mammalian subject in need of being treated according to the present invention is suitable. Human subjects are preferred. Human subjects of both genders and at any stage of development (i.e., neonate, infant, juvenile, adolescent, adult) can be treated according to the present invention.

Illustrative avians according to the present invention include chickens, ducks, turkeys, geese, quail, pheasant, ratites (e.g., ostrich) and domesticated birds (e.g., parrots and canaries), and birds in ovo.

The present invention is primarily concerned with the treatment of human subjects, but the invention can also be carried out on animal subjects, particularly mammalian subjects such as mice, rats, dogs, cats, livestock and horses for veterinary purposes, and for drug screening and drug development purposes.

In therapeutic use for treatment of conditions in mammals (i.e. humans or animals) for which an antagonist of the motilin receptor is effective, the compounds of the present invention or an appropriate pharmaceutical composition thereof may be administered in an effective amount. Since the activity of the compounds and the degree of the therapeutic effect vary, the actual dosage administered will be determined based upon generally recognized factors such as age, condition of the subject, route of delivery and body weight of the subject. The dosage can be from about 0.10 to about 100 mg/kg, administered orally 1-4 times per day. In addition, compounds can be administered by injection at approximately 0.01-20 mg/kg per dose, with administration 1-4 times per day. Treatment could continue for weeks, months or longer. Determination of optimal dosages for a particular situation is within the capabilities of those skilled in the art.

### 5. Methods of Use

The compounds of formula I of the present invention can be used for the prevention and treatment of a range of gastrointestinal disorders including irritable bowel syndrome (IBS), dyspepsia, including gallbladder dyspepsia, gastroesophogeal reflux disorders, Crohn's disease, ulcerative colitis, pancreatitis, infantile hypertrophic pyloric stenosis, malabsorption syndrome, carcinoid syndrome, diarrhea, atrophic colitis or gastritis, gastrointestinal dumping syndrome and postgastroenterectomy syndrome.

According to a further aspect of the invention, there is provided a method for the treatment of gastrointestinal disorders characterized by a disturbed migrating motor complex including irritable bowel syndrome, dyspepsia, including gallbladder dyspepsia, gastroesophogeal reflux disorders, Crohn's disease, ulcerative colitis, pancreatitis, infantile hypertrophic pyloric stenosis, malabsorption syndrome, carcinoid syndrome, diarrhea, atrophic colitis or gastritis, gastrointestinal dumping syndrome, postgastroenterectomy syndrome and other gastrointestinal disorders, which method comprises administering to said patient an effective amount of at least one member selected from the compounds disclosed herein having the ability to antagonize the motilin receptor.

In particular embodiments, the macrocyclic compounds of the present invention can be used to treat diarrhea, cancer treatment-related diarrhea, cancer-induced diarrhea, chemotherapy-induced diarrhea, radiation enteritis, radiation-induced diarrhea, stress-induced diarrhea, chronic diarrhea, AIDS-related diarrhea, C. difficile associated diarrhea, traveller's diarrhea, acute infectious diarrhea, diarrhea induced by graph versus host disease, and other types of diarrhea.

In another embodiment, the macrocyclic compounds of the present invention can be used to treat irritable bowel syndrome, dyspepsia, functional gastrointestinal disorders, chemotherapy-induced nausea and vomiting (emesis), post-operative nausea and vomiting, cyclic vomiting syndrome and functional vomiting.

As used herein, “treatment” is not necessarily meant to imply cure or complete abolition of the disorder or symptoms associated therewith.

According to other embodiments of the present invention, the compounds described herein provide methods of modulating the migrating motor complex in humans and other mammals.

The compounds of the present invention can further be utilized for the preparation of a medicament for the treatment of a range of medical conditions involving gastrointestinal motility disorders.

The compounds of the present invention can also be utilized for the preparation of a medicament for the treatment of a range of medical conditions involving poor stomach or intestinal absorption.

Further embodiments of the present invention will now be described with reference to the following examples. It should be appreciated that these examples are for the purposes of illustrating embodiments of the present invention, and do not limit the scope of the invention.

### EXAMPLE 1

### Effect of Compound 502 on the Migrating Motor Complex in Dogs

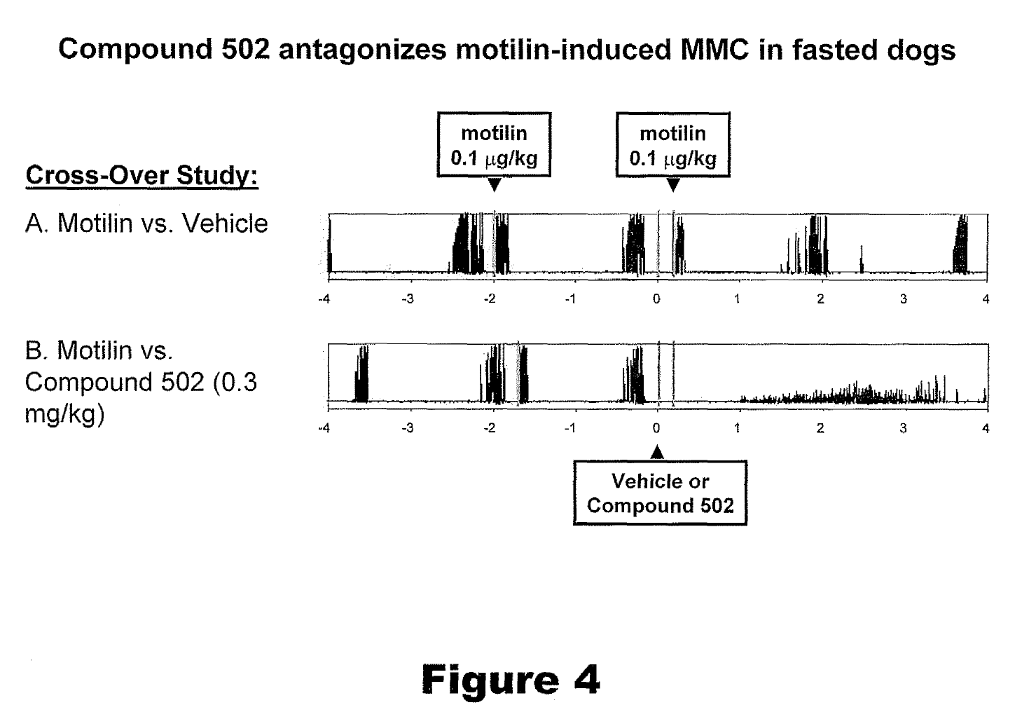

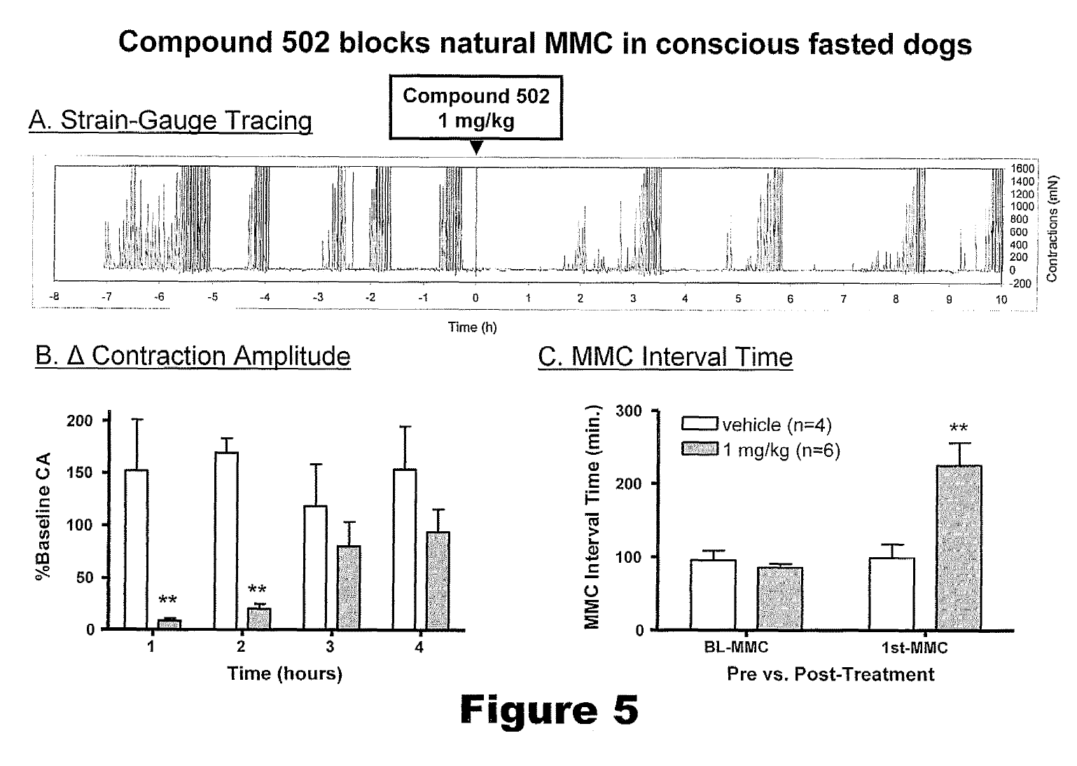

In the first series of experiments according to Method F (FIGS. 4 and 5), Compound 502 (1 mg/kg, i.v.) was observed to delay the migrating motor complex (MMC) in fasted beagle dogs N=6). This effect appears to be dose-dependent as data indicates a lesser delay of MMCs at a dose of 0.30 mg/kg (i.v.). This effect on the MMC may be relevant to IBS-diarrhea type (IBS-d) as these patients are known to have higher frequency and amplitude in MMCs. Alosetron (Lotronex™) is a serotonin (5-HT₃) agonist currently available for restricted use for IBS-d that is also known to block the MMC in dog.

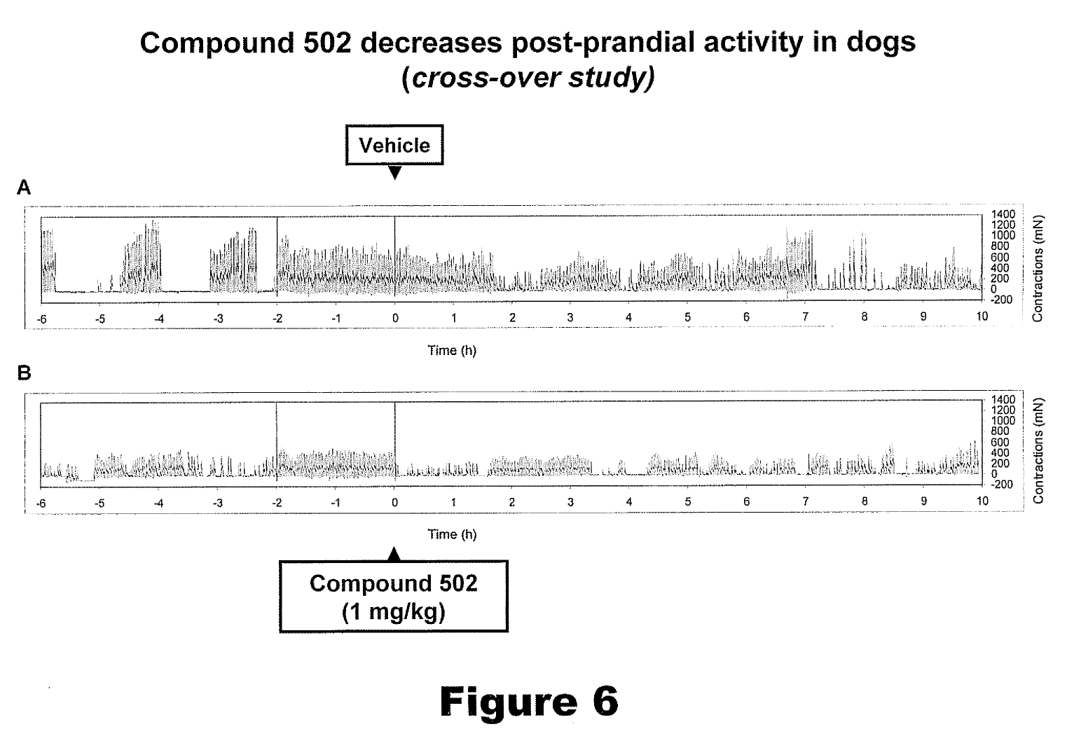

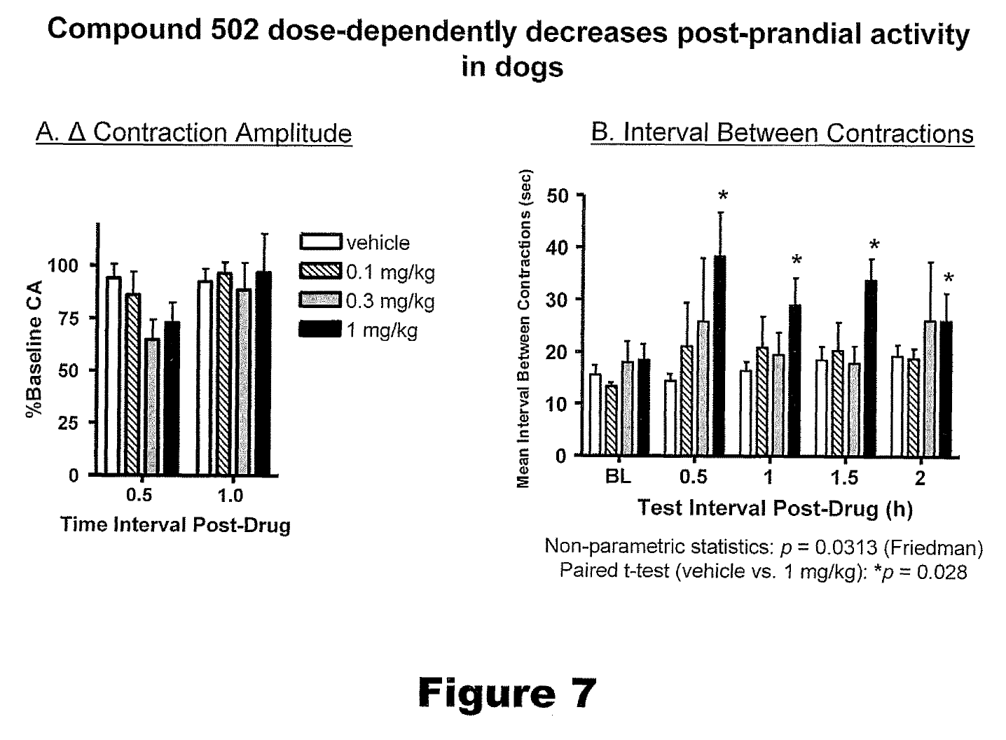

In the second series of experiments conducted according to Method F (FIGS. 6 and 7), Compound 502 (0.30 mg/kg, i.v.) was shown to briefly attenuate post-prandial activity in fed dogs. This observation is relevant to both IBS-d and functional dyspepsia (FD) since (1) IBS-d patients are known to have hyper-responsive gastrointestinal activity in response to a meal and (2) some FD patients are known to have poor gastric accommodation in response to a meal. This strain-gauge result indicates that the normal muscle contractions in the stomach in response to meal are muted.

Following an overnight fast, compound 502 (0.10, 0.30, 1.0 mg/kg, i.v.) suppressed the regular occurrence of phase III activity of the MMC in a dose-dependent manner. Baseline MMC-interval during the 4 hr period before drug was 86±5 min and average contraction amplitude (CA) was 276.7±66.0 mN (mean±SEM, n=6). After 1.0 mg/kg dose, time to first MMC was 225±31 min vs. 99±15 min in controls (P<0.01, n=4) and CA was reduced to 5±2% of baseline CA over the first hour and recovered to 52±21% after 4 h. The 0.10 and 0.30 mg/kg doses also suppressed MMC activity where CA returned to baseline levels after 3 h (n=2). After a standard 75 g meal, compound 502 (0.30 mg/kg, i.v., admin. 2 h after meal) suppressed post-prandial CA during the first hour to 58±4% of pre-drug baseline (n=2) whereas vehicle treatment maintained CA at 106±10% (n=4).

### EXAMPLE 2

### Effect of Compound 502 on Fundic Accommodation in Dogs

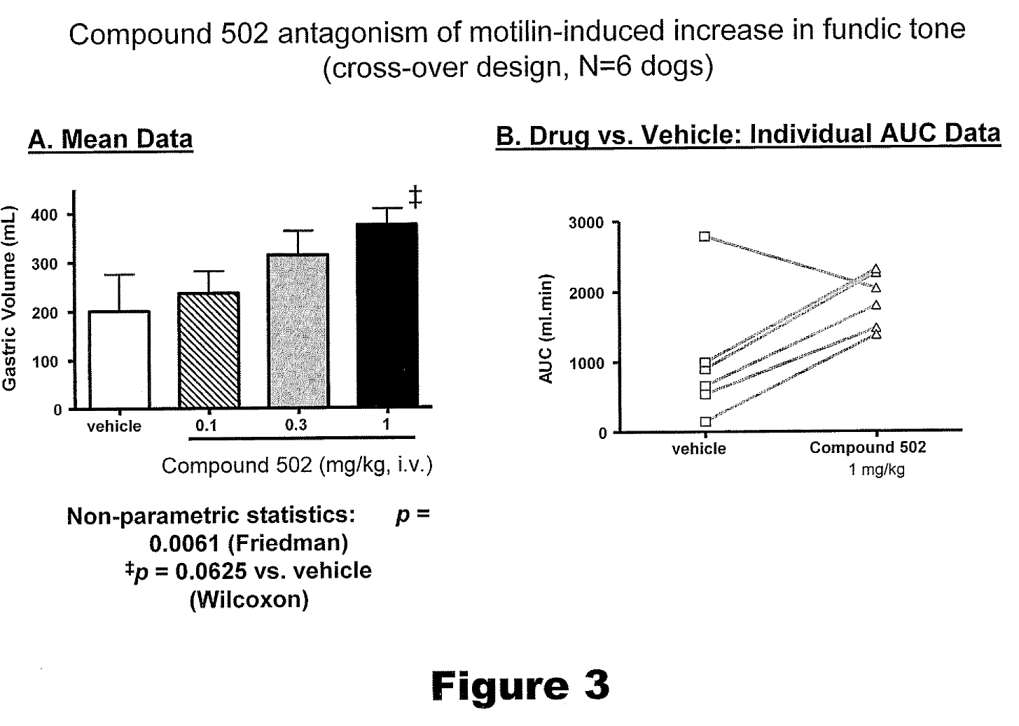

Compound 502 (0.30 mg/kg, i.v.) antagonized the effects of exogenous motilin in the short period before the meal and further improved gastric accommodation in response to the meal (FIGS. 2 and 3). The brief improvement in gastric accommodation in response to Compound 502 may be due to the fact that it was administered by i.v. bolus whereas the motilin is being administered by continuous infusion through the duration of the experiment.

In response to a milk meal as described in Method E, Compound 502 (0.30 mg/kg, i.v.) antagonized the fundic contraction induced by infusion of exogenous motilin (0.010 mg/kg/h, i.v., FIG. 2); AUC of fundic relaxation (0-to-5 min.) for vehicle administration was 18.7±3.3 vs. 27.3±3.5 mL/h after compound 502 (n=4, p<0.05). Compound 502 further prevented this increase in a dose-dependent manner as shown in FIG. 3.

It is appreciated that although specific experimental methods have been described herein for the purposes of illustration, various modifications to these experimental methods as well as alternate methods of experimentation may be used without departing from the scope of this invention.

What is claimed is:

1. A method comprising administering to a subject an effective amount of a compound of formula I or a pharmaceutical composition comprising an effective amount of a compound of formula I 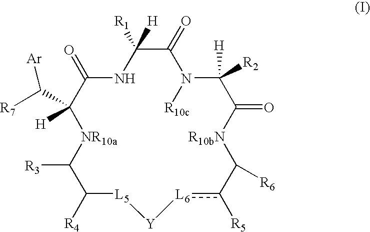
or pharmaceutically acceptable salts, hydrates or solvates thereof wherein:
Y is 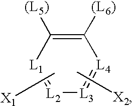
wherein (L₅) and (L₆) indicate the bonds to L₅ and L₆ of formula I, respectively;
Ar is selected from the group consisting of: 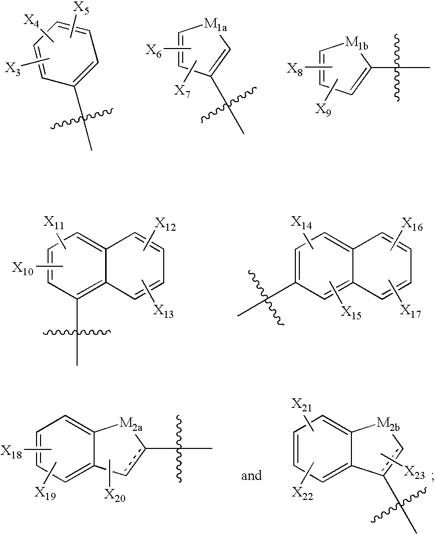
R₁ is selected from the group consisting of: —(CH₂)ₛCH₃, —CH(CH₃)(CH₂)ₜCH₃, —(CH₂)ᵤCH(CH₃)₂, —C(CH₃)₃, and 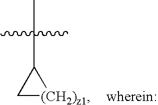
s is 0, 1, 2 or 3;
t is 1 or 2;
u is 0 or 1; and
z1 is 1, 2, 3 or 4;
R₂ is selected from the group consisting of hydrogen, —(CH₂)ₐₐCH₃, —CH₂SCH₃, —CH₂CH₂SCH₃, —(CH₂)_{bb}CH(CH₃)₂, —CH(CH₃)(CH₂)_{cc}CH₃, —(CH₂)_{dd}—NR₁₁R₁₂, and —(CH₂)ₑₑR₁₃; wherein
aa and bb are independently 0, 1, 2 or 3;
cc and dd are independently 1, 2, 3 or 4;
ee is 0, 1, 2, 3 or 4;
R₁₁ is selected from the group consisting of hydrogen, lower alkyl, formyl, acyl, carboxyalkyl, carboxyaryl, amido, amidino, sulfonyl and sulfonamido;
R₁₂ is selected from the group consisting of hydrogen and lower alkyl
R₁₃ is selected from the group consisting of: 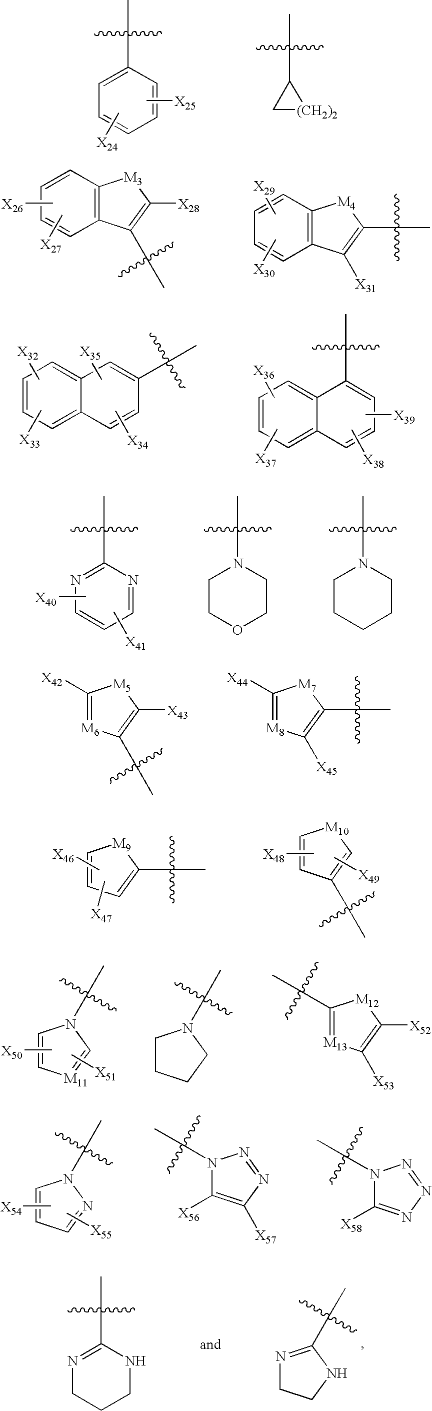
wherein z2 is 1, 2, 3 or 4;
and, when ee is 1, 2, 3 or 4, R₁₃ is further selected from the group consisting of hydroxy, alkoxy, amidino, and azido;
R₃, R₄, R₅, and R₆ are independently selected from the group consisting of hydrogen, methyl, ethyl, isopropyl and hydroxymethyl;
R₇ is selected from the group consisting of hydrogen, methyl, hydroxy and amino;
R₁₀ₐ and R_{10b} are independently selected from the group consisting of hydrogen and methyl;
X₁, X₂, X₆, X₇, X₈ and X₉ are independently selected from the group consisting of hydrogen, halogen, trifluoromethyl and lower alkyl;
X₃, X₄, X₅, X₁₀, X₁₁, X₁₂, X₁₃, X₁₄, X₁₅, X₁₆, X₁₇, X₁₈, X₁₉, X₂₁, X₂₂, X₂₄, X₂₅, X₂₆, X₂₇, X₂₈, X₂₉, X₃₀, X₃₁, X₃₂, X₃₃, X³⁴, X₃₅, X₃₆, X₃₇, X₃₈, X₃₉, X₄₀, X₄₁, X₄₂, X₄₃, X₄₄, X₄₅, X₄₆, X₄₇, X₄₈, X₄₉, X₅₀, X₅₁, X₅₂, X₅₃, X₅₄ and X₅₅ are independently selected from the group consisting of hydrogen, hydroxy, alkoxy, amino, halogen, trifluoromethyl and lower alkyl;
X₂₀ and X₂₃ are independently selected from the group consisting of hydrogen, trifluoromethyl and lower alkyl;
X₅₆, X₅₇ and X₅₈ are independently selected from the group consisting of hydrogen and lower alkyl;
L₁, L₂, L₃ and L₄ are independently selected from the group consisting of CH and N; with the proviso that the total number of nitrogens in the ring must be 0 or 1;
L₅ and L₆ are independently selected from the group consisting of O, CR₈ₐR_{8b} and NR₉ₐ; wherein R₈ₐ and R_{8b} are independently selected from the group consisting of hydrogen and methyl; and R₉ₐ is selected from the group consisting of hydrogen, lower alkyl, formyl, acyl and sulfonyl; with the proviso that when L₆ is CR₈ₐ when a double bond is present between L₆ and CHR₅;
M₁ₐ, M_{1b}, M₂ₐ, M_{2b}, M₃, M₄, M₅, M₇, M₉, M₁₀ and M₁₂ are independently selected from the group consisting of O, S and NR_{9b} wherein R_{9b} is selected from the group consisting of hydrogen, lower alkyl, formyl, acyl and sulfonyl; and
M₆, M₈, M₁₁, and M₁₃ are independently selected from the group consisting of N and CR_{9c}, wherein R_{9c} is selected from the group consisting of hydrogen and lower alkyl,
wherein the subject is in need of prevention or treatment of a disorder characterized by dysfunction of the migrating motor complex.

2. The method of claim 1, wherein the compound is selected from the group consisting of:

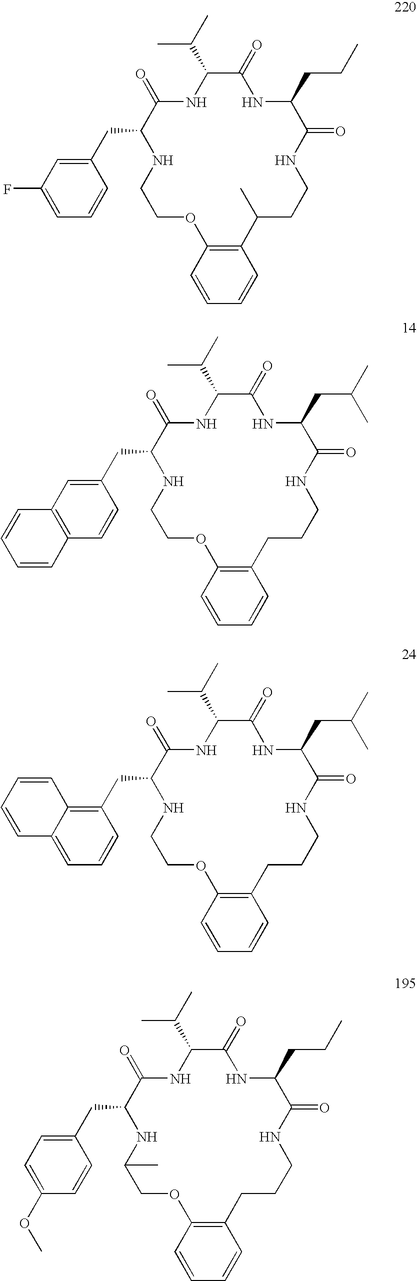

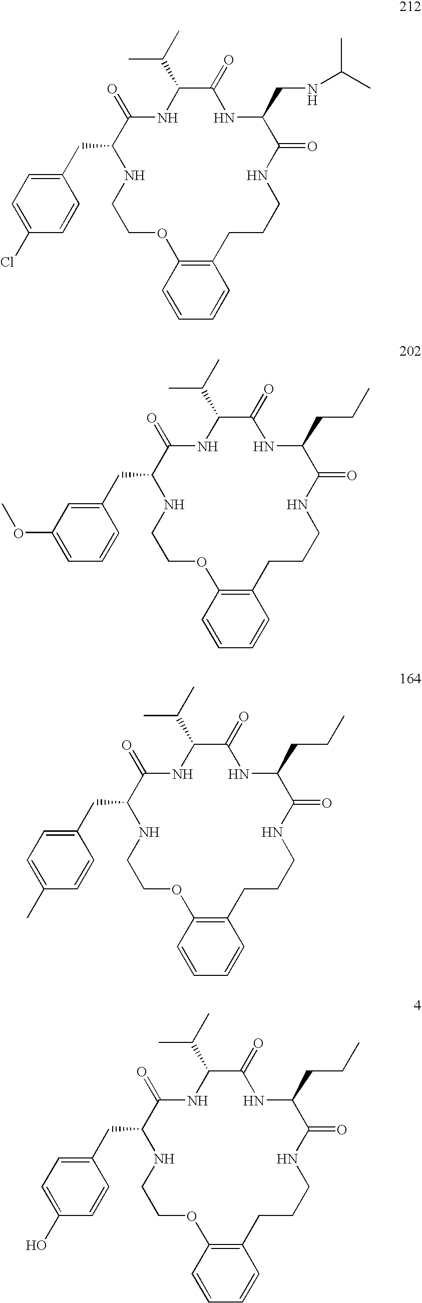

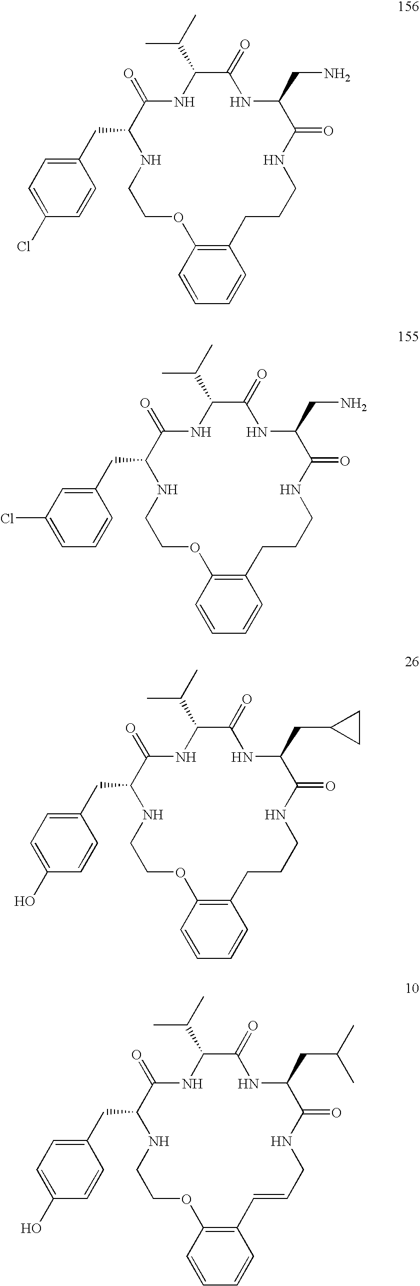

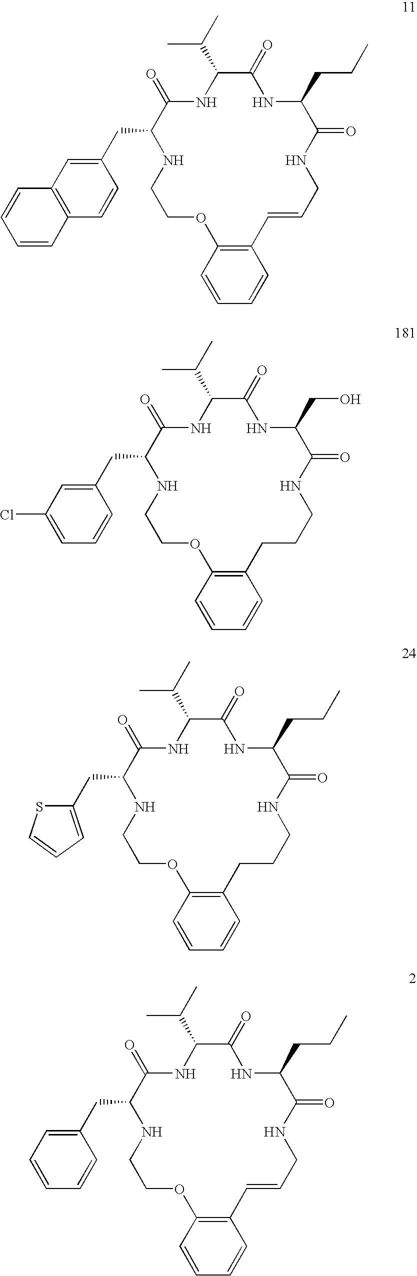

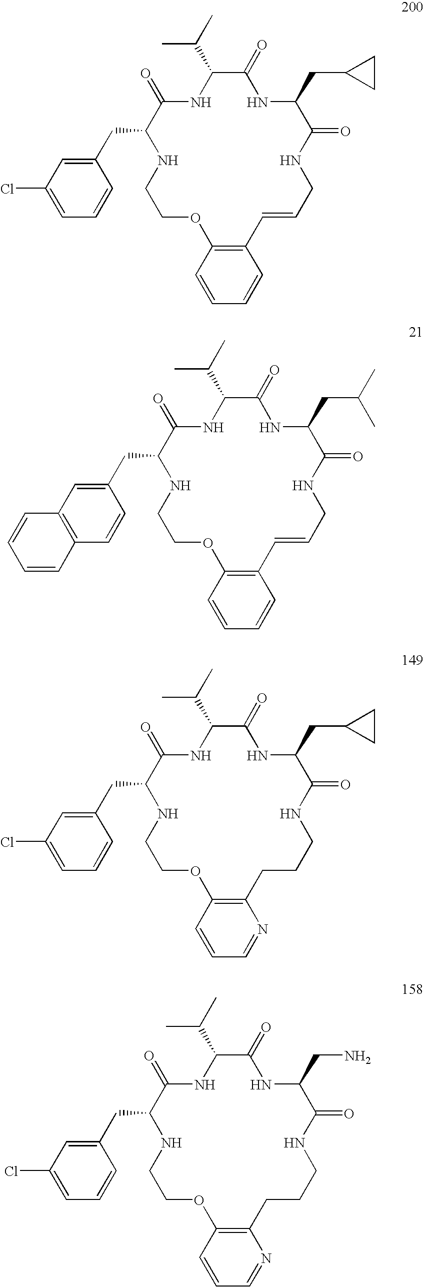

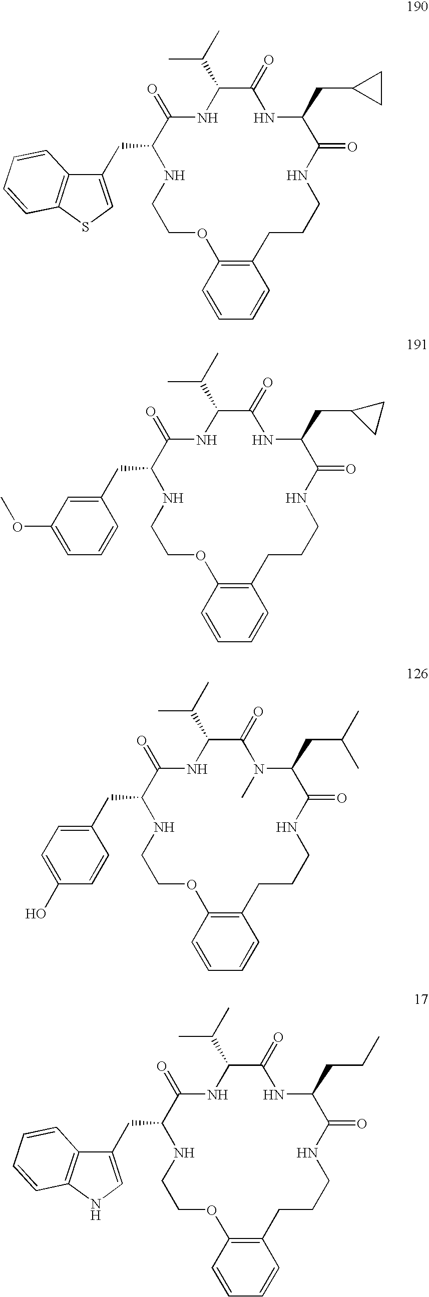

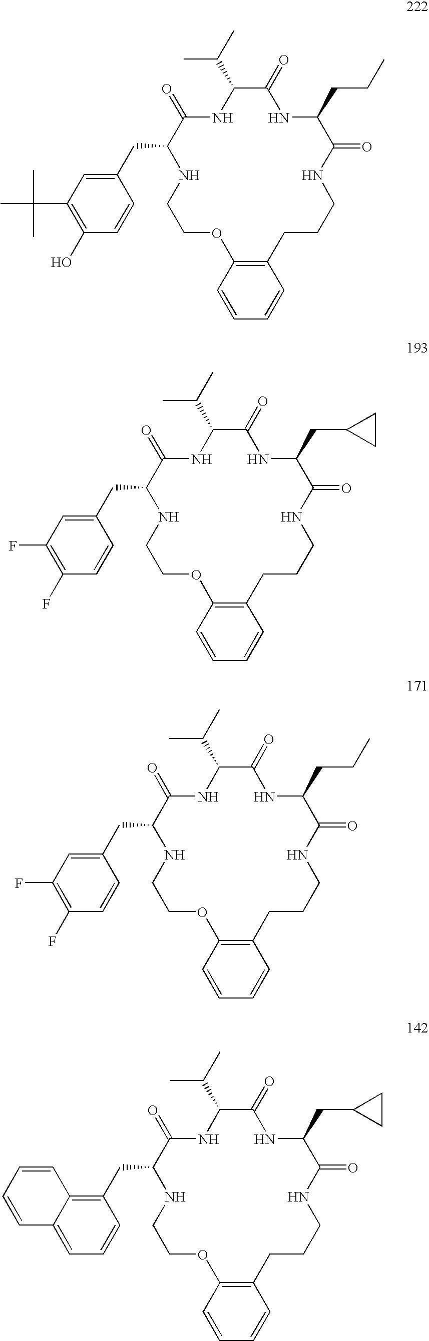

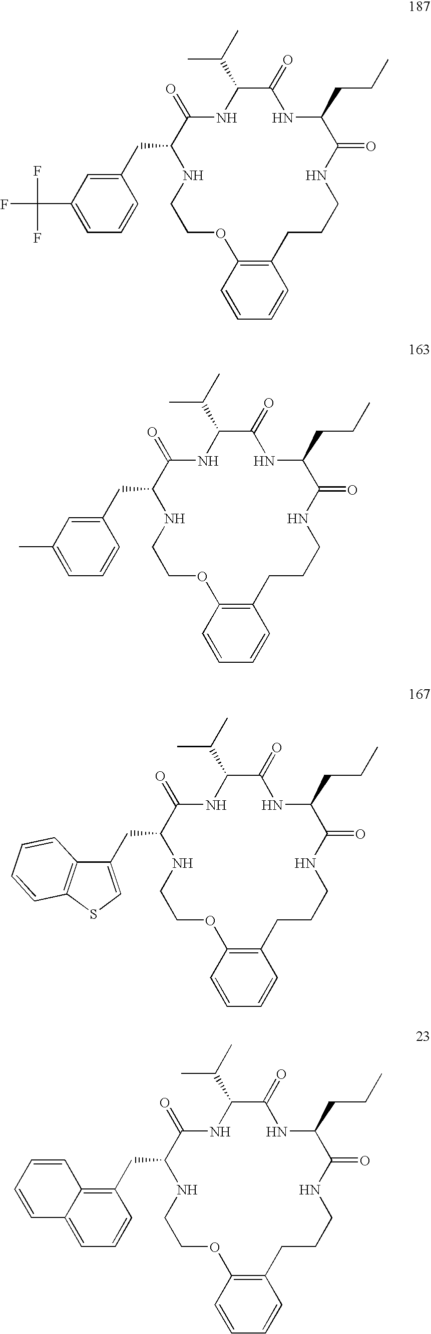

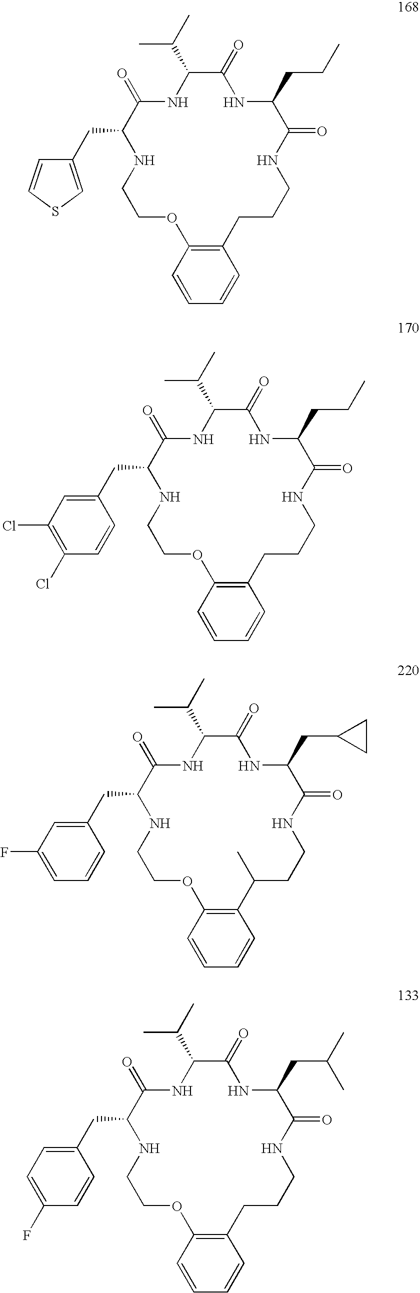

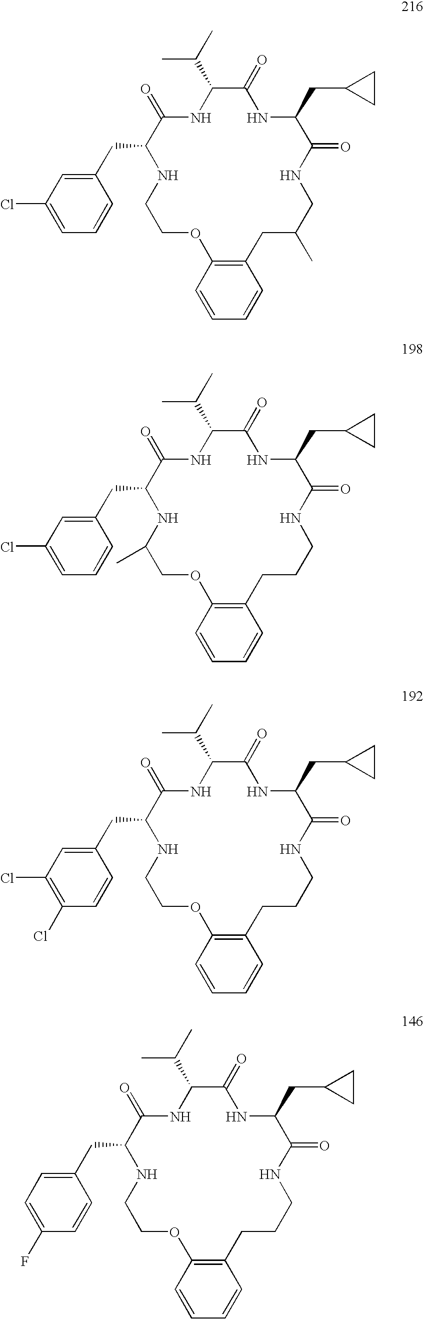

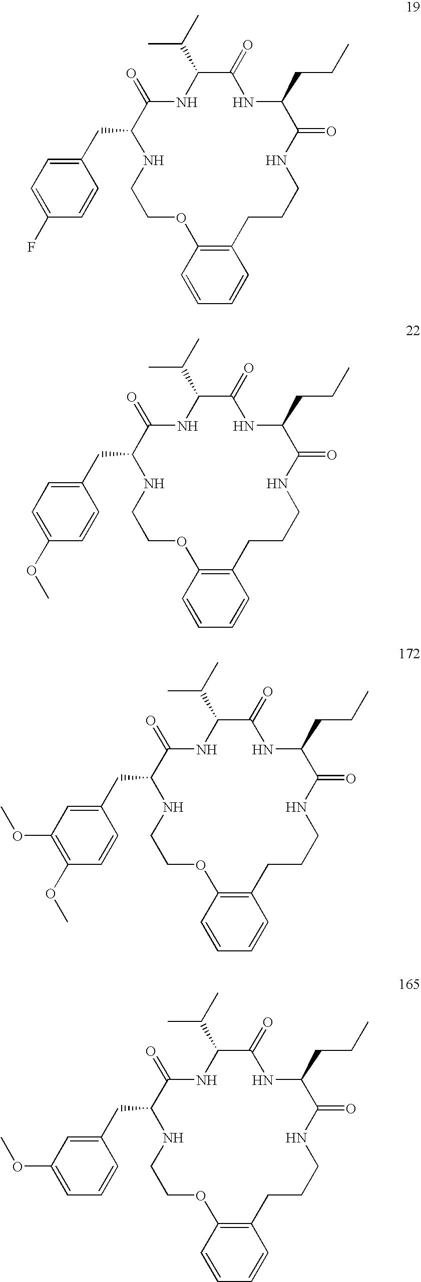

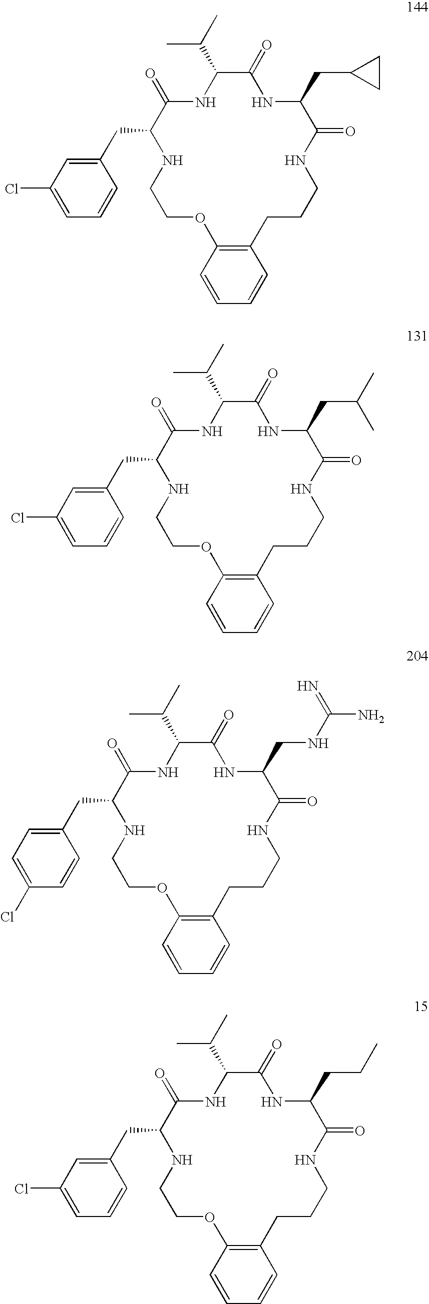

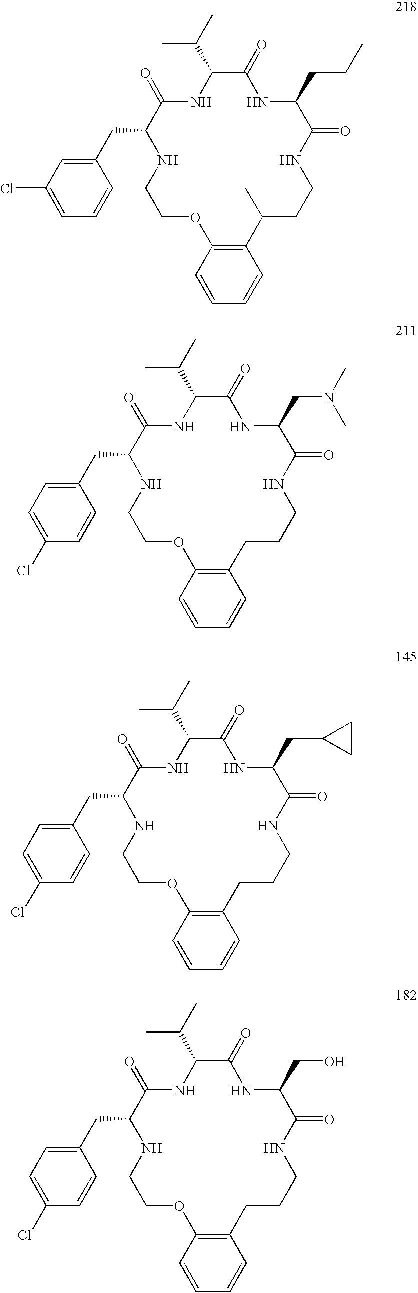

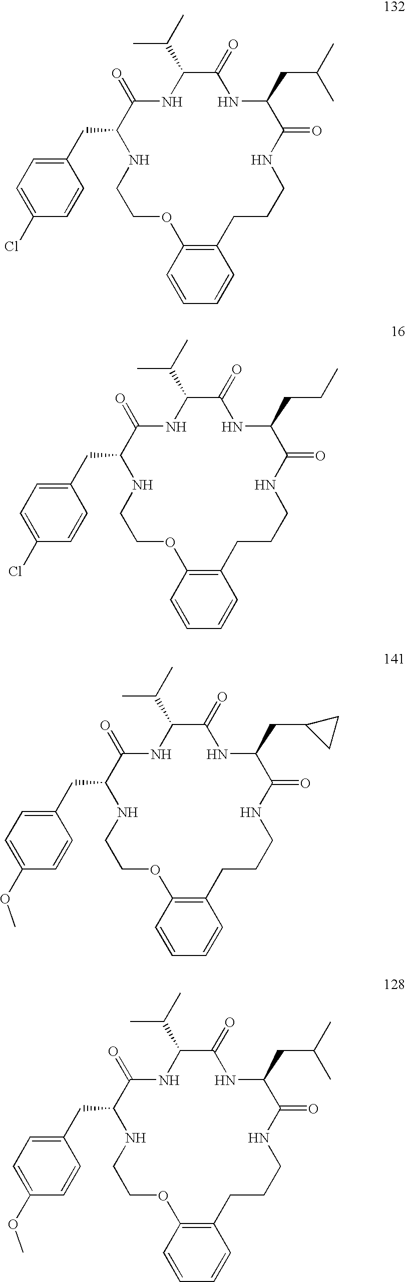

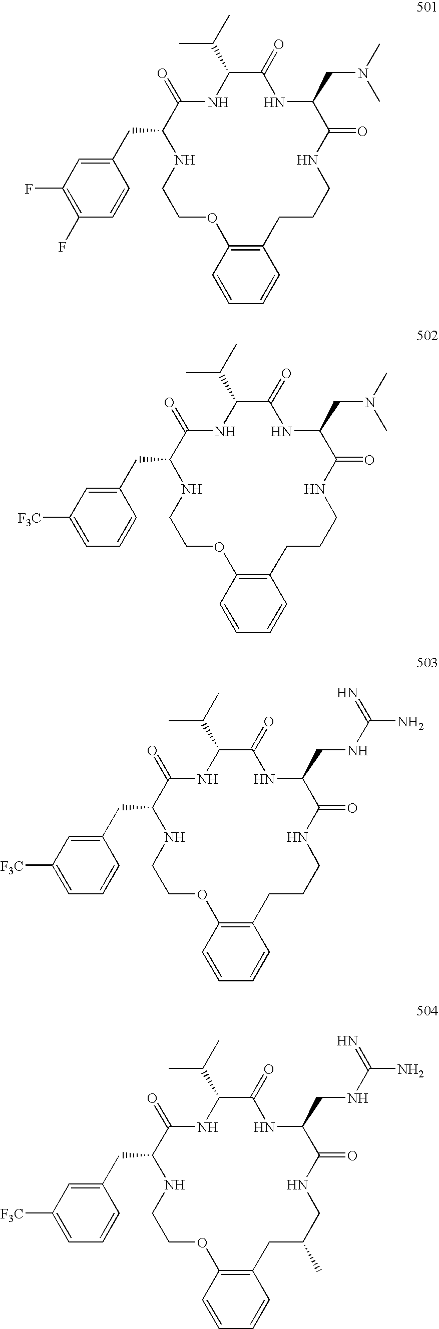

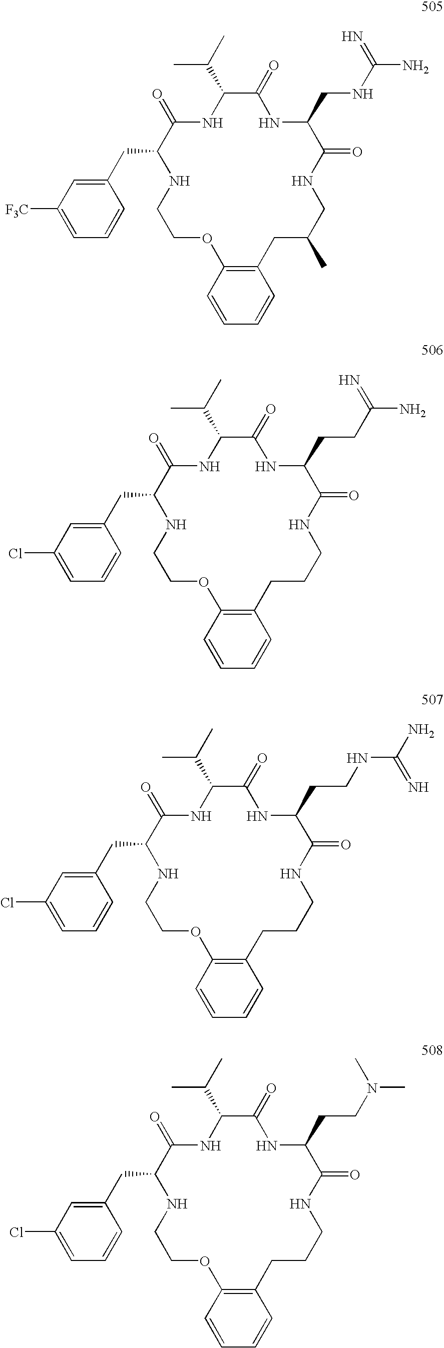

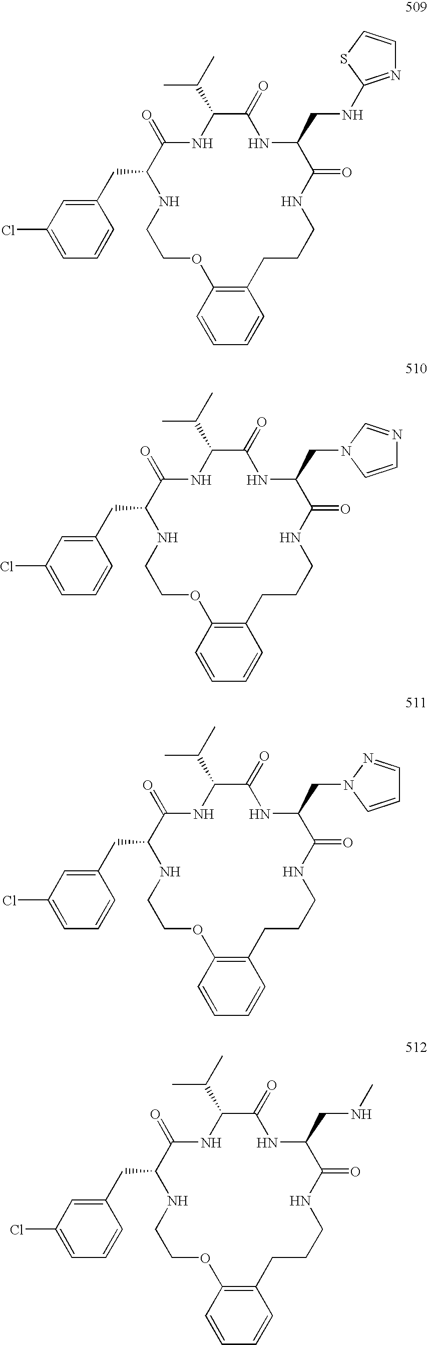

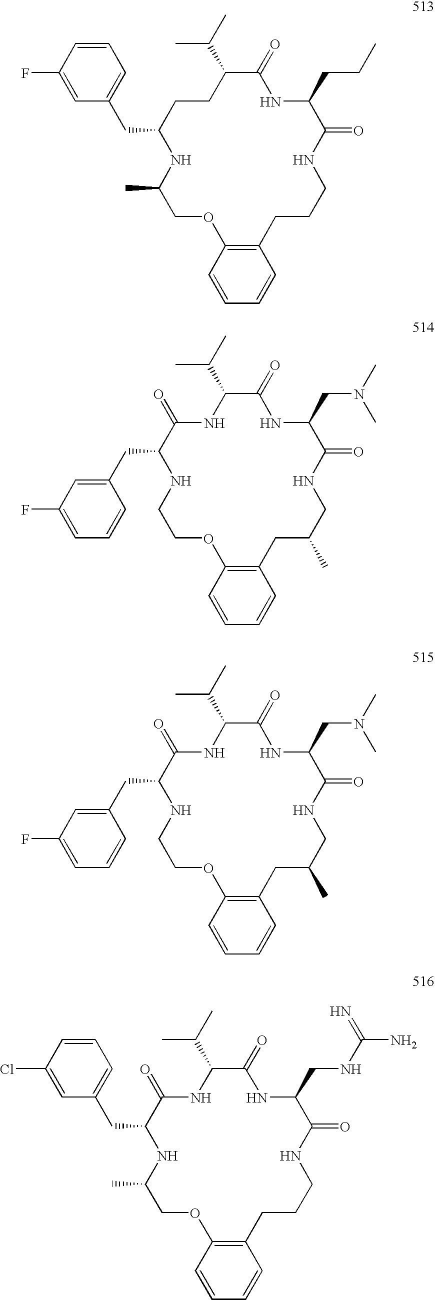

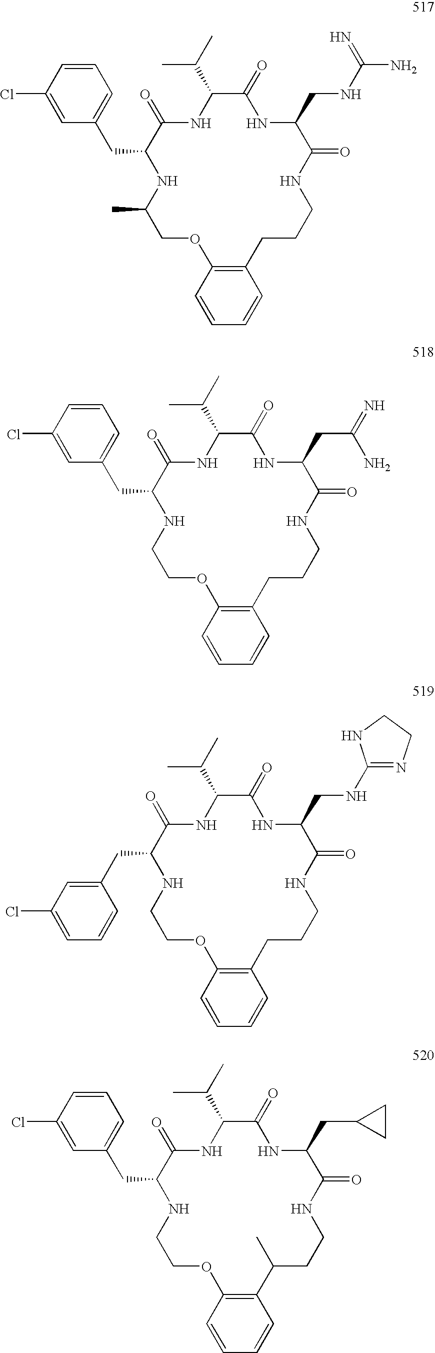

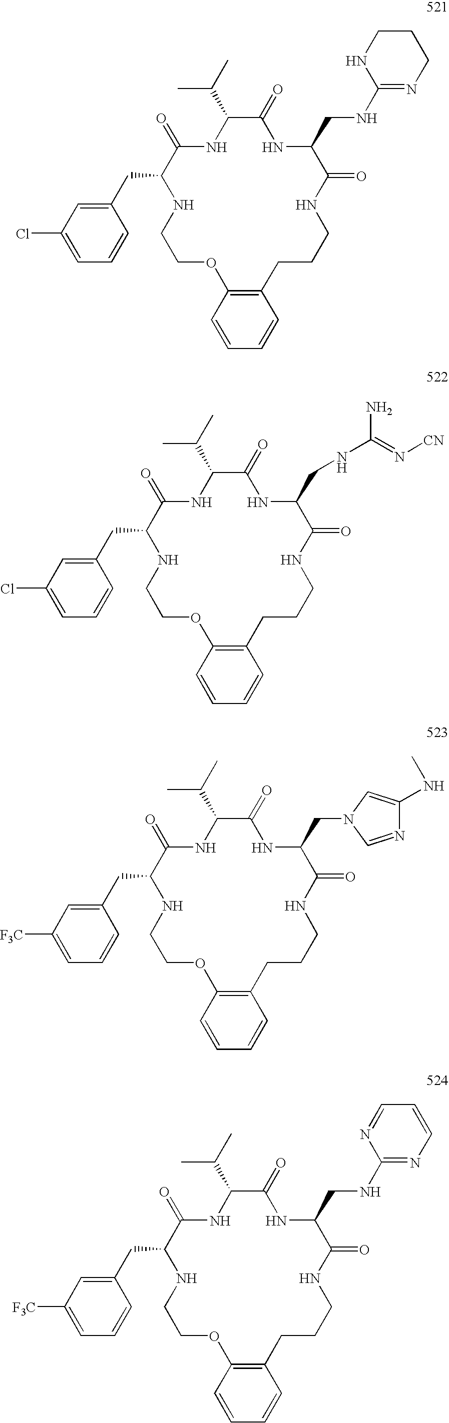

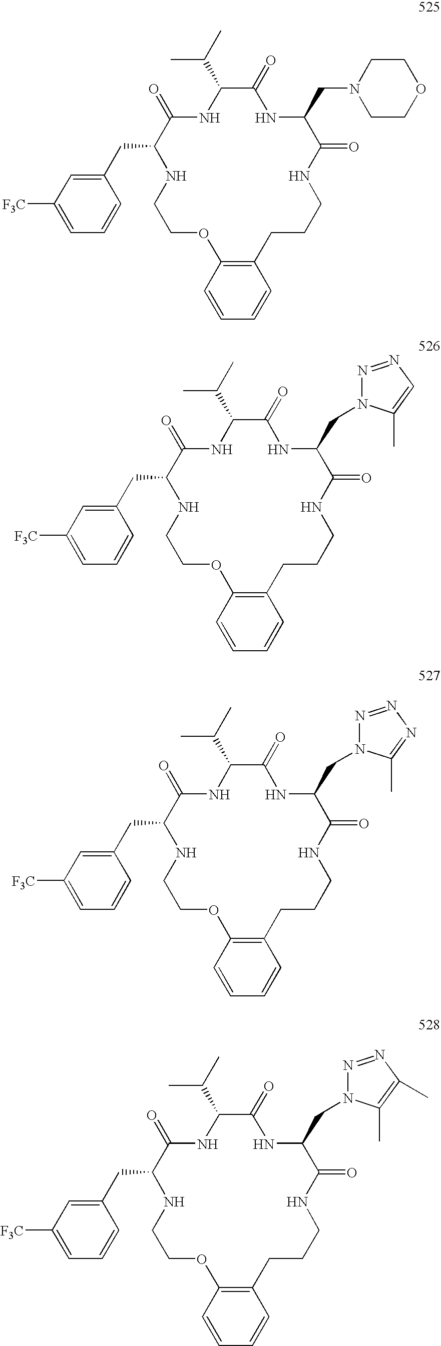

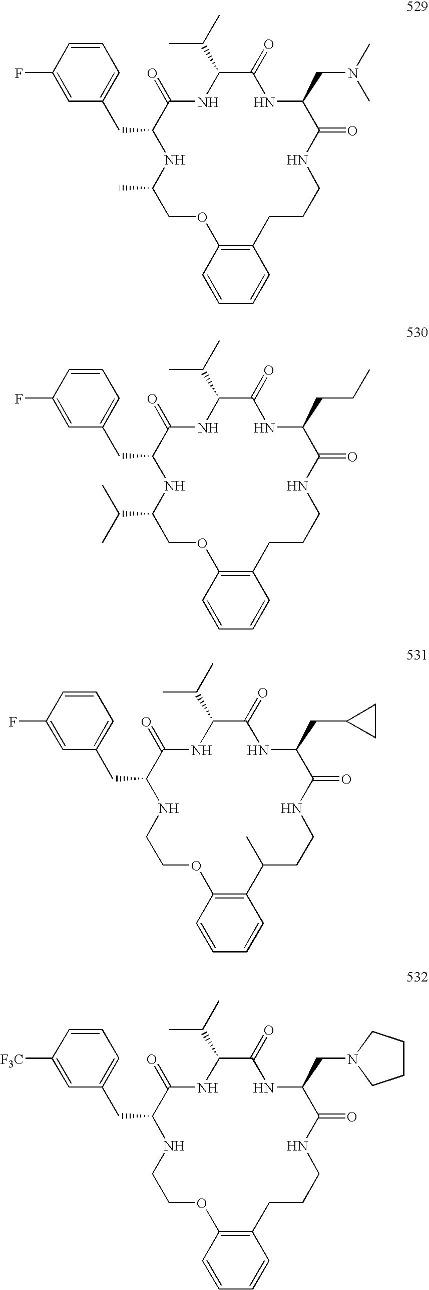

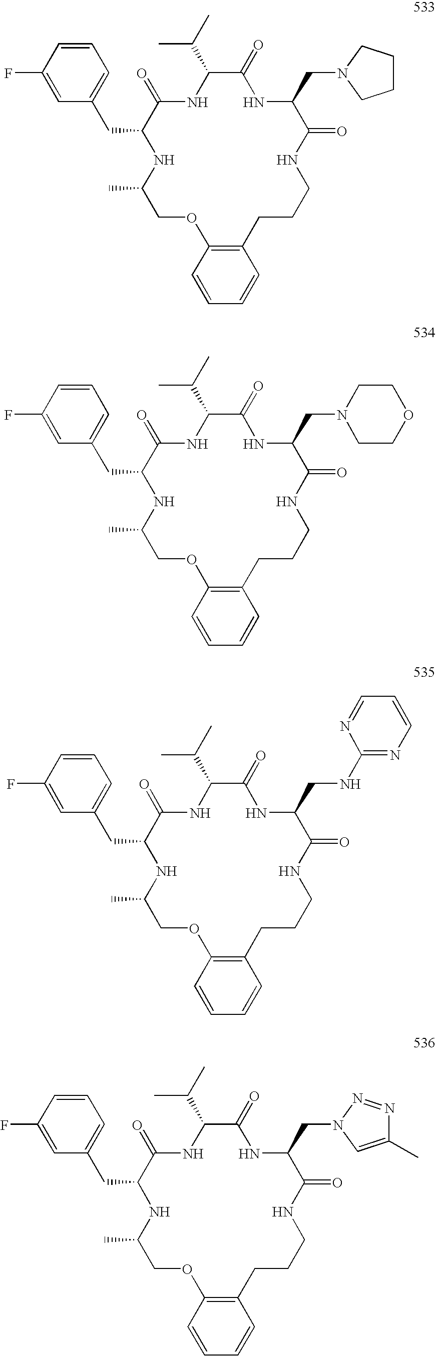

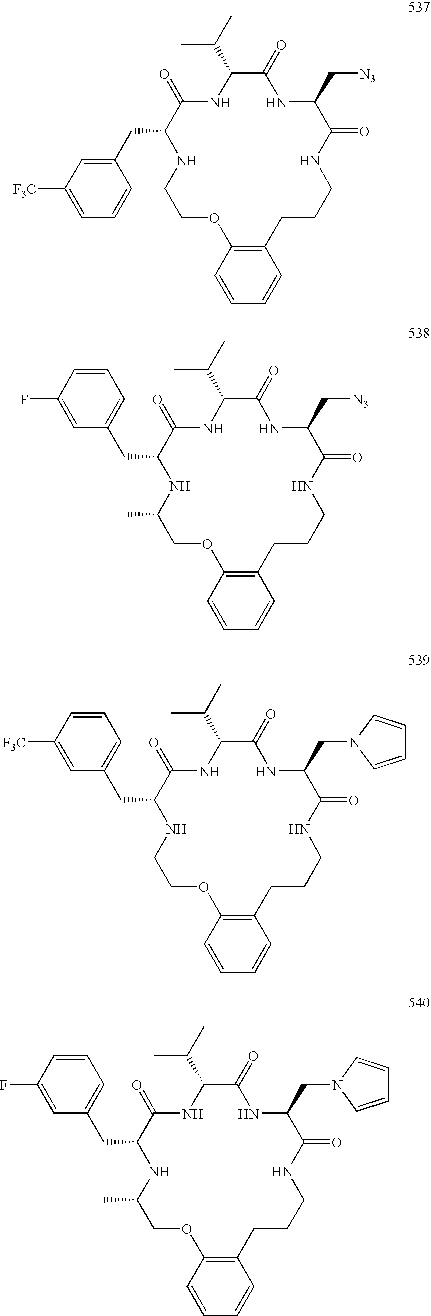

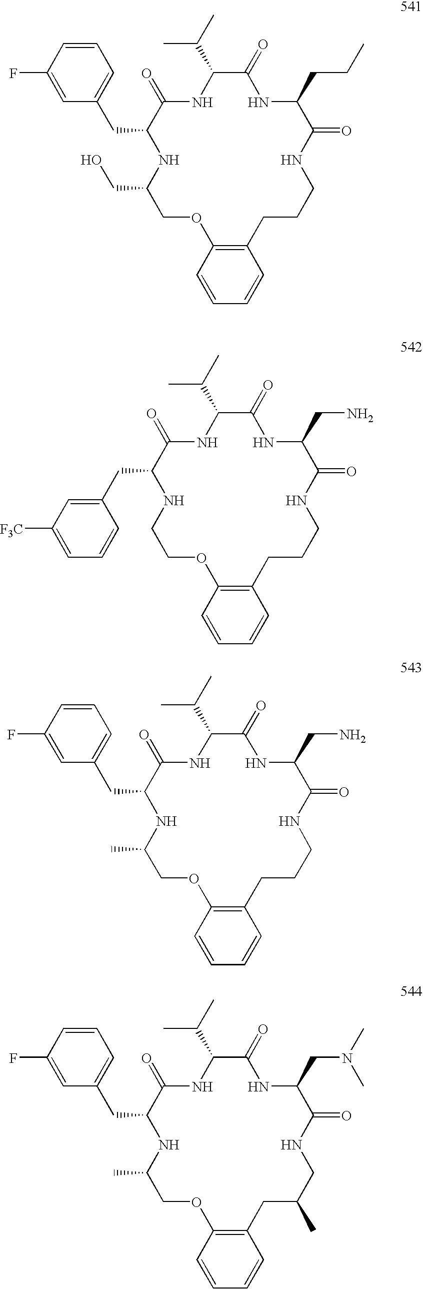

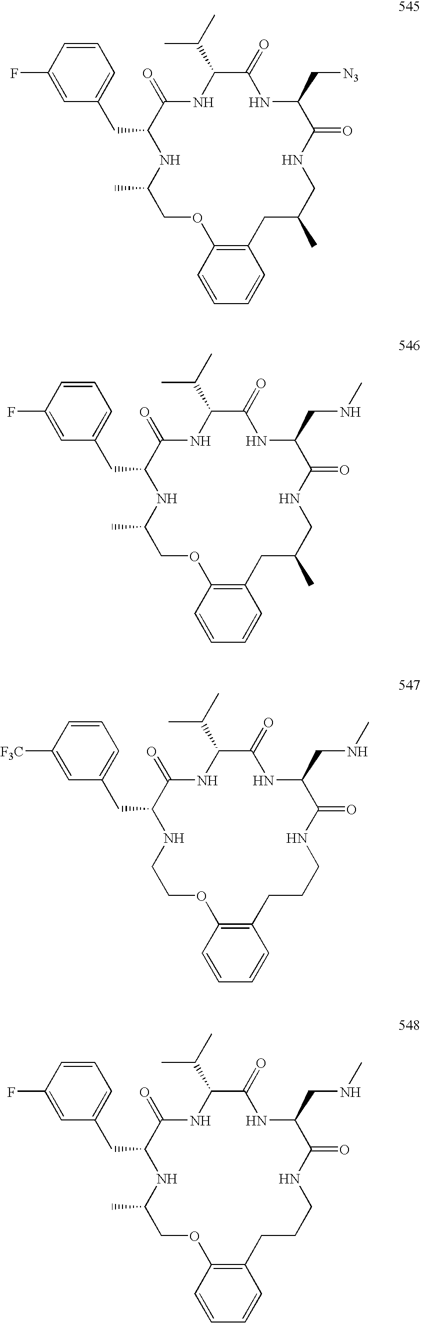

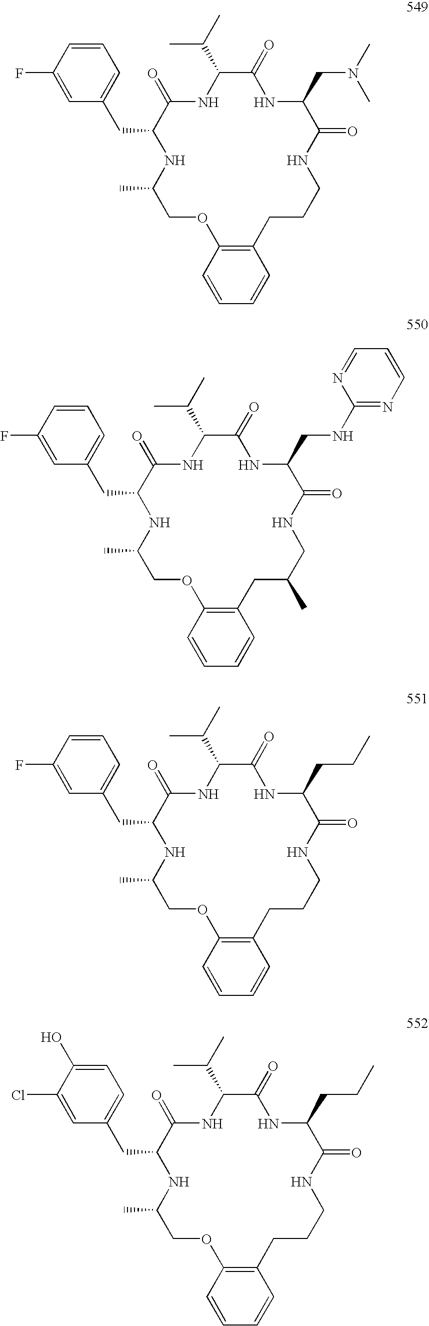

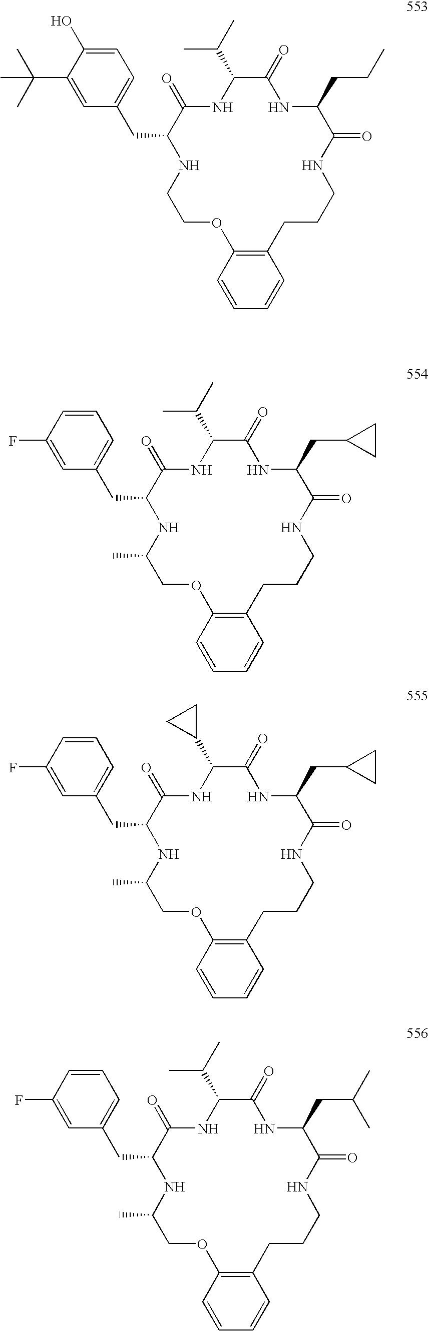

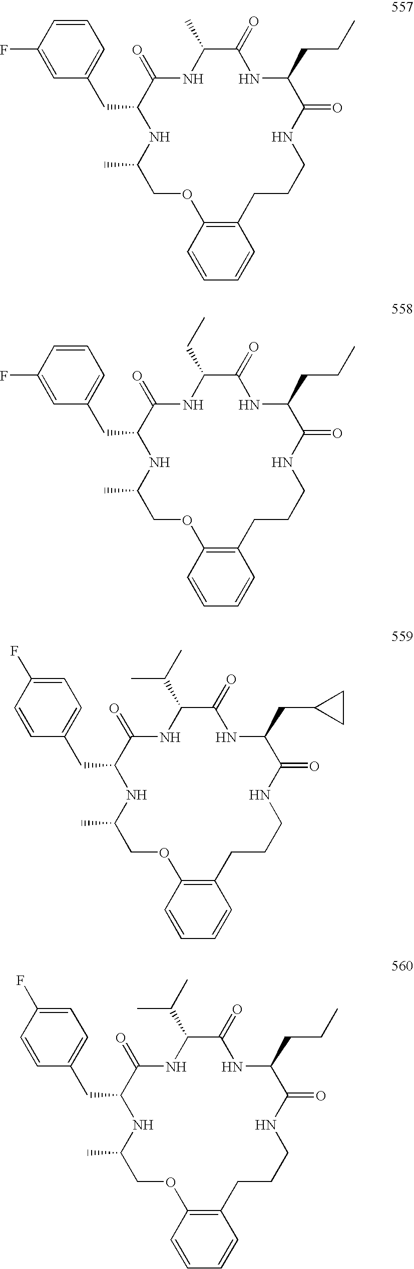

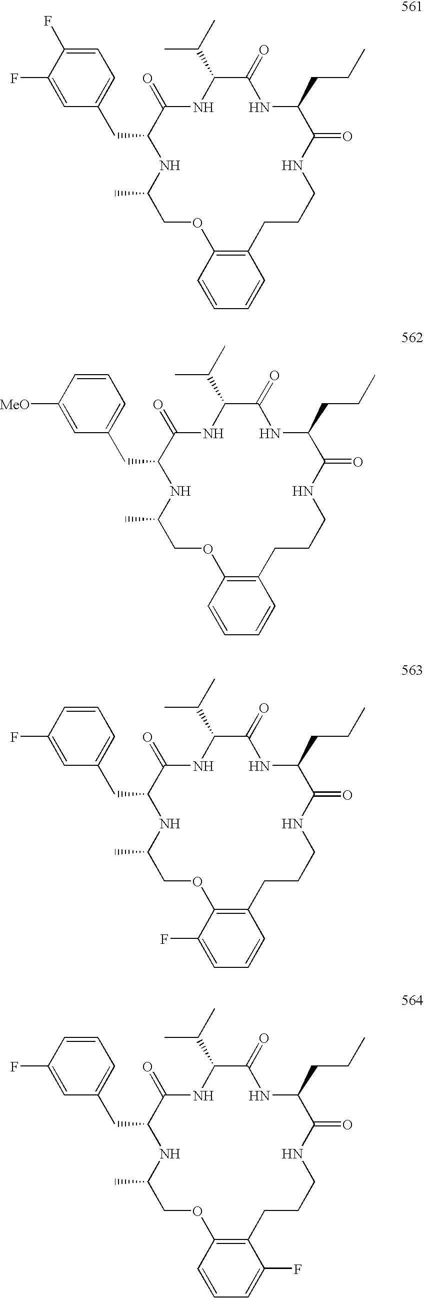

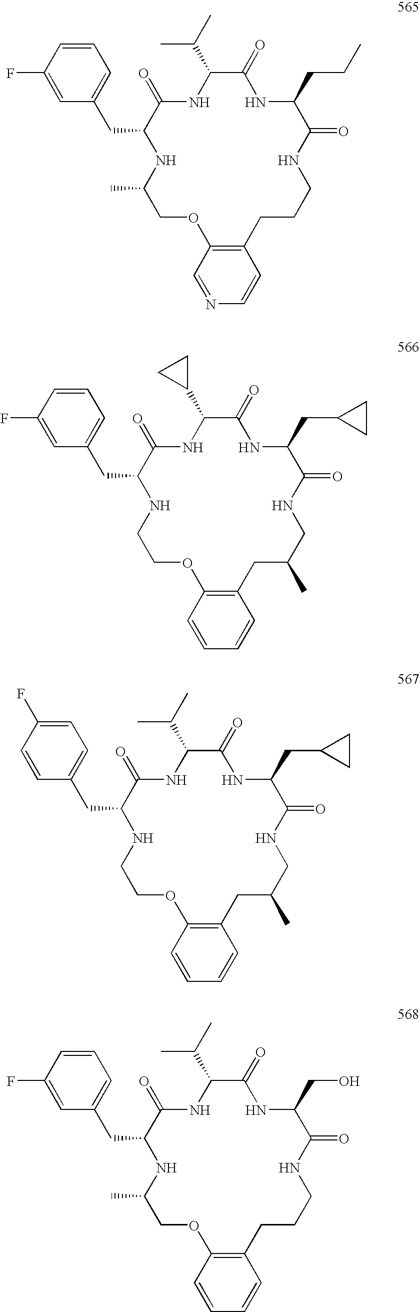

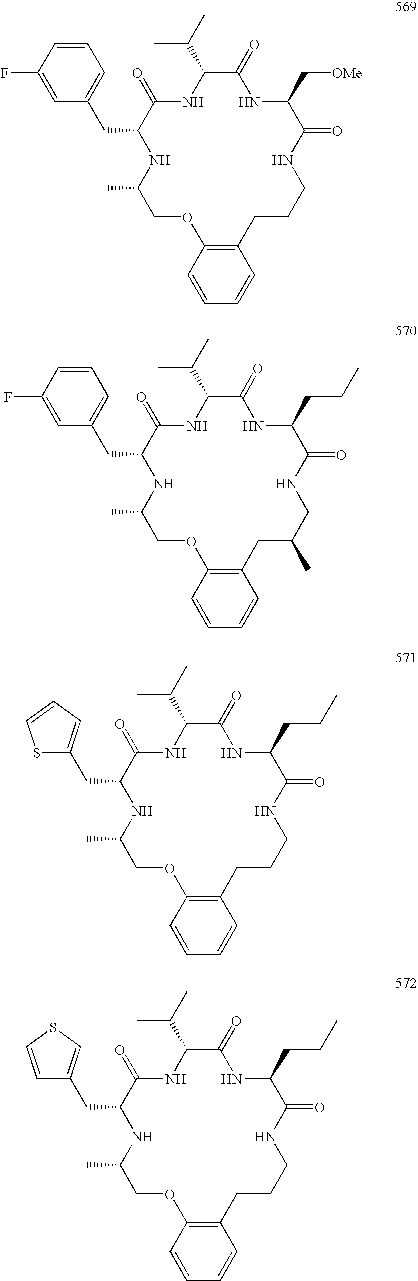

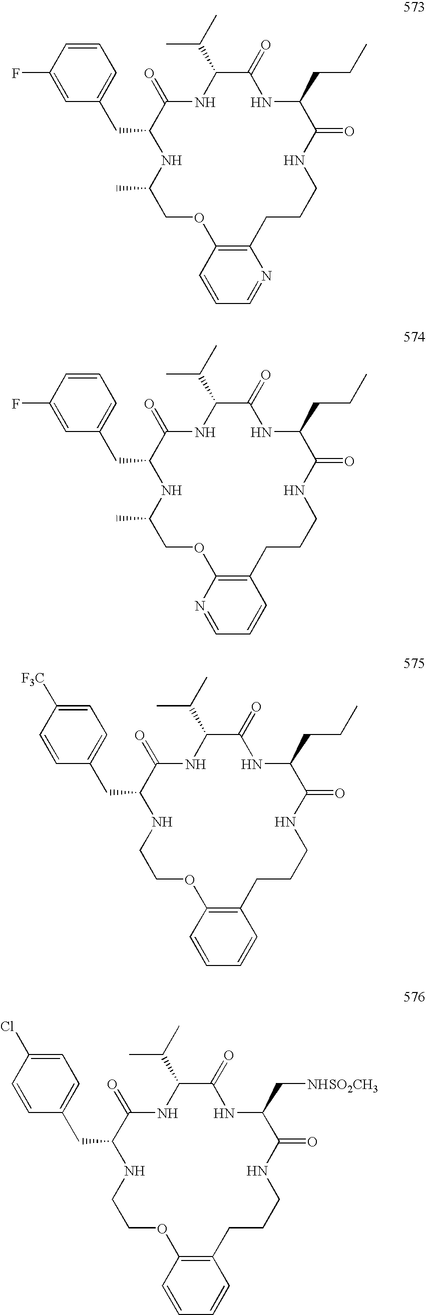

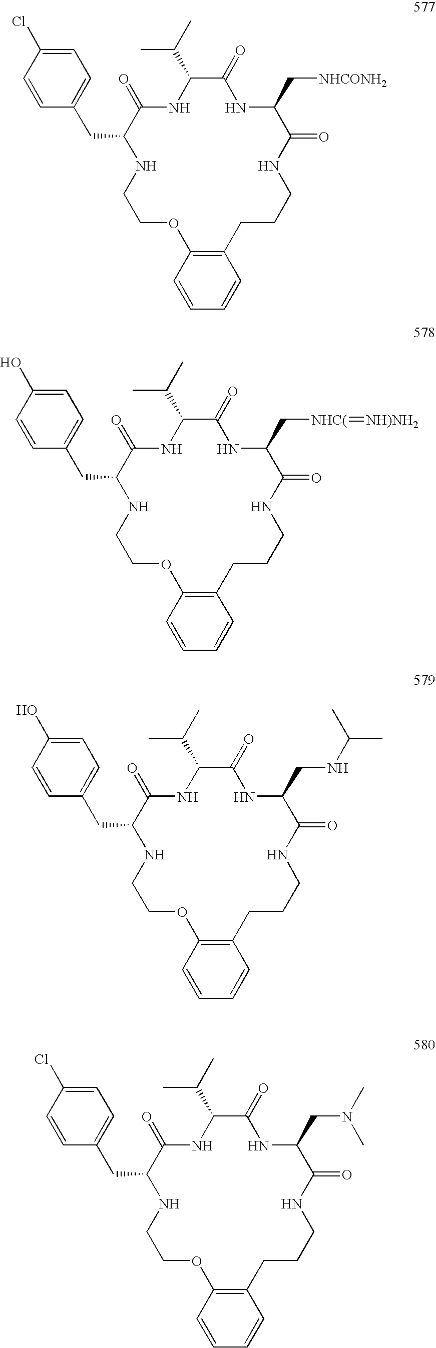

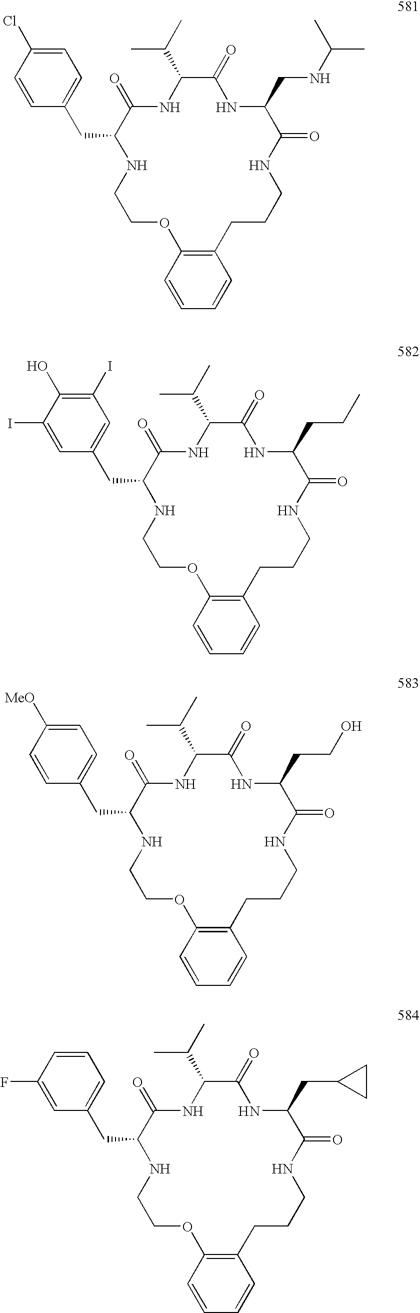

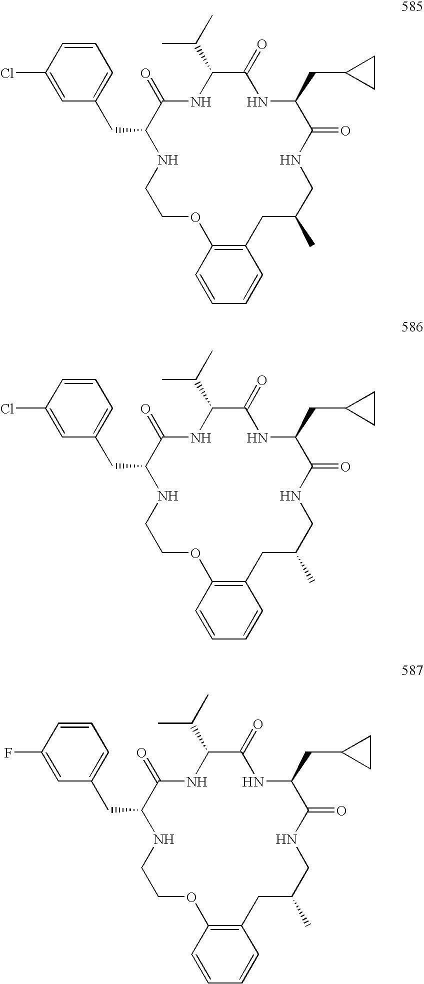

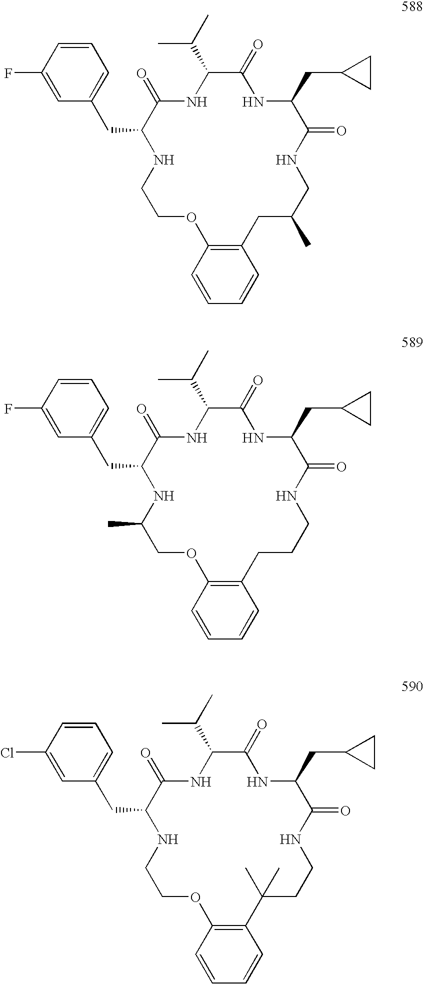

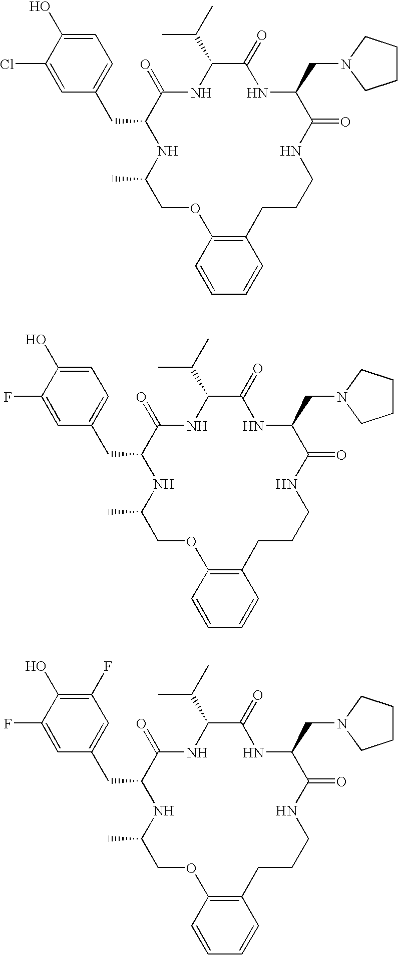

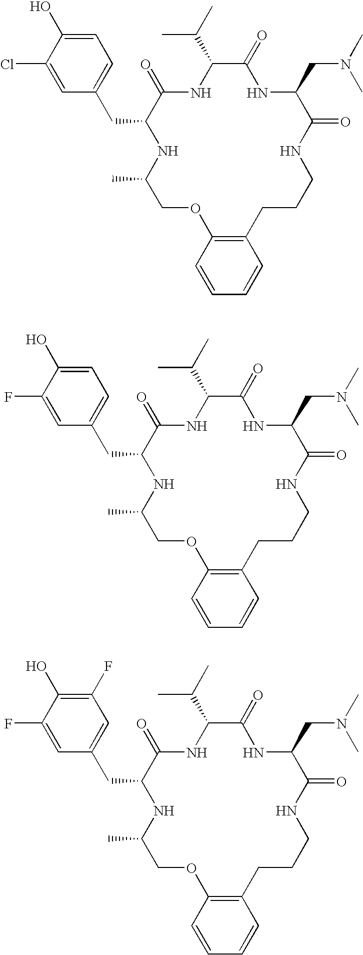

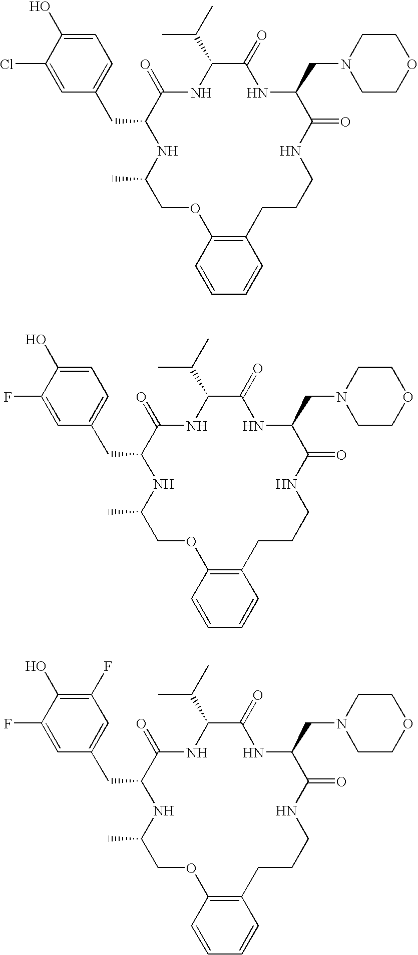

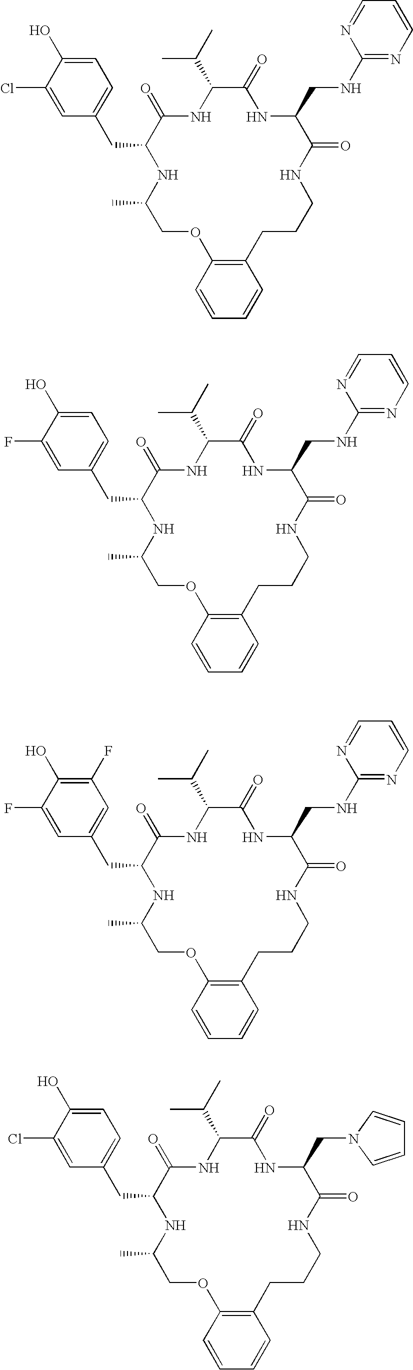

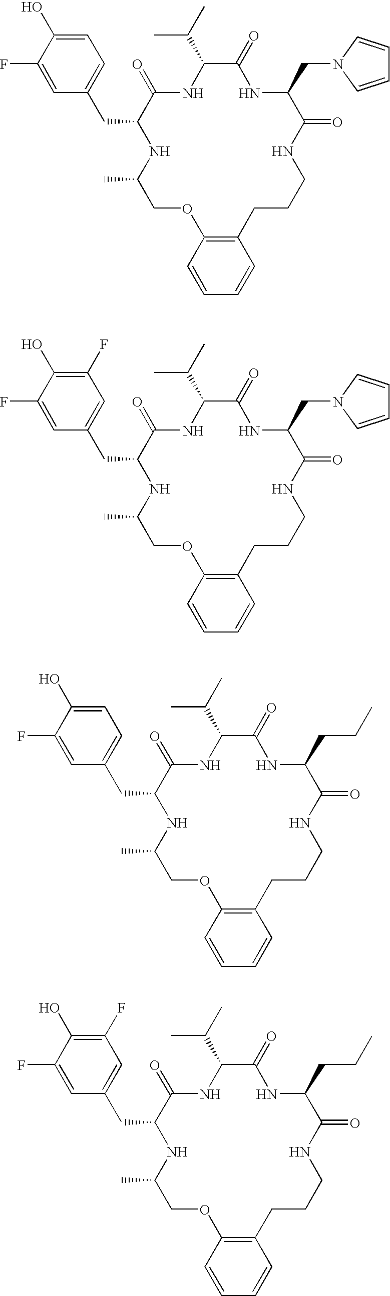

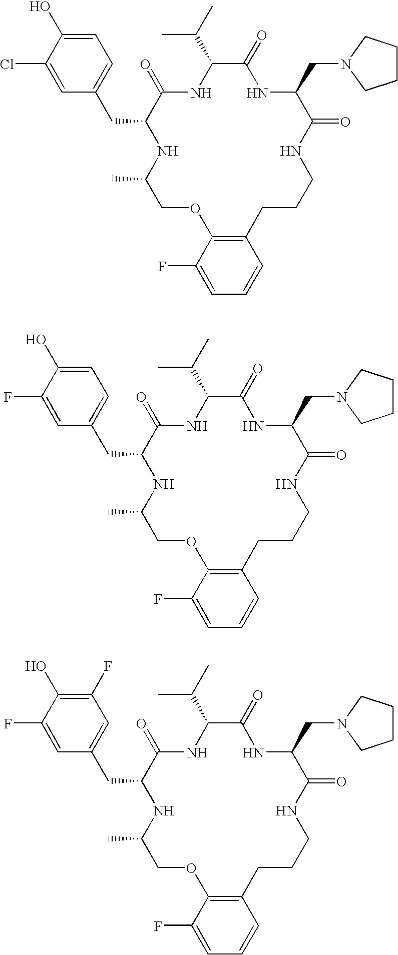

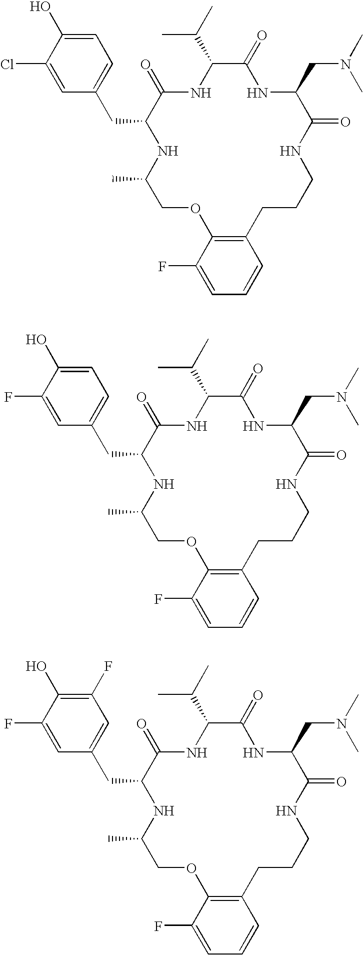

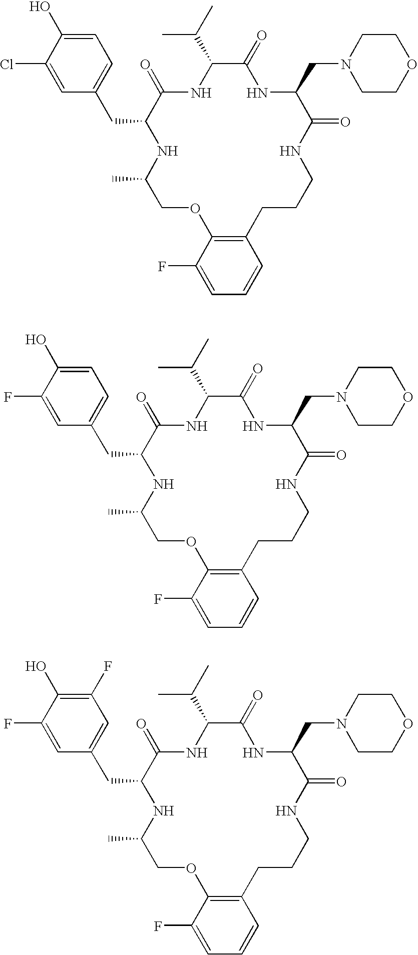

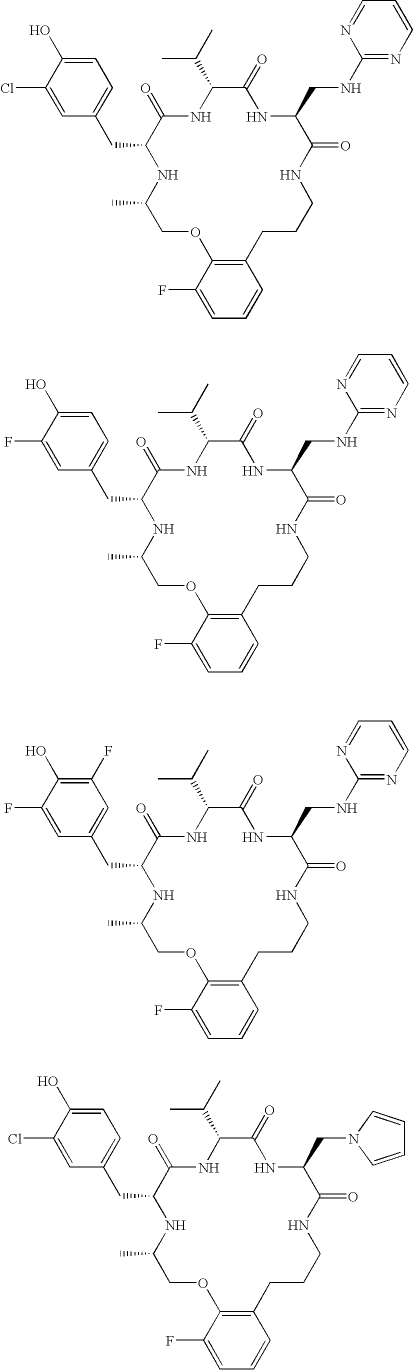

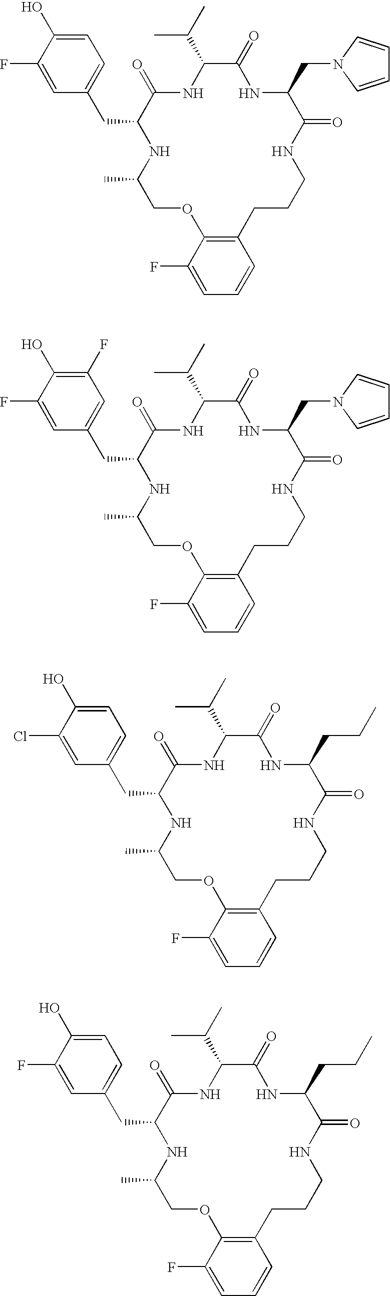

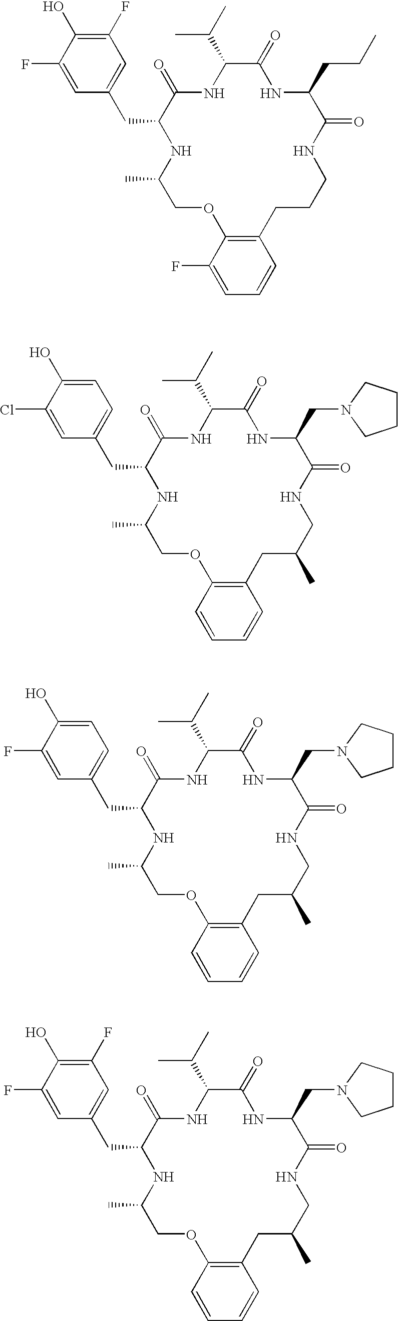

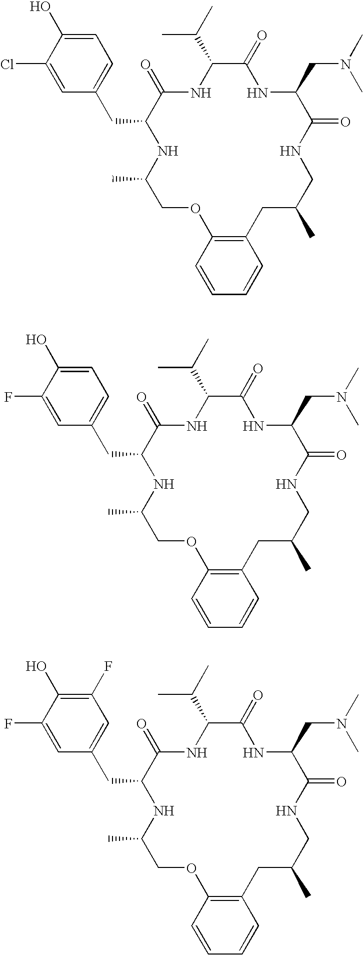

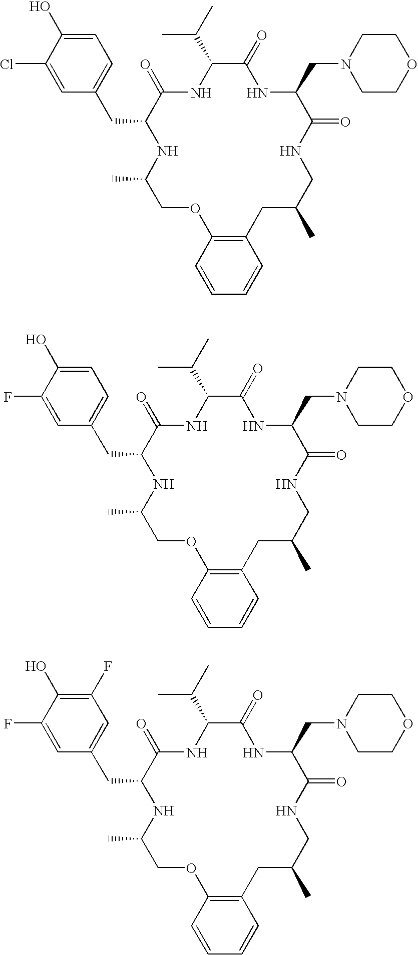

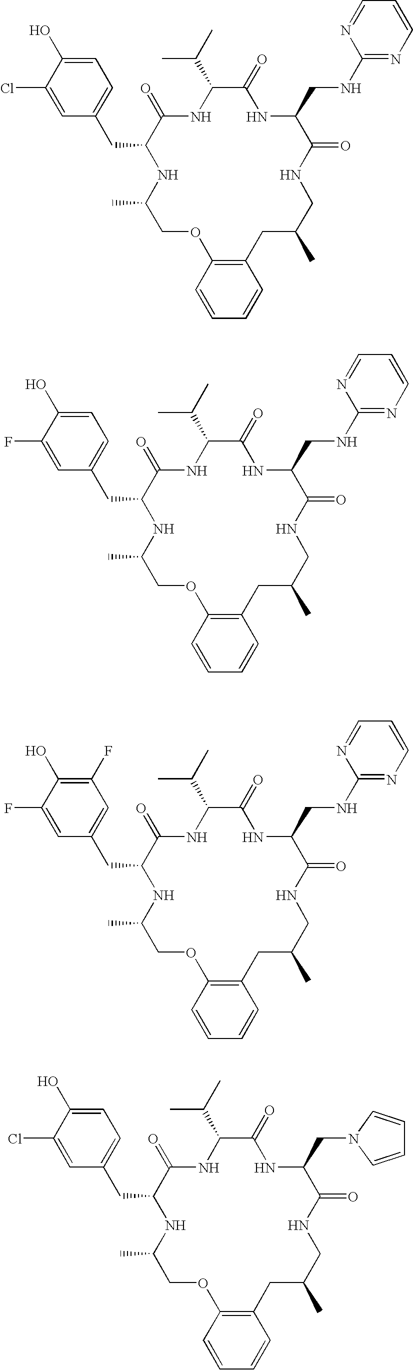

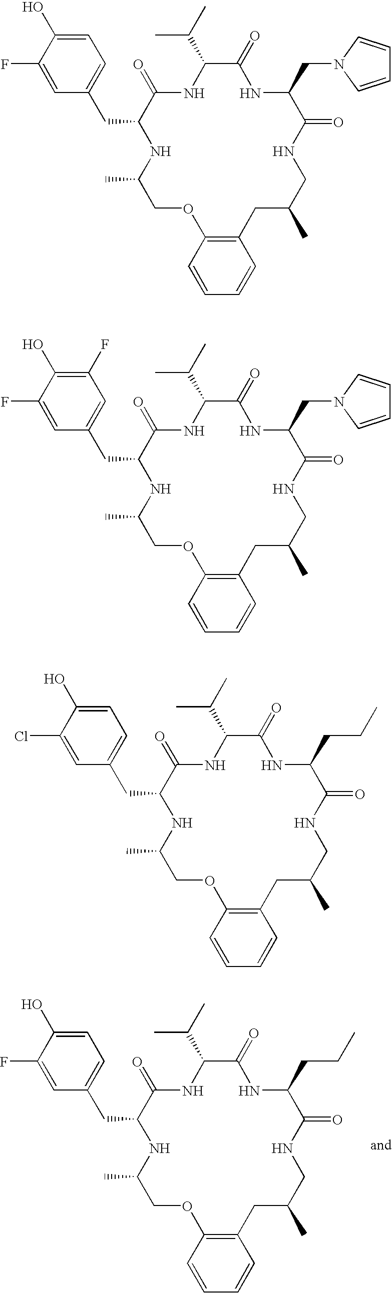

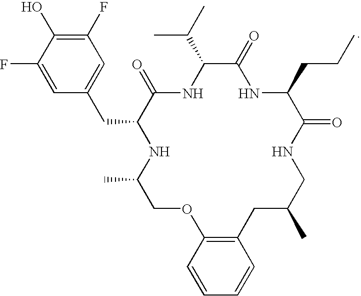

3. The method of claim 1, wherein the disorder is irritable bowel syndrome, dyspepsia, gallbladder dyspepsia, or functional Gastrointestinal disorders.

4. The method of claim 1, wherein the disorder is diarrhea, cancer treatment-related diarrhea, cancer-induced diarrhea, chemotherapy-induced diarrhea, radiation enteritis, radiation-induced diarrhea, stress-induced diarrhea, chronic diarrhea, AIDS-related diarrhea, C. difficile associated diarrhea, traveller's diarrhea, acute infectious diarrhea, diarrhea induced by graph versus host disease.

5. The method of claim 1, wherein the disorder is Crohn's diseases gastroesophogeal reflux disorders, ulcerative colitis, pancreatitis, infantile hypertrophic pyloric stenosis, carcinoid syndrome, postgastroenterectomy syndrome, atrophic colitis or gastritis, gastrointestinal dumping syndrome, chemotherapy-induced nausea and vomiting (emesis), post-operative nausea and vomiting, cyclic vomiting syndrome or functional vomiting.

6. The method of claim 1, wherein the subject is a mammal.

7. The method of claim 1, wherein the subject is a human.

8. The method of claim 1, wherein the subject is treated with an additional compound that modulates gastrointestinal motility.

9. A method of suppressing the migrating motor complex by administering to a subject an effective amount of a compound of formula I 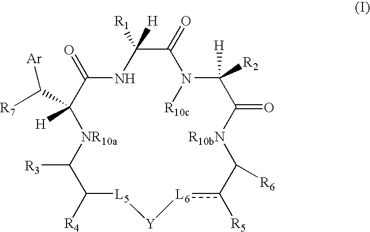
or pharmaceutically acceptable salts, hydrates or solvates thereof, wherein:
Y is 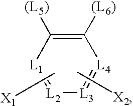
wherein (L₅) and (L₆) indicate the bonds to L₅ and L₆ Of formula I, respectively;
Ar is selected from the group consisting of: 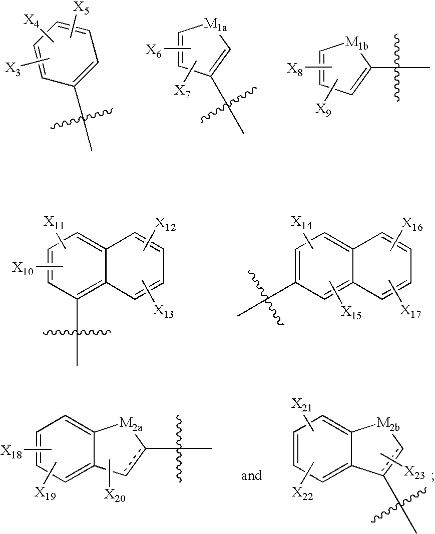
R₁ is selected from the group consisting of: —(CH₂)ₛCH₃, —CH(CH₃)(CH₂)ₜCH₃, —(CH₂)ᵤCH(CH₃)₂, —C(CH₃)₃, and 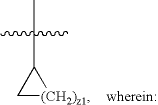
s is 0, 1, 2 or 3;
t is 1 or 2;
u is 0 or 1; and
z1 is 1, 2, 3 or 4;
R₂ is selected from the group consisting of hydrogen, —(CH₂)ₐₐCH₃, —CH₂SCH₃, —CH₂CH₂SCH₃, —(CH₂)_{bb}CH(CH₃)₂, —CH(CH₃)(CH₂)_{cc}CH₃, —(CH₂)_{dd}—NR₁₁R₁₂, and —(CH₂)ₑₑR₁₃; wherein
aa and bb are independently 0, 1, 2 or 3;
cc and dd are independently 1, 2, 3 or 4;
ee is 0, 1, 2, 3 or 4;
R₁₁ is selected from the group consisting of hydrogen, lower alkyl, formyl, acyl, carboxyalkyl, carboxyaryl, amido, amidino, sulfonyl and sulfonamido;
R₁₂ is selected from the group consisting of hydrogen and lower alkyl;
R₁₃ is selected from the group consisting of: 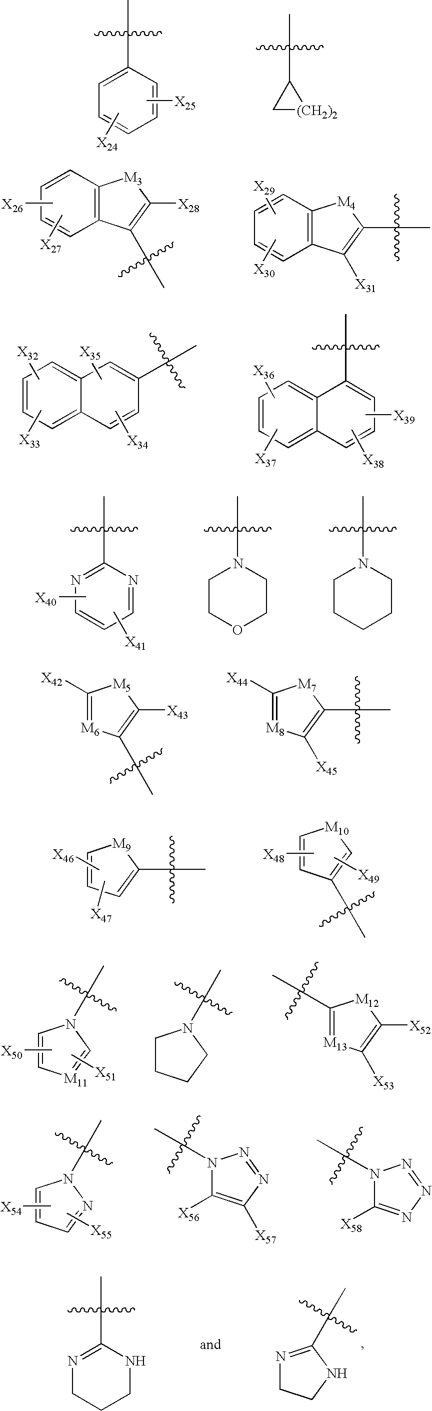
wherein z2 is 1, 2, 3 or 4;
and, when ee is 1, 2, 3 or 4, R₁₃ is further selected from the group consisting of hydroxy, alkoxy, amidino, and azido;
R₃, R₄, R₅, and R₆ are independently selected from the group consisting of hydrogen, methyl, ethyl, isopropyl and hydroxymethyl;
R₇ is selected from the group consisting of hydrogen, methyl, hydroxy and amino;
R₁₀ₐ and R_{10b} are independently selected from the group consisting of hydrogen and methyl;
X₁, X₂, X₆, X₇, X₈ and X₉ are independently selected from the group consisting of hydrogen, halogen, trifluoromethyl and lower alkyl;
X₃, X₄, X₅, X₁₀, X₁₁, X₁₂, X₁₃, X₁₄, X₁₅, X₁₆, X₁₇, X₁₈, X₁₉, X₂₁, X₂₂, X₂₄, X₂₅, X₂₆, X₂₇, X₂₈, X₂₉, X₃₀, X₃₁, X₃₂, X₃₃, X³⁴, X₃₅, X₃₆, X₃₇, X₃₈, X₃₉, X₄₀, X₄₁, X₄₂, X₄₃, X₄₄, X₄₅, X₄₆, X₄₇, X₄₈, X₄₉, X₅₀, X₅₁, X₅₂, X₅₃, X₅₄ and X₅₅ are independently selected from the group consisting of hydrogen, hydroxy, alkoxy, amino, halogen, trifluoromethyl and lower alkyl;
X₂₀ and X₂₃ are independently selected from the group consisting of hydrogen, trifluoromethyl and lower alkyl;
X₅₆, X₅₇ and X₅₈ are independently selected from the group consisting of hydrogen and lower alkyl;
L₁, L₂, L₃ and L₄ are independently selected from the group consisting of CH and N; with the proviso that the total number of nitrogens in the ring must be 0 or 1;
L₅ and L₆ are independently selected from the group consisting of O, CR₈ₐR_{8b} and NR₉ₐ; wherein R₈ₐ and R_{8b} are independently selected from the group consisting of hydrogen and methyl; and R₉ₐ is selected from the group consisting of hydrogen, lower alkyl, formyl, acyl and sulfonyl; with the proviso that when L₆ is CR₈ₐ when a double bond is present between L₆ and CHR₅;
M₁ₐ, M_{1b}, M₂ₐ, M_{2b}, M₃, M₄, M₅, M₇, M₉, M₁₀ and M₁₂ are independently selected from the group consisting of O, S and NR_{9b} wherein R_{9b} is selected from the group consisting of hydrogen, lower alkyl, formyl, acyl and sulfonyl; and
M₆, M₈, M₁₁ and M₁₃ are independently selected from the group consisting of N and CR_{9c}, wherein R_{9c} is selected from the group consisting of hydrogen and lower alkyl.

10. The method of claim 9, wherein the compound is selected from the group consisting of:

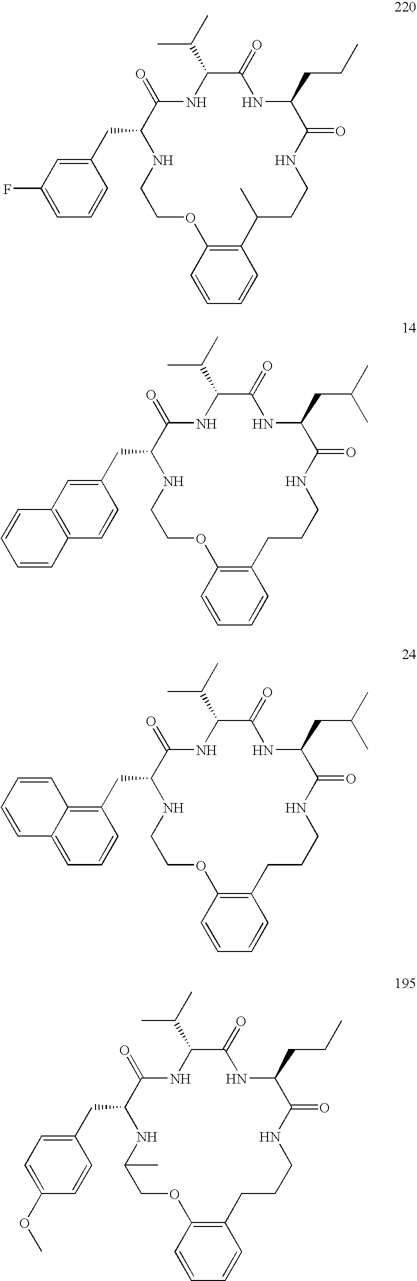

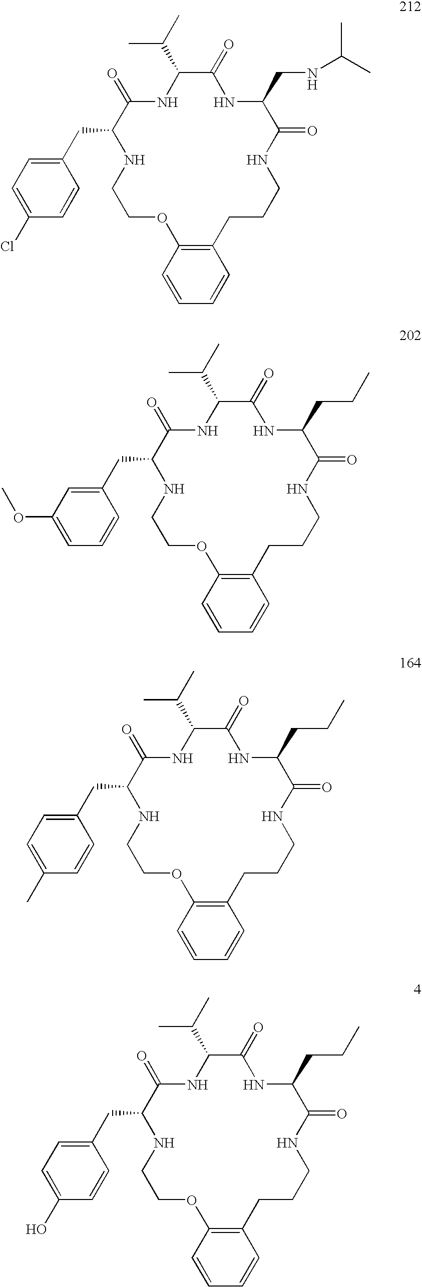

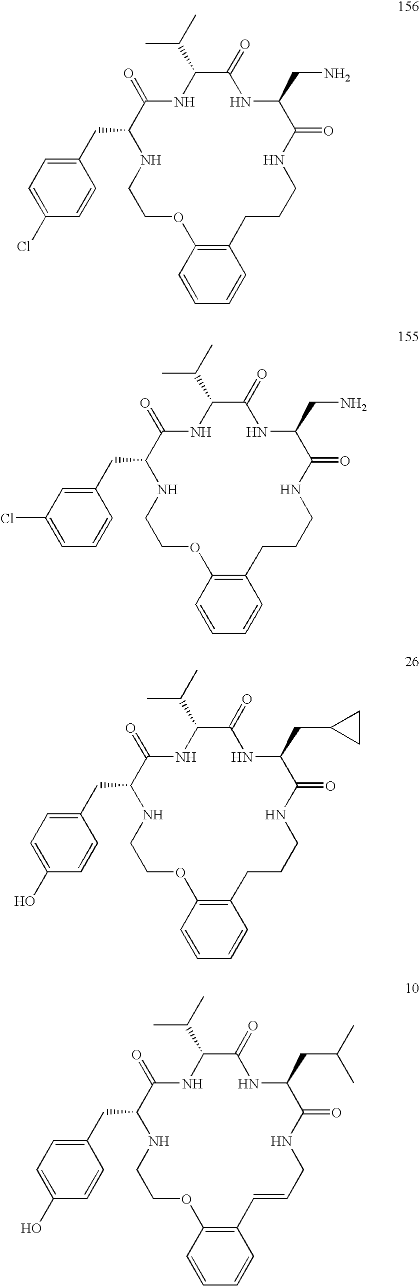

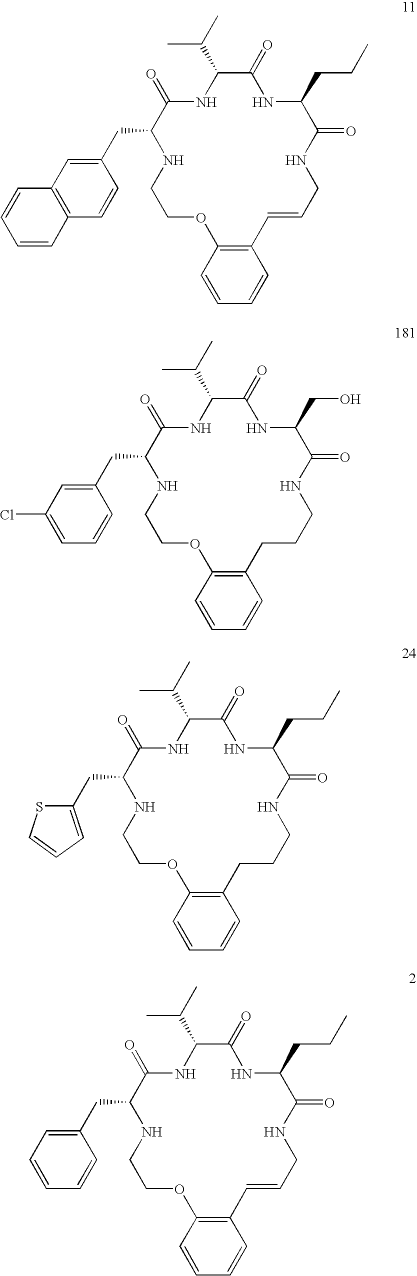

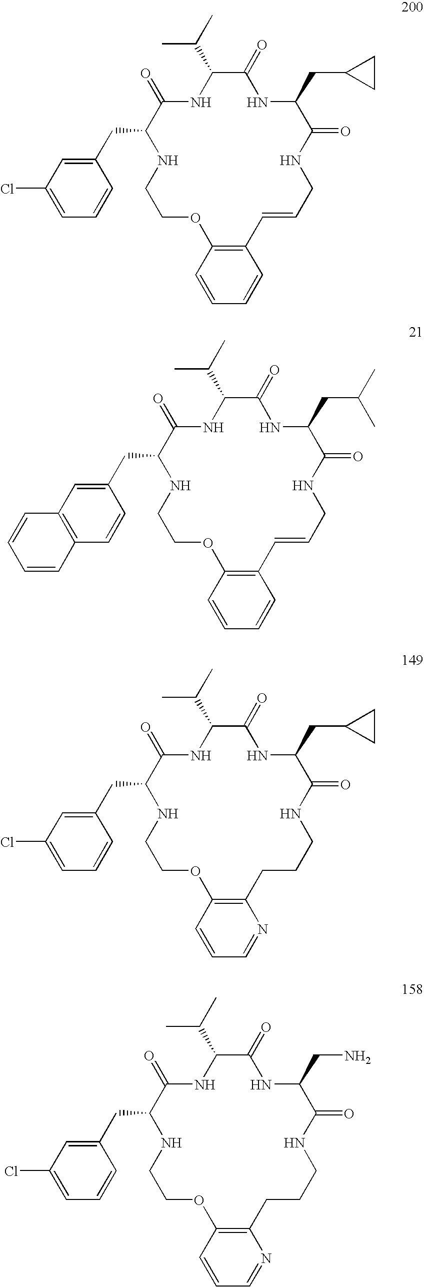

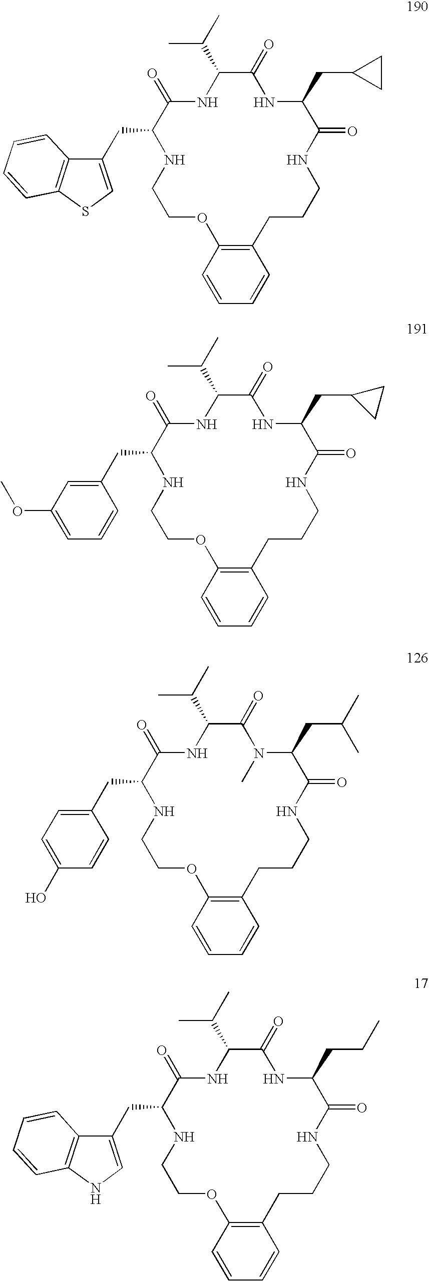

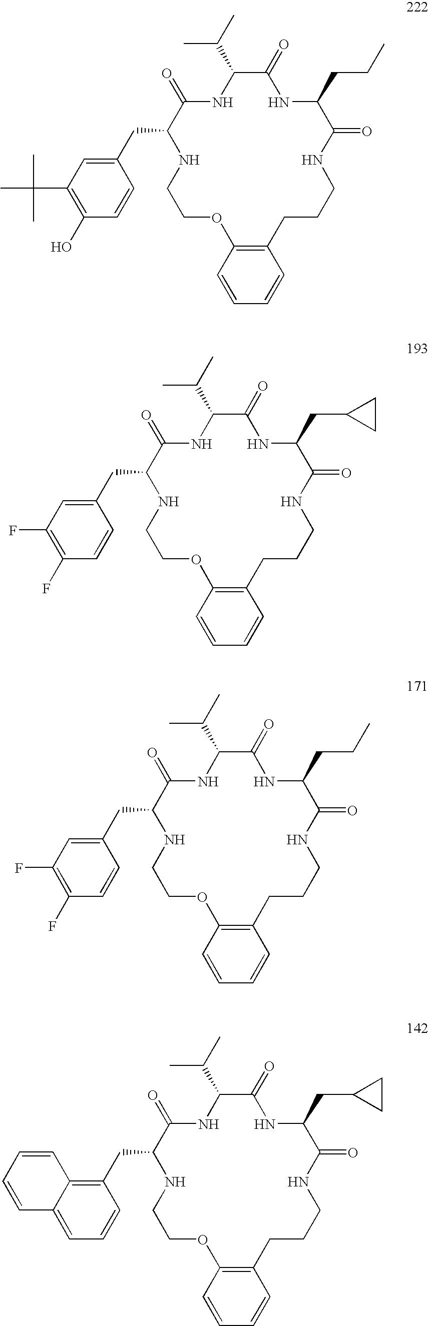

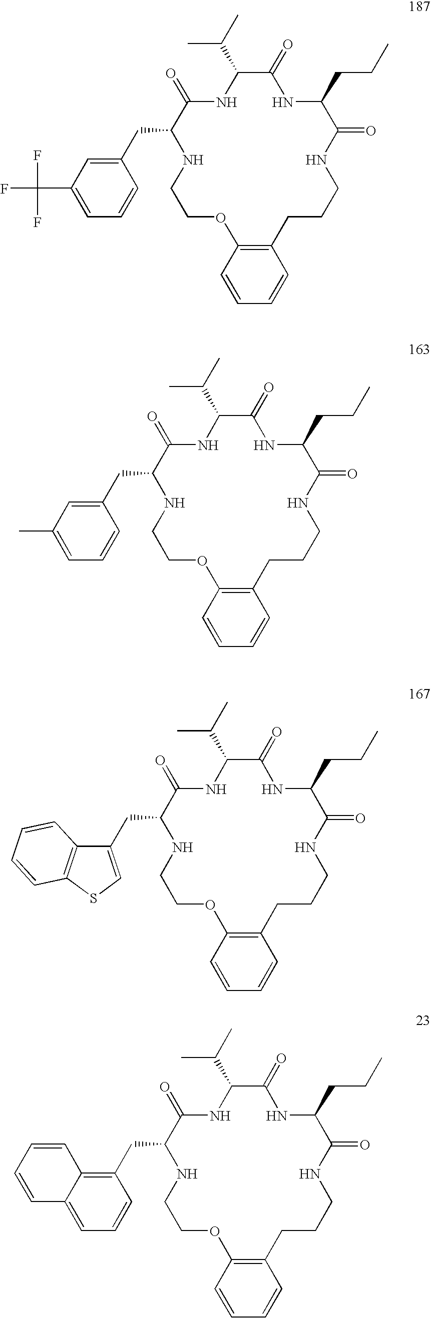

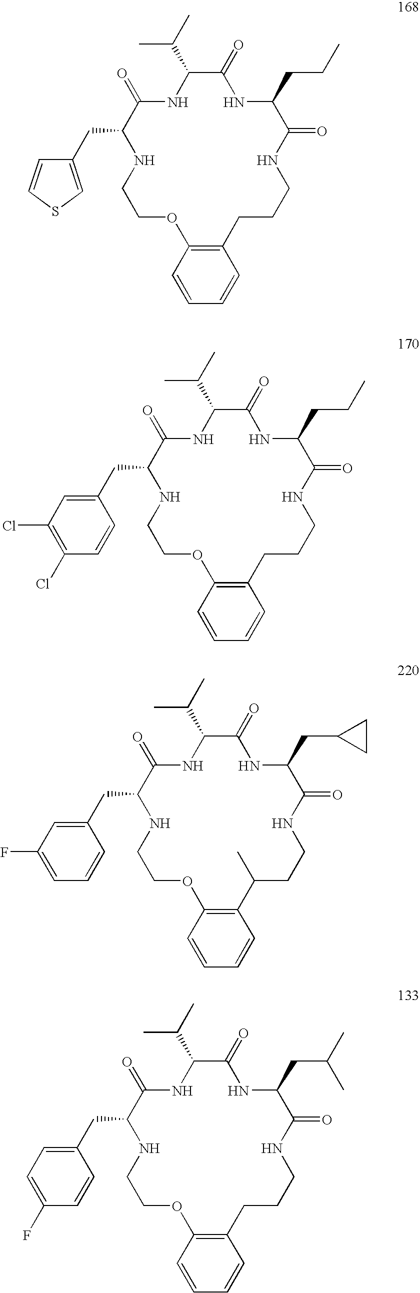

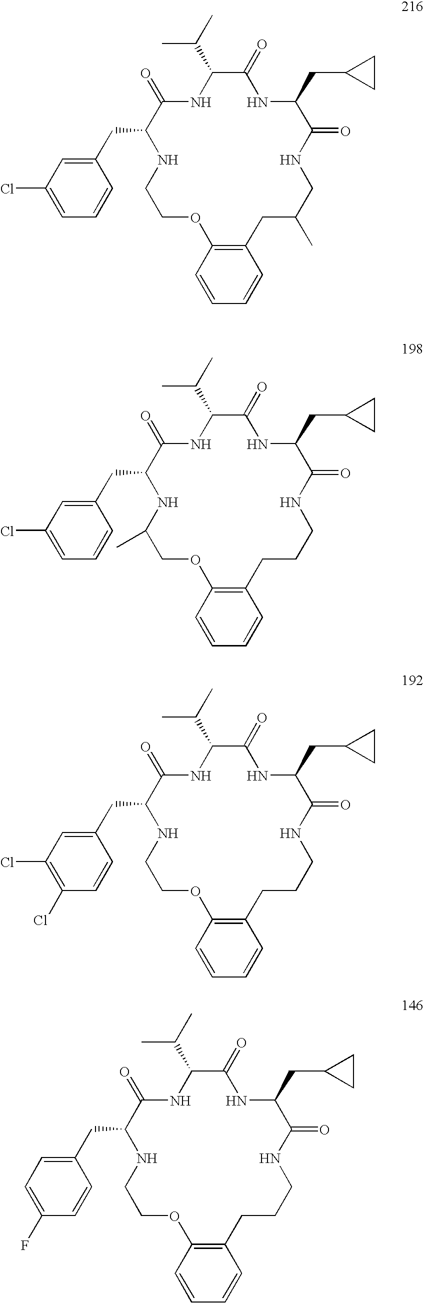

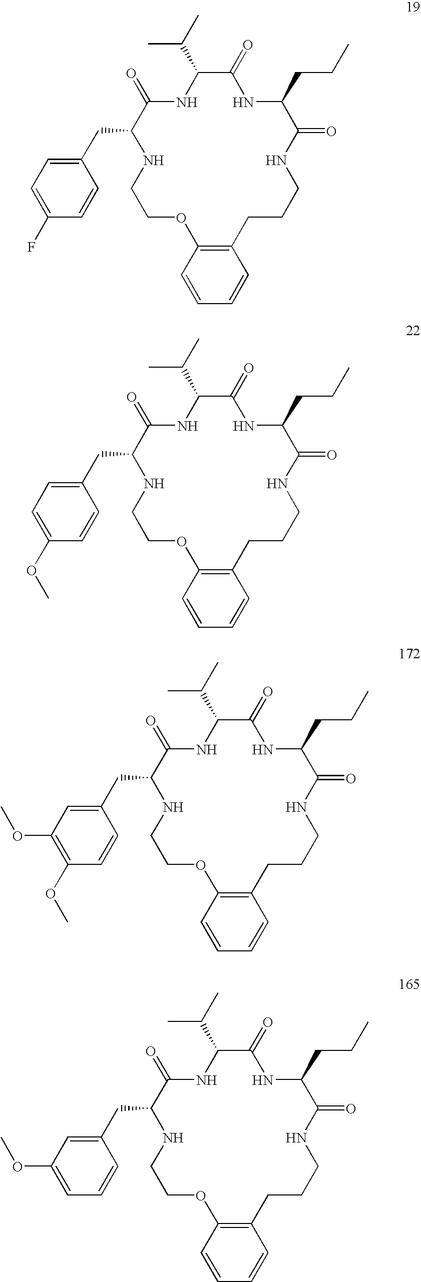

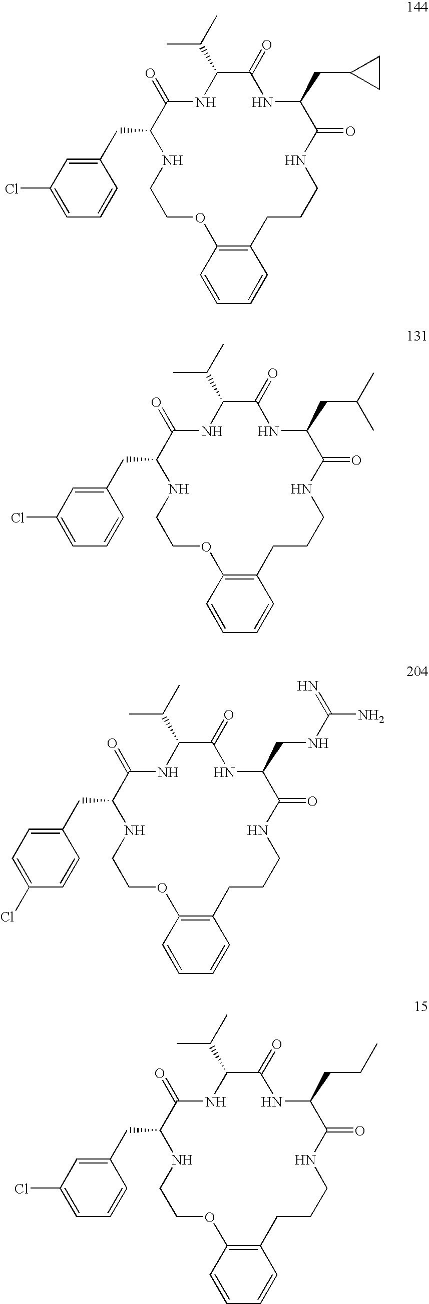

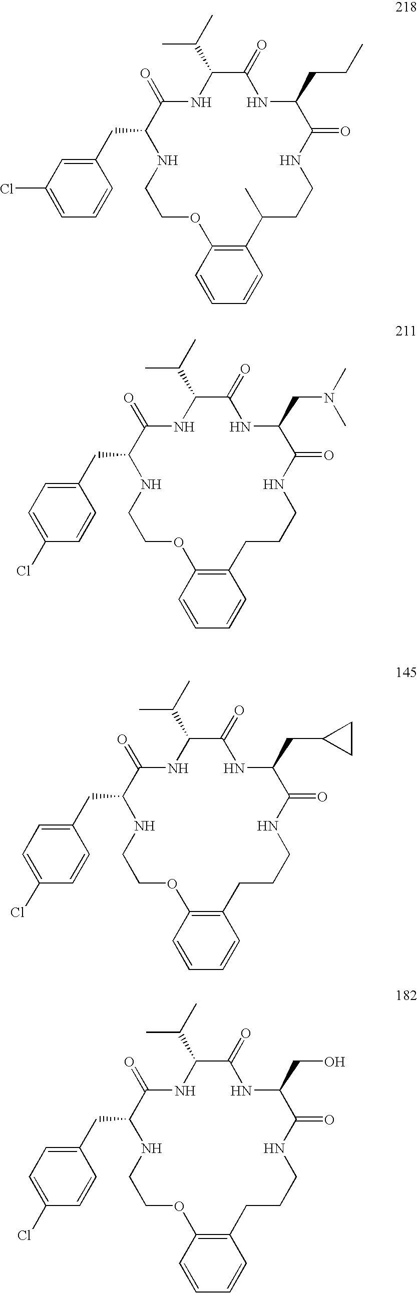

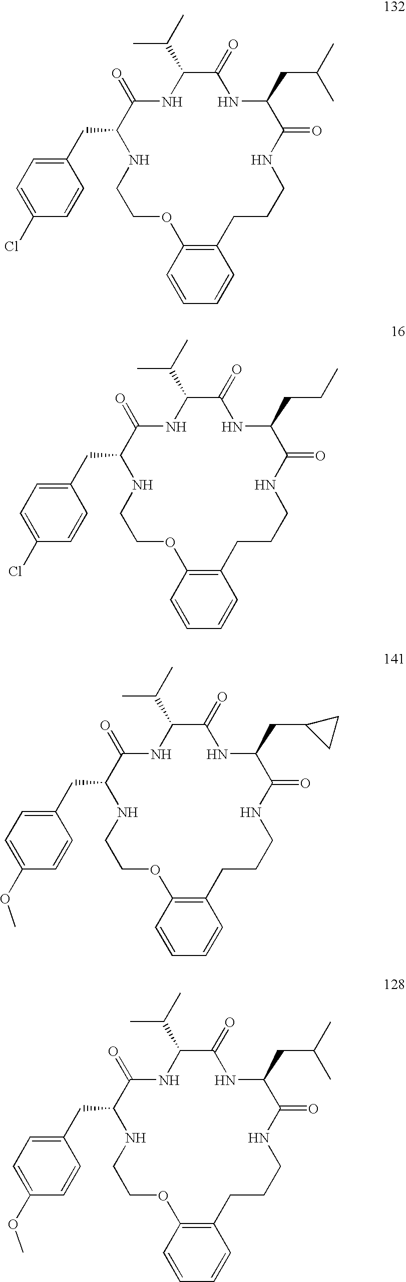

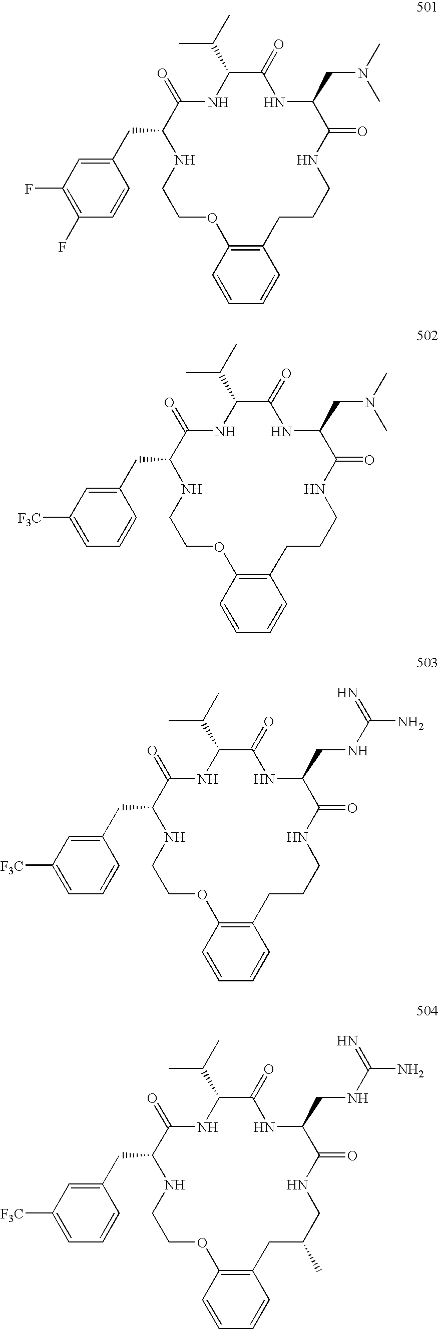

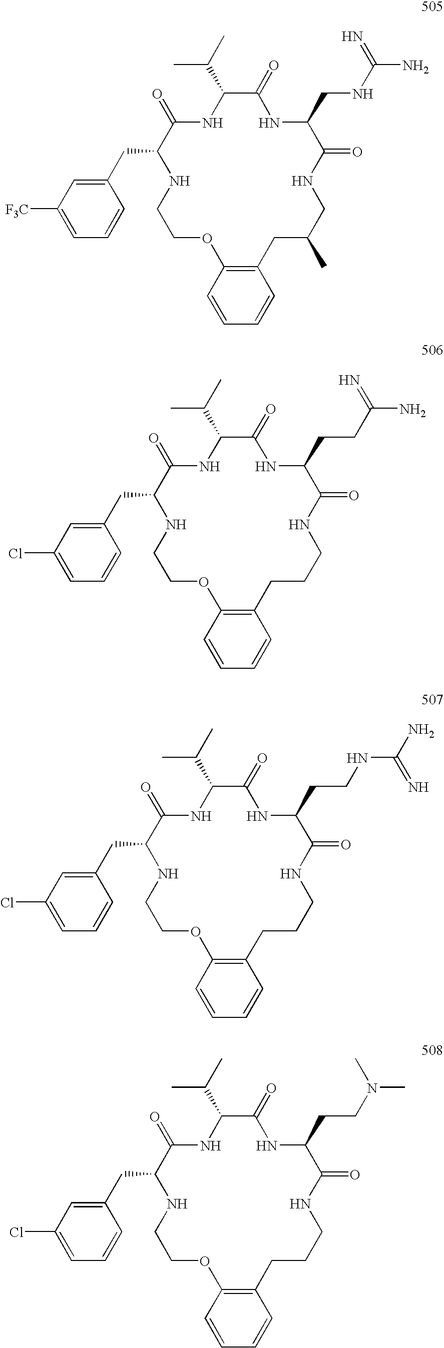

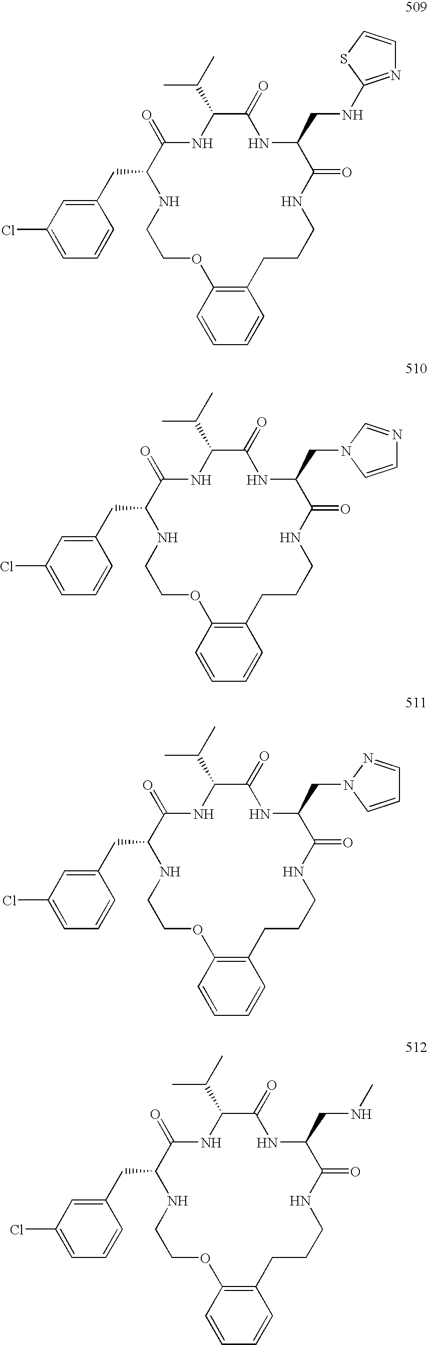

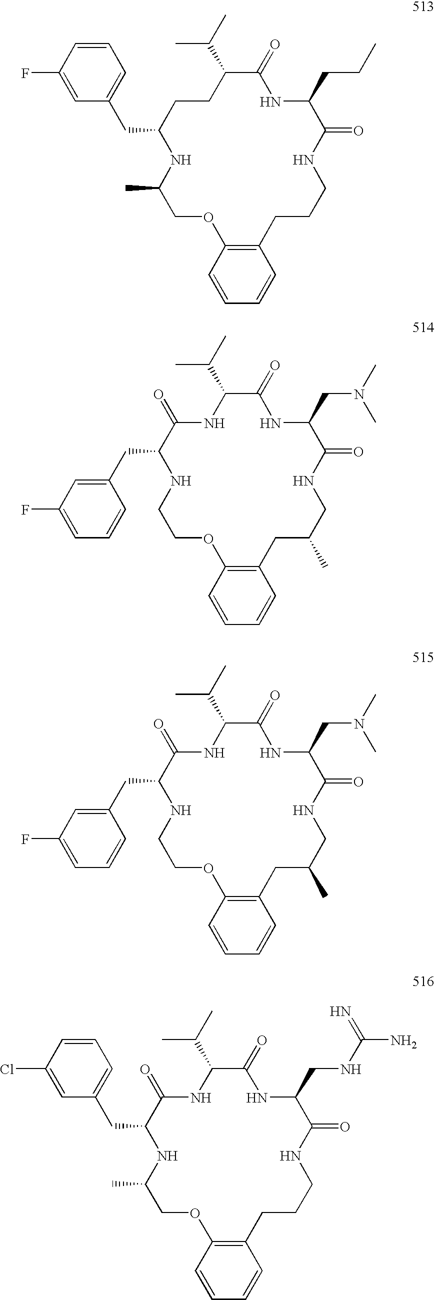

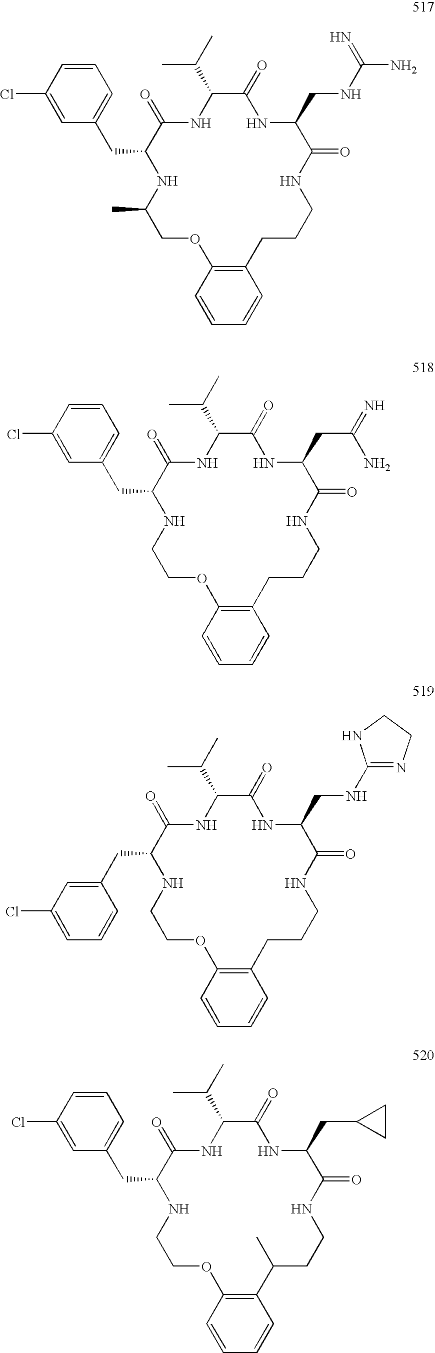

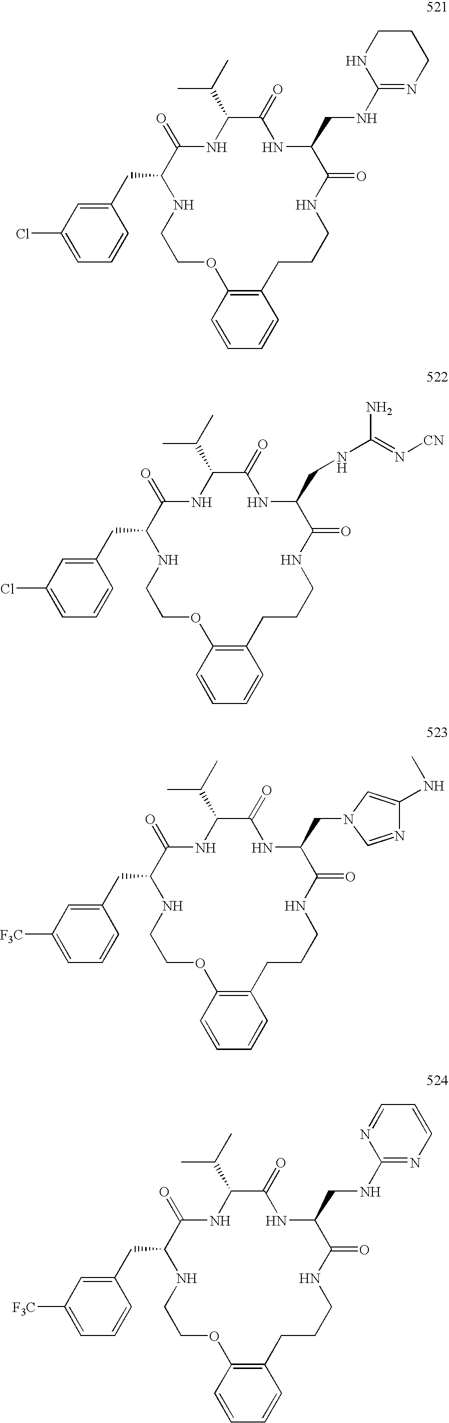

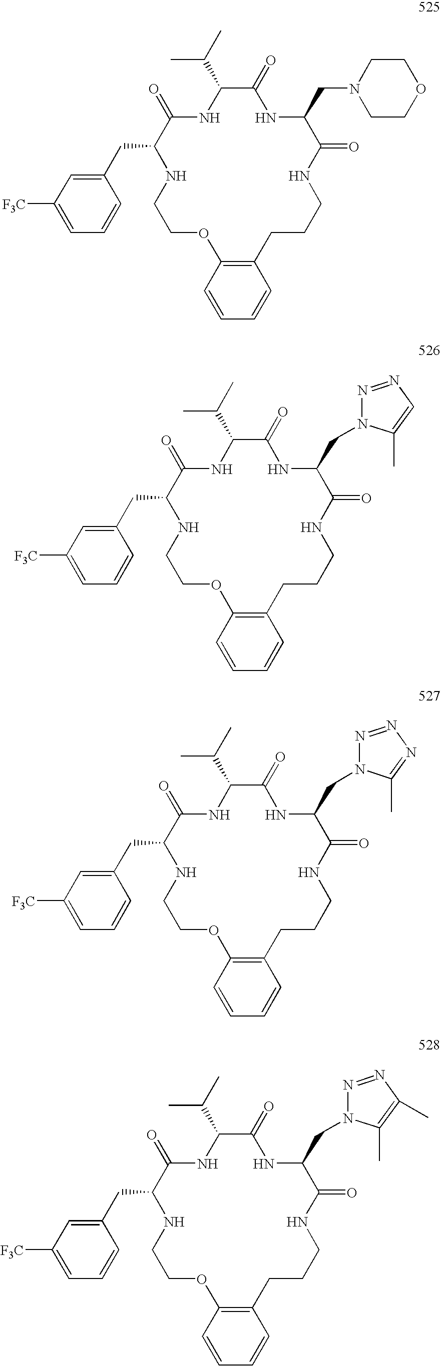

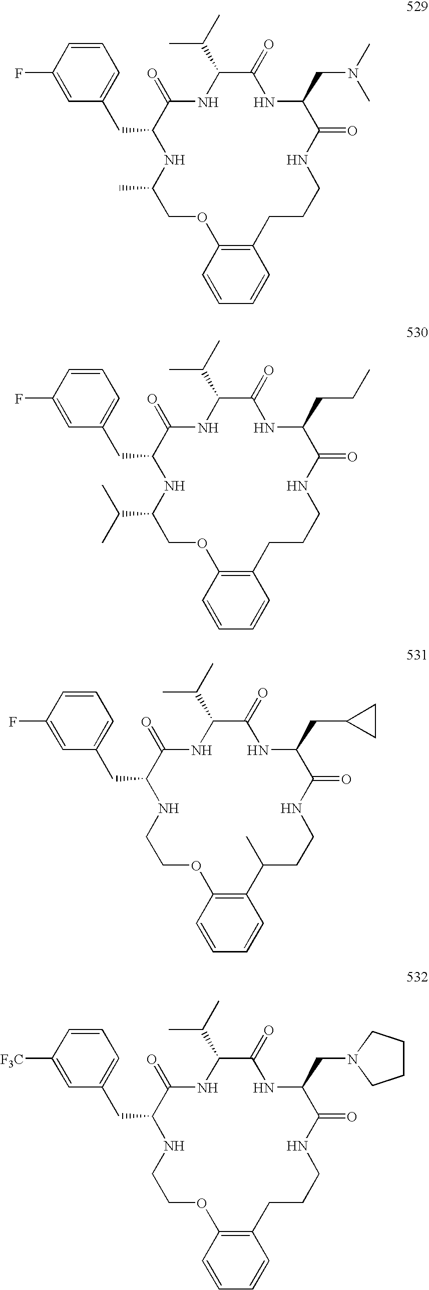

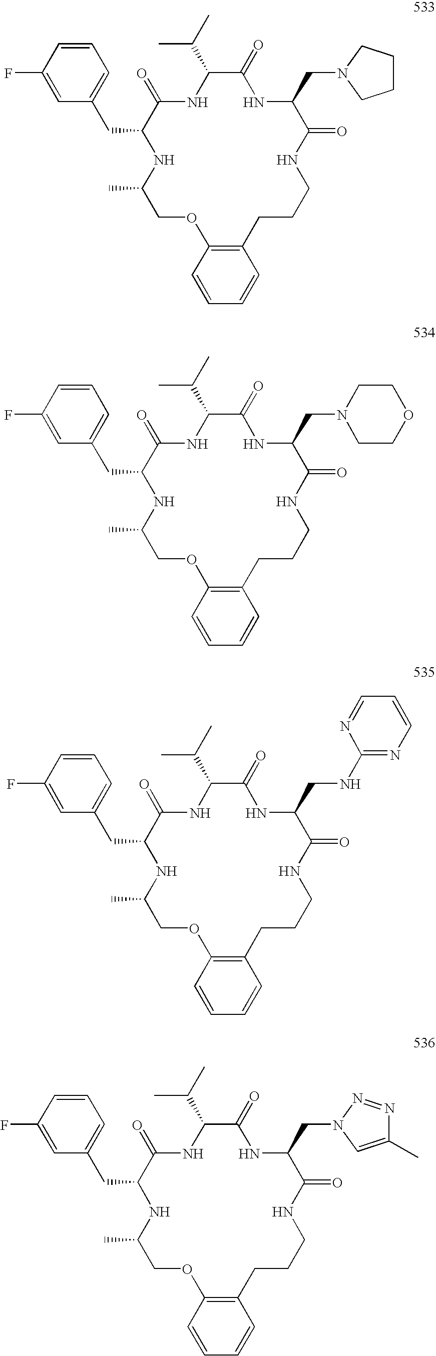

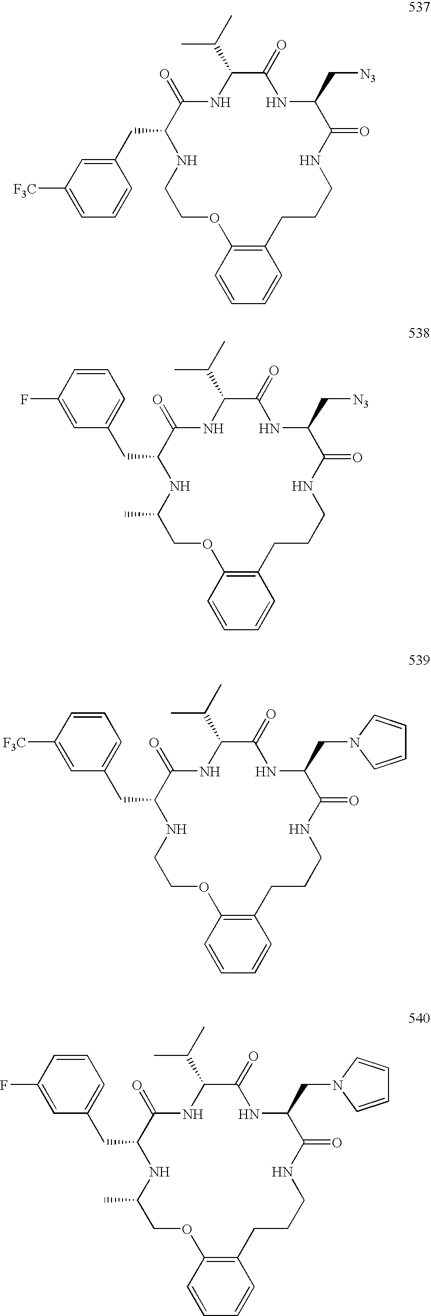

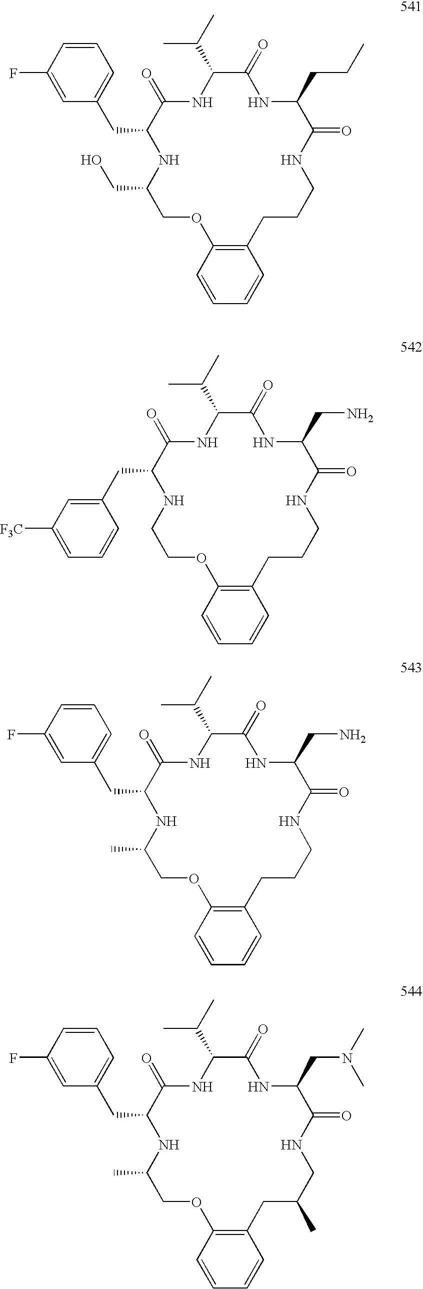

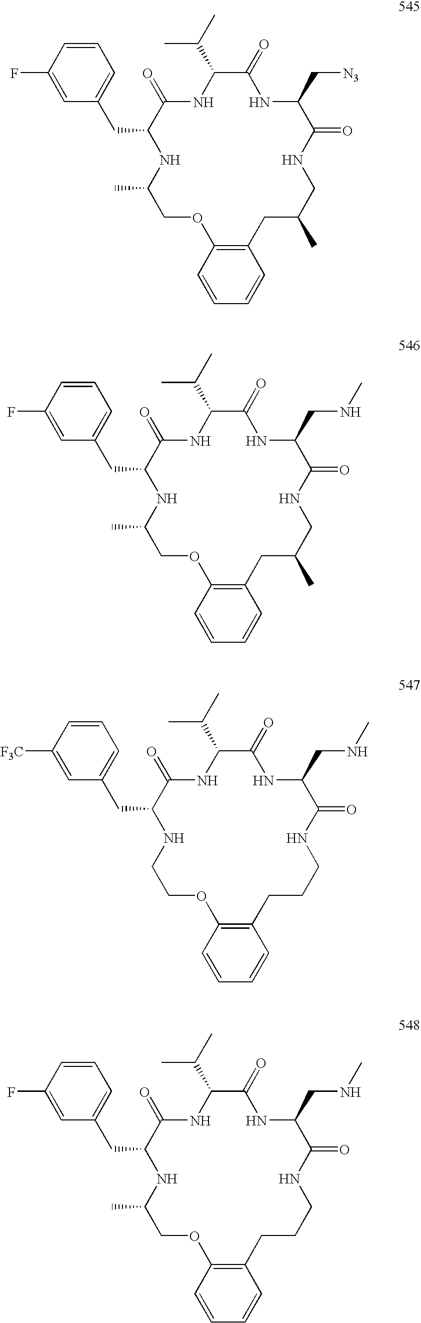

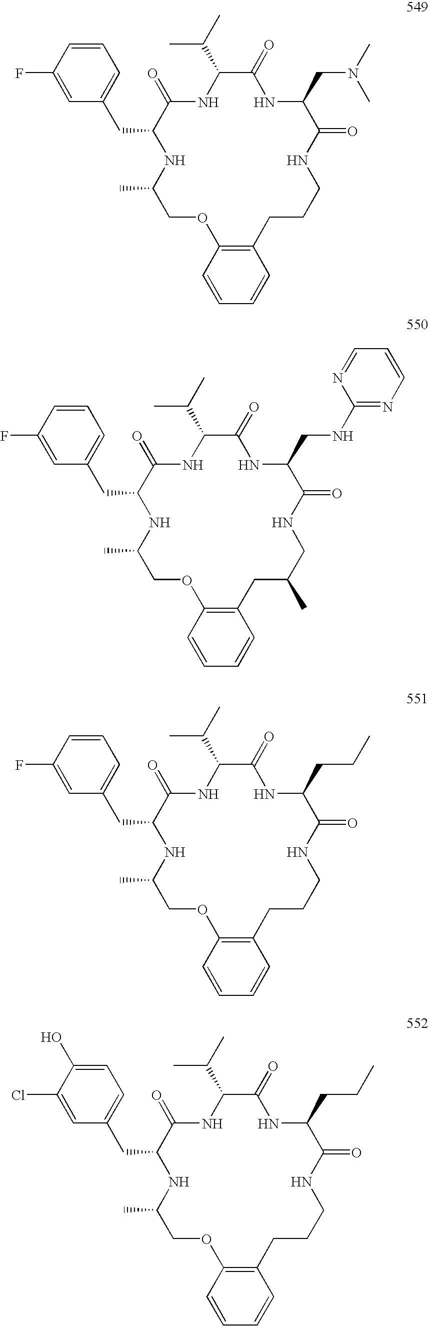

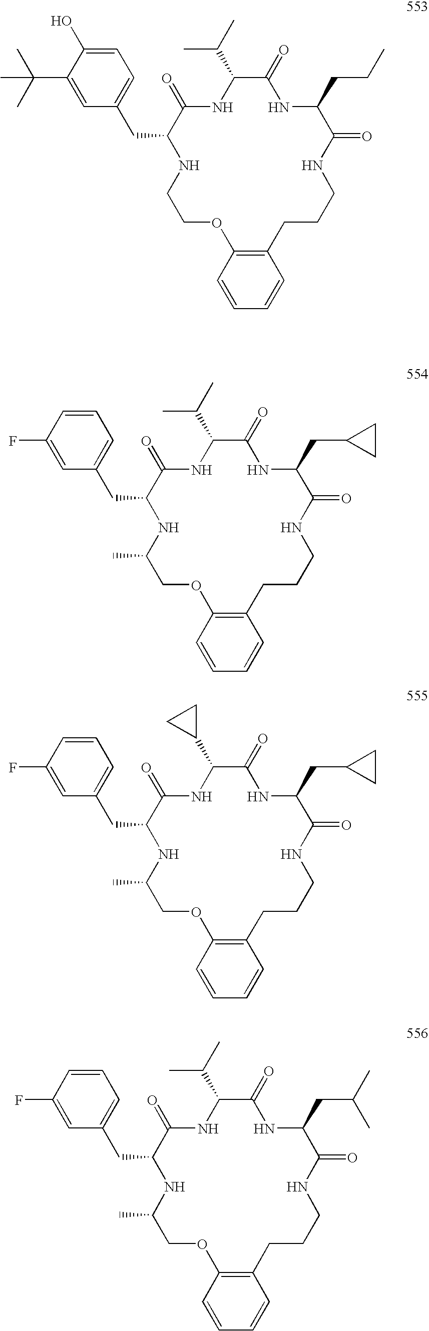

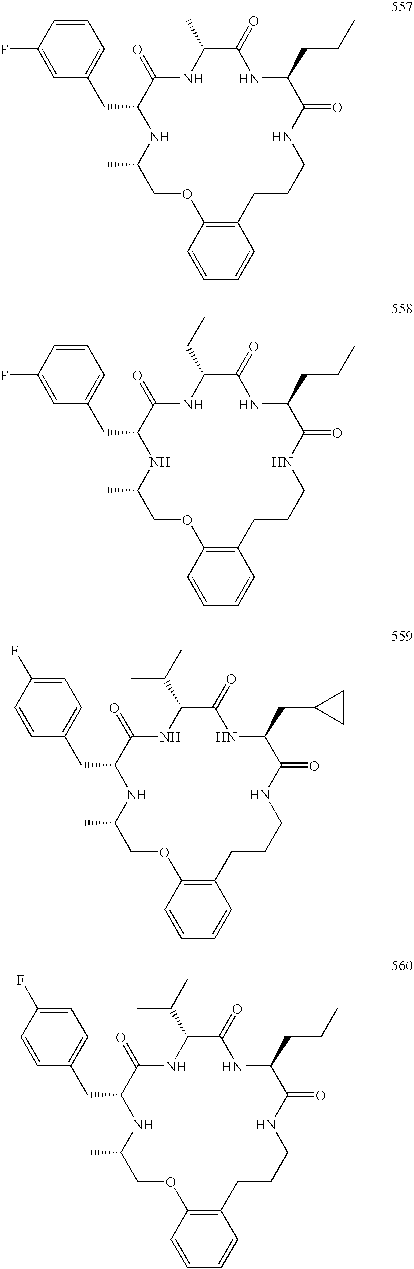

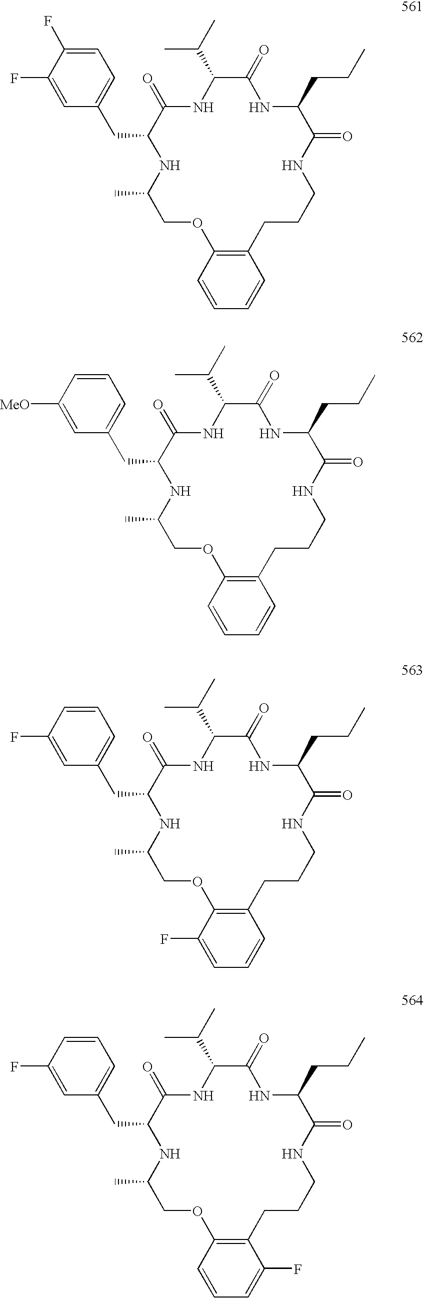

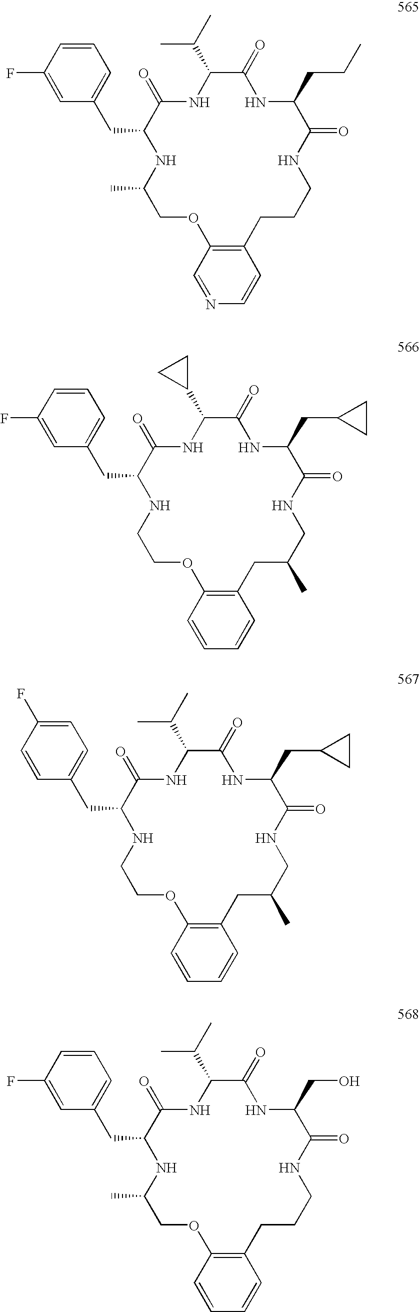

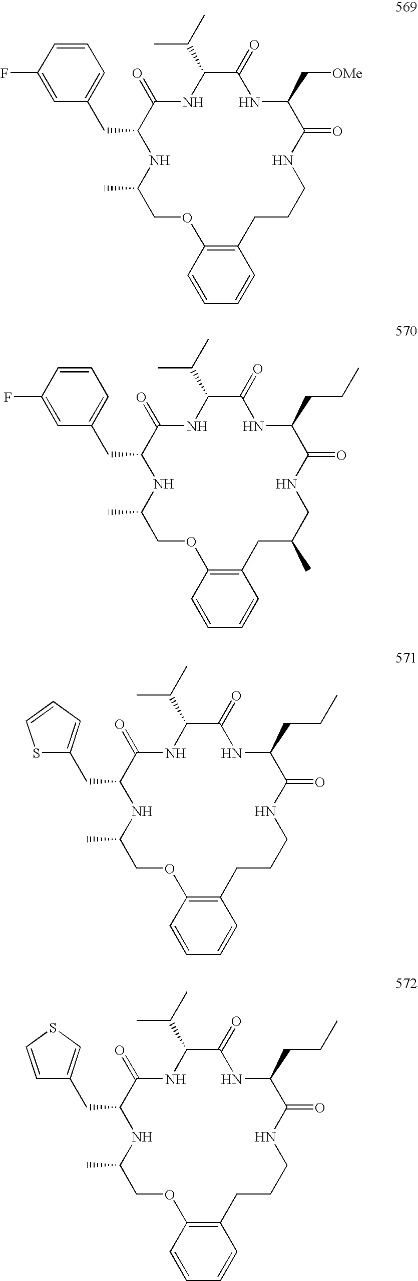

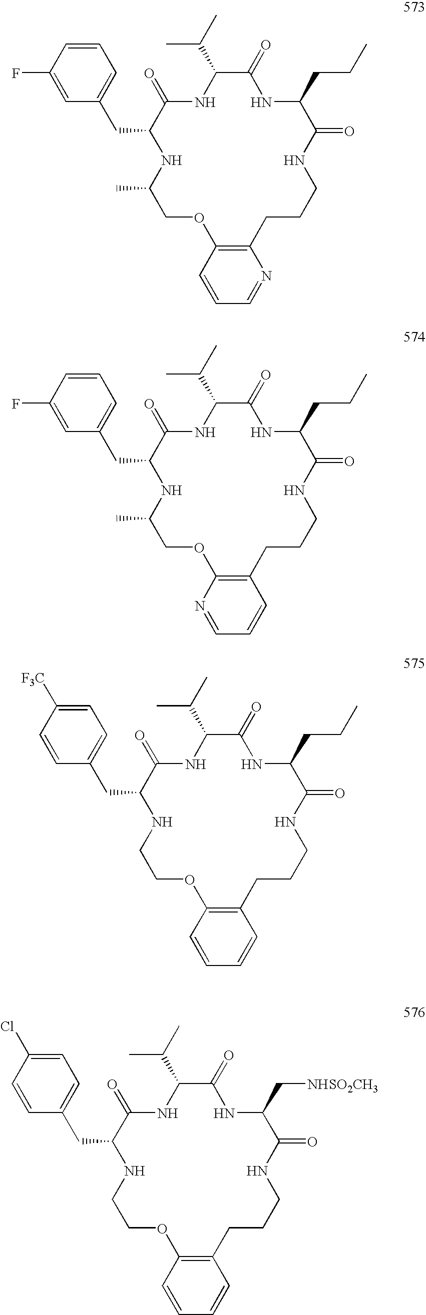

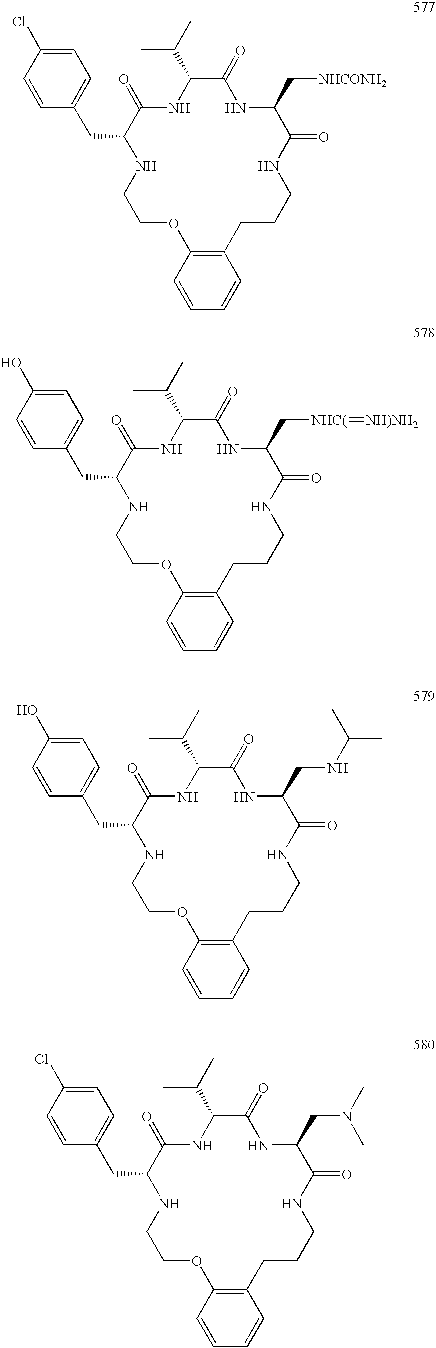

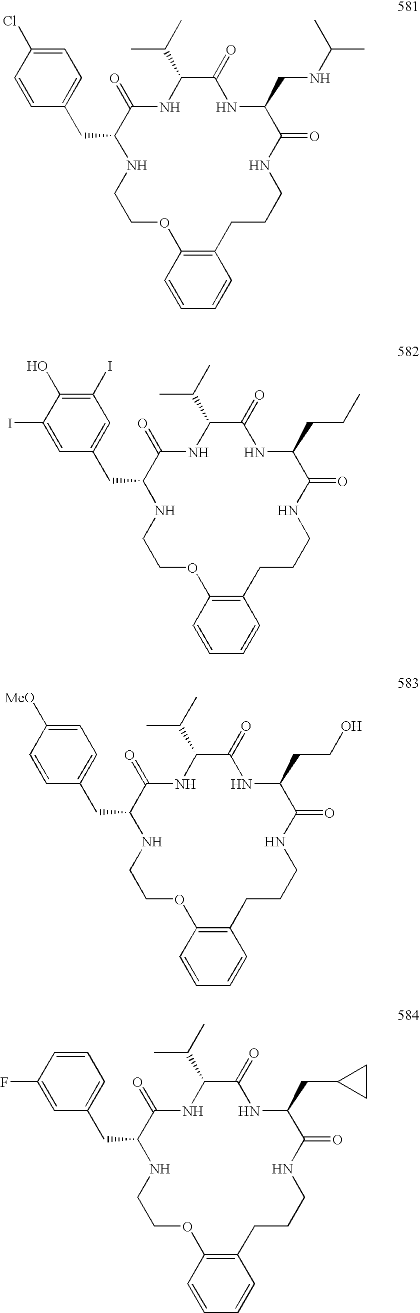

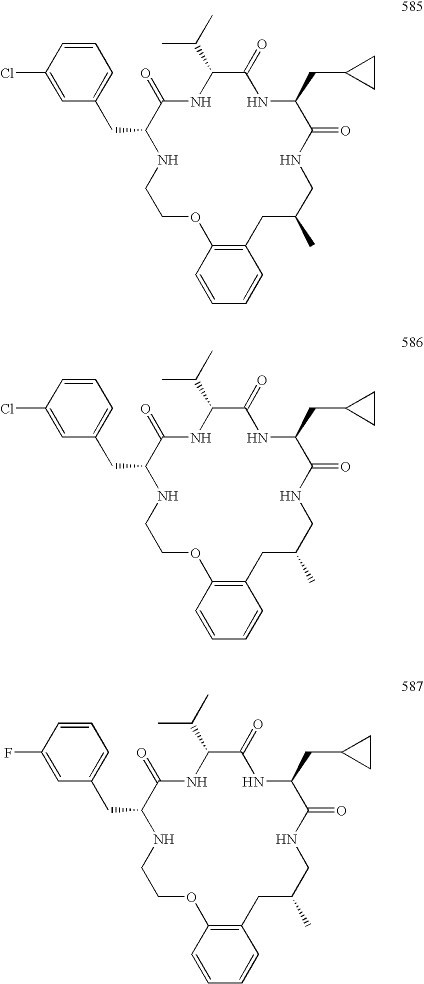

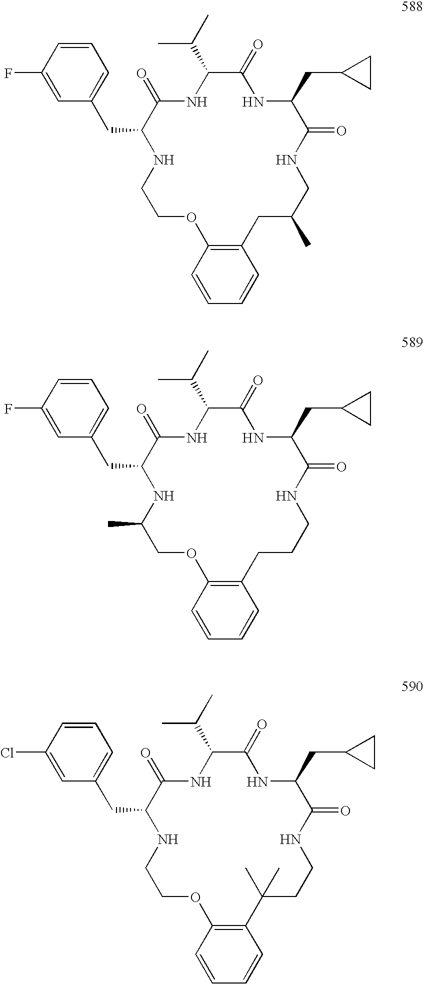

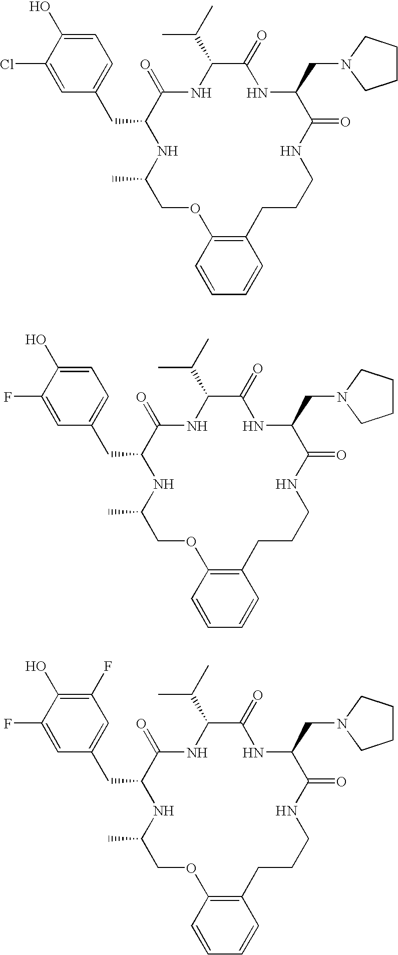

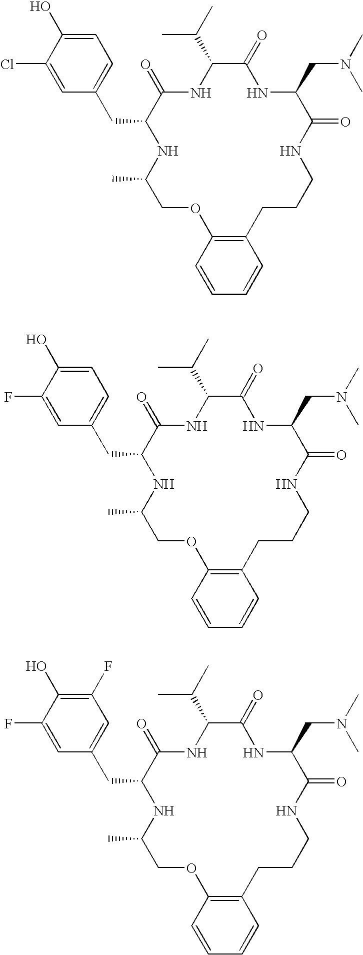

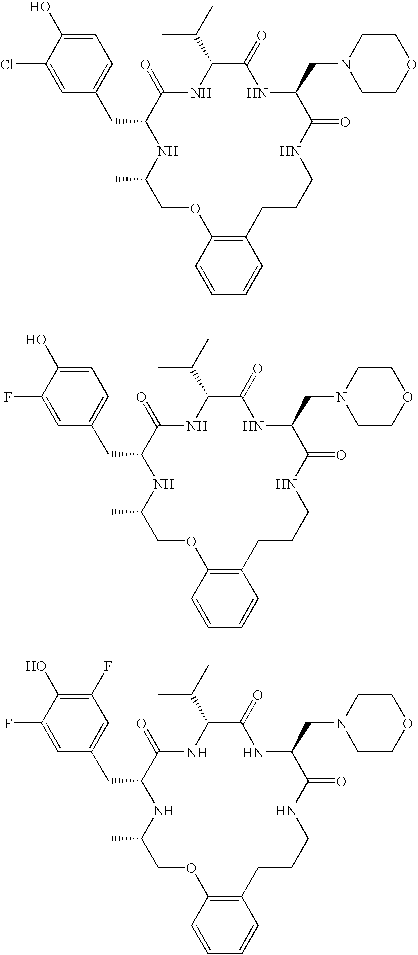

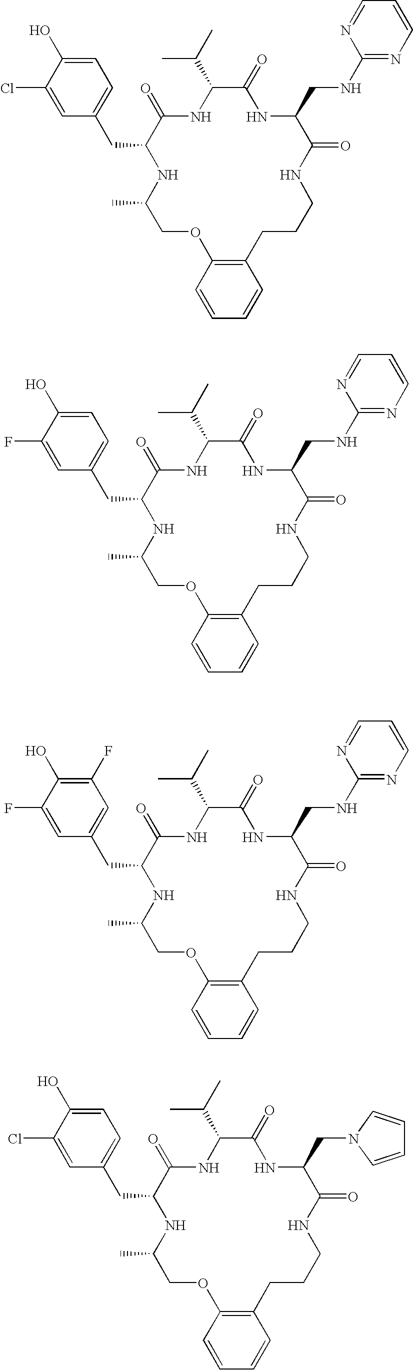

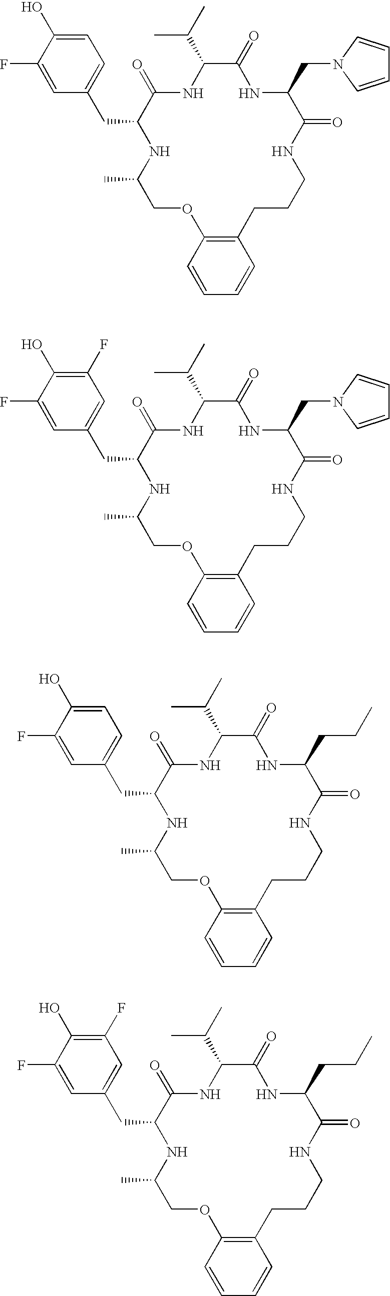

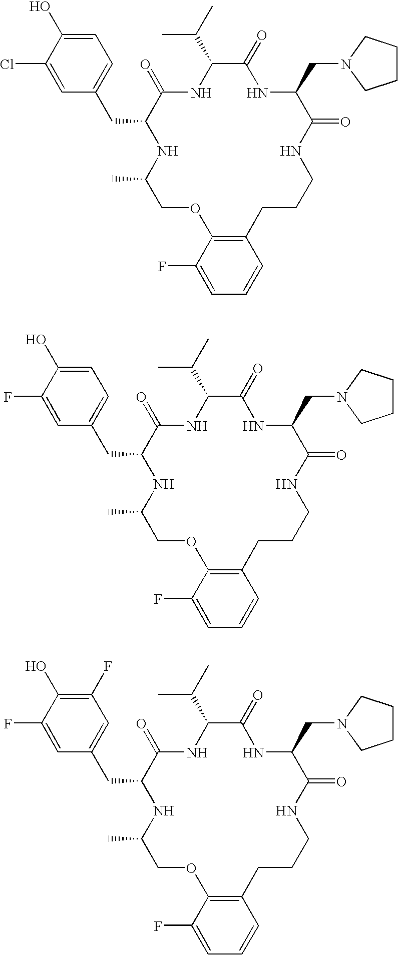

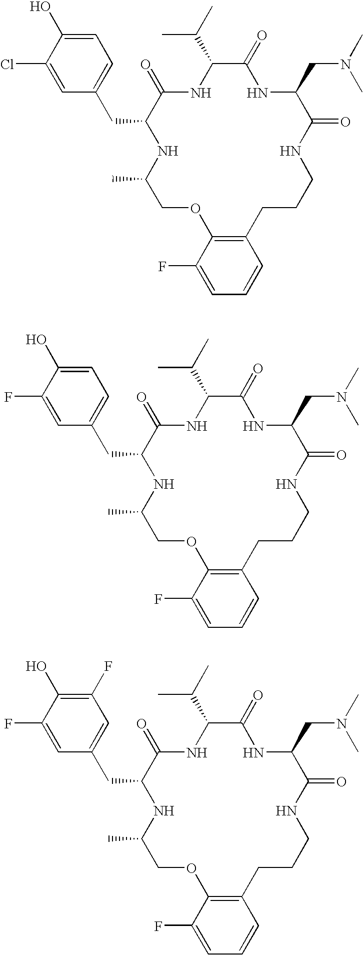

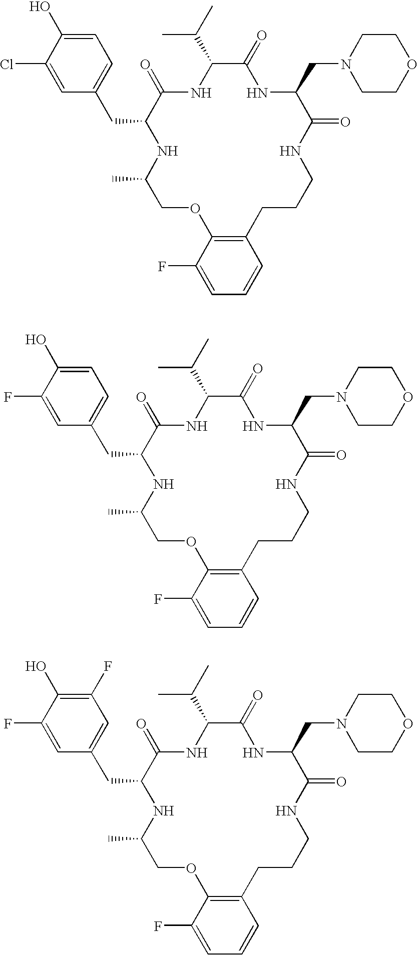

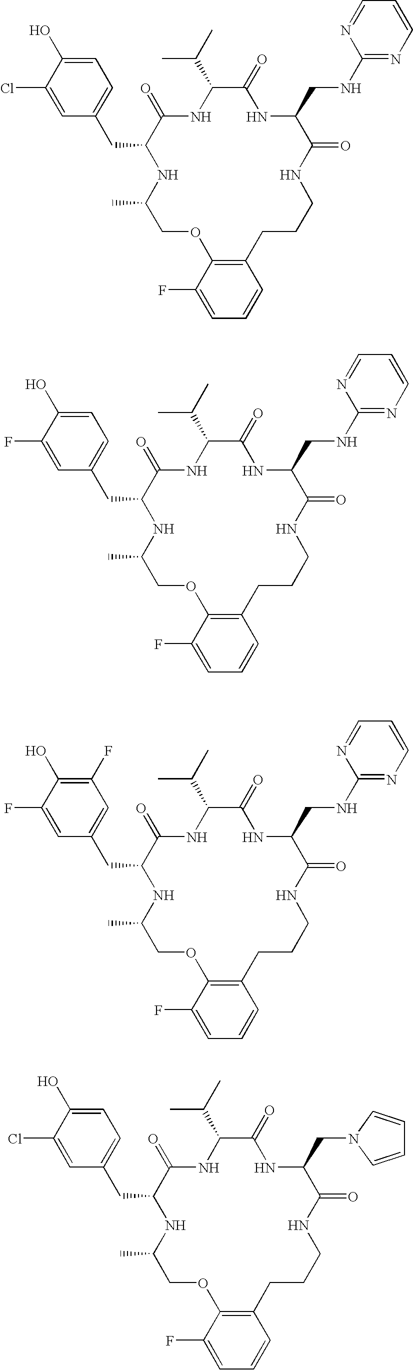

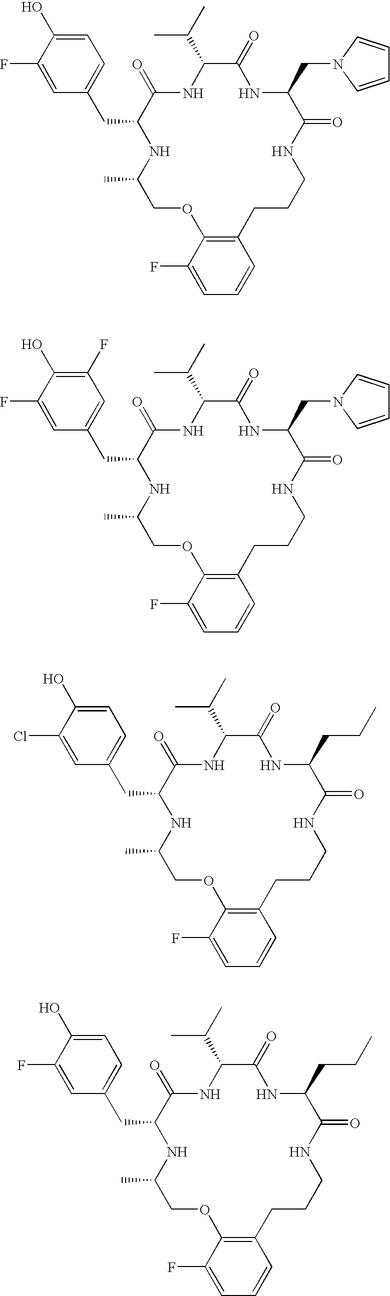

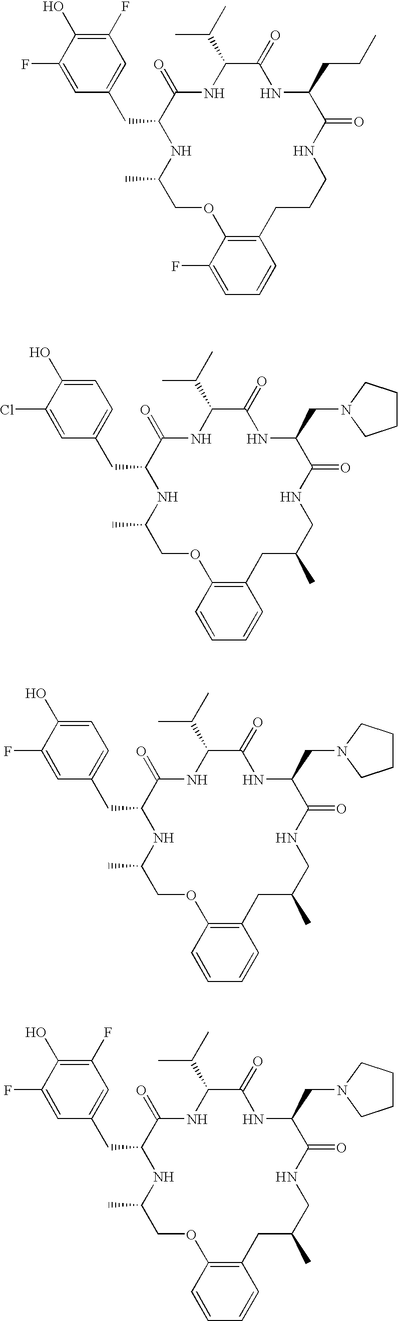

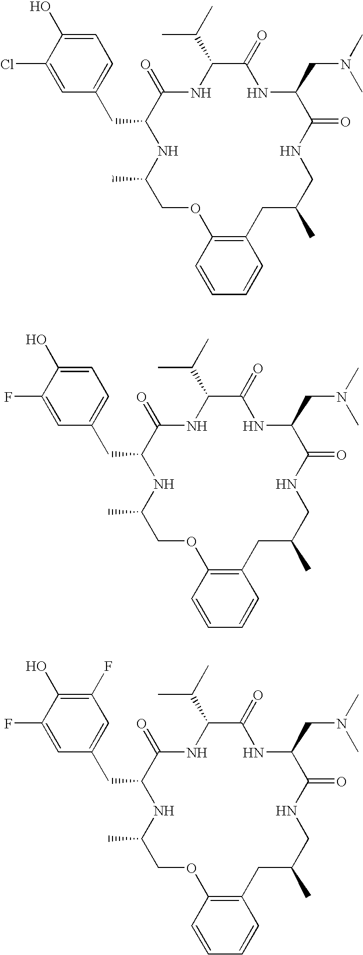

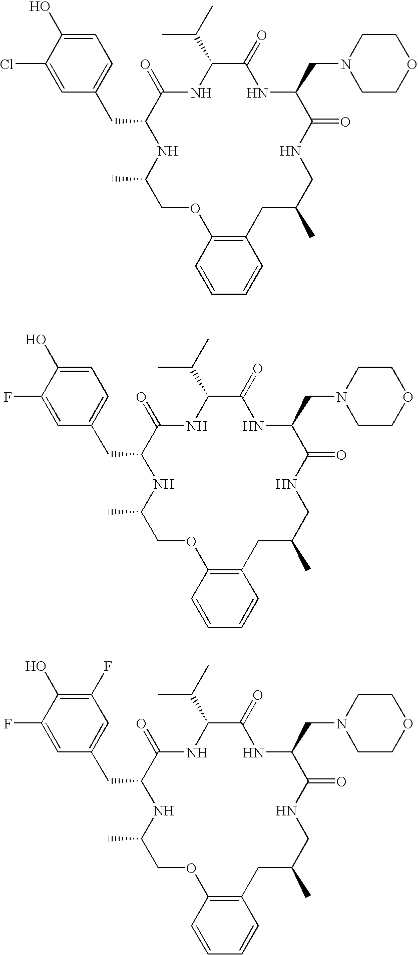

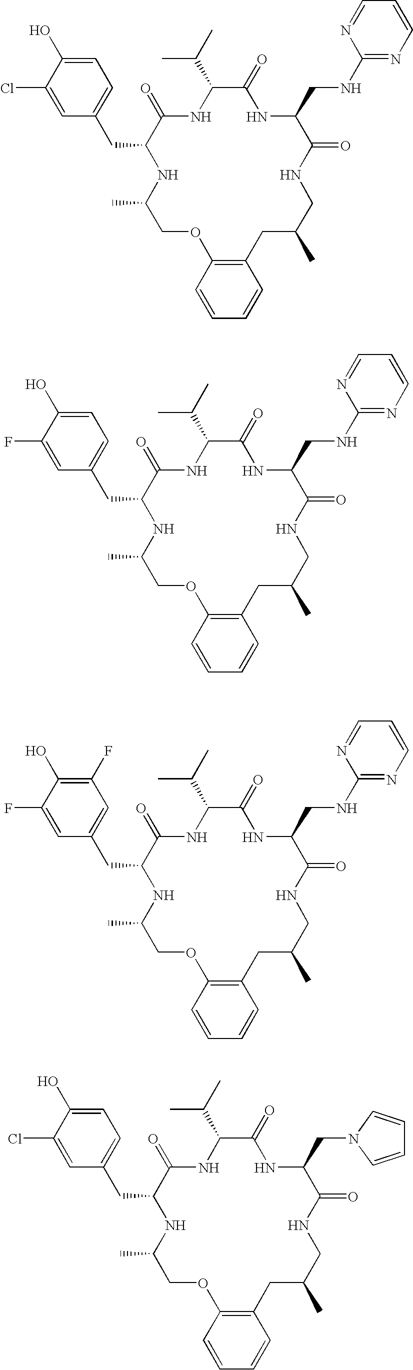

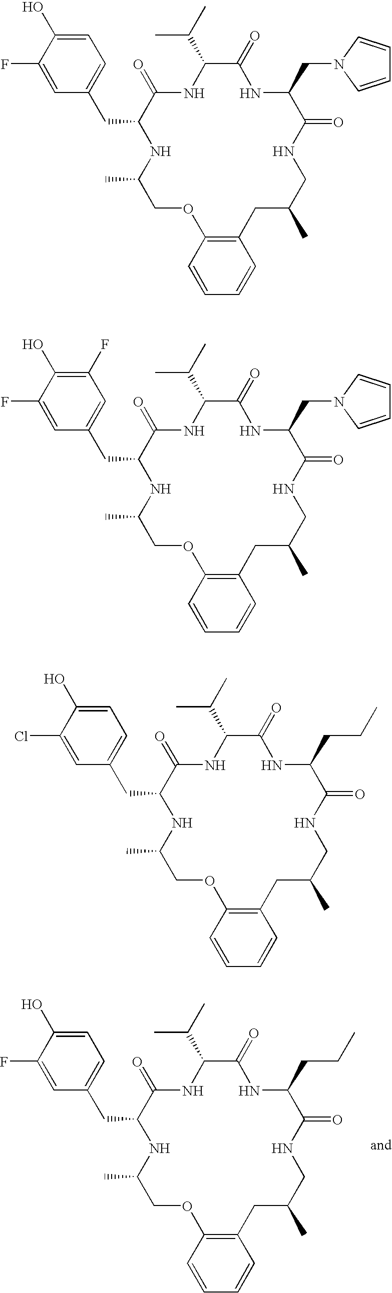

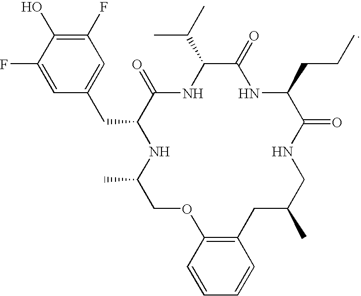

11. The method of claim 9, wherein the subject is a mammal.

12. The method of claim 9, wherein the subject is a human.

13. The method of claim 9, wherein the subject is treated with an additional compound that modulates Gastrointestinal motility.

14. A method of treating a disorder associated with abnormal stomach or intestinal absorption in a subject comprising administering an effective amount of a compound of formula I 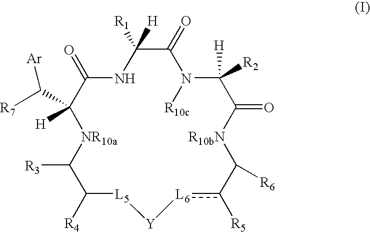
or pharmaceutically acceptable salts, hydrates or solvates thereof, wherein:
Y is 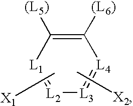
wherein (L₅) and (L₆) indicate the bonds to L₅ and L₆ of formula I, respectively;
Ar is selected from the group consisting of: 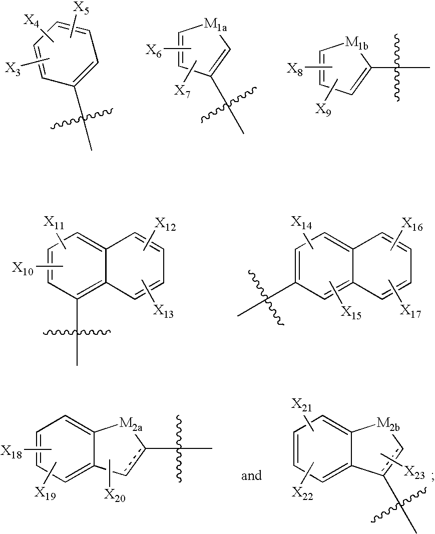
R₁ is selected from the group consisting of: —(CH₂)ₛCH₃, —CH(CH₃)(CH₂)ₜCH₃, —(CH₂)ᵤCH(CH₃)₂, —C(CH₃)₃, and 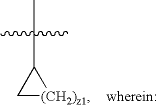
s is 0, 1, 2 or 3;
t is 1 or 2;
u is 0 or 1; and
z1 is 1, 2, 3 or 4;
R₂ is selected from the group consisting of hydrogen, —(CH₂)ₐₐCH₃, —CH₂SCH₃, —CH₂CH₂SCH₃, —(CH₂)_{bb}CH(CH₃)₂, —CH(CH₃)(CH₂)_{cc}CH₃, —(CH₂)_{dd}—NR₁₁R₁₂, and —(CH₂)ₑₑR₁₃; wherein
aa and bb are independently 0, 1, 2 or 3;
cc and dd are independently 1, 2, 3 or 4;
ee is 0, 1, 2, 3 or 4;
R₁₁ is selected from the group consisting of hydrogen, lower alkyl, formyl, acyl, carboxyalkyl, carboxyaryl, amido, amidino, sulfonyl and sulfonamido;
R₁₂ is selected from the group consisting of hydrogen and lower alkyl;
R₁₃ is selected from the group consisting of: 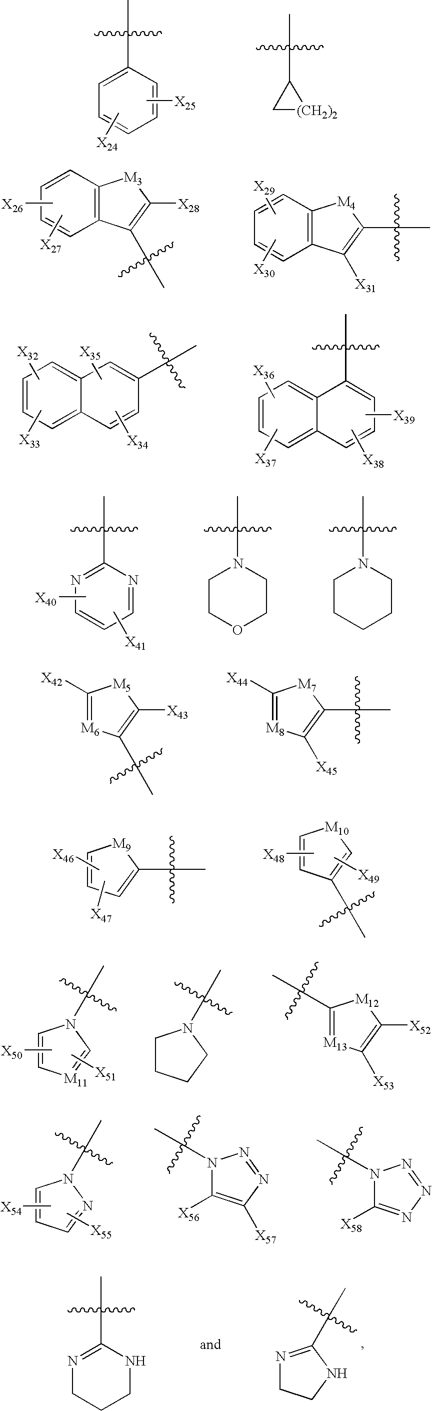
wherein z2 is 1, 2, 3 or 4;
and, when ee is 1, 2, 3 or 4, R₁₃ is further selected from the group consisting of hydroxy, alkoxy, amidino, and azido;
R₃, R₄, R₅, and R₆ are independently selected from the group consisting of hydrogen, methyl, ethyl, isopropyl and hydroxymethyl;
R₇ is selected from the group consisting of hydrogen, methyl, hydroxy and amino;
R₁₀ₐ and R_{10b} are independently selected from the group consisting of hydrogen and methyl;
X₁, X₂, X₆, X₇, X₈ and X₉) are independently selected from the group consisting of hydrogen, halogen, trifluoromethyl and lower alkyl;
X₃, X₄, X₅, X₁₀, X₁₁, X₁₂, X₁₃, X₁₄, X₁₅, X₁₆, X₁₇, X₁₈, X₁₉, X₂₁, X₂₂, X₂₄, X₂₅, X₂₆, X₂₇, X₂₈, X₂₉, X₃₀, X₃₁, X₃₂, X₃₃, X₃₄, X₃₅, X₃₆, X₃₇, X₃₈, X₃₉, X₄₀, X₄₁, X₄₂, X₄₃, X₄₄, X₄₅, X₄₆, X₄₇, X₄₈, X₄₉, X₅₀, X₅₁, X₅₂, X₅₃, X₅₄ and X₅₅ are independently selected from the group consisting of hydrogen, hydroxy, alkoxy, amino, halogen, trifluoromethyl and lower alkyl;
X₂ and X₂₃ are independently selected from the group consisting of hydrogen, trifluoromethyl and lower alkyl;
X₅₆, X₅₇ and X₅₈ are independently selected from the group consisting of hydrogen and lower alkyl;
L₁, L₂, L₃ and L₄ are independently selected from the group consisting of CH and N; with the proviso that the total number of nitrogens in the ring must be 0 or 1;
L₅ and L₆ are independently selected from the group consisting of O, CR₈ₐR_{8b} and NR₉ₐ; wherein R₈ₐ and R_{8b} are independently selected from the group consisting of hydrogen and methyl; and R₉ₐ is selected from the group consisting of hydrogen, lower alkyl, formyl, acyl and sulfonyl; with the proviso that when L₆ is CR₈ₐ when a double bond is present between L₆ and CHR₅;
M₁ₐ, M_{1b}, M₂ₐ, M_{2b}, M₃, M₄, M₅, M₇, M₉, M₁₀ and M₁₂ are independently selected from the group consisting of O, S and NR_{9b} wherein R_{9b} is selected from the group consisting of hydrogen, lower alkyl, formyl, acyl and sulfonyl; and
M₆, M₈, M₁₁ and M₁₃ are independently selected from N and CR_{9c}, wherein R_{9c} is selected from the group consisting of hydrogen and lower alkyl.

15. The method of claim 14, wherein the compound is selected from the group consisting of:

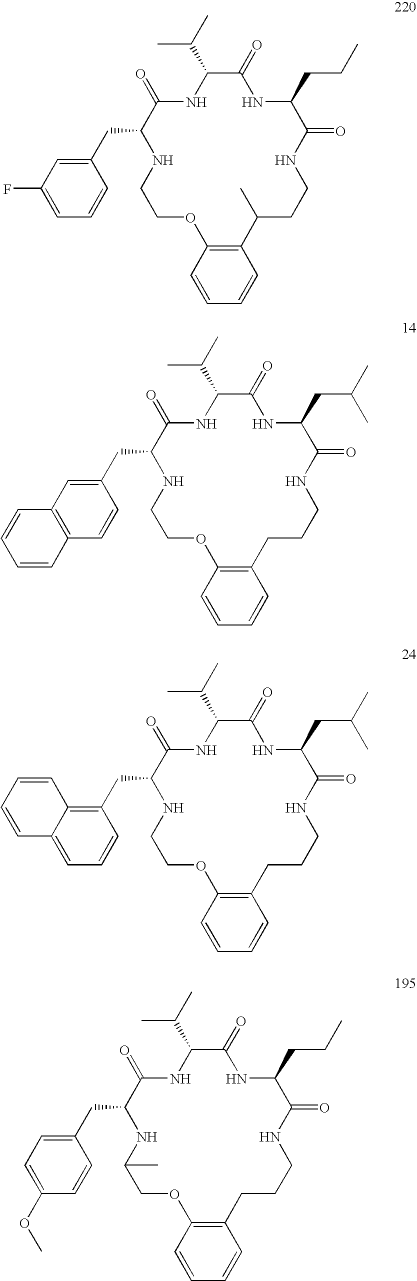

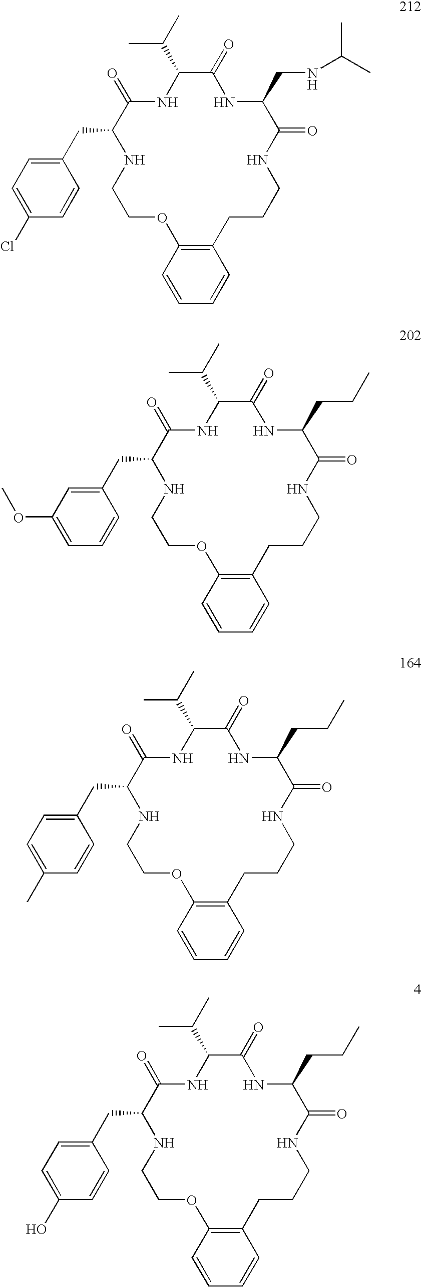

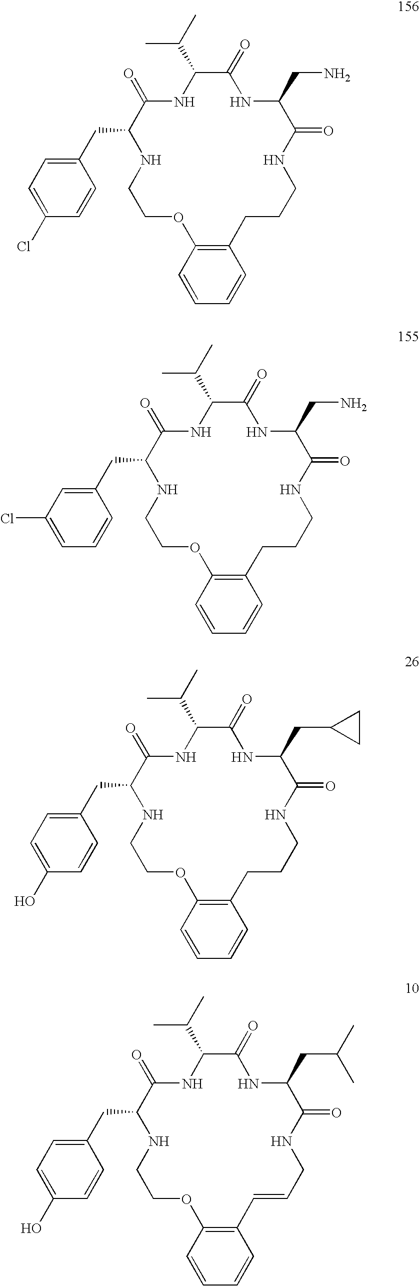

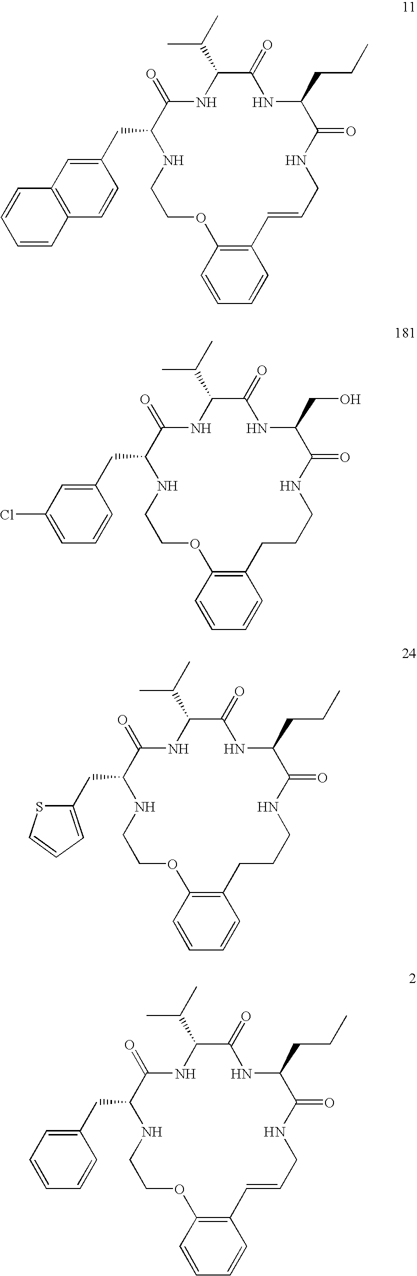

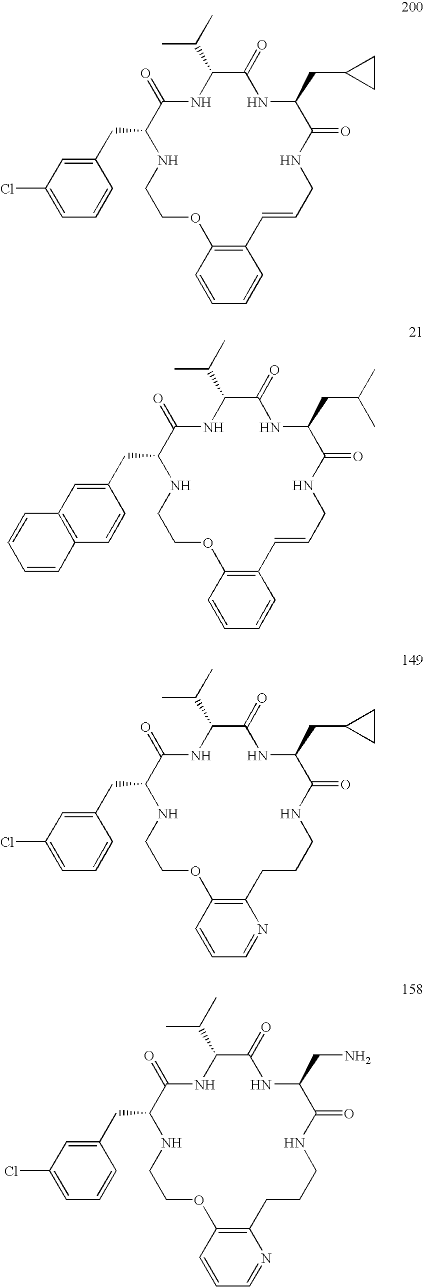

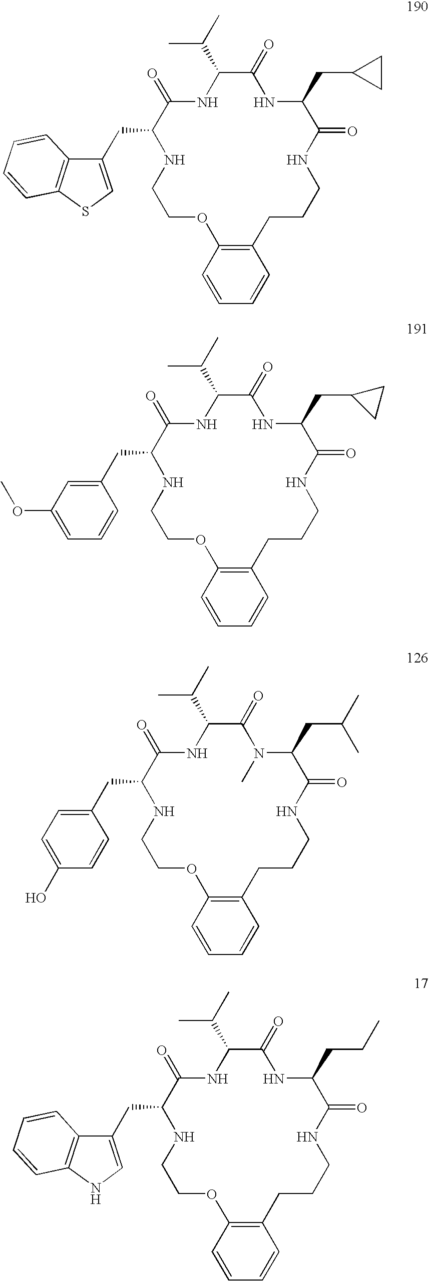

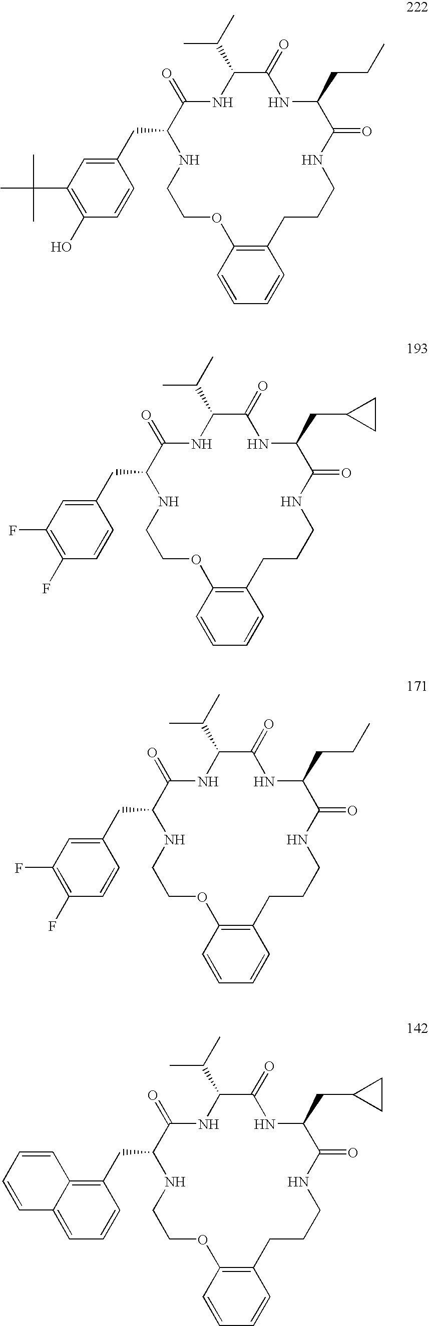

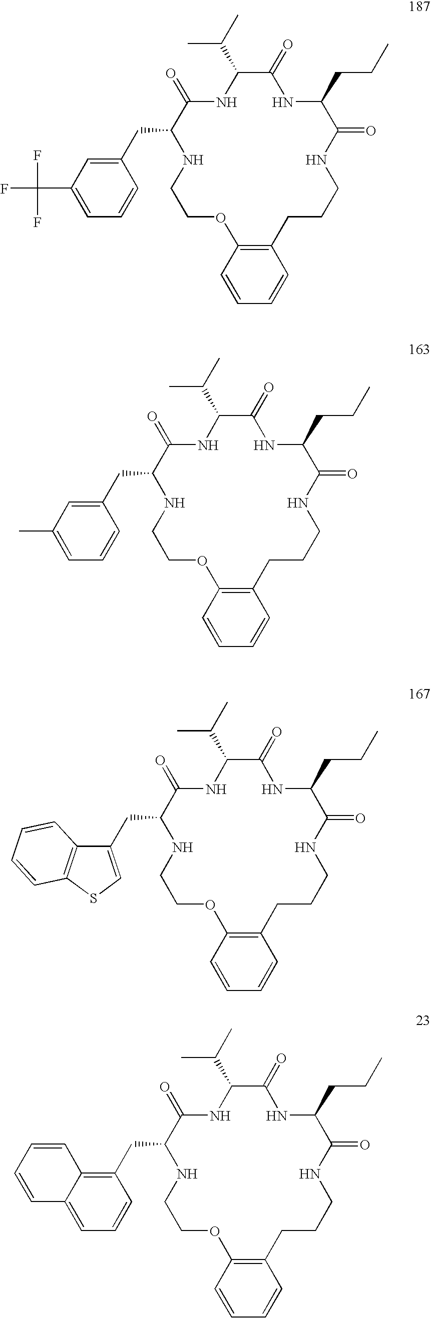

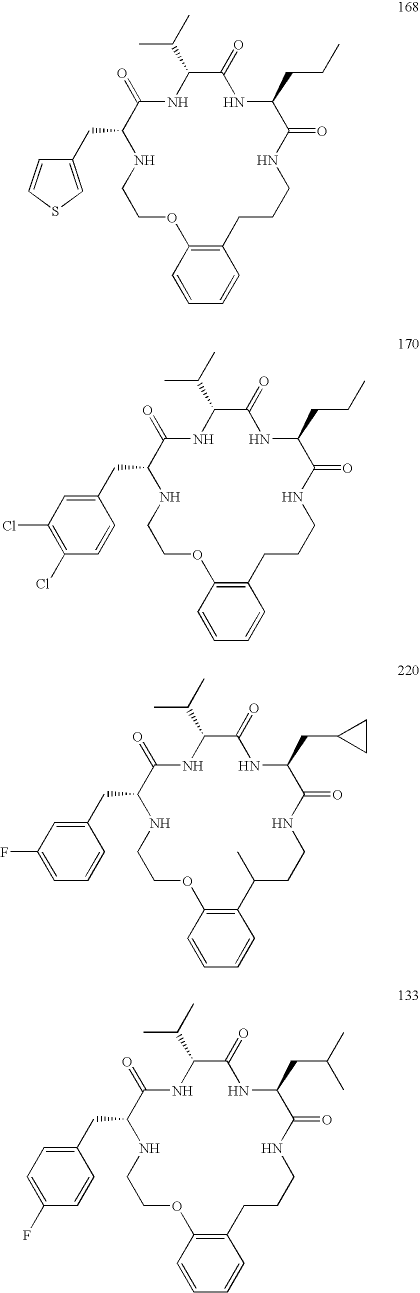

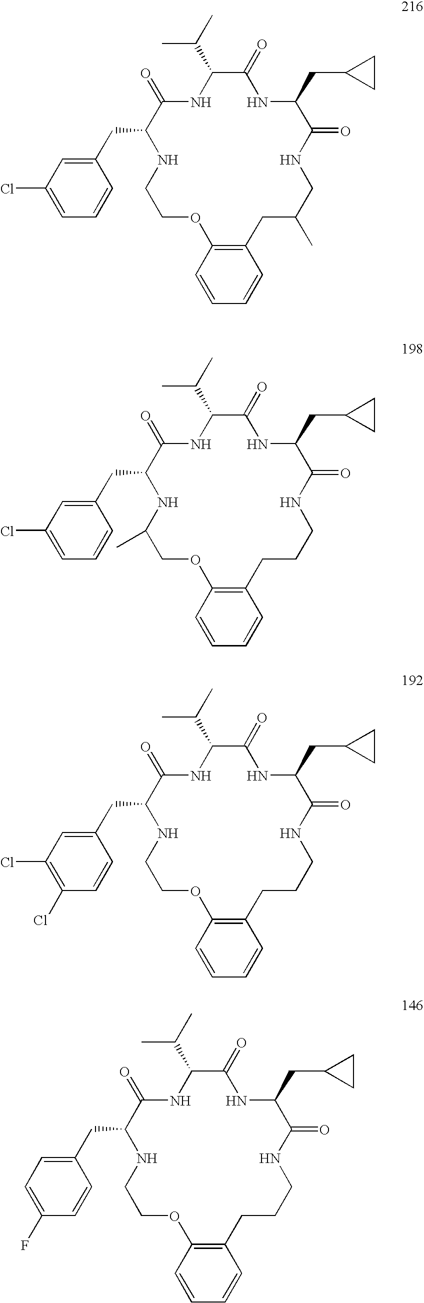

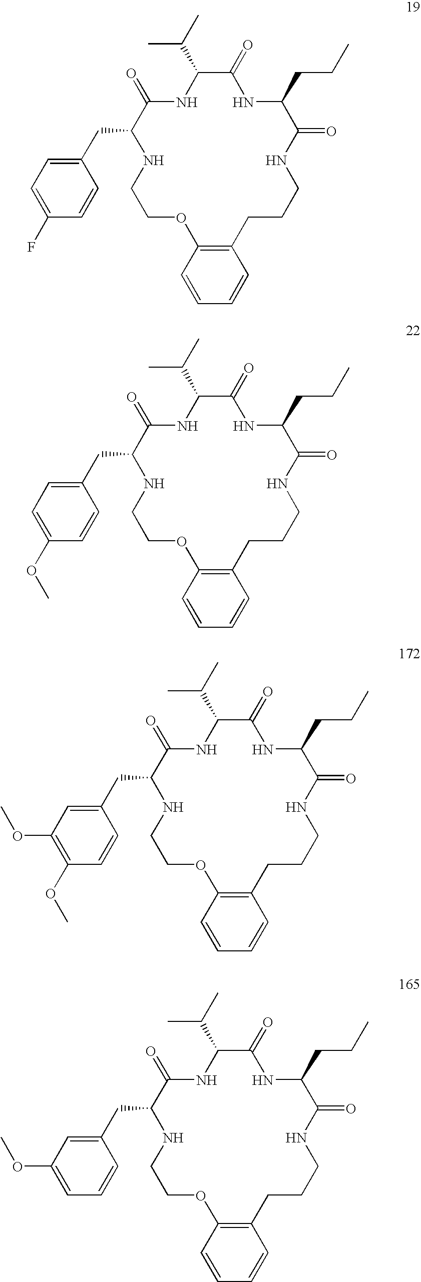

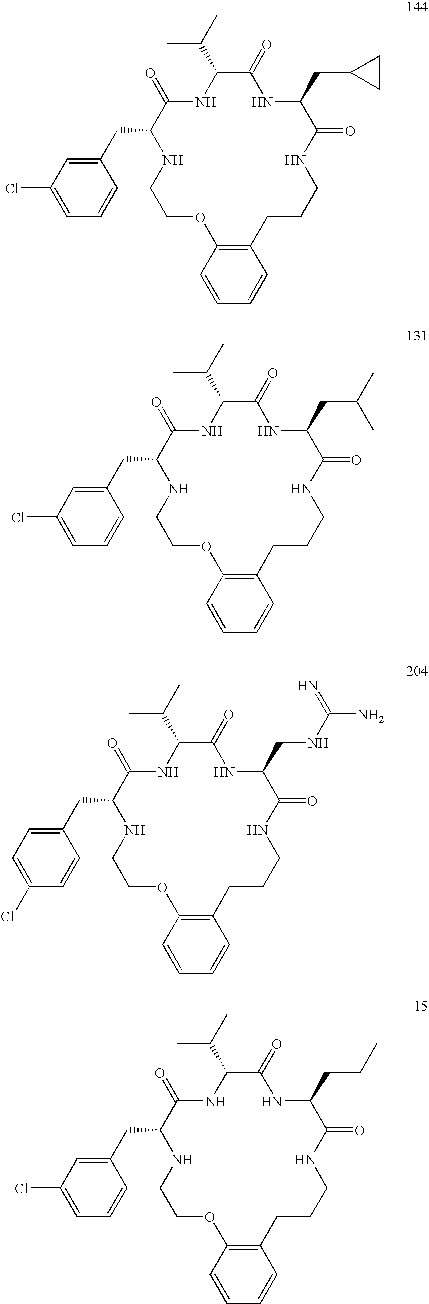

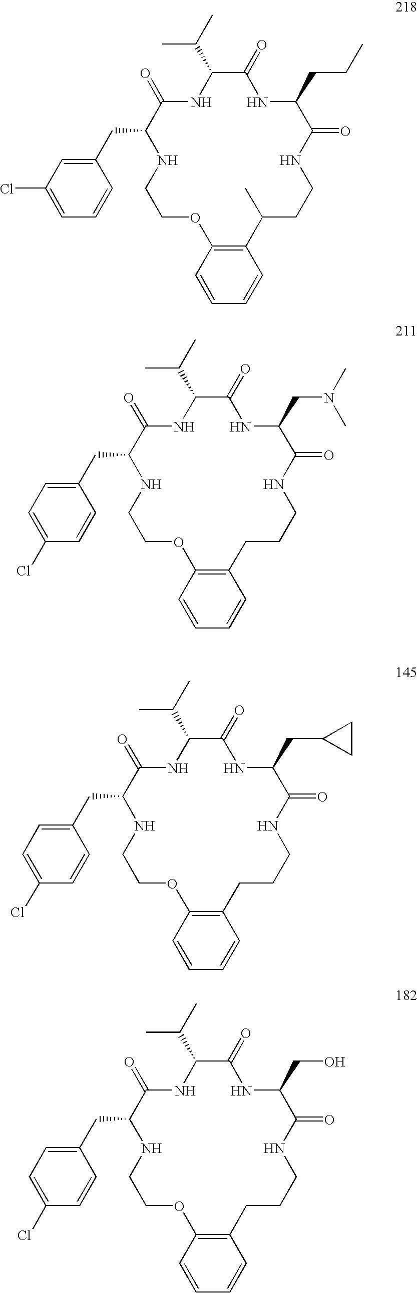

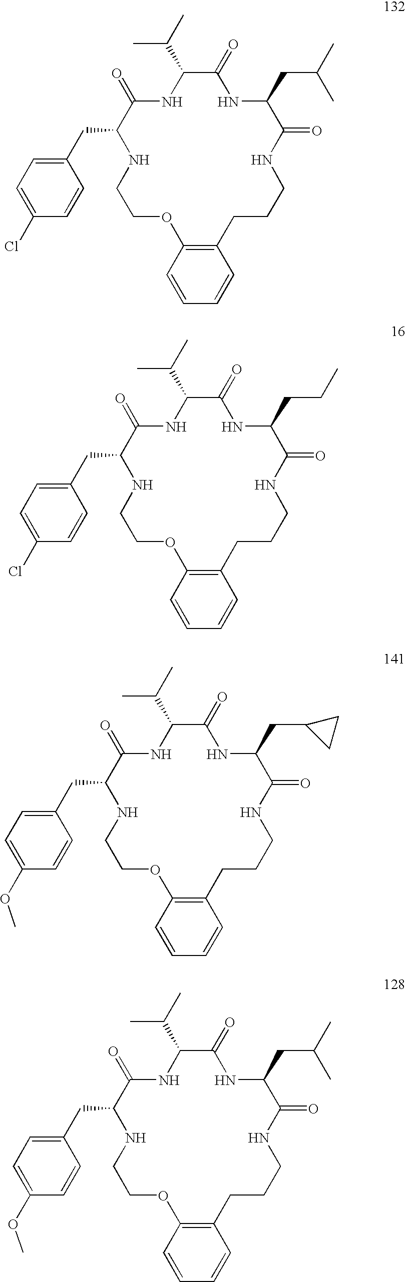

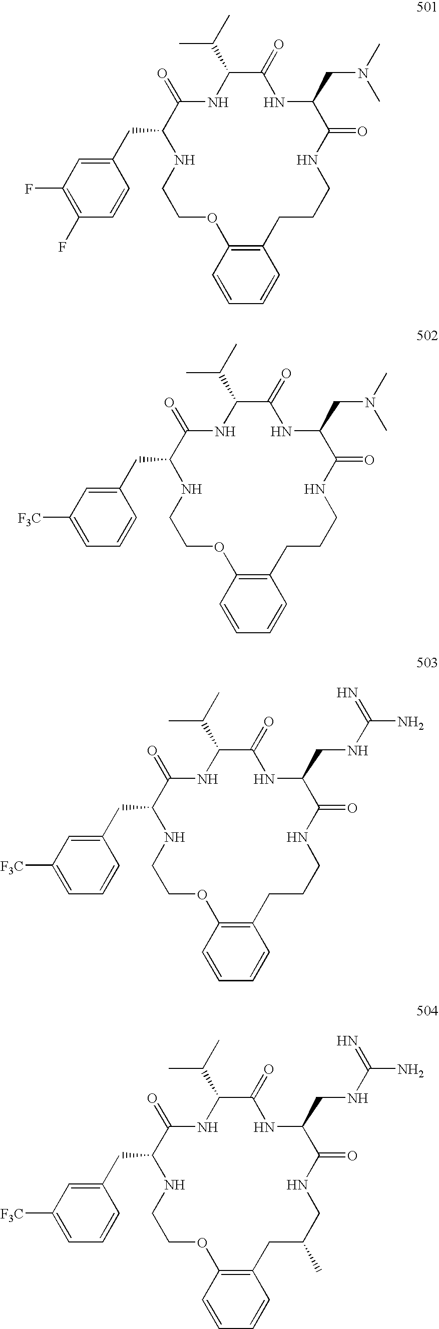

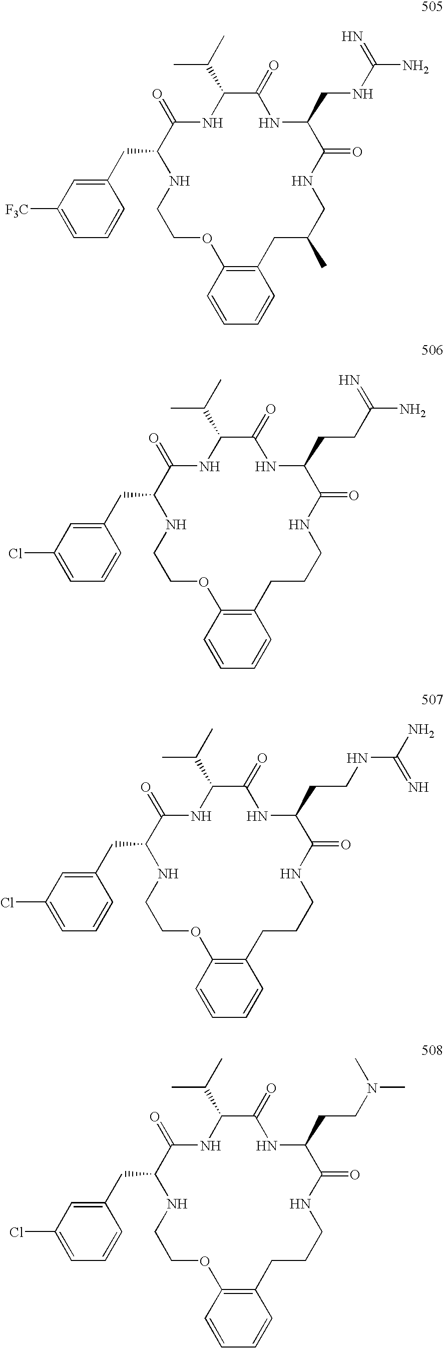

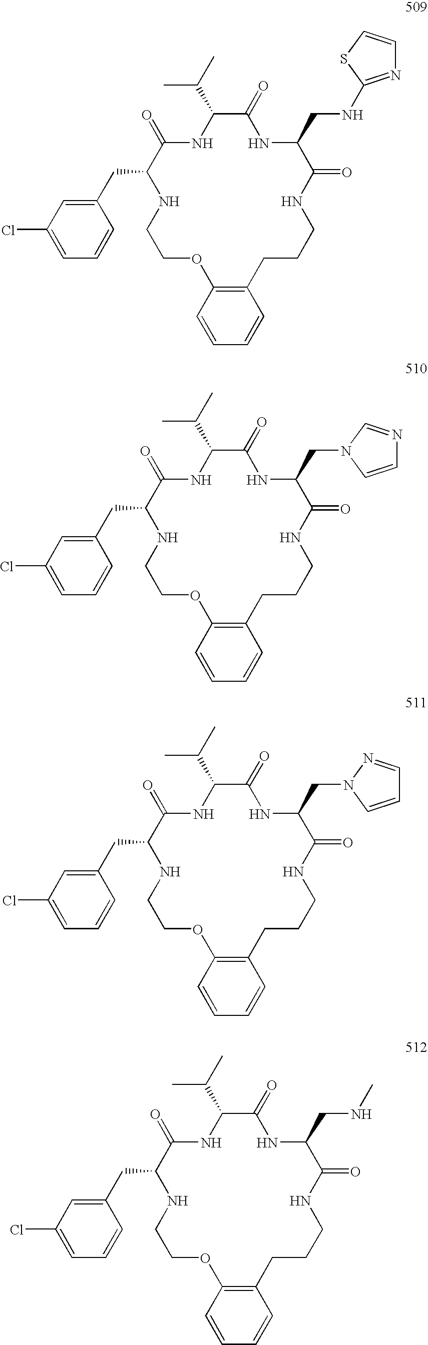

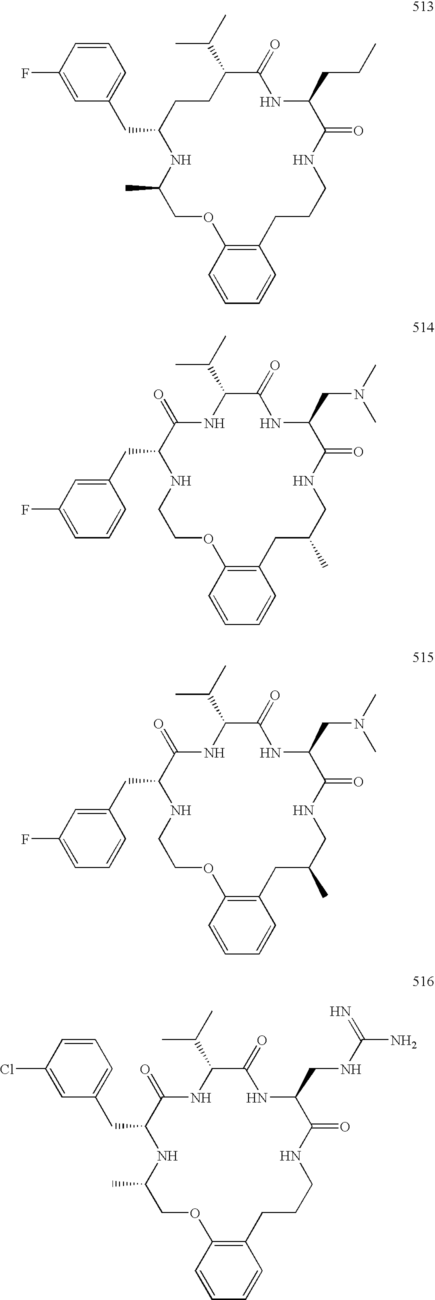

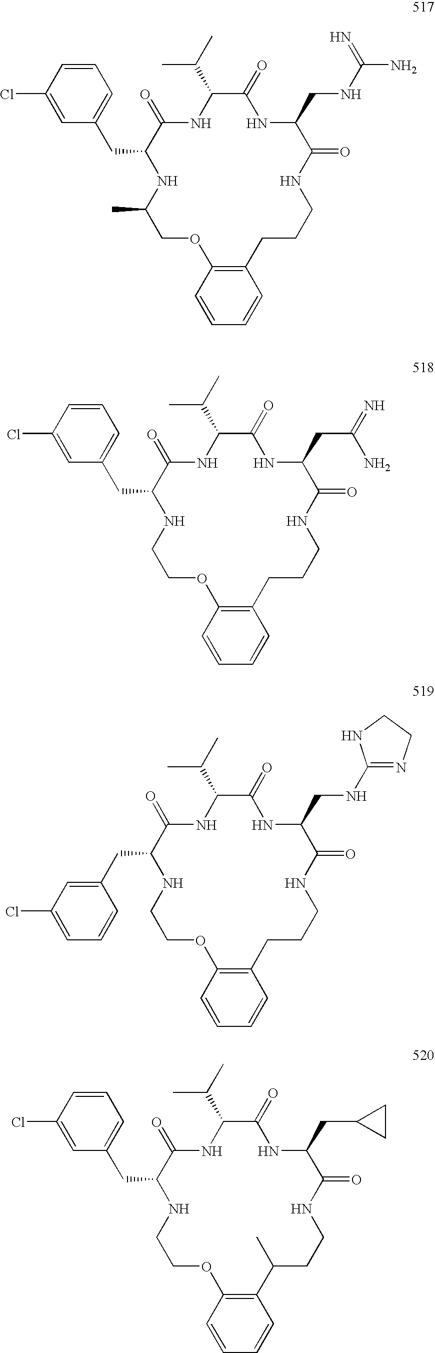

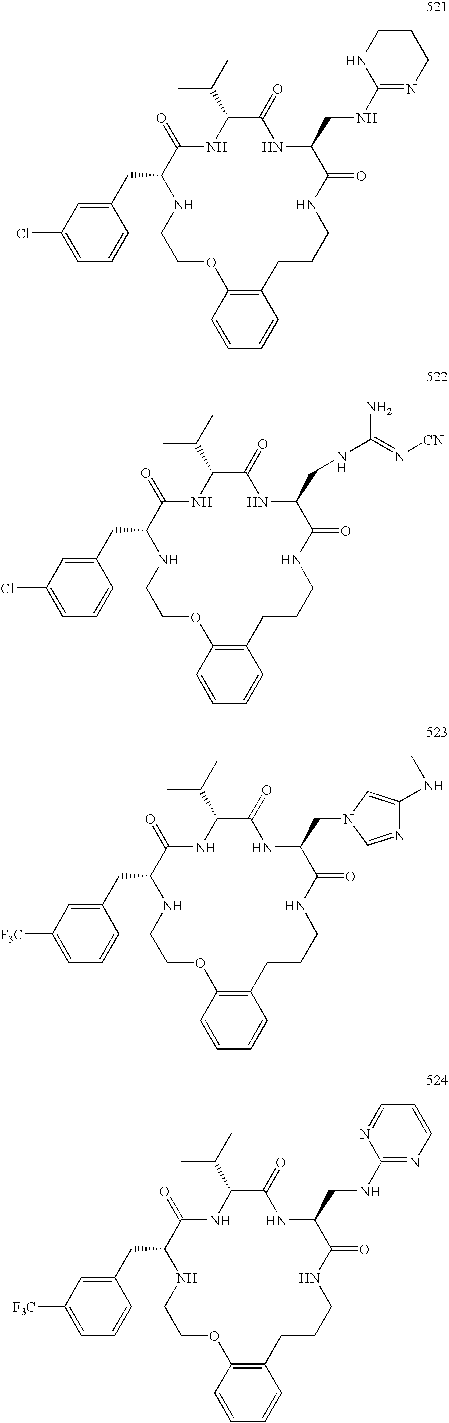

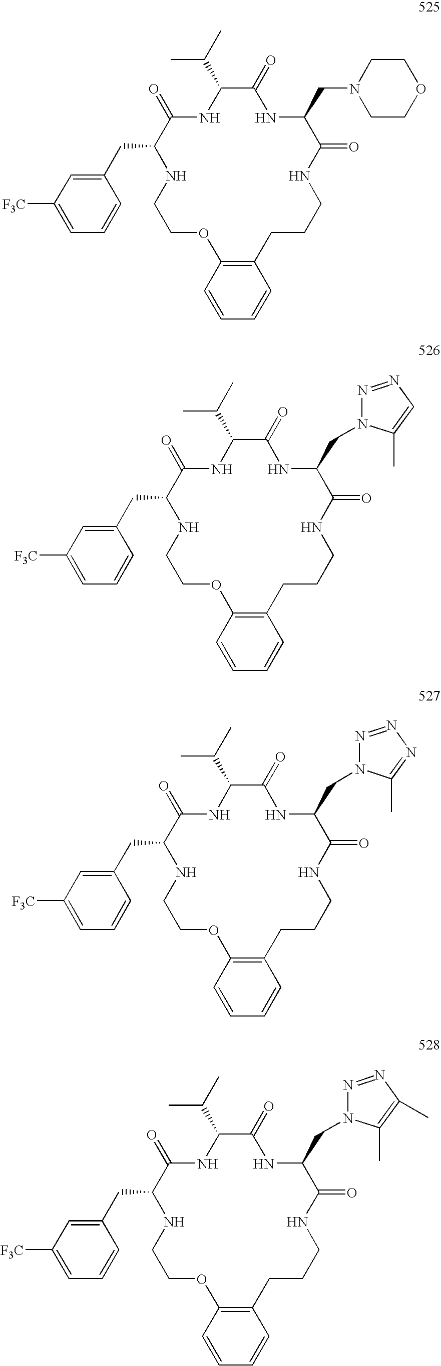

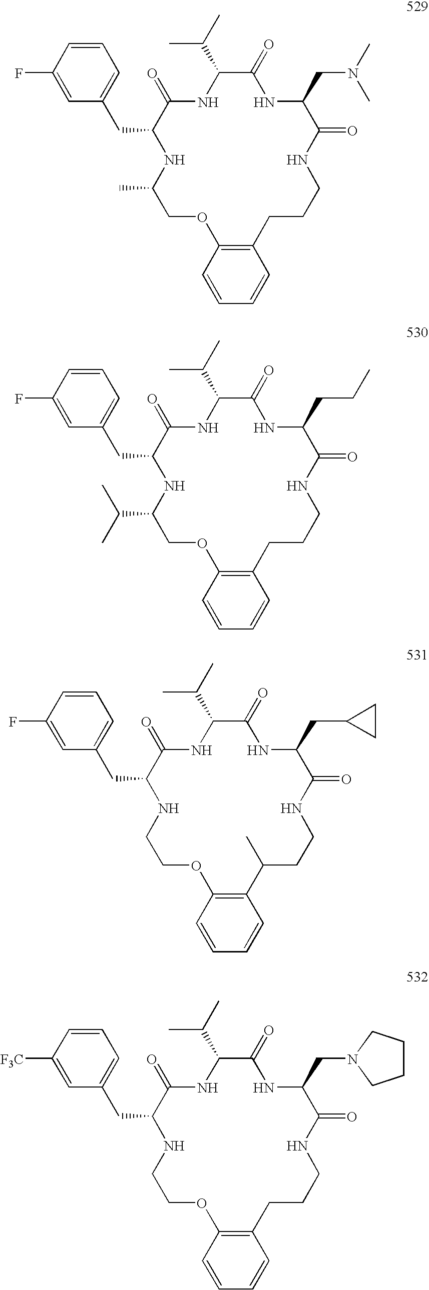

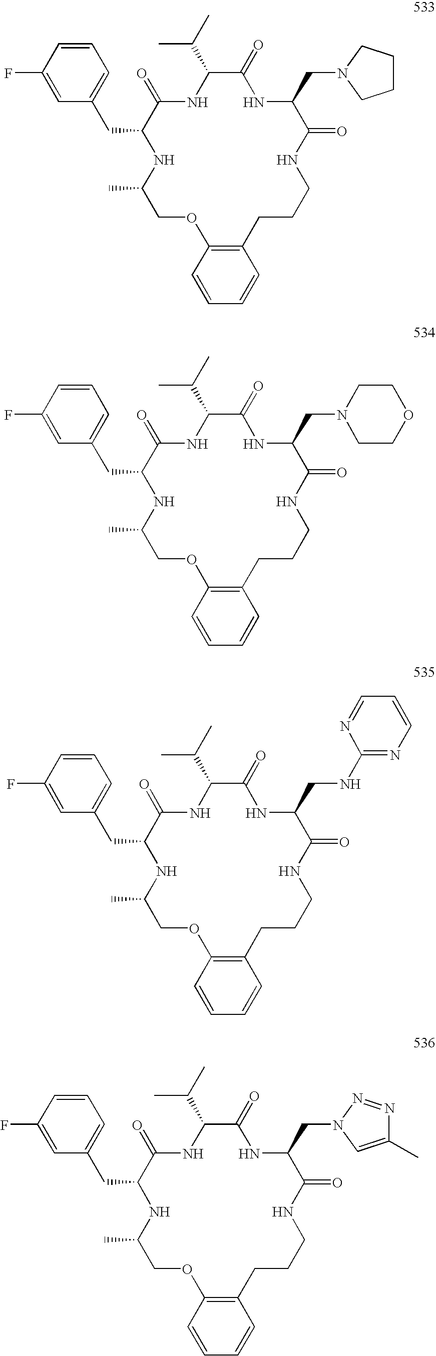

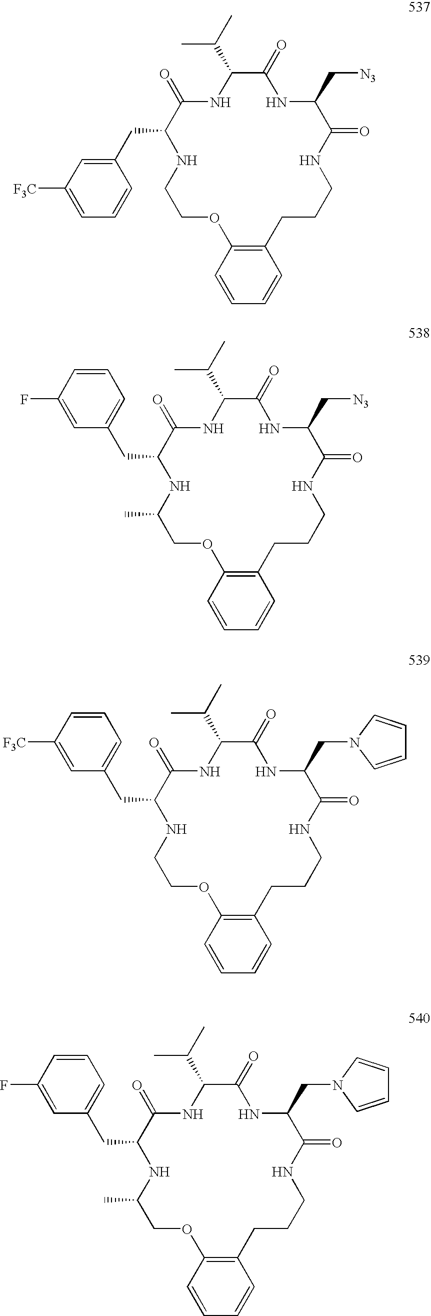

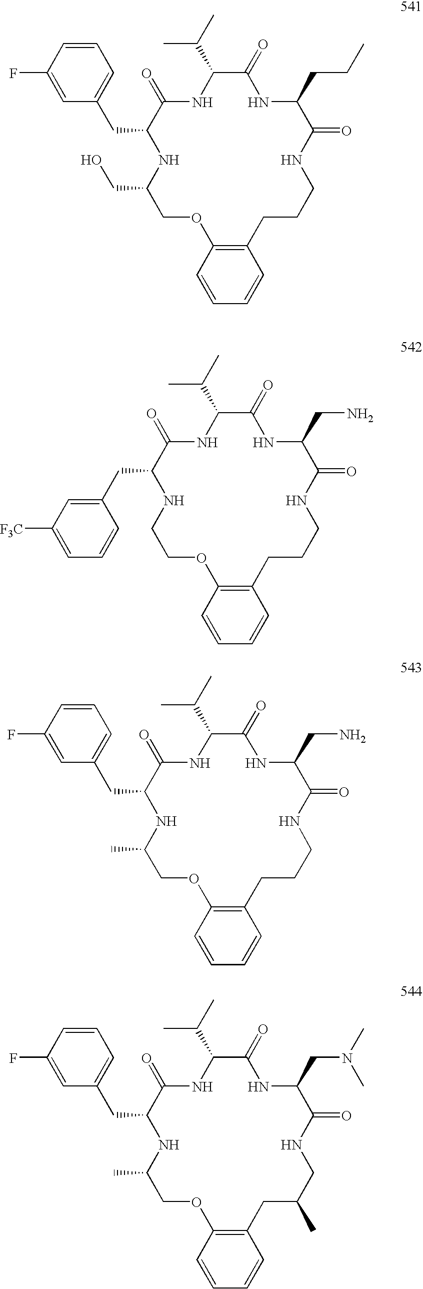

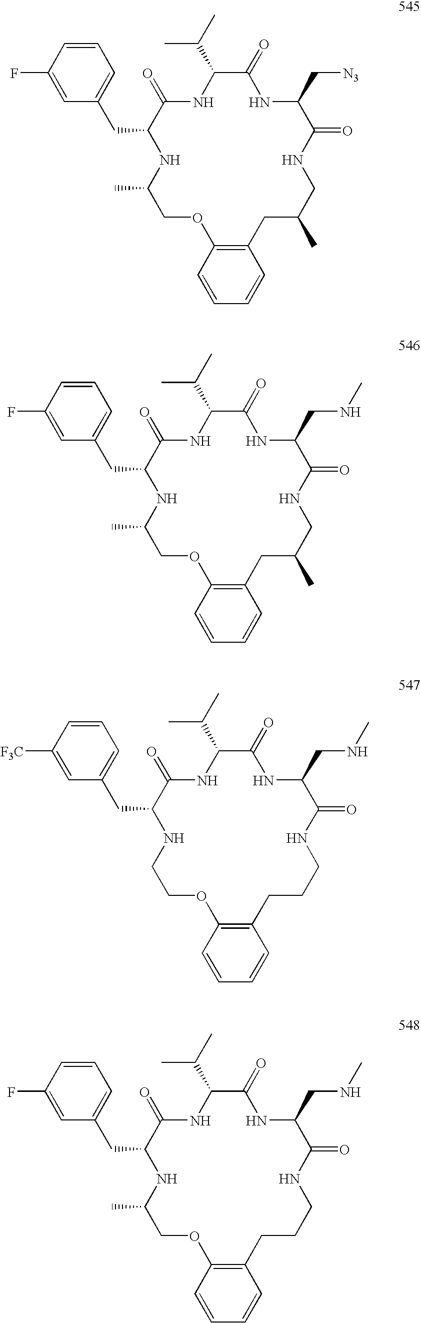

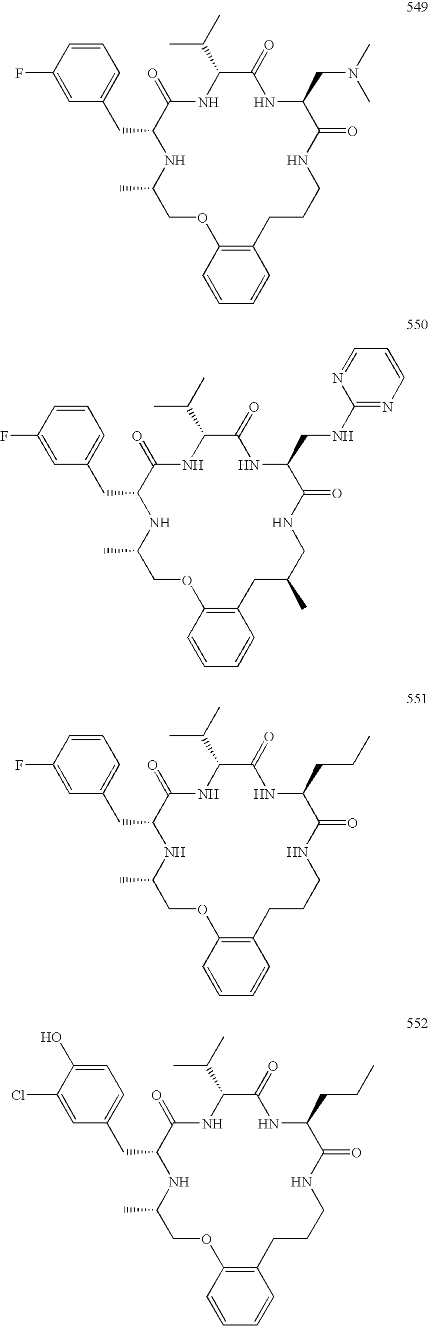

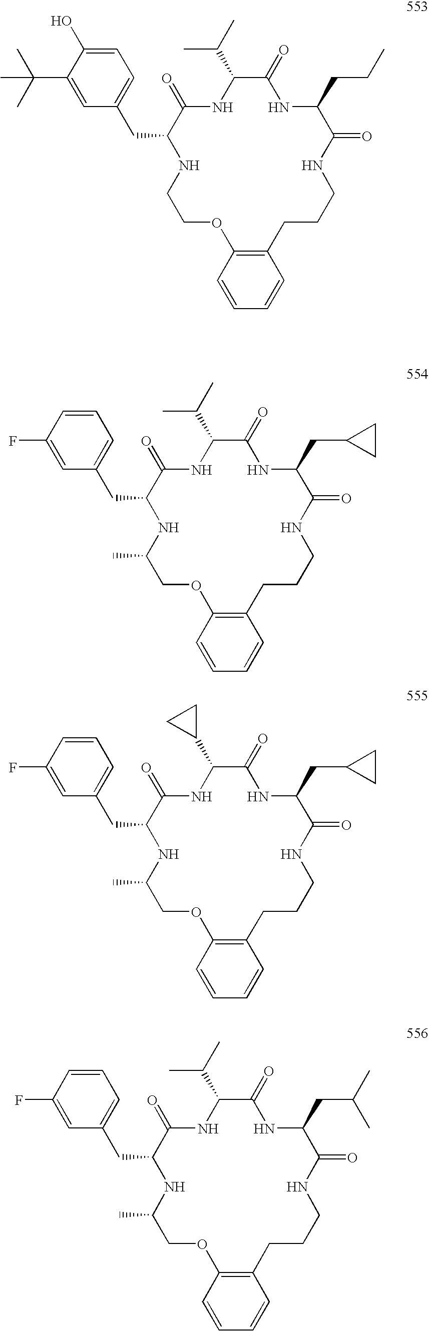

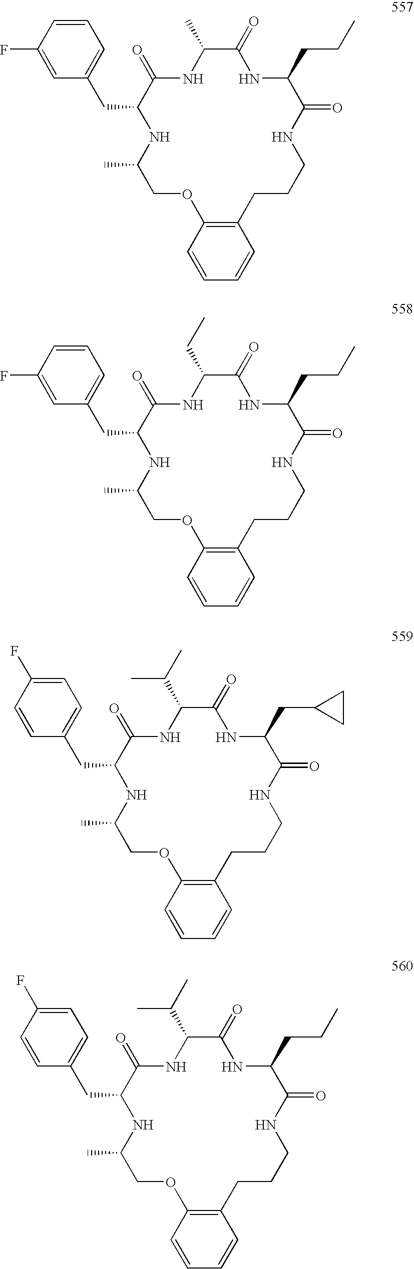

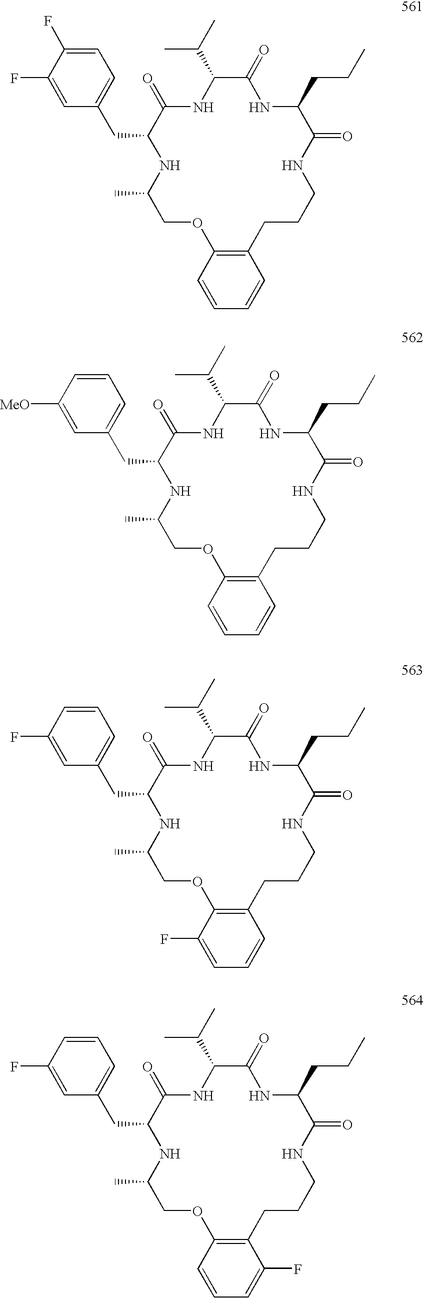

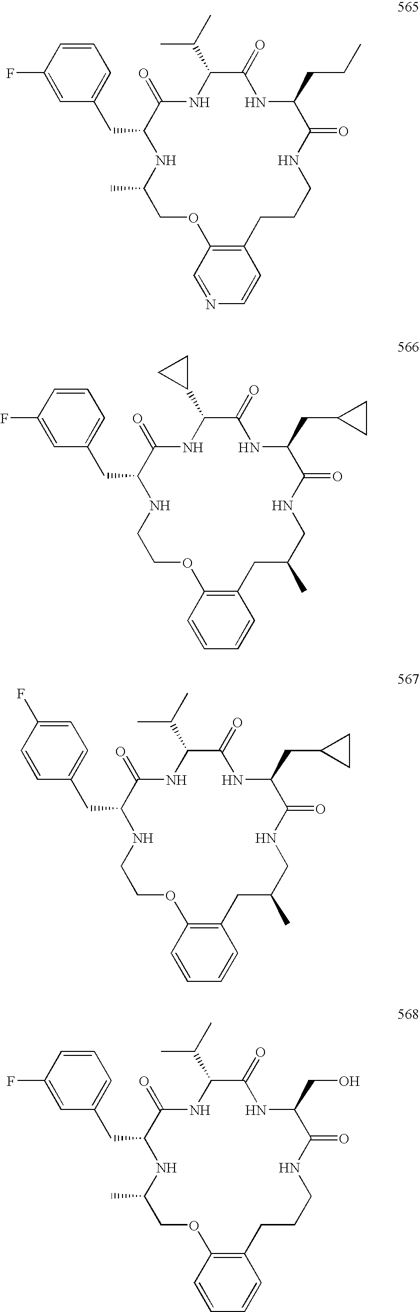

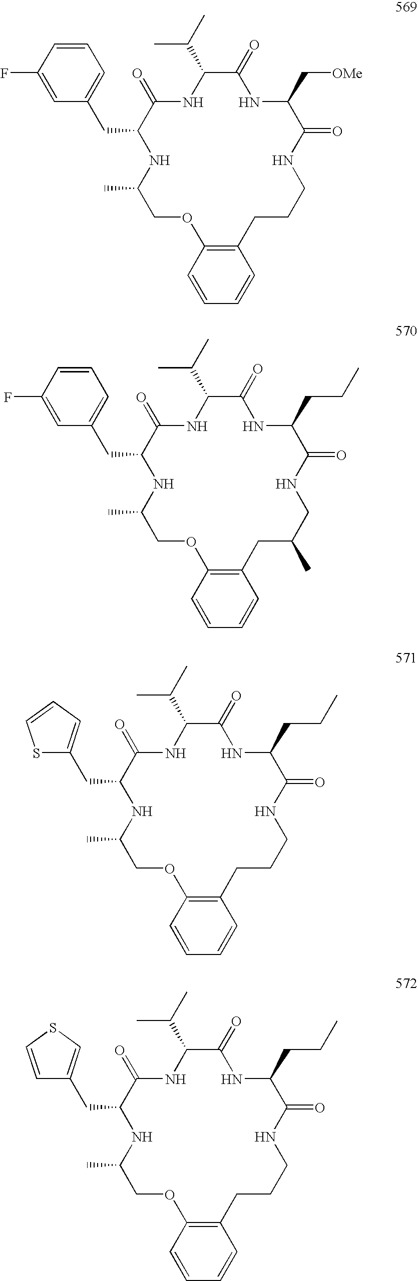

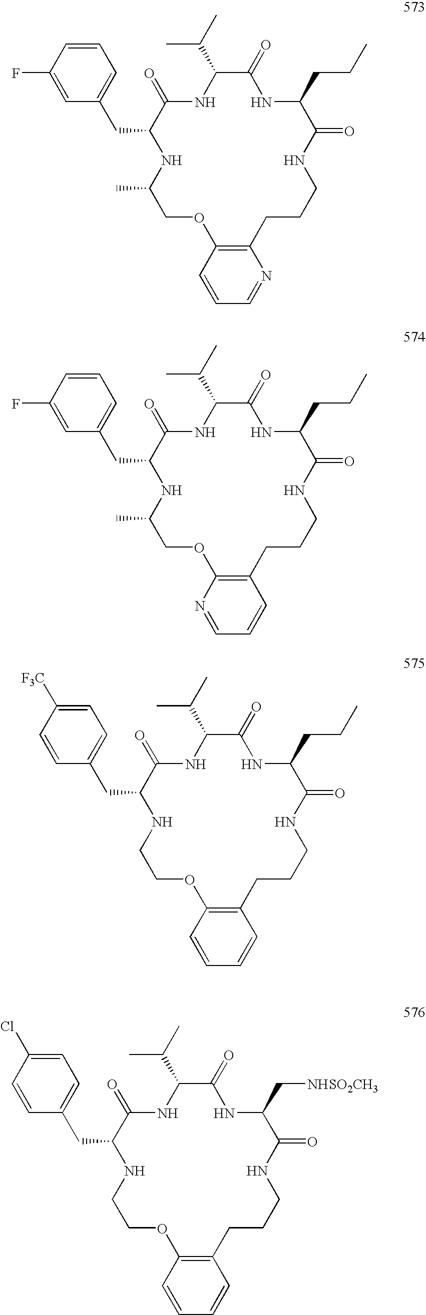

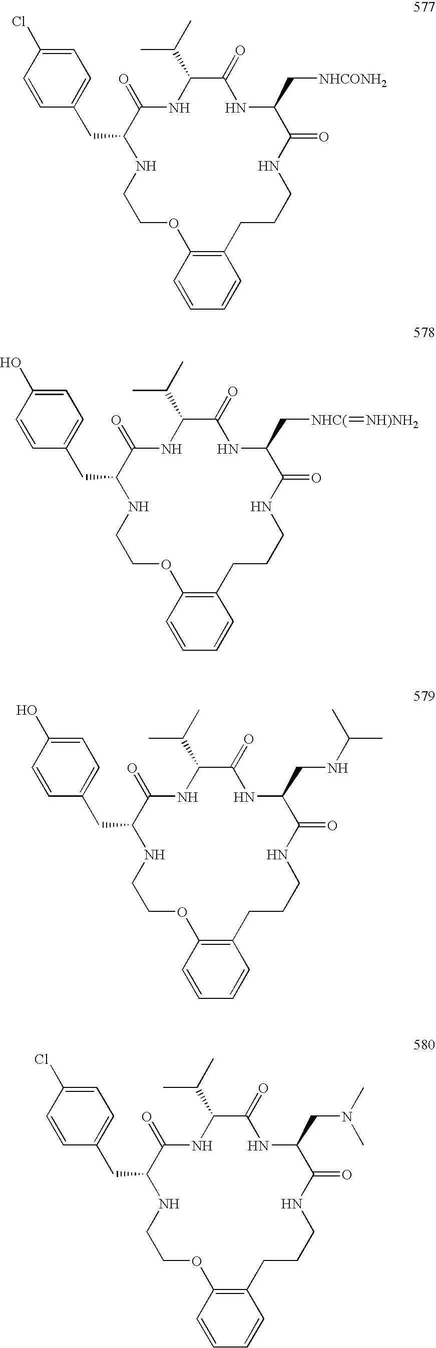

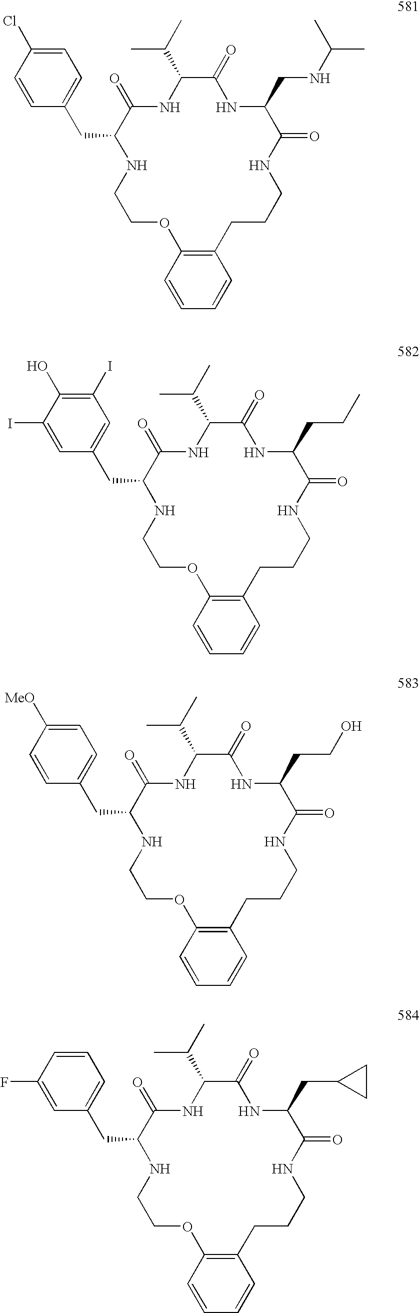

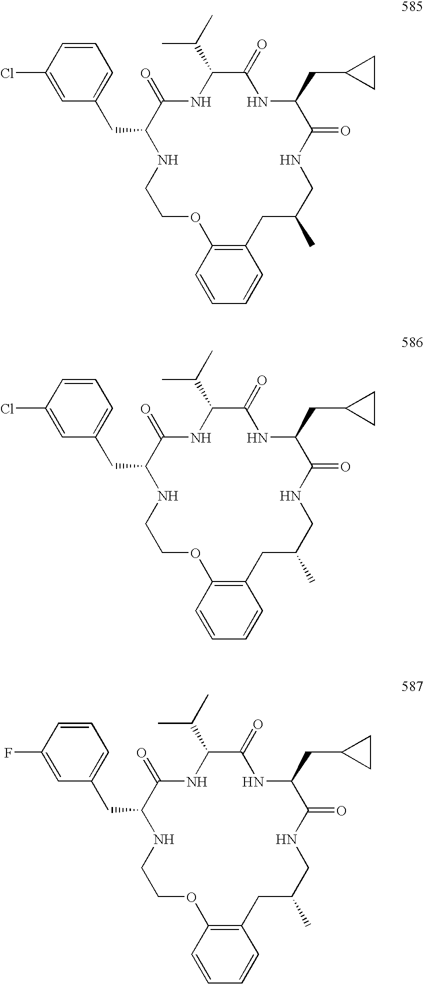

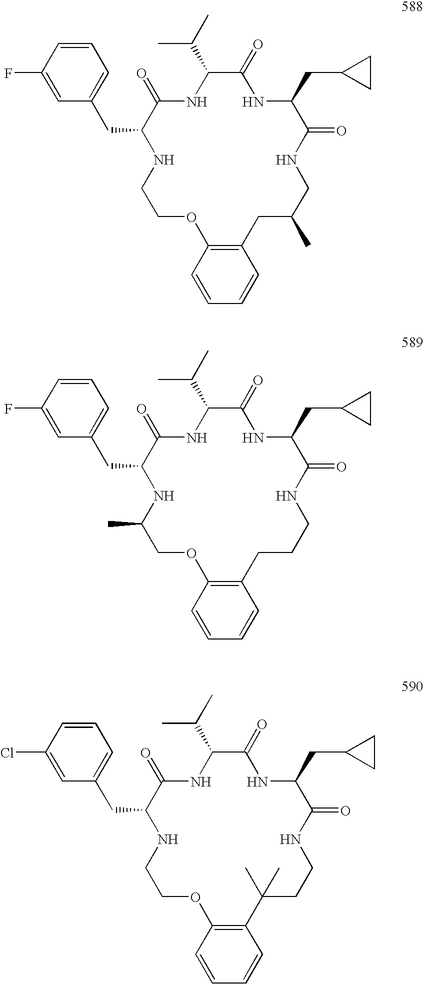

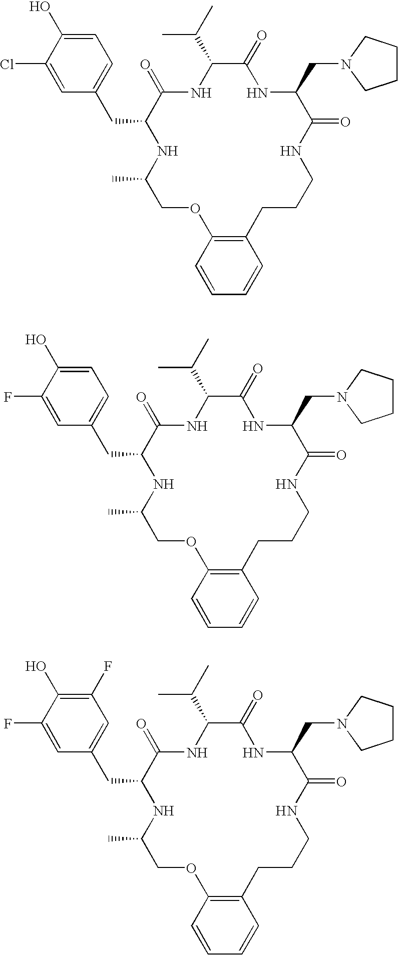

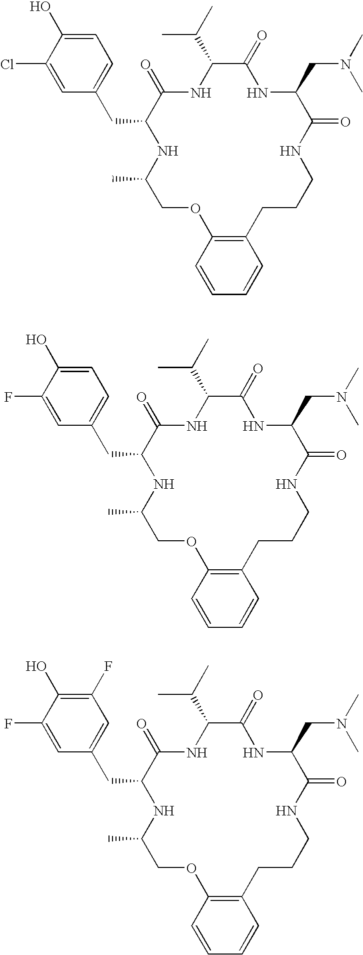

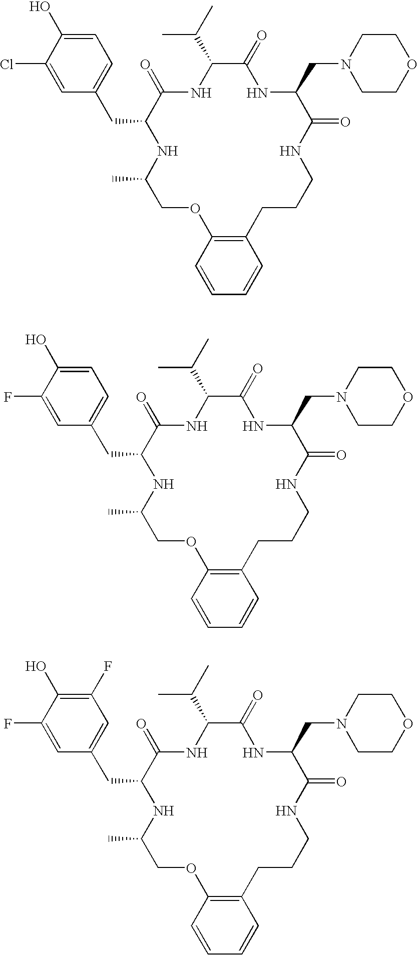

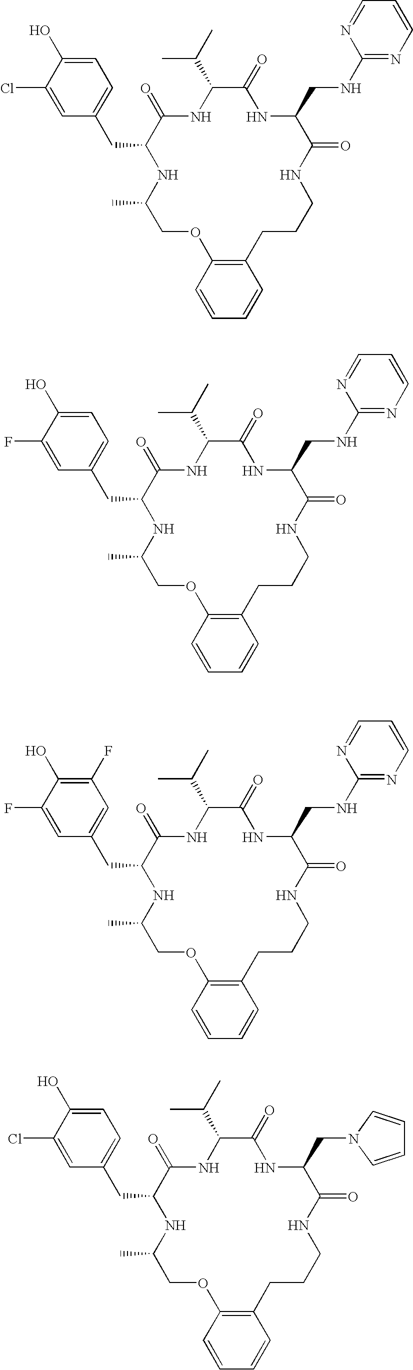

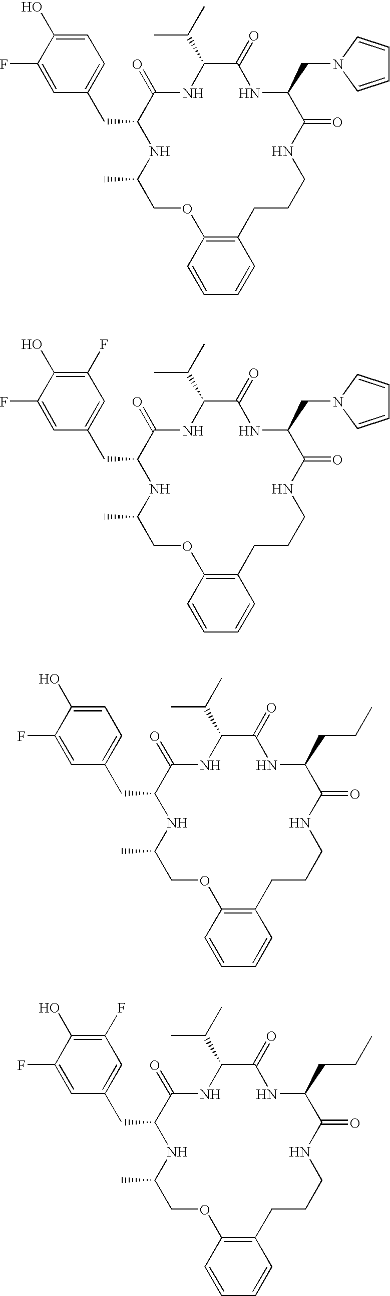

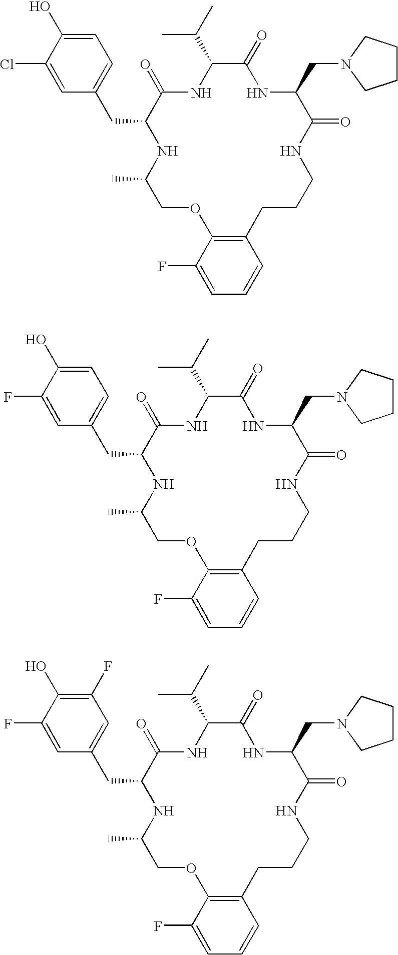

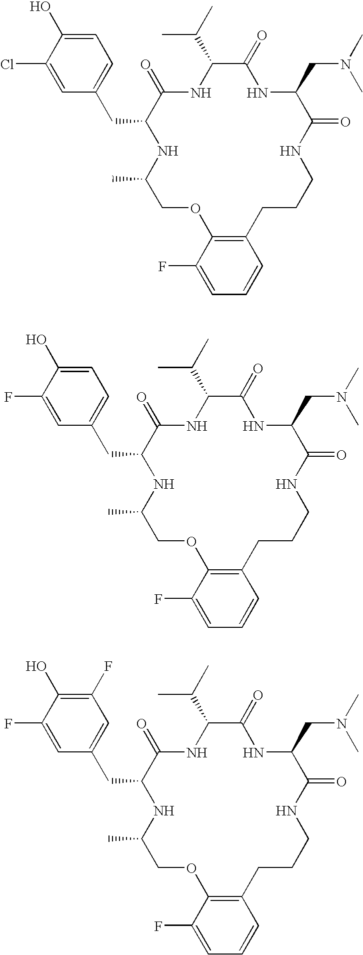

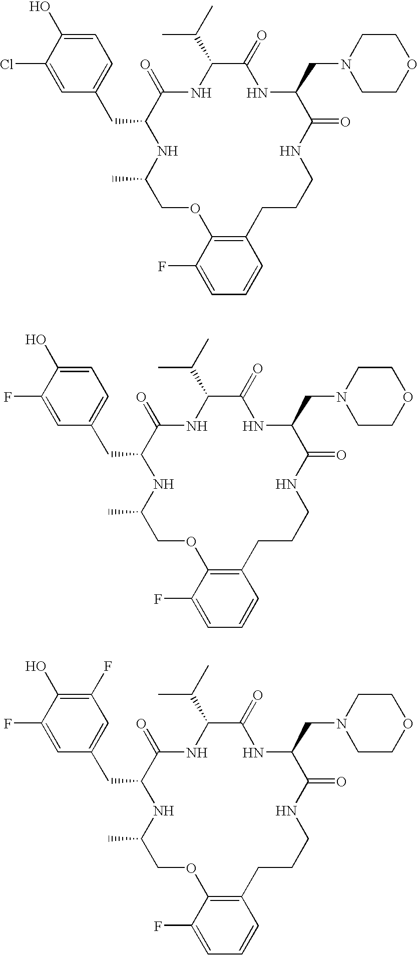

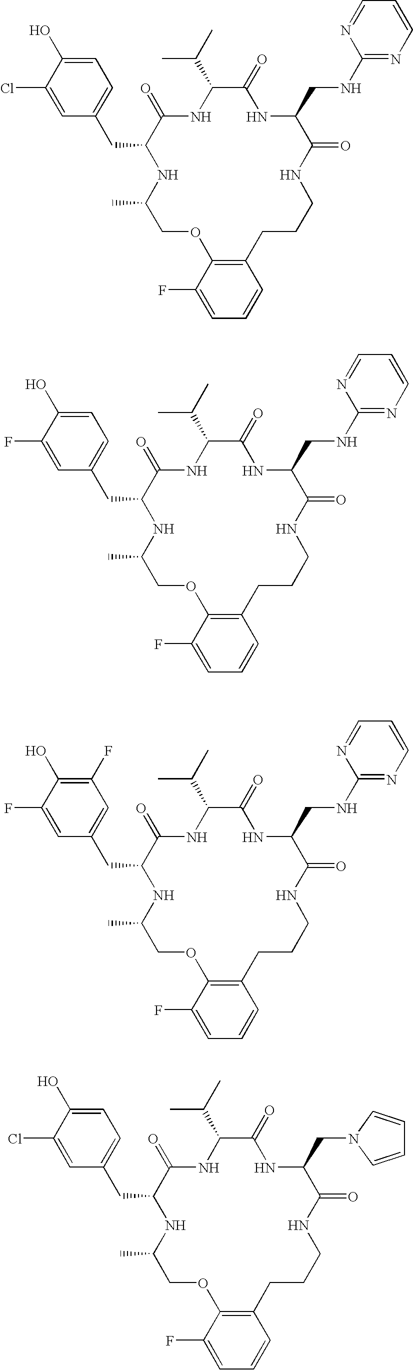

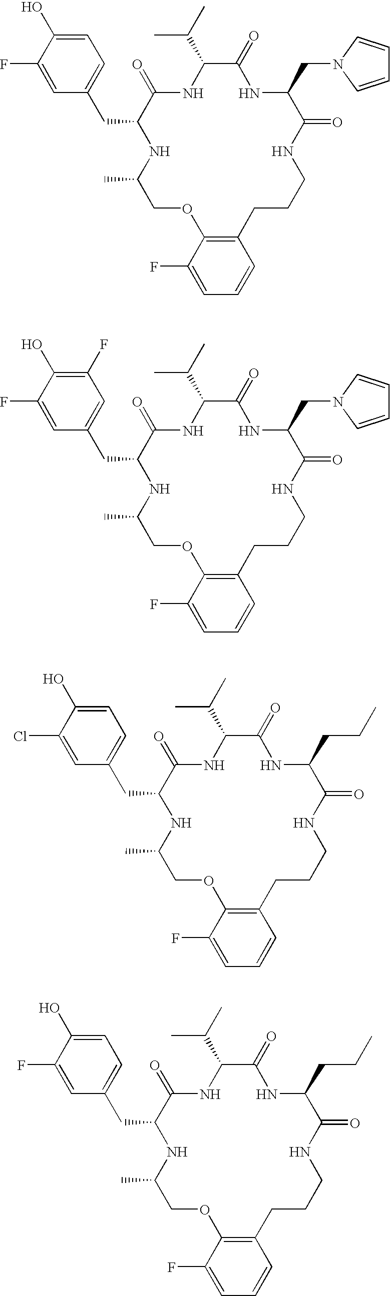

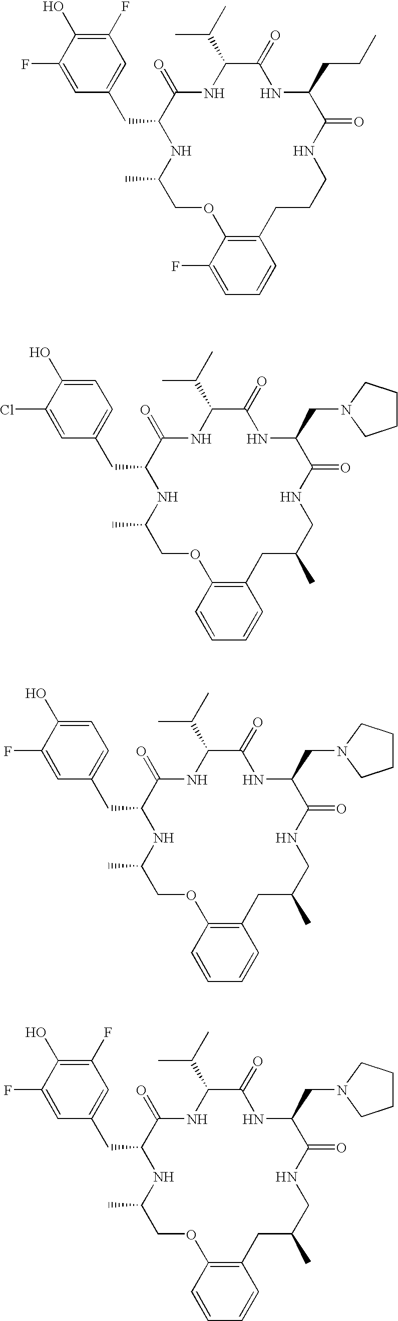

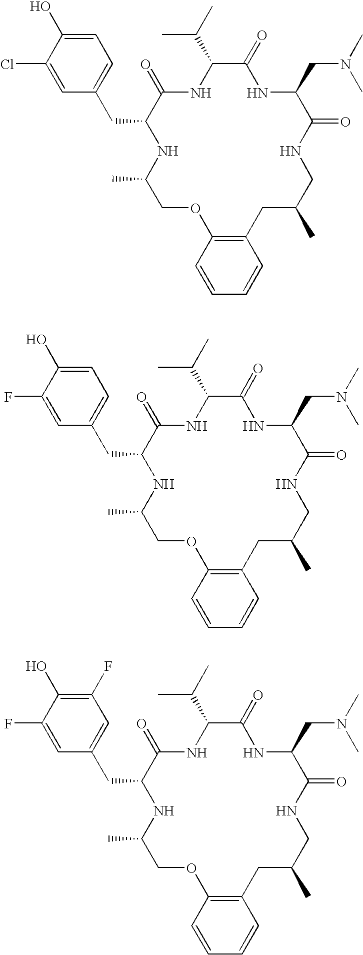

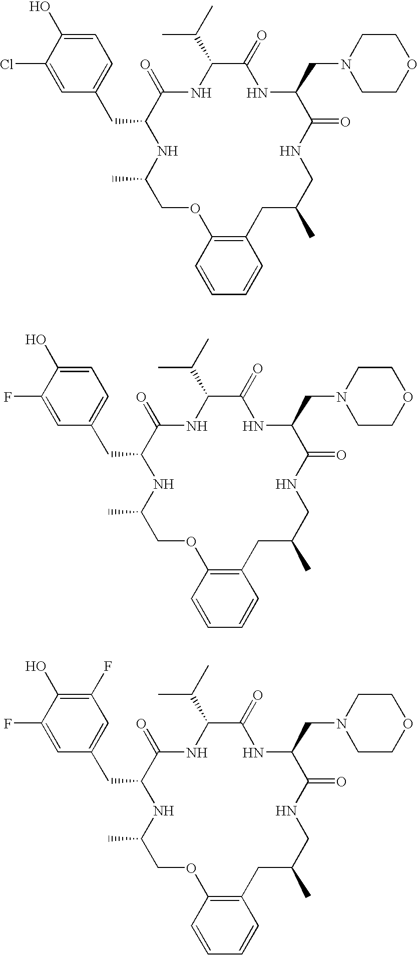

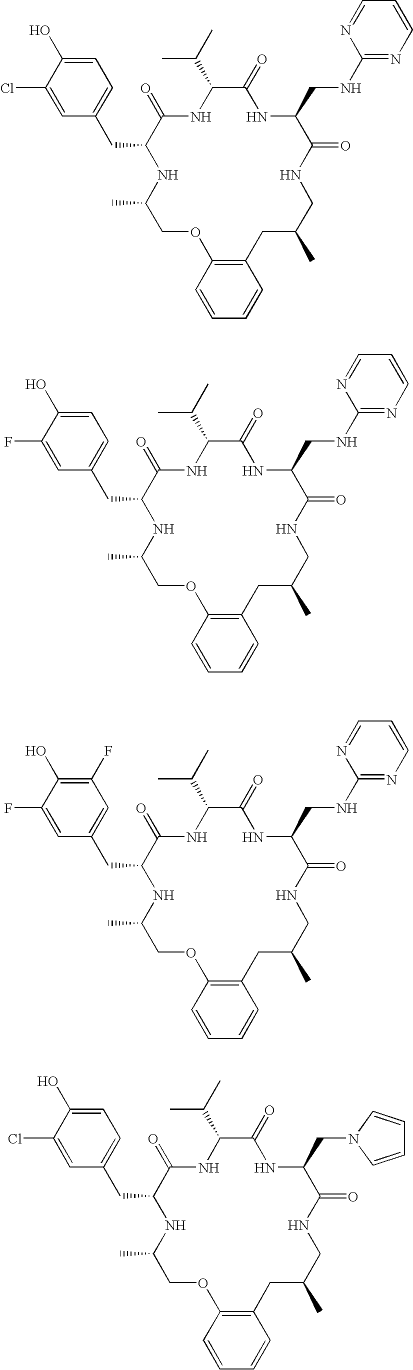

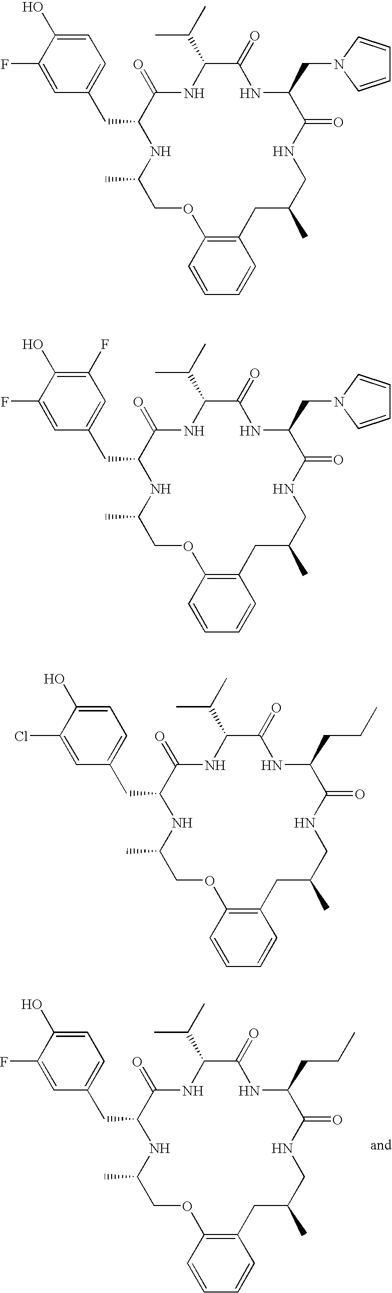

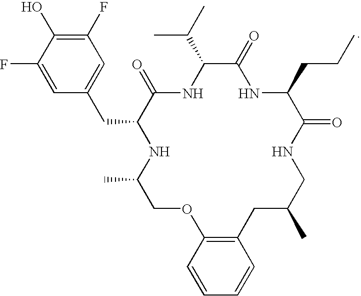

16. The method of claim 14, wherein the disorder is short bowel syndrome, celiac disease or malabsorption syndrome.

17. The method of claim 14, wherein the disorder is cachexia.

18. The method of claim 17, wherein said cachexia is cancer-related cachexia, AIDS-related cachexia, cardiac cachexia, age-related cachexia, or cachexia caused by renal or other disease.

19. The method of claim 14, wherein the subject is a mammal.

20. The method of claim 14, wherein the subject is a human.

21. The method of claim 14, wherein the subject is treated with an additional compound that modulates stomach or intestinal absorption.
